(19) United States
(12) Patent Application Publication
(10) Pub. No.: US 2024/0209010 A1
(43) Pub. Date: Jun. 27, 2024

(54) PRODRUGS OF AZO COMPOUNDS AND THEIR SALTS

(71) Applicant: KASHIV BIOSCIENCES, LLC, Piscataway, NJ (US)

(72) Inventors: Parva Yogeshchandra PUROHIT, Ahmedabad (IN); Ganesh Vishwanath SANGLE, Dist - Buldhana (IN); Vishal Bharatbhai UNADKAT, Ahmedabad (IN); Vishalgiri Gunvantgiri GOSWAMI, Ahmedabad (IN); Heta Nishil PANDYA, Ahmedabad (IN); Chirag Chimanlal MEHTA, Ahmedabad (IN)

(21) Appl. No.: 18/278,530

(22) PCT Filed: Feb. 24, 2022
(86) PCT No.: PCT/IB2022/051628

§ 371 Date: Aug. 23, 2023

(30) Foreign Application Priority Data
Feb. 24, 2021 (IN) 202121007881

(51) Int. Cl. C07F 9/58; A61K 31/4427; A61K 31/675; A61P 13/10; A61P 25/04; C07D 213/85; C07D 401/12; C07D 401/14; C07D 471/10; C07D 487/10

(52) U.S. Cl. CPC C07F 9/58; A61K 31/4427; A61K 31/675; A61P 13/10; A61P 25/04; C07D 213/85; C07D 401/12; C07D 401/14; C07D 471/10; C07D 487/10

(57) ABSTRACT

The present invention relates to compounds of formula I and therapeutic use of the same. The present invention also discloses pharmaceutical composition of compounds of formula I and method of treatment using the same.

### FIELD OF THE INVENTION

The present invention relates to compounds of general formula I and therapeutic use of the same. The present disclosure also relates to pharmaceutical composition of compounds of general formula I and method of treatment using the same.

### BACKGROUND OF THE INVENTION

Pain is a complex multidimensional concept that facilitates the initiation of the signaling cascade in response to any noxious stimuli. Numerous types of receptors are activated in pain sensations which vary in their signaling pathway. These signaling pathways can be regarded as a site for modulation of pain by targeting the pain transduction molecules to produce analgesia out of several pain syndromes.

Interstitial Cystitis/Bladder Pain Syndrome (IC/BPS), over active bladder (OAB) often has comorbidities of other chronic pelvic pain conditions, such as IBS and endometriosis, as well as more systemic pain conditions. Peripheral inflammation produces multiple inflammatory mediators, such as bradykinin, prostaglandins (PGE2), purines (ATP), proteases and NGF that act on their cognate receptors expressed in nociceptive sensory neurons to activate intracellular signal transduction pathways.

Therefore, the present invention provides the compounds useful in treatment of interstitial cystitis/bladder pain syndrome (IC/BPS), over active bladder (OAB), chronic cough, endometriosis, neuropathic pain, migraine, pruritus, postherpetic neuralgia, urinary tract infection-related pain, osteoarthritis, arthritis and urinary incontinence.

### DESCRIPTION OF FIGURES

FIG. 1—Gene expression analysis was done by quantitative RT-PCR using RNA isolated (subsequent cDNA synthesis) from RT4 cells treated with test drug compounds in combination with cyclophosphamide. Bar graph represents fold change in gene expression of P2RX3. All the values are represented as mean±SEM.

FIG. 2—Effect of compound XI on CFA induced mechanical allodynia in male SD rats using the up-and-down method % MPE; Day 2 Post CFA injection. Data is represented as % MPE (Mean±SEM), ****p<0.0001 as compared with CFA control group in Two way ANOVA followed by Bonferroni multiple comparisons test, n=08.

FIG. 3. Effect of compound XI on average paw withdrawal threshold in mechanical allodynia. Data is represented as Mean±SEM, n=8, One-way ANOVA followed by Dunnett's multiple comparison test *P<0.05, ***P<0.0001 Vs Vehicle control.

FIG. 4: Effect of Compound No. 449 and Compound No. 54 against acute cyclophosphamide (CYP) induced cystitis model. Data is presented as AUC % Pain Score (Mean±SEM). ^{###}p<0.0001 vs NC and *P<0.05, **P<0.001 vs CYP group in One-way ANOVA test followed by Dunnett's test, n=9.

FIG. 5: Effect of Compound XI against Chronic cyclophosphamide (CYP) induced cystitis model. Data is presented as AUC % Pain Score (Mean±SEM). ^{###}p<0.0001 vs NC and **P<0.001, ***P<0.0001 vs CYP group in One-way ANOVA test followed by Dunnett's test, n=8.

FIG. 6: Effect of Compound No. 43 against acute cyclophosphamide (CYP) induced cystitis model. Data is presented as AUC % Pain Score (Mean±SE). ^{###}p<0.001 vs NC and *p<0.05, **p<0.01 vs CYP group in Two-Way RM ANOVA followed by Bonferroni test, n=9.

FIG. 7: Effect of Compound XIII against acute cyclophosphamide (CYP) induced cystitis model. Data is presented as AUC % Pain Score (Mean±SEM). ^{###}p<0.001 vs NC and *P<0.05, **P<0.01, ***p<0.001 vs CYP group in Two-way ANOVA test followed by Bonferroni test, n=8.

FIG. 8: Mean±SD concentration—time profile of Compound No. 12 and Compound XI following single oral administration of Compound No. 12 in Sprague Dawley rat.

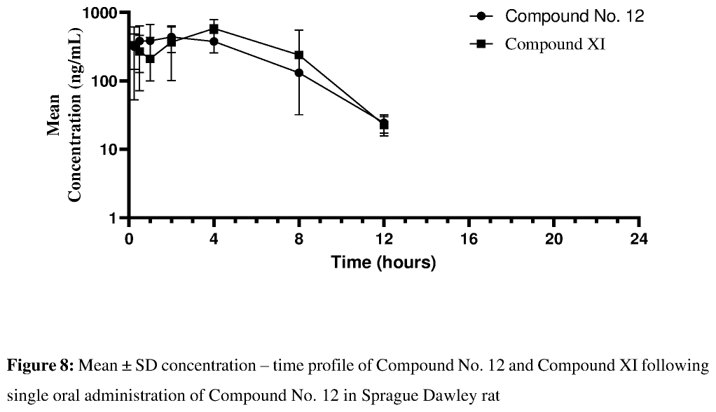

FIG. 9: Mean±SD concentration—time profile of Compound No. 43 and Compound XI following single oral administration of Compound No. 43 in Sprague Dawley rat.

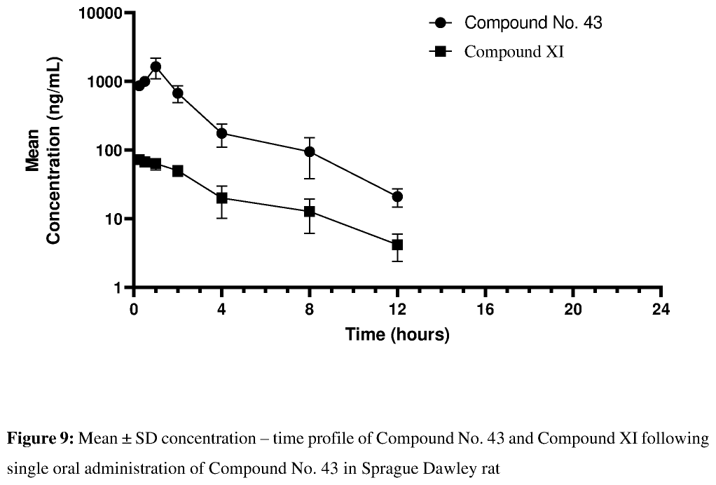

FIG. 10: Mean±SD concentration—time profile of Compound No. 446 and Compound XI following single oral administration of Compound No. 446 in Sprague Dawley rat.

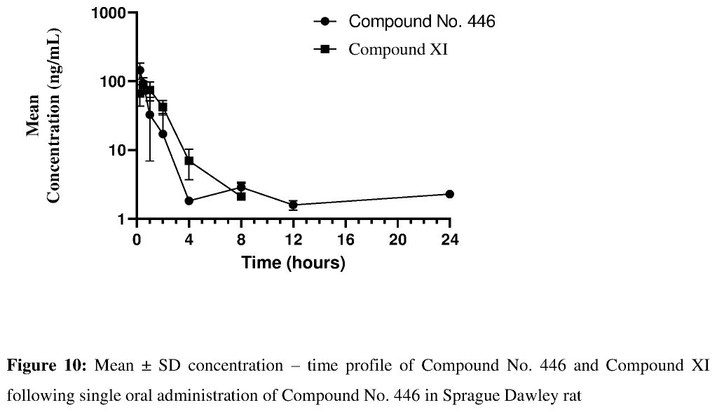

FIG. 11: Mean±SD concentration—time profile of Compound No. 449 and Compound XI following single oral administration of Compound No. 449 in Sprague Dawley rat.

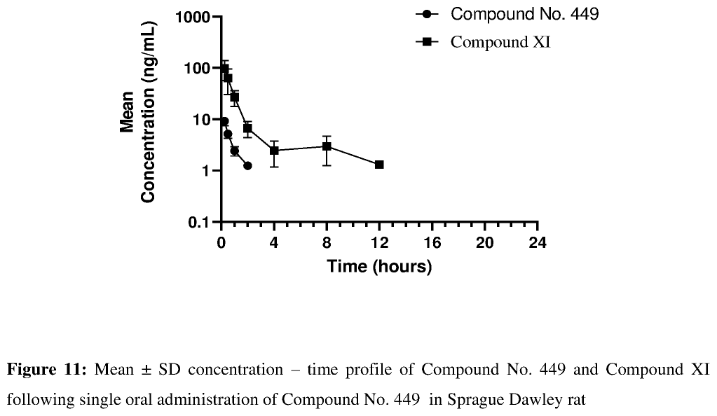

FIG. 12: Mean±SD concentration—time profile of Compound XIII and Compound XI following single oral administration of Compound XIII in Sprague Dawley rat.

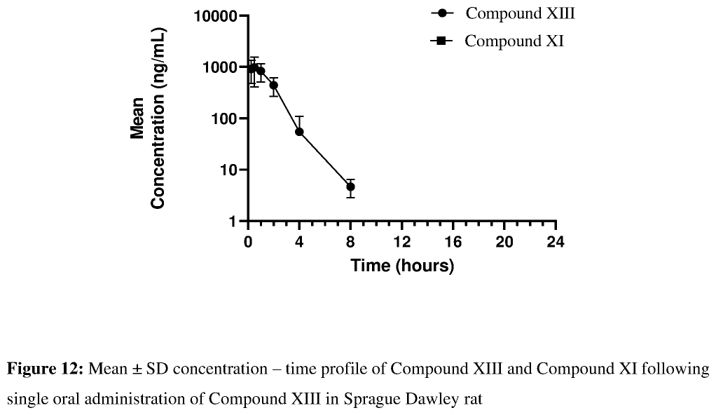

FIG. 13: Mean±SD concentration—time profile of Compound No. 54 and Compound XI following single oral administration of Compound No. 54 in Sprague Dawley rat.

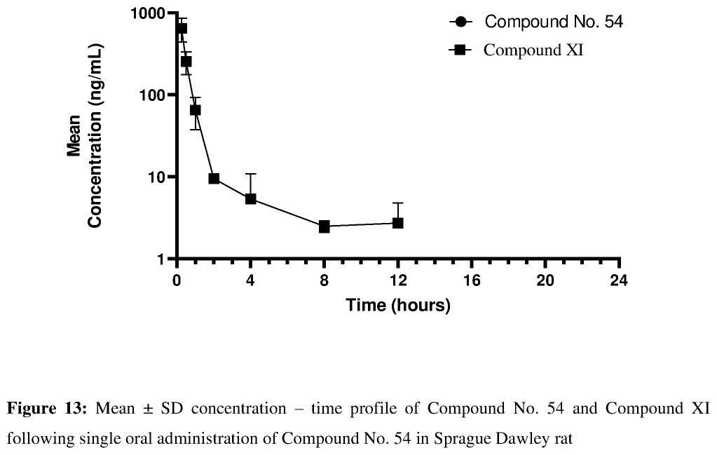

FIG. 14: Effect of Compound XI against acute cyclophosphamide (CYP) induced cystitis model. Data is presented as bladder pressure (mm hg) (Mean±SE). *p<0.05, **p<0.01 vs CYP group in One-Way ANOVA followed by Dunnett's multiple comparison test, n=10.

### SUMMARY OF THE INVENTION

The present invention provides valuable prodrugs which overcome the disadvantage of the active substances available, namely inadequate absorption of the active substance by biological membranes or the unfavorable metabolism of parent compound. Furthermore, these novel prodrugs have improved pharmacokinetic characteristics compared with parent compounds.

In one embodiment, the present invention provides compounds of general formula (I), pharmaceutically acceptable salts or solvates thereof:

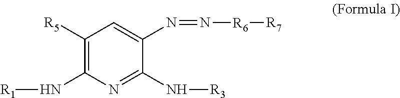

wherein;
R₁ and R₃ are the same or different and independently selected from hydrogen.

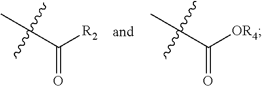

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20;
R₅ is selected from hydrogen, —OH, —OR₈, and

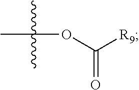

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ—, or NR₁₀R₁₁, where n is from 1-20, R₁₀ and R₁₁ are the same or different and independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₆ is a phenylene group; wherein the phenylene group is zero to four times substituted by R₇, wherein R₇ is selected from hydrogen, —OH, —OR₁₂,

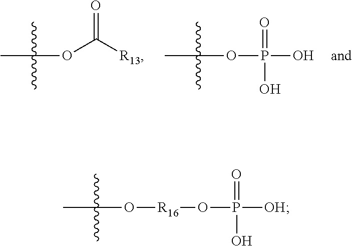

wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group; with the proviso that all substitutions at R₁, R₃, R₅, R₇ are not hydrogen when R₆ is phenylene; when anyone substitution at R1 or R3 is other than hydrogen and R6 is phenylene, then R5 and R7 is not hydrogen; when substitution at R1, R3, R7 is hydrogen then R5 is not —OH; when substitution at R1, R3, R5 is hydrogen and R6 is phenylene then single R7 substitution on phenylene is not —OH or —O-(alkyl₁₋₃) at either ortho or para position; when substitution at R1, R3 is hydrogen, R5 is —OH and R6 is phenylene then R7 substitution on phenylene is not —OH para position.

In one embodiment, the present invention provides compounds of general formula I-A, pharmaceutically acceptable salts or solvates thereof:

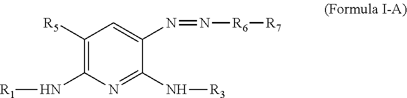

wherein; R₁ and R₃ are the same or different and independently selected from hydrogen,

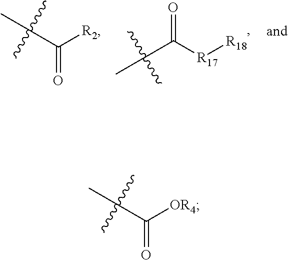

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl, where n is from 1-20; R₁₇ is selected from optionally substituted linear or branched alkylene group, wherein alkylene is optionally substituted with amino, alkylamino and dialkylamino group; Rig is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₅ is selected from hydrogen, —OH, —OR₈, and

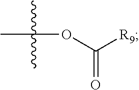

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ—, or NR₁₀R₁₁, where n is from 1-20; R₁₀ and R₁₁ are the same or different and independently selected from hydrogen or optionally substituted alkyl, cycloalkyl, aryl, heterocycloalkyl, and heteroaryl group; R₈ is selected from the group consisting of optionally substituted alkyl, alkenyl, alkynyl, cycloalkyl, heterocycloalkyl, aryl, or heteroaryl;
R₆ is a phenylene group, wherein the phenylene group is zero to four times substituted by R₇, wherein R₇ is selected from hydrogen, —OH, —OR₁₂,

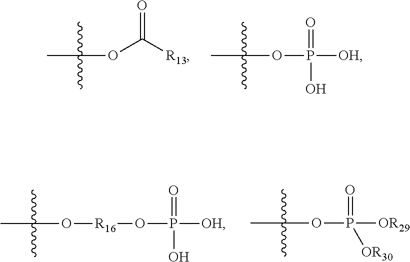

and—R₁₉; wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20; R₁₄ and R₁₅ are each independently selected from hydrogen, optionally substituted alkyl, heterocycloalkyl, aryl group; Rib selected from optionally substituted alkylene_{(C1-6)} group; R₁₉ is Selected from —O—R₂₃—O—C(O)—R₂₄, —O—C(O)—R₂₃—R₂₇, and —O—R₂₃—R₂₈; wherein R₂₃ is optionally substituted linear or branched alkylene; R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group;
R₂₉ and R₃₀ are each independently selected from hydrogen, and —R₂₃—O—C(O)—O—R₃₁; wherein R₃₁ is selected from optionally substituted alkyl, cycloalkyl, heterocycloalkyl, aryl, and heteroaryl group; with the proviso that all substitutions at R1, R3, R5, R7 are not hydrogen when R6 is phenylene; when anyone substitution at R1 or R3 is other than hydrogen and R6 is phenylene, then R5 and R7 is not hydrogen; when substitution at R1, R3, R7 is hydrogen then R5 is not —OH; when substitution at R1, R3, R5 is hydrogen and R6 is phenylene then single R7 substitution on phenylene is not —OH or —O-(alkyl₁₋₃) at either ortho or para position; when substitution at R1, R3 is hydrogen, R5 is —OH and R6 is phenylene then R7 substitution on phenylene is not —OH para position.

In another aspect, the present invention provides a method for the treatment of interstitial cystitis/bladder pain syndrome (IC/BPS), over active bladder (OAB), chronic cough, endometriosis, neuropathic pain, migraine, pruritus, postherpetic neuralgia, urinary tract infection-related pain, osteoarthritis, arthritis and urinary incontinence which comprises administering a compound selected from general formula (I) pharmaceutically acceptable salts or solvates thereof.

In another aspect, the present invention provides a method for the treatment of interstitial cystitis/bladder pain syndrome (IC/BPS), over active bladder (OAB), chronic cough, endometriosis, neuropathic pain, migraine, pruritus, postherpetic neuralgia, urinary tract infection-related pain, osteoarthritis, arthritis and urinary incontinence which comprises administering a compound selected from general formula (I-A) pharmaceutically acceptable salts or solvates thereof.

In another aspect, the present invention provides a method of use for interstitial cystitis/bladder pain syndrome (IC/BPS), over active bladder (OAB), chronic cough, endometriosis, neuropathic pain, migraine, pruritus, postherpetic neuralgia, urinary tract infection-related pain, osteoarthritis, arthritis and urinary incontinence which comprises administering a compound selected from general formula (I) pharmaceutically acceptable salts, or solvates thereof.

In another aspect, the present invention provides a method of use for interstitial cystitis/bladder pain syndrome (IC/BPS), over active bladder (OAB), chronic cough, endometriosis, neuropathic pain, migraine, pruritus, postherpetic neuralgia, urinary tract infection-related pain, osteoarthritis, arthritis and urinary incontinence which comprises administering a compound selected from general formula (I-A) pharmaceutically acceptable salts, or solvates thereof.

In another aspect, the present invention provides a method for the symptomatic relief of pain, burning, urgency, frequency, and other discomforts arising from irritation of the lower urinary tract mucosa caused by infection, trauma, surgery, endoscopic procedures, or the passage of sounds or catheters comprises administering compound selected from general formula (I) pharmaceutically acceptable salts, or solvates thereof.

In another aspect, the present invention provides a method for the symptomatic relief of pain, burning, urgency, frequency, and other discomforts arising from irritation of the lower urinary tract mucosa caused by infection, trauma, surgery, endoscopic procedures, or the passage of sounds or catheters comprises administering compound selected from general formula (I-A) pharmaceutically acceptable salts, or solvates thereof.

In one embodiment, the present invention relates to pharmaceutical composition comprising at least one compound selected from compounds of general formula (I) pharmaceutically acceptable salts, solvates thereof; and a pharmaceutically acceptable excipient.

In one embodiment, the present invention relates to pharmaceutical composition comprising at least one compound selected from compounds of general formula (I-A) pharmaceutically acceptable salts, solvates thereof; and a pharmaceutically acceptable excipient.

### DETAILED DESCRIPTION OF THE INVENTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

As used herein, the term “prodrug” refers to a precursor compound that, following administration, releases a biologically active compound in vivo via a chemical or physiological process. In an embodiment the prodrug has a desired biological activity. In another embodiment prodrug converts into therapeutic active compound which has desired biological activity.

The pharmaceutical compositions of the present disclosure can be in any form known to those of skill in the art. The pharmaceutical compositions of this invention may be administered orally, parenterally, by inhalation spray, topically, rectally, nasally, buccally or via an implanted reservoir, preferably oral administration, or administration by injection. For instance, in some embodiments the pharmaceutical compositions are in a form of a product for oral delivery, said product form being selected from a group consisting of a tablets, concentrate, dried powder, liquid, capsule, pellet, and pill. The pharmaceutical compositions disclosed herein may also further comprise carriers, binders, diluents, and excipients.

The terms “a” and “an” do not denote a limitation of quantity, but rather denote the presence of at least one of the referenced items.

The term “about” as used herein, when referring to a measurable value is meant to encompass variations of ±10%, preferably ±5%, more preferably ±1% and still more preferably ±0.1% from the specified value.

As used herein, the term “pharmaceutical composition” for the purpose of the invention, means a composition in the form of pharmaceutical preparation wherein said compositions can be administered via route selected from group consisting of oral, parenteral, nasal, topical, rectal, buccal, ophthalmic, vaginal, otic or an implanted reservoir.

As used herein, the term “pharmaceutically acceptable” means salt, carriers, excipients, and other composition ingredients that are compatible with all other pharmaceutical ingredients of a composition and are not deleterious to an individual treated with composition.

The term “pharmaceutically acceptable carrier” refers to a non-toxic carrier that may be administered to a patient, together with a compound of this invention, and which does not destroy the pharmacological activity thereof.

The term “pharmaceutically acceptable excipient” as used herein includes vehicles, adjuvants, or diluents or other auxiliary substances, such as those conventional in the art, which are readily available to the public. For example, pharmaceutically acceptable excipients include pH adjusting and buffering agents, tonicity adjusting agents, stabilizers, wetting agents and the like.

As used herein, the term “solvate” is physical form of the compound formula (I) or a pharmaceutically acceptable salt thereof and either a stoichiometric or a non-stoichiometric amount of a solvent.

As used herein, the term “salt” refers to an acid or base salt of a compound of the invention. Salts of basic compounds are salts formed with mineral acids, organic carboxylic acids, organic sulfonic acids, and the like. Examples of suitable acids include hydrochloric, hydrobromic, sulfuric, nitric, perchloric, fumaric, maleic, phosphoric, glycollic, lactic, salicylic, succinic, toluene-p-Sulfonic, tartaric, acetic, citric, methanesulfonic, formic, benzoic, malonic, naphthalene-2-sulfonic, trifluoracetic acid and benzenesulfonic acids. Salts of acidic compounds are formed with bases, namely cationic species such as alkali and alkaline earth metal cations e.g., sodium, lithium, potassium, calcium, and magnesium ions, as well as ammonium cations e.g., ammonium, trimethylammonium and diethylammonium.

As used herein, the term “alkyl” refers to a straight or branched, saturated, aliphatic radical having from 1 to about 12 carbon atoms, for example, methyl, ethyl, propyl, isopropyl, n-butyl, i-butyl, t-butyl and the like. Alkyl also refers to having 1 to about 2 carbon atom, having 1 to about 3 carbon atom, having 1 to about 4 carbon atom, having 1 to about 5 carbon atom, having 1 to about 6 carbon atom, having 1 to about 7 carbon atom, having 1 to about 8 carbon atom, having 1 to about 9 carbon atom, having 1 to about 10 carbon atom.

As used herein, the term “alkylene” refers to a straight or branched, saturated, bivalent aliphatic radical derived from alkyl group having from 1 to about 6 carbon atoms such as —CH2-R or —CH(Me)-R.

The term “alkenyl” refers to a straight chain or branched hydrocarbon having at least 2 carbon atoms and at least one carbon-carbon double bond. Alkenyl groups can have any suitable number of double bonds, including, but not limited to 1, 2, 3, 4, 5 or more. Preferable alkenyl groups include ethenyl (—CH═CH₂), 2-propenyl (allyl, —CH2-CH═CH₂) and the like.

The term “alkynyl” denotes an alkynyl groups having from 2 to 12 carbon atoms and having at least 1-2 sites of alkynyl unsaturation, alkynyl groups include ethynyl (—C≡CH), propargyl (—CH₂—C≡CH), 1-butenyl, 2-butenyl, isobutenyl, butadienyl and the like.

The term “cycloalkyl” denotes a saturated carbocyclic group of from 3 to 12 carbon atoms having a single ring (e.g., cyclohexyl) or multiple condensed rings (e.g., norbornyl). Cycloalkyl include cyclopropyl, cyclobutyl, cyclopentyl, cyclohexyl, cycloheptyl, norbornyl and the like.

The term “heterocyclic Ring” denotes a ring containing C3-C12 atoms, in which up to 4 carbon atoms are replaced by heteroatoms chosen from the group consisting of O, S or N. Heterocyclic ring further contain “heterocycloalkyl” or “heteroaryl”.

The term “heterocycloalkyl” denotes a C3-C12 cycloalkyl group according to the definition above, in which up to 4 carbon atoms are replaced by heteroatoms chosen from the group consisting of O, S or N. Heterocycloalkyl include pyrrolidine, piperidine, piperazine, morpholine, tetrahydrofuran, tetrahydrothiophenyl, 3-methyl-3,9-diazaspiro[5.5]undecane, 2-methyl-2,6-diazaspiro[3.3]heptane, dioxole and the like. The heterocycloalkyl can be monocyclic, bicyclic or spirocyclic.

The term “aryl” denotes a cyclic aromatic hydrocarbon radical consisting of one or more fused rings containing 6-14 carbon atoms in which at least one ring is aromatic in nature, for example phenyl, naphthyl, 1,2,3,4-tetrahydronaphthalenyl, indanyl and the like.

The term “heteroaryl” denotes a cyclic aromatic hydrocarbon radical consisting of one or more fused rings containing 5-14 ring atoms, preferably containing 5-10 ring atoms, in which at least one ring is aromatic in nature, and which contains at least one heteroatom, selected from N, O or S, for example pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, benzothienyl, benzotriazolyl, isobenzothienyl, indolyl, isoindolyl, benzimidazolyl, imidazo[1,2-a]pyridyl, benzothiazolyl, benzoxazolyl, quinolizinyl, quinazolinyl, benzoquinolyl and the like.

The term amino acid as used herein refers to two stereoisomeric forms, called “D” and “L.” The D and L forms of any amino acid have identical physical properties and chemical reactivities, but rotate the plane of plane-polarized light equally but in opposite directions and react at different rates with asymmetric reagents. All naturally occurring amino acids in proteins are in the L form. Amino acid comprises lysine, valine, tryptophan, phenylalanine, methionine, leucine, threonine, isoleucine, arginine, histidine, tyrosine, carnitine, serine, glutamine, aspartic acid, proline, glycine, cysteine, alanine, glutamic acid. Amino acid may be present as either “D” or “L” enantiomer.

The term “halogen” denotes chlorine, iodine, fluorine and bromine.

As used herein, the term “leaving group” or “L” or “L₁” can be defined as part of a substrate that is cleaved by the action of a nucleophile. Examples of leaving groups include, but are not limited to: halogen (F, Cl, Br, and I), tosylate, mesylate, triflate, acetate, hydroxyl, camphorsulfonate, aryloxide, and the like.

The term “amino” refers to the group —NH₂.

The term “alkylamino” refers to the group —NHalkyl.

The term “dialkylamino” refers to the group —N(alkyl)₂

The term “alkoxy” refers to the group —O-alkyl.

The term “aryloxy” refers to the group —O-aryl.

The term “cycloalkoxy” refers to the group —O-cycloalkyl.

The term “heterocycloalkyloxy” refers to the group —O-heterocycloalkyl.

The term “heteroaryloxy” refers to the group —O-heteraryl.

The term “alkyl-ester” refers to the group -alkylene-O—(CO)—R.

The term “aminoaryl” refers to the group -arylene-amino.

The term “alkylalcohol” or “hydroxyalkyl” refers to alkyl group substituted with one or more —OH group.

The term “amino acid residue” or “amino acid side chain” refer to the fragments —C(O)—C(H/R)₂—N(H/R)₂ or —N(H/R)— C(H/R)₂—C(O)(O—H/R), or N(H/R)₂—C(H/R)— C(O)(O—H/R). where “R” refers to hydrogen or any other variable value except hydrogen.

The term “optionally” means the subsequently described event or circumstance can or cannot occur, and that the description includes instances where the event or circumstance occurs and instances where it does not.

For purposes of the present invention, the term “substituted” shall be understood to include adding or replacing one or more atoms contained within a functional group or compound with one or more different atoms.

Unless otherwise constrained by the definition of the individual substituent, the above set out groups, like “alkyl”, “alkenyl”, “alkynyl”, “cycloalkyl”, “heterocyclic ring”, “heterocycloalkyl”, “substituted heterocycloalkyl”, “aryl” and “heteroaryl” etc. groups can optionally be substituted with one or more substituents selected from the group consisting of “C1-C8-alkyl”, “C2-C8-alkenyl”, “C2-C8-alkynyl”, “cycloalkyl”, “heterocycloalkyl”, “aryl”, “heteroaryl”, “amino”, “alkylamino”, “dialkylamino”, “acyl”, “aryloxy”, “acylamino”, “aminocarbonyl”, “alkoxycarbonyl”, “alkoxyalkyloxy”, “alkoxycarbonyloxy”, “carbamate,” “sulfinyl”, “sulfonyl”, “alkoxy”, “sulfanyl”, “halogen”, “carboxy”, “trihalomethyl”, “cyano”, “hydroxy”, “mercapto”, “nitro”, “phosphate”, “alkylphosphate” and the like. The preferred one or more substituents is selected from 1 to 10 in number; more preferably from 1 to 5 in number.

In one embodiment, the prodrug itself may either lack or possess the desired biological activity. In one embodiment of the present invention the compound as disclosed in the specification may possess the desired biological activity on their own.

In one embodiment, the compounds of this invention contain one or more asymmetric carbon atoms and thus occur as racemate and racemic mixtures, single enantiomers, diastereomeric mixtures and individual diastereomers. All such isomeric forms of these compounds are expressly included in the present invention. Each stereo genic carbon may be of the R or S configuration. In one embodiment, enantiomers, diastereomers, racemates of the compounds are expressly included in the present invention.

In one embodiment, the present invention provides compounds of general formula I-A, pharmaceutically acceptable salts or solvates thereof:

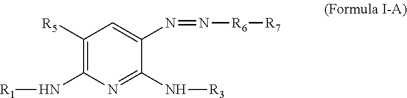

wherein; R₁ and R₃ are the same or different and independently selected from hydrogen,

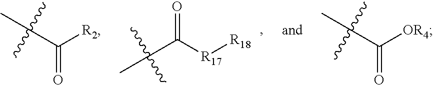

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl, where n is from 1-20; R₁₇ is selected from optionally substituted linear or branched alkylene group, wherein alkylene is optionally substituted with amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₅ is selected from hydrogen, —OH, —OR₈, and

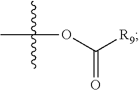

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ, or NR₁₀R₁₁, where n is from 1-20; R₁₀ and R₁₁ are the same or different and independently selected from hydrogen or optionally substituted alkyl, cycloalkyl, aryl, heterocycloalkyl, and heteroaryl group; R₈ is selected from the group consisting of optionally substituted alkyl, alkenyl, alkynyl, cycloalkyl, heterocycloalkyl, aryl, or heteroaryl;
R₆ is a phenylene group, wherein the phenylene group is zero to four times substituted by R₇. wherein R₇ is selected from hydrogen, —OH, —OR₁₂,

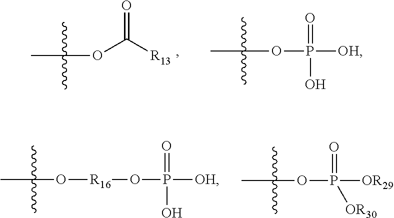

and—R₁₉; wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20; R₁₄ and R₁₅ are each independently selected from hydrogen, optionally substituted alkyl, heterocycloalkyl, aryl group; R₁₆ selected from optionally substituted alkylene_{(C1-6)} group; R₁₉ is Selected from —O—R₂₃—O—C(O)—R₂₄, —O—C(O)—R₂₃—R₂₇, and —O—R₂₃—R₂₈; wherein R₂₃ is optionally substituted linear or branched alkylene; R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group;
R₂₉ and R₃₀ are each independently selected from hydrogen, and —R₂₃—O—C(O)—O—R₃₁; wherein R₃₁ is selected from optionally substituted alkyl, cycloalkyl, heterocycloalkyl, aryl, and heteroaryl group; with the proviso that all substitutions at R1, R3, R5, R7 are not hydrogen when R6 is phenylene; when anyone substitution at R1 or R3 is other than hydrogen and R6 is phenylene, then R5 and R7 is not hydrogen; when substitution at R1, R3, R7 is hydrogen then R5 is not —OH; when substitution at R1, R3, R5 is hydrogen and R6 is phenylene then single R7 substitution on phenylene is not —OH or —O-(alkyl₁₋₃) at either ortho or para position; when substitution at R1, R3 is hydrogen, R5 is —OH and R6 is phenylene then R7 substitution on phenylene is not —OH para position.

In one embodiment, the present invention provides compounds of general formula I-A, pharmaceutically acceptable salts or solvates thereof; wherein R₄ is alkyl which is substituted with the group selected from amino, alkylamino, dialkylamino or optionally substituted heterocycloalkyl or R₄ is aryl which is substituted with optionally substituted amino acid side chain.

In one embodiment, the present invention provides compounds of general formula I-A, pharmaceutically acceptable salts or solvates thereof; wherein R₉ is alkyl which is substituted with alkylester, amino, and optionally substituted heterocycloalkyl, amino, and optionally substituted heterocycloalkyl; or R₉ is heterocycloalkyl which is substituted with the group selected from alkyl, amino, alkylamino, dialkylamino, aryl, or optionally substituted heterocyclic ring wherein the optionally substituted heterocyclic ring is substituted by alkyl; or R₉ is aryloxy is optionally substituted with amino acid residue or amino acid side chain.

In one embodiment, the present invention provides compounds of general formula I-A, pharmaceutically acceptable salts or solvates thereof; wherein R₁₀ and R₁₁ are independently selected as alkyl which is substituted with the group selected from amino, alkylamino, dialkylamino and optionally substituted heterocycloalkyl.

In one embodiment, the present invention provides compounds of general formula I-A, pharmaceutically acceptable salts or solvates thereof; wherein R₁₃ is alkyl which is substituted with the group selected from hydroxy, aryl, amino, alkylamino, dialkylamino, alkylalcohol, heterocycloalkyl or substituted heterocycloalkyl; or R₁₃ is alkoxy which is substituted with group selected from amino, alkylamino, dialkylamino, heterocycloalkyl or substituted heterocycloalkyl; or R₁₃ is heterocycloalkyl which is substituted with hydroxy, amino, carboxy, alkyl, alkylamino, dialkylamino, heterocyclic ring, substituted heterocyclic ring; wherein the substituted heterocyclic ring is substituted with alkyl; or R₁₃ is heterocycloalkloxy which is substituted with alkyl.

In one embodiment, the present invention provides compounds of general formula I-A, pharmaceutically acceptable salts or solvates thereof; wherein R₁₄ and R₁₅ are independently selected from alkyl which is substituted with the group selected from carboxy, amino, alkylamino, dialkylamino, heterocycloalkyl, or substituted heterocycloalkyl group; or R₁₄ and R₁₅ are independently selected from heterocycloalkyl which is substituted by alkyl, heterocyclic ring or substituted heterocyclic ring, wherein the substituted heterocyclic ring is substituted with alkyl.

In one embodiment, the present invention provides compounds of general formula I-A, pharmaceutically acceptable salts or solvates thereof; wherein R₁₄ and R₁₅ are independently selected from aryl which is substituted with the group selected from heterocyclic or substituted heterocycloalkyl group; wherein the substituted heterocyclic ring is substituted with alkyl.

In one embodiment, the present invention provides compounds of general formula I, pharmaceutically acceptable salts or solvates thereof:

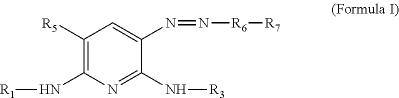

wherein;
R₁ and R₃ are the same or different and independently selected from hydrogen.

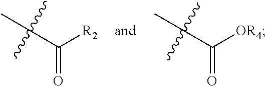

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20;
R₅ is selected from hydrogen, —OH, —OR₈, and

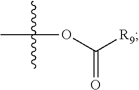

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ—, or NR₁₀R₁₁, where n is from 1-20, R₁₀ and R₁₁ are the same or different and independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₆ is a phenylene group; wherein the phenylene group is zero to four times substituted by R₇, wherein R₇ is selected from hydrogen, —OH, —OR₁₂,

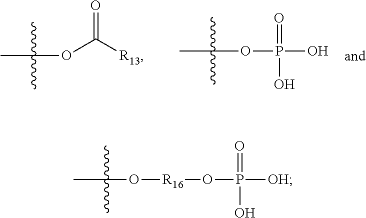

wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20;
where R₁₄ and R₁₅ are each independently selected from hydrogen, optionally substituted alkyl, heterocycloalkyl, aryl group; R₁₆ is selected from optionally substituted alkylene(C₁₋₆) group; with the proviso that all substitutions at R₁, R₃, R₅, R₇ are not hydrogen when R₆ is phenylene; when anyone substitution at R1 or R3 is other than hydrogen and R6 is phenylene, then R5 and R7 is not hydrogen; when substitution at R1, R3, R7 is hydrogen then R5 is not —OH; when substitution at R1, R3, R5 is hydrogen and R6 is phenylene then single R7 substitution on phenylene is not —OH or —O-(alkyl₁₋₃) at either ortho or para position; when substitution at R1, R3 is hydrogen, R5 is —OH and R6 is phenylene then R7 substitution on phenylene is not —OH para position.

In one embodiment, the present invention provides compounds of general formula I, pharmaceutically acceptable salts or solvates thereof; wherein R₄ is alkyl which is substituted with the group selected from amino, alkylamino, dialkylamino or optionally substituted heterocycloalkyl or R₄ is aryl which is substituted with optionally substituted amino acid side chain.

In one embodiment, the present invention provides compounds of general formula I, pharmaceutically acceptable salts or solvates thereof; wherein R₉ is alkyl which is substituted with alkylester, amino, and optionally substituted heterocycloalkyl, amino, and optionally substituted heterocycloalkyl; or R₉ is heterocycloalkyl which is substituted with the group selected from alkyl, amino, alkylamino, dialkylamino, aryl, or optionally substituted heterocyclic ring wherein the optionally substituted heterocyclic ring is substituted by alkyl; or R₉ is aryloxy is optionally substituted with amino acid residue or amino acid side chain.

In one embodiment, the present invention provides compounds of general formula I, pharmaceutically acceptable salts or solvates thereof; wherein R₁₀ and R₁₁ are independently selected as alkyl which is substituted with the group selected from amino, alkylamino, dialkylamino and optionally substituted heterocycloalkyl.

In one embodiment, the present invention provides compounds of general formula I, pharmaceutically acceptable salts or solvates thereof; wherein R₁₃ is alkyl which is substituted with the group selected from hydroxy, aryl, amino, alkylamino, dialkylamino, alkylalcohol, heterocycloalkyl or substituted heterocycloalkyl; or Rn is alkoxy which is substituted with group selected from amino, alkylamino, dialkylamino, heterocycloalkyl or substituted heterocycloalkyl; or R₁₃ is heterocycloalkyl which is substituted with hydroxy, amino, carboxy, alkyl, alkylamino, dialkylamino, heterocyclic ring, substituted heterocyclic ring; wherein the substituted heterocyclic ring is substituted with alkyl; or R₁₃ is heterocycloalkloxy which is substituted with alkyl.

In one embodiment, the present invention provides compounds of general formula I, pharmaceutically acceptable salts or solvates thereof; wherein R₁₄ and R₁₅ are independently selected from alkyl which is substituted with the group selected from carboxy, amino, alkylamino, dialkylamino, heterocycloalkyl, or substituted heterocycloalkyl group; or R₁₄ and R₁₅ are independently selected from heterocycloalkyl which is substituted by alkyl, heterocyclic ring or substituted heterocyclic ring, wherein the substituted heterocyclic ring is substituted with alkyl.

In one embodiment, the present invention provides compounds of general formula I, pharmaceutically acceptable salts or solvates thereof; wherein R₁₄ and R₁₅ are independently selected from aryl which is substituted with the group selected from heterocyclic or substituted heterocycloalkyl group; wherein the substituted heterocyclic ring is substituted with alkyl.

In one embodiment, the present invention relates compounds of general formula (II), pharmaceutically acceptable salts or solvates thereof:
wherein;

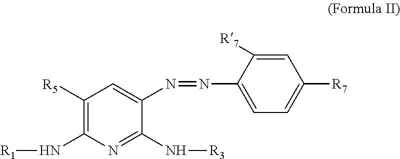

R₁ and R₃ are independently selected from hydrogen

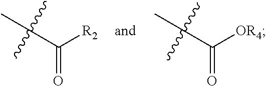

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, cycloalkyl, heterocycloalkyl, aryl, —(CH₂—CH₂—O)ₙ—, or heteroaryl; where n is from 1-20;
R5 is selected from hydrogen, OH, —OR₈, and

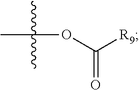

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁, where n is from 1-20; wherein R₁₀ and R₁₁ are the same or different and independently selected from hydrogen or optionally substituted alkyl; R₈ is selected from the group consisting of optionally substituted alkyl, alkenyl, alkynyl, cycloalkyl, heterocycloalkyl, aryl, or heteroaryl; R₇ and R′₇ are the same or different and independently selected from hydrogen, OH, —OR₁₂,

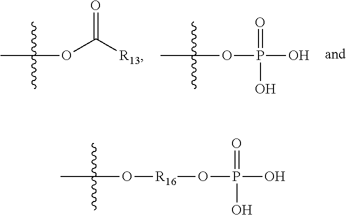

wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, heterocycloalkyloxy, aminoaryl, or amino acid linked via ester or amide linkage at the point of attachment, —(CH2-CH2-O)n-, or NR₁₄R₁₅, where n is from 1-20; wherein R₁₄ and R15 each independently selected from hydrogen, optionally substituted alkyl, heterocycloalkyl, aryl group; R16 is selected from optionally substituted alkylene_{(C1-6)} group;
with the proviso that all substitutions at R1, R3, R5, R7 and R′7 is not hydrogen at the same time; when any one substitution at R1 or R3 is not hydrogen then R5, R7 and R′7 is not hydrogen; when substitution at R1, R3, R7 and R′₇ is hydrogen then R₅ is not —OH; when substitution at R1, R3, R5 and R′7 is hydrogen then R7 is not —OH or —O-(alkyl₁₋₃); when substitution at R1, R3, R5 and R7 is hydrogen then R′7 is not —OH or —O-(alkyl₁₋₃); and when substitution at R1, R3 and R7 is hydrogen then both R5 and R′7 is not —OH.

In one embodiment, the present invention provides compounds of general formula II; pharmaceutically acceptable salts or solvates thereof; wherein R₄ is alkyl which is substituted with the group selected from amino, alkylamino, dialkylamino or optionally substituted heterocycloalkyl; wherein aryl group represented by R₄ is aryl which is substituted with optionally substituted amino acid side chain.

In one embodiment, the present invention provides compounds of general formula II; pharmaceutically acceptable salts or solvates thereof; wherein alkyl group represented by R9 is substituted by alkylester, amino, and optionally substituted heterocycloalkyl; wherein heterocycloalkyl group represented by R₉ is substituted with the group selected from alkyl, amino, alkylamino, dialkylamino, aryl, or optionally substituted heterocyclic ring; wherein the optionally substituted heterocyclic ring is substituted by alkyl, wherein the aryloxy group of R9 is optionally substituted with amino acid residue or amino acid side chain.

In one embodiment, the present invention provides compounds of general formula II; pharmaceutically acceptable salts or solvates thereof; wherein R₁₀ and R₁₁ are independently selected as alkyl which is substituted with the group selected from amino, alkylamino, dialkylamino and optionally substituted heterocycloalkyl.

In one embodiment, the present invention provides compounds of general formula II; pharmaceutically acceptable salts or solvates thereof; wherein R₁₃ is alkyl which is substituted with the group selected from hydroxy, aryl, amino, alkylamino, dialkylamino, alkylalcohol, heterocycloalkyl or substituted heterocycloalkyl; or R₁₃ is alkoxy which is substituted with group selected from amino, alkylamino, dialkylamino, heterocycloalkyl or substituted heterocycloalkyl; or R₁₃ is heterocycloalkyl which is substituted with hydroxy, amino, carboxy, alkyl, alkylamino, dialkylamino, heterocyclic ring, substituted heterocyclic ring; wherein the substituted heterocyclic ring is substituted with alkyl; or R₁₃ is heterocycloalkloxy which is substituted with alkyl.

In one embodiment, the present invention provides compounds of general formula II; pharmaceutically acceptable salts or solvates thereof;

wherein R₁₄ and R₁₅ are independently selected from alkyl which is substituted with the group selected from carboxy, amino, alkylamino, dialkylamino, heterocycloalkyl, or substituted heterocycloalkyl group; or R₁₄ and R₁₅ are independently selected from heterocycloalkyl which is substituted by alkyl, heterocyclic ring or substituted heterocyclic ring, wherein the substituted heterocyclic ring is substituted with alkyl.

In one embodiment, the present invention provides compounds of general formula II; pharmaceutically acceptable salts or solvates thereof;

wherein R₁₄ and R₁₅ are independently selected from aryl which is substituted with the group selected from heterocyclic or substituted heterocycloalkyl group; wherein the substituted heterocyclic ring is substituted with alkyl.

In one aspect, the present invention relates compounds of general formula (III), pharmaceutically acceptable salts or solvates thereof:

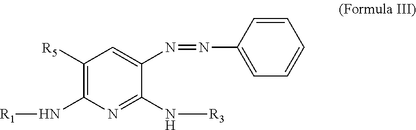

R₁ selected from hydrogen,

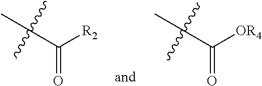

and R₃ is selected from

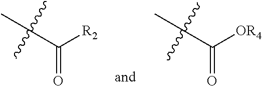

wherein R₂ is selected from the group consisting of optionally substituted alkyl alkenyl, alkynyl, cycloalkyl, heterocycloalkyl, or heteroaryl; R₄ is selected from the group consisting of optionally substituted alkyl, alkenyl, alkynyl, —(CH2-CH2-O)n-, cycloalkyl, heterocycloalkyl, or heteroaryl; where n is from 1-20;
R₅ selected from —OR₈, and

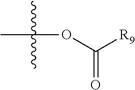

wherein R₉ is selected from the group consisting of optionally substituted alkyl, alkenyl, alkynyl, cycloalkyl, heterocycloalkyl, aryl, heteroaryl, alkyl-ester, alkoxy, heterocycloalkoxy, heteroaryloxy, cycloalkoxy, aryloxy, amino acid linked via ester or amide linkage at the point of attachment, —(CH2-CH2-O)n-, or N₁₀R₁₁, wherein R₁₀ repand R₁₁ are the same or different and are independently selected from hydrogen, optionally substituted alkyl; where n is from 1-20; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl alkenyl, alkynyl, cycloalkyl, heterocycloalkyl, aryl, or heteroaryl.

In one embodiment, the present invention provides compounds of general formula III; pharmaceutically acceptable salts or solvates thereof;

wherein R₄ is alkyl which is substituted with the group selected from amino, alkylamino, dialkylamino or optionally substituted heterocycloalkyl or R₄ is aryl which is substituted with optionally substituted amino acid side chain.

In one embodiment, the present invention provides compounds of general formula III; pharmaceutically acceptable salts or solvates thereof;

wherein R₉ is alkyl which is substituted with alkylester, amino, and optionally substituted heterocycloalkyl, amino, and optionally substituted heterocycloalkyl; or R₉ is heterocycloalkyl which is substituted with the group selected from alkyl, amino, alkylamino, dialkylamino, aryl, or optionally substituted heterocyclic ring wherein the optionally substituted heterocyclic ring is substituted by alkyl; or R₉ is aryloxy is optionally substituted with amino acid residue or amino acid side chain.

In one embodiment, the present invention provides compounds of general formula III; pharmaceutically acceptable salts or solvates thereof;

wherein R₁₀ and R₁₁ are independently selected as alkyl which is substituted with the group selected from amino, alkylamino, dialkylamino and optionally substituted heterocycloalkyl.

In one embodiment the present invention provides the compound selected from the group consisting of:

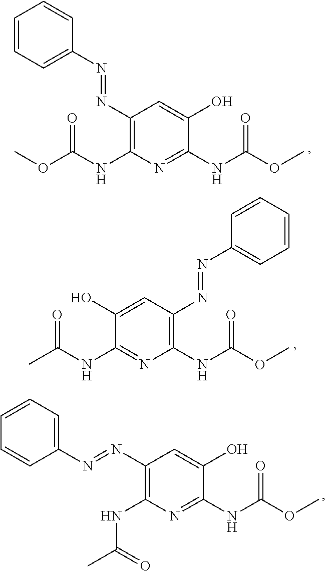

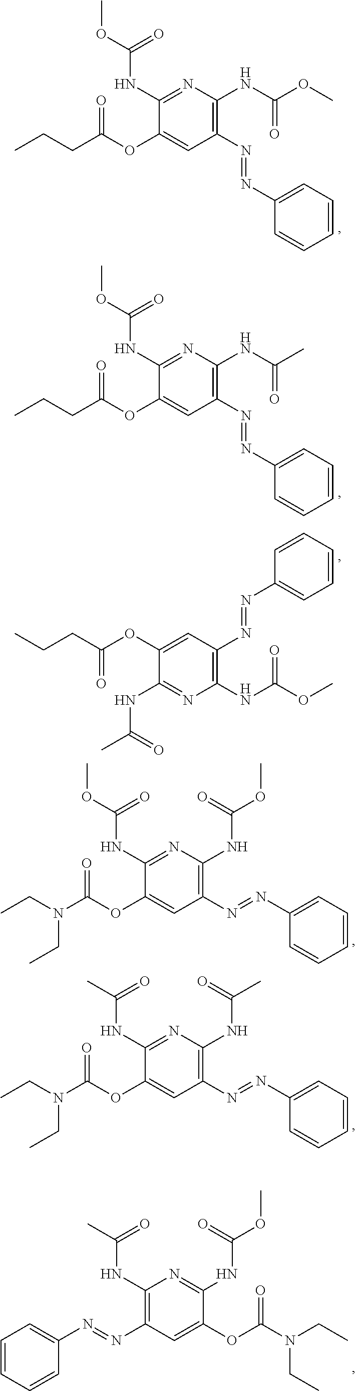

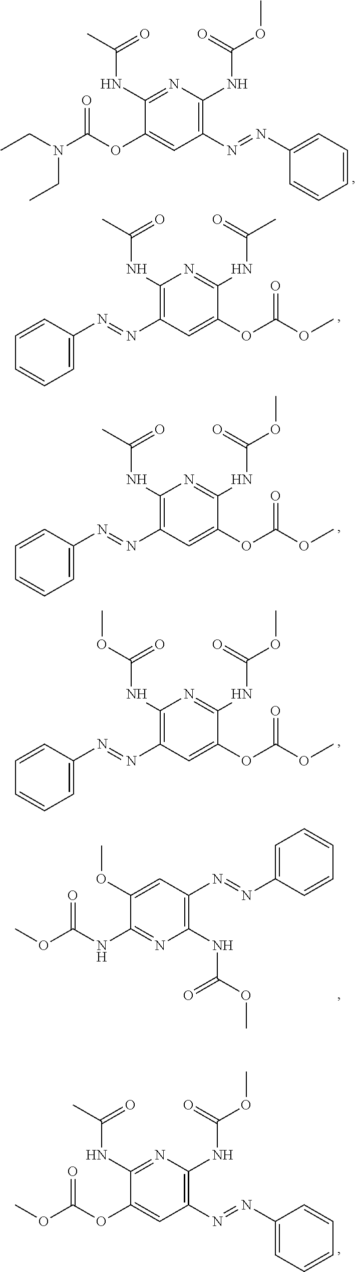

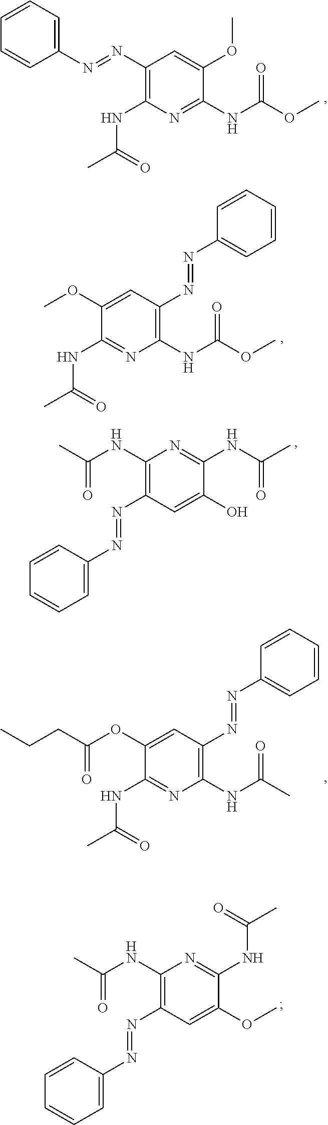

pharmaceutically acceptable salts or solvates thereof.

In another aspect, compounds of general formula (III-A), pharmaceutically acceptable salts or solvates thereof:

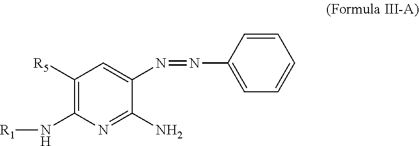

wherein;
R₁ selected from

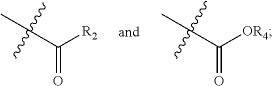

wherein R₂ is selected from the group consisting of optionally substituted alkyl, alkenyl, alkynyl, cycloalkyl, heterocycloalkyl, or heteroaryl and; R₄ is selected from the group consisting of optionally substituted alkyl, alkenyl, alkynyl, —(CH2-CH2-O)n-, cycloalkyl, heterocycloalkyl, or heteroaryl; where n is from 1-20;
R₅ selected from —OR₈, and

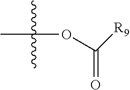

wherein R₉ is selected from the group consisting of optionally substituted alkyl, alkenyl, alkynyl, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment, —(CH2-CH2-O)n-, or N₁₀R₁₁, where n is from 1-20; wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl, alkenyl, alkynyl, cycloalkyl, heterocycloalkyl, aryl, or heteroaryl.

In one embodiment the present invention provides the compound selected from the group consisting of:

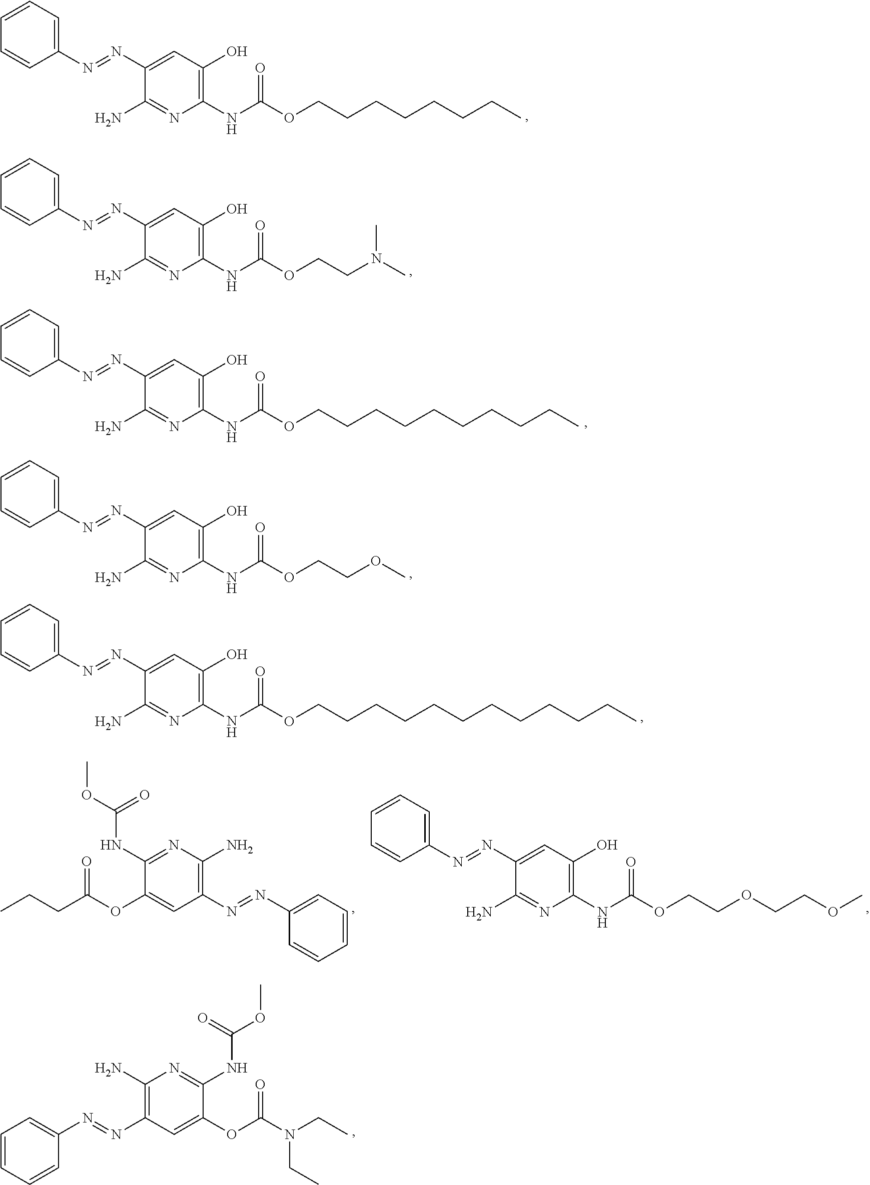

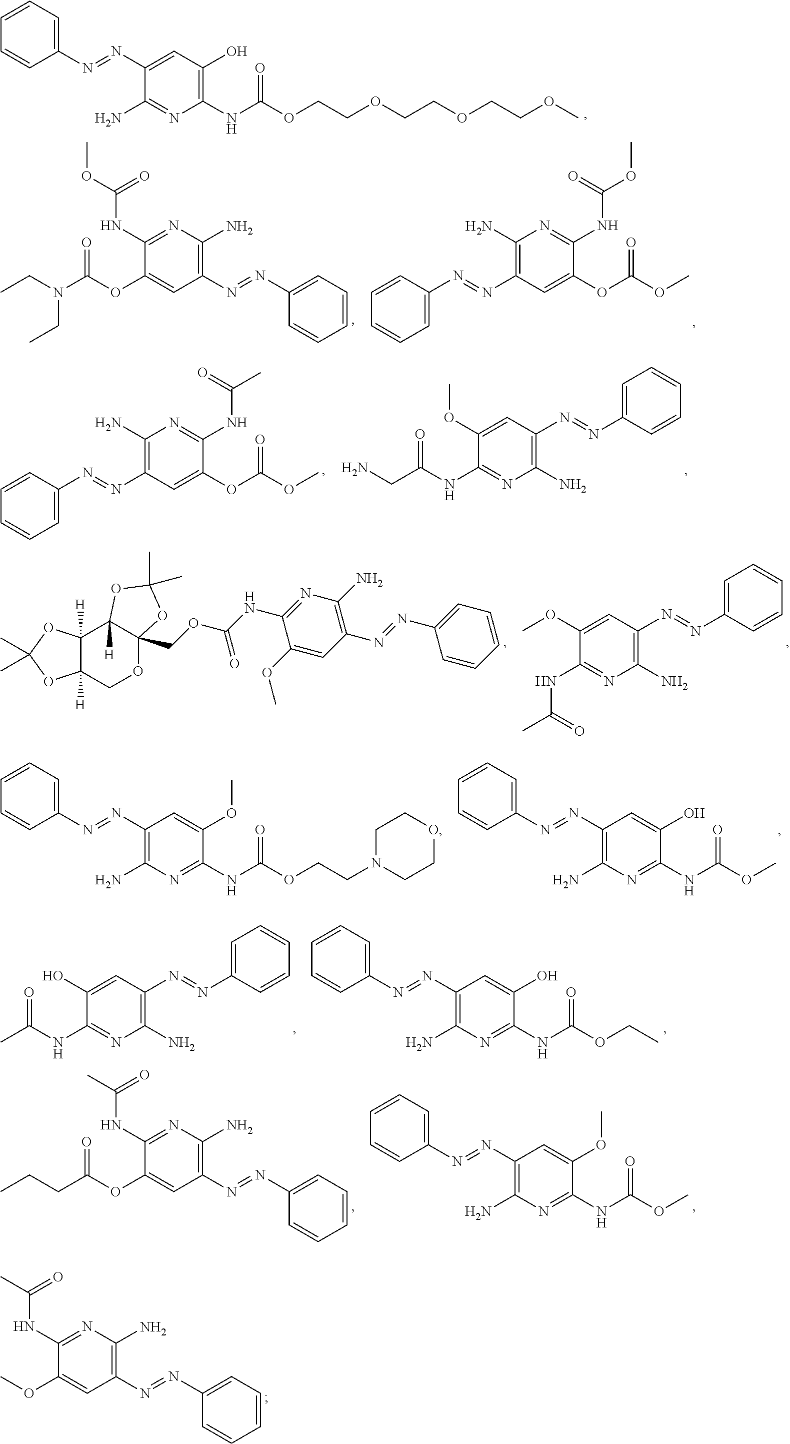

pharmaceutically acceptable salts or solvates thereof.

In another aspect, compounds of general formula (III-B), pharmaceutically acceptable salts or solvates thereof:

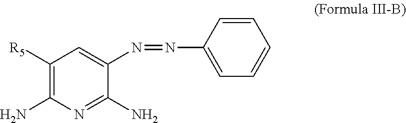

wherein; R₅ is selected from —OR₈, and

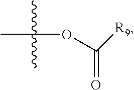

wherein R₉ is selected from the group consisting of optionally substituted alkyl, alkenyl, alkynyl, —(CH2-CH2-O)n-, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment or N₁₀R₁₁; where n is from 1-20, wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of optionally substituted alkyl, alkenyl, alkynyl, cycloalkyl, heterocycloalkyl, aryl, or heteroaryl.

In one embodiment the present invention provides the compound selected from the group consisting of:

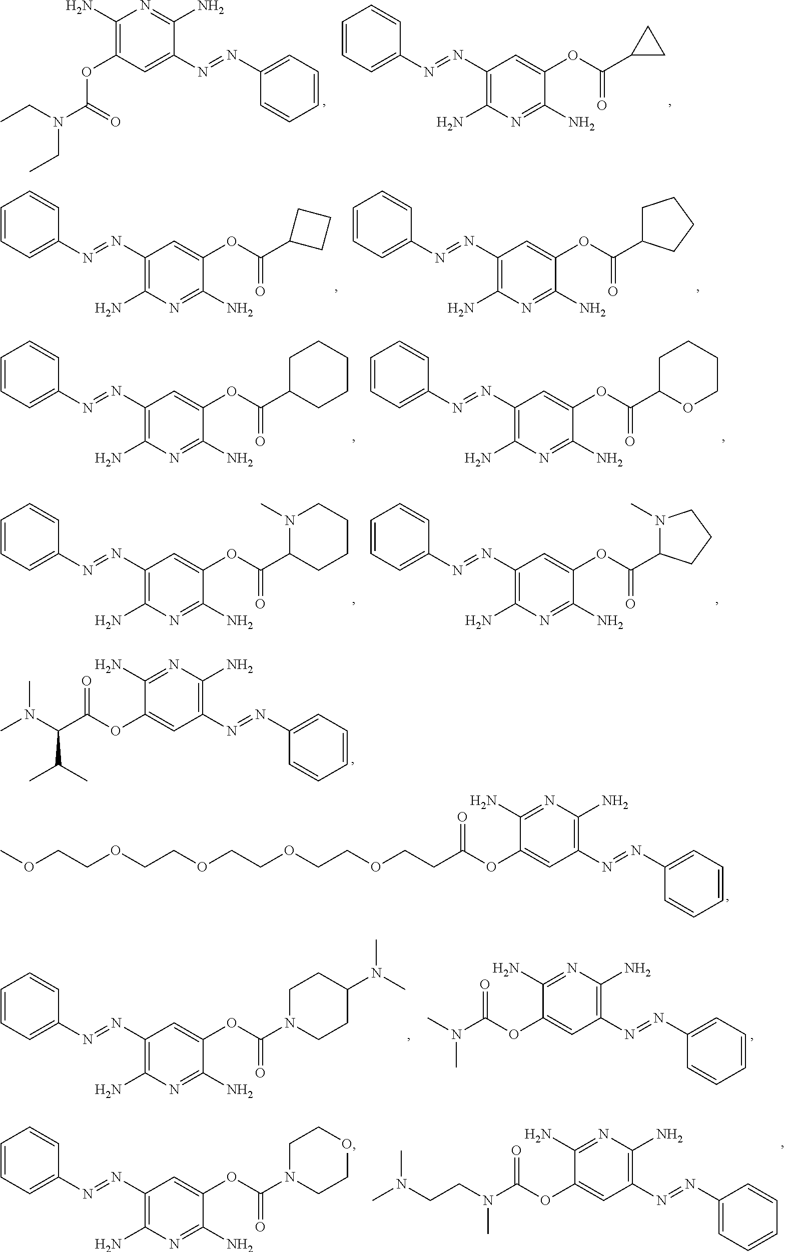

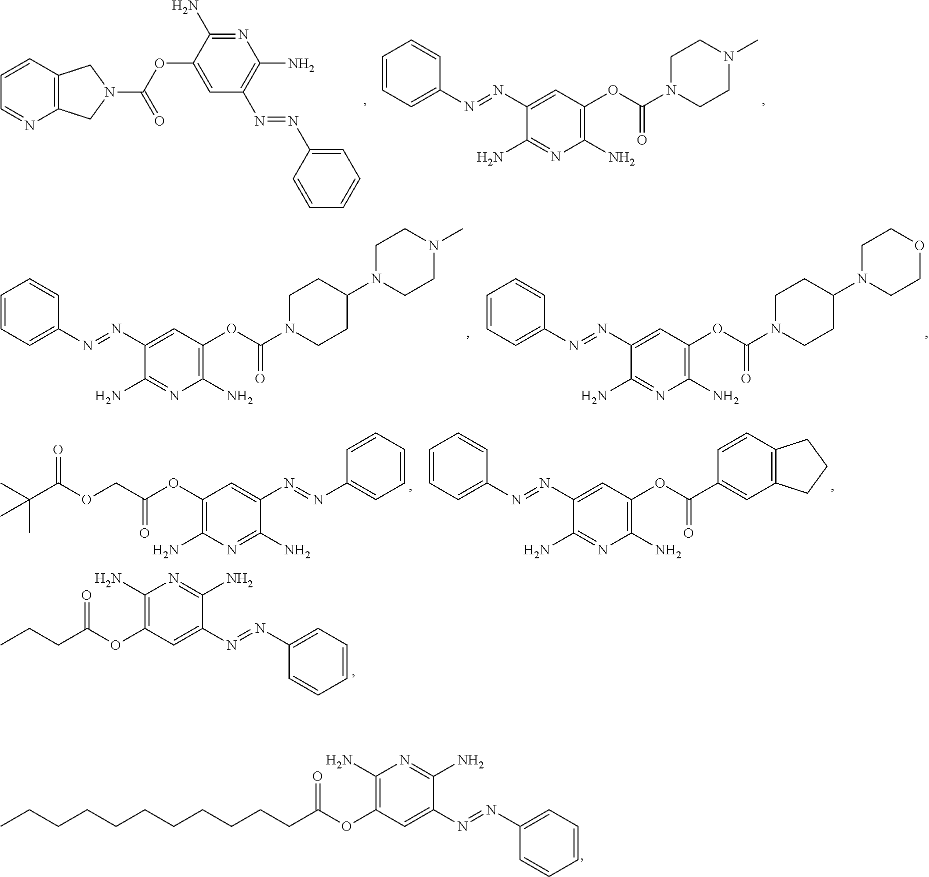

pharmaceutically acceptable salts or solvates thereof.

In another aspect, compounds of general formula (III-C), pharmaceutically acceptable salts or solvates thereof:

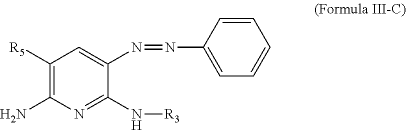

wherein;
R₃ is selected from

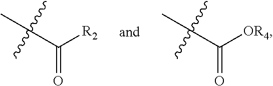

wherein R₂ is selected from the group consisting of optionally substituted alkyl, alkenyl, alkynyl, cycloalkyl, heterocycloalkyl, or heteroaryl and, R₄ is selected from the group consisting of optionally substituted alkyl, alkenyl, alkynyl, —(CH2-CH2-O)n-, cycloalkyl, heterocycloalkyl, or heteroaryl; where n is from 1-20;

R5 selected from —OR₈, and

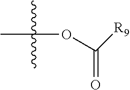

wherein R₉ is selected from the group consisting of optionally substituted alkyl, alkenyl, alkynyl, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment, —(CH2-CH2-O)n-, or N₁₀R₁₁, where n is from 1-20; wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl, alkenyl, alkynyl, cycloalkyl, heterocycloalkyl, aryl, or heteroaryl.

In one embodiment the present invention provides the compound selected from the group consisting of:

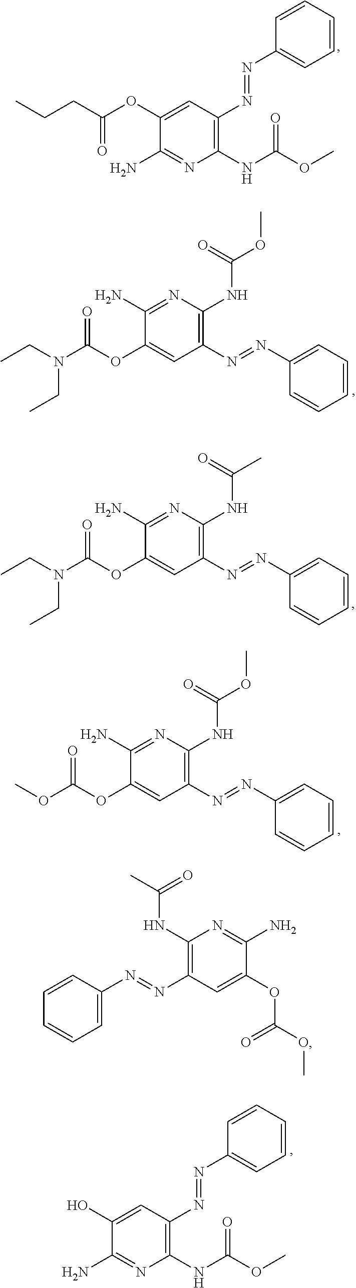

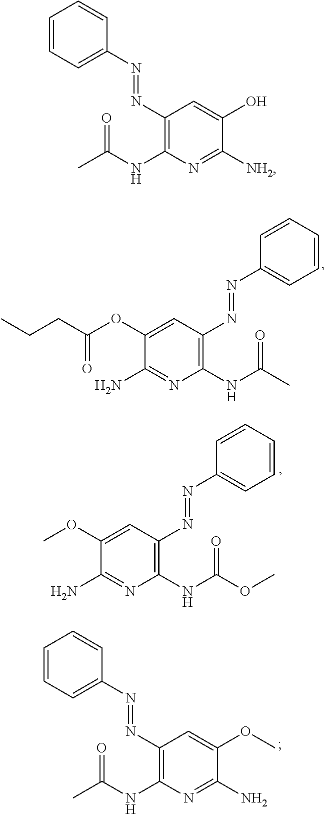

pharmaceutically acceptable salts or solvates thereof.

In one embodiment, the present invention relates to compounds of general formula (IV), pharmaceutically acceptable salts or solvates thereof;

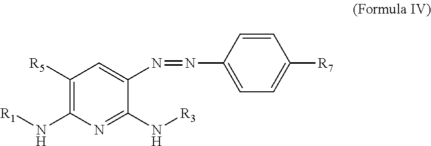

wherein;
R₁ and R₃ are independently selected from hydrogen

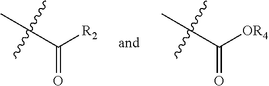

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from hydrogen, —OR₈, halogen and

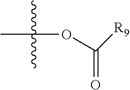

wherein, R₉ is selected from the group consisting of optionally substituted alkyl, alkenyl, alkynyl, cycloalkyl, heterocycloalkyl, aryl, heteroaryl, amino, alkoxy, heterocycloalkoxy, heteroaryloxy, cycloalkoxy, aryloxy, alkyl-ester, alkoxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R₇ is selected from hydrogen, OH, —OR₁₂,

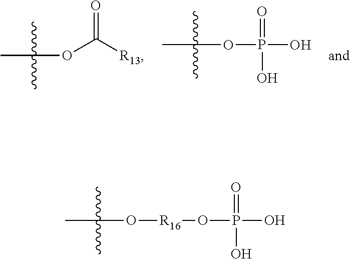

wherein, R₁₂ is selected from the group consisting of optionally substituted alkyl, alkenyl, alkynyl, cycloalkyl, heterocycloalkyl, aryl, or heteroaryl; Ria is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group;
with the proviso that all substitutions at R1, R3, R5 and R7 is not hydrogen at the same time; when any one substitution at R1 or R3 is not hydrogen then R5 and R7 is not hydrogen; when substitution at R1, R3, R7 is hydrogen then R5 is not —OH; when substitution at R1, R3, R5 is hydrogen then R7 is not —OH or —O-(alkyl₁₋₃); when substitution at R1, R3 is hydrogen then both R5 and R7 is not —OH.

In one embodiment, the present invention relates to compounds of general formula (IV-A), pharmaceutically acceptable salts or solvates thereof;

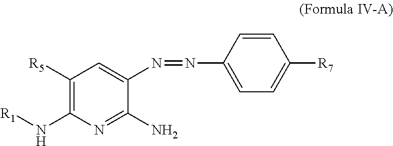

wherein;
R₁ is selected from

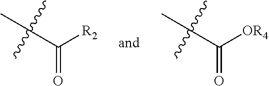

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20;
R₅ is selected from —OR₈, and

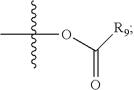

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R₇ is selected from —OH, —OR₁₂,

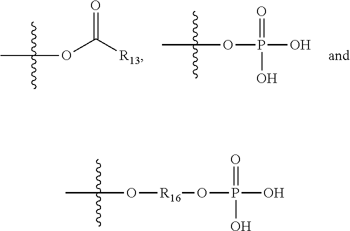

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅, where n is from 1-20; where R₁₄ and R₁₅ are each independently selected from hydrogen, optionally substituted alkyl, heterocycloalkyl, aryl group; R₁₆ is optionally substituted alkylene(C1-6) group.

In one embodiment the present invention provides the compound selected from the group consisting of:

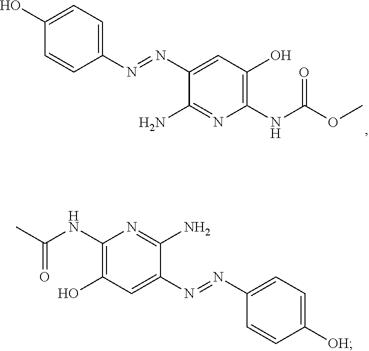

pharmaceutically acceptable salts or solvates thereof.

In one embodiment, the present invention relates to compounds of general formula (IV-B), pharmaceutically acceptable salts or solvates thereof;

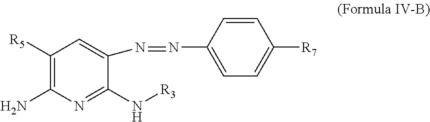

wherein;
R₃ is selected from

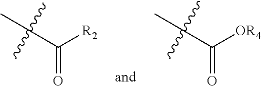

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from —OH, —OR₈, and

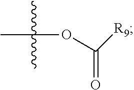

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₇ is selected from —OH, —OR₁₂,

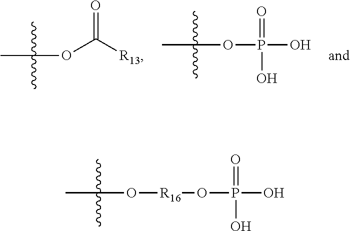

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is optionally substituted alkylene(C₁₋₆) group.

In one embodiment, the present invention relates to compounds of general formula (IV-C), pharmaceutically acceptable salts or solvates thereof;

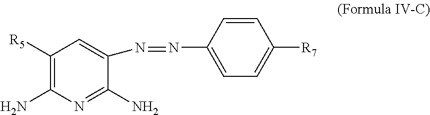

wherein;
R₅ is selected from —OH, —OR₈, and

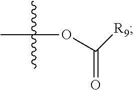

wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₇ is selected from —OH, —OR₁₂,

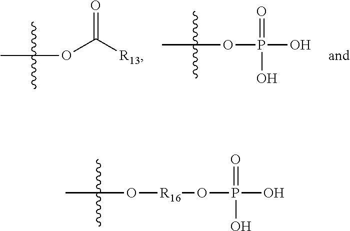

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is optionally substituted alkylene(C₁₋₆) group;
with proviso that both R₅ and R₇ is not —OH.
In one embodiment the present invention provides the compound selected from the group consisting of:

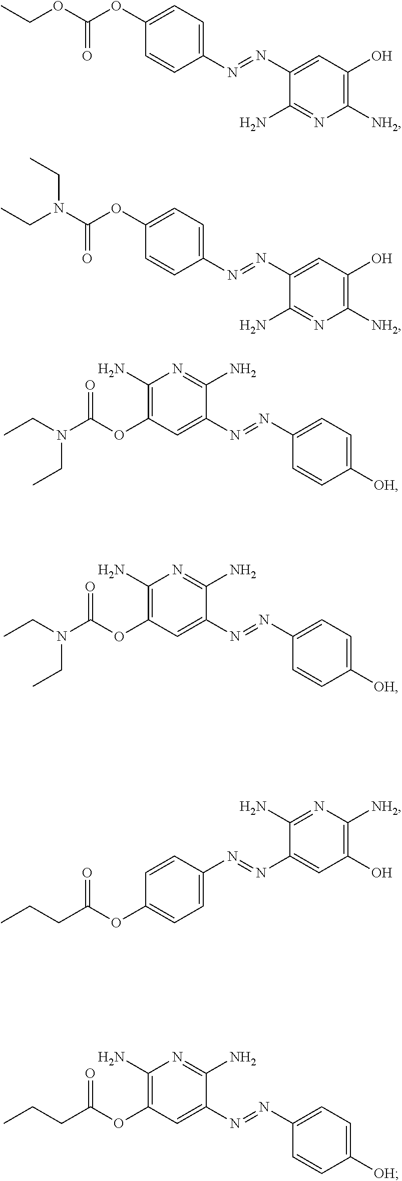 [SMILES: CCOC(=O)Oc1ccc(/N=N/c2cc(O)c(N)nc2N)cc1 ; CCN(CC)C(=O)Oc1ccc(/N=N/c2cc(O)c(N)nc2N)cc1 ; CCN(CC)C(=O)Oc1cc(/N=N/c2ccc(O)cc2)c(N)nc1N ; CCN(CC)C(=O)Oc1cc(/N=N/c2ccc(O)cc2)c(N)nc1N ; CCCC(=O)Oc1ccc(/N=N/c2cc(O)c(N)nc2N)cc1 ; CCCC(=O)Oc1cc(/N=N/c2ccc(O)cc2)c(N)nc1N]

pharmaceutically acceptable salts or solvates thereof:

In one aspect, the present invention relates to compounds of general formula (V),

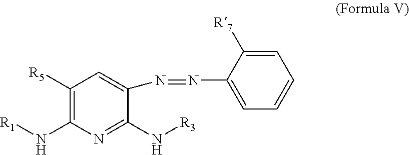

wherein;
R₁ and R₃ are the same or different and are independently selected from hydrogen,

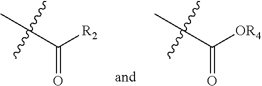

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)n-, cycloalkyl, heterocycloalkyl, aryl, or heteroaryl;
where n is from 1-20;
R₅ is selected from hydrogen, OH, —OR₈, halogen and

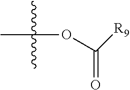

wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)n-, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R′₇ is selected from hydrogen, OH, —OR₁₂,

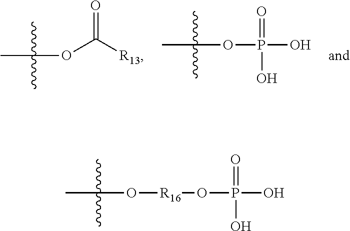

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20;
wherein R₁₄ and R₁₅ are each independently selected from hydrogen, optionally substituted alkyl, heterocycloalkyl, aryl group; R₁₆ is selected from optionally substituted alkylene(C₁₋₆) group;
with the proviso that all substitutions at R1, R3, R5 and R′7 is not hydrogen at the same time; when any one substitution at R1 or R3 is not hydrogen then R5 and R′7 is not hydrogen; when substitution at R1, R3, R′7 is hydrogen then R5 is not —OH; when substitution at R1, R3, R5 is hydrogen then R′7 is not —OH or —O-(alkyl₁₋₃).

In one embodiment, the present invention relates to compounds of general formula (V-A), pharmaceutically acceptable salts or solvates thereof;

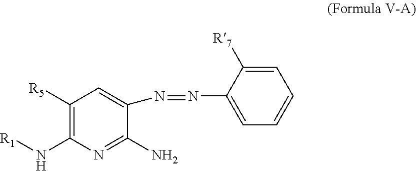

wherein;
R₁ is selected from

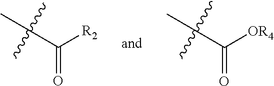

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)n-, cycloalkyl, heterocycloalkyl, aryl, or heteroaryl; where n is from 1-20;
R₅ is selected from —OR₈ and

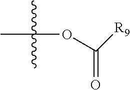

wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R′7 is selected from —OH, —OR₁₂,

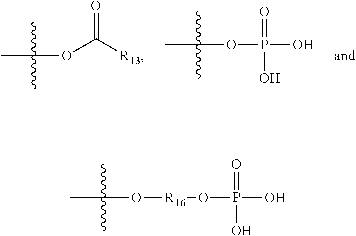

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or NR₁₄R₁₅; where n is from 1-20; wherein R₁₄ and R₁₅ each independently selected from hydrogen, optionally substituted alkyl, heterocycloalkyl, aryl group; R₁₆ is selected from optionally substituted alkylene(C₁₋₆) group.

In one embodiment, the present invention relates to compounds of general formula (V-B), pharmaceutically acceptable salts or solvates thereof;

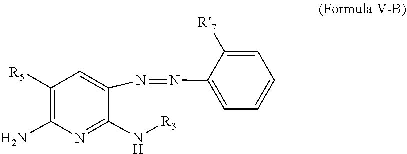

wherein;
R₃ is selected from

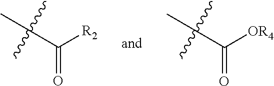

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20;
R₅ is selected from ˜OR₈ and

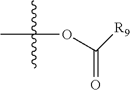

wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)n-, or NR₁₀R₁₁, where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl; R′₇ is selected from —OH, —OR₁₂,

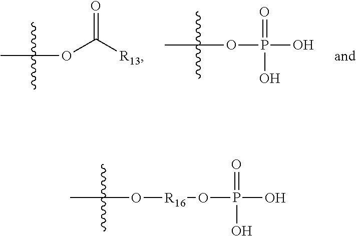

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment; —(CH₂—CH₂—O)ₙ—, or NR₁₄R₁₅; where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group.

In one embodiment, the present invention relates to compounds of general formula (V-C), pharmaceutically acceptable salts or solvates thereof;

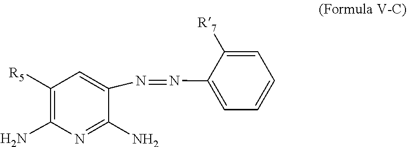

wherein;
R₅ is selected from —OH, —OR₈ and

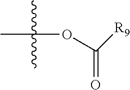

wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)n-, or NR₁₀R₁₁, wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl; where n is from 1-20;
R′7 is selected from —OH, —OR₁₂,

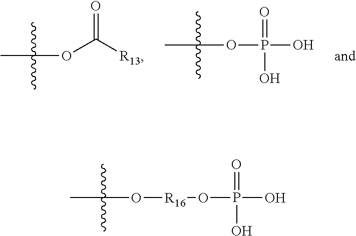

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C₁₋₆) group.

In one aspect, the present invention relates to compounds of general formula (VI), pharmaceutically acceptable salts or solvates thereof:

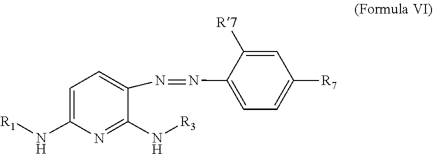

wherein;
R₁ and R₃ are the same or different and are independently selected from hydrogen,

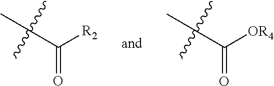

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl;
where n is from 1-20;
R₇ and R′₇ is selected from hydrogen, OH, —OR₁₂,

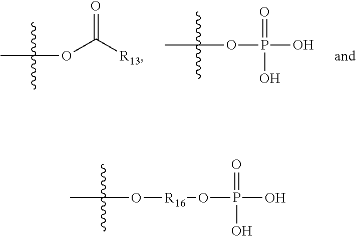

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or R₁₃ selected from by NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C₁₋₆) group;
with the proviso that all substitutions at R1, R3, R7 and R′7 is not hydrogen at the same time; when any one substitution at R1 or R3 is not hydrogen then R7 and R′7 is not hydrogen; when substitution at R1, R3, R7 is hydrogen then R′7 is not —OH or —O-(alkyl₁₋₃); when substitution at R1, R3, R′7 is hydrogen then R7 is not —OH or —O-(alkyl₁₋₃).

In one embodiment, the present invention relates to compounds of general formula (VI-A), pharmaceutically acceptable salts or solvates thereof;

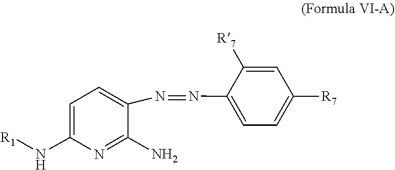

wherein;
R₁ is selected from

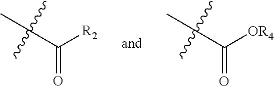

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)n-, cycloalkyl, heterocycloalkyl, aryl, or heteroaryl; where n is from 1-20;
R₇ and R′₇ is selected independently at each point of substitution from —OH, —OR₁₂,

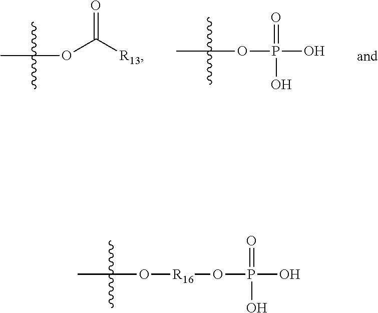

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group.

In one embodiment, the present invention relates to compounds of general formula (VI-B), pharmaceutically acceptable salts or solvates thereof;

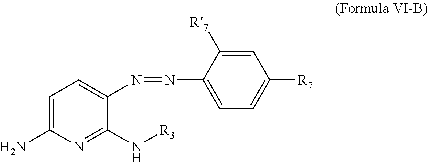

wherein;
R₃ is selected from

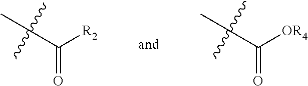

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)n-, cycloalkyl, heterocycloalkyl, aryl, or heteroaryl; where n is from 1-20;
R₇ and R′₇ is selected independently at each point of substitution from —OH, —OR₁₂,

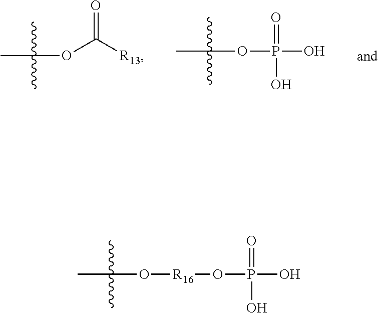

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C₁₋₆) group.

In one embodiment, the present invention relates to compounds of general formula (VI-C) pharmaceutically acceptable salts or solvates thereof:

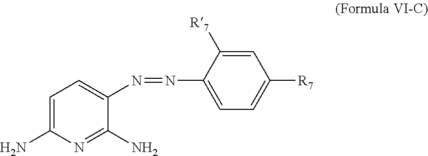

wherein,
R₇ and R′₇ is selected independently at each point of substitution from —OH, —OR₁₂,

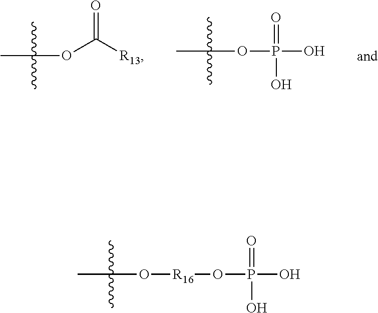

R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R14 and R15 each independently selected from hydrogen, optionally substituted alkyl, heterocycloalkyl, aryl group. R16 is selected from optionally substituted alkylene_{(C1-6)}group.

In one aspect, the present invention relates to compounds of general formula (VII), pharmaceutically acceptable salts or solvates thereof:

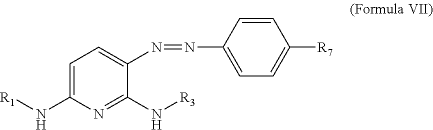

wherein;
R₁ and R₃ are independently selected from hydrogen,

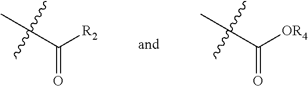

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ is selected from hydrogen, OH, —OR₁₂,

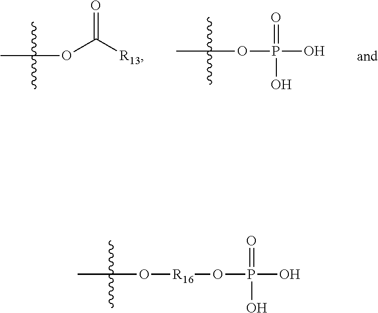

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C1-6) group;
with the proviso that all substitutions at R1, R3 and R7 is not hydrogen at the same time; when any one substitution at R1 or R3 is not hydrogen then R7 is not hydrogen; when substitution at R1, R3 is hydrogen then R7 is not —OH, —O-(alkyl₁₋₃).

In one embodiment, the present invention relates to compounds of general formula (VII-A), pharmaceutically acceptable salts or solvates thereof;

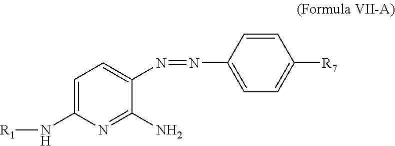

wherein;
R₁ is selected from

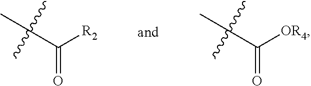

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;

R₇ is selected from —OH, —OR₁₂,

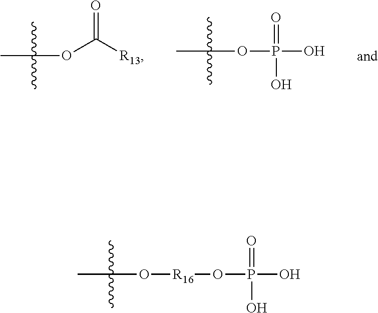

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group. R₁₆ is selected from optionally substituted alkylene(C₁₋₆) group.

In one embodiment the present invention provides the compound selected from the group consisting of:

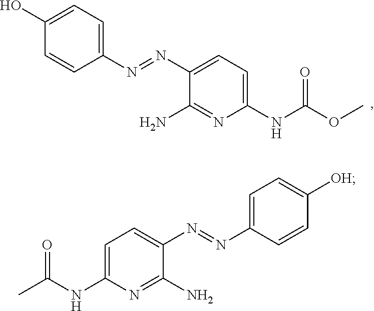

pharmaceutically acceptable salts or solvates thereof.

In one embodiment, the present invention relates to compounds of general formula (VII-B), pharmaceutically acceptable salts or solvates thereof;

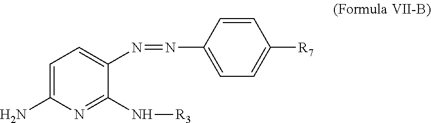

wherein;
R₃ is selected from

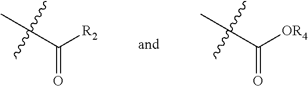

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;

R₇ is selected from —OH, —OR₁₂,

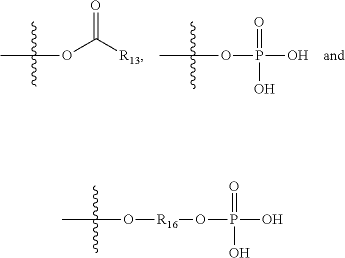

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group;

In one embodiment, the present invention relates to compounds of general formula (VII-C), pharmaceutically acceptable salts or solvates thereof;

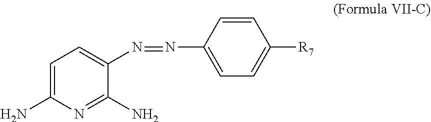

Wherein R₇ is selected from −OR₁₂,

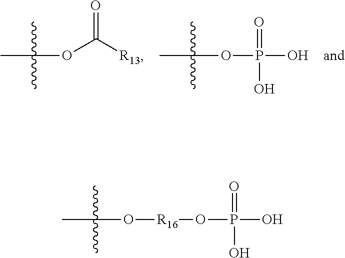

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group, R₁₆ is selected from optionally substituted alkylene(C₁₋₆) group.

In one embodiment the present invention provides the compound selected from the group consisting of:

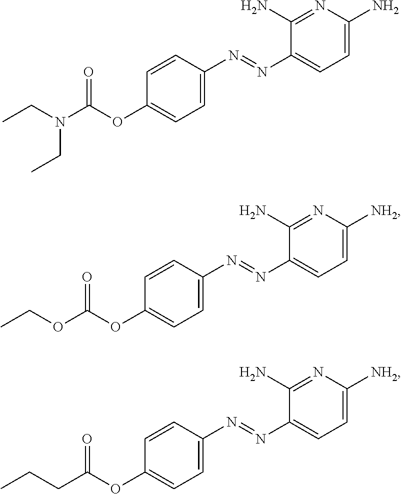 [SMILES: CCN(CC)C(=O)Oc1ccc(/N=N/c2ccc(N)nc2N)cc1 ; CCOC(=O)Oc1ccc(/N=N/c2ccc(N)nc2N)cc1 ; CCCC(=O)Oc1ccc(/N=N/c2ccc(N)nc2N)cc1]

pharmaceutically acceptable salts or solvates thereof.

In one aspect, the present invention relates to compounds of general formula (VIII) pharmaceutically acceptable salts or solvates thereof:

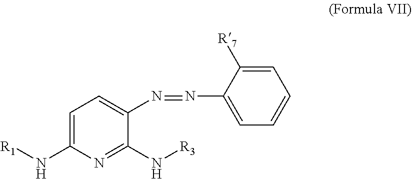

wherein;
R₁ and R₃ are independently selected from hydrogen,

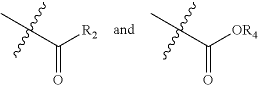

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)ₙ—, cycloalkyl, heterocycloalkyl, aryl, or heteroaryl; where n is from 1-20;
R′₇ is selected from hydrogen, OH, —OR₁₂,

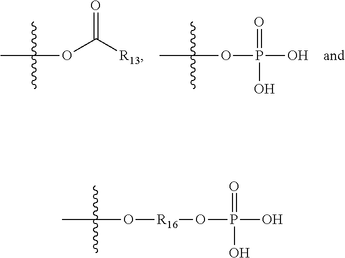

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene_{(C1-6)} group;
with the proviso that all substitutions at R₁, R₃ and R′₇ is not hydrogen at the same time; when any one substitution at R₁ or R₃ is not hydrogen then R′₇ is not hydrogen; when substitution at R₁, R₃ is hydrogen then R′₇ is not —OH or —O-(alkyl₁₋₃).

In one embodiment the present invention provides the compound selected from the group consisting of:

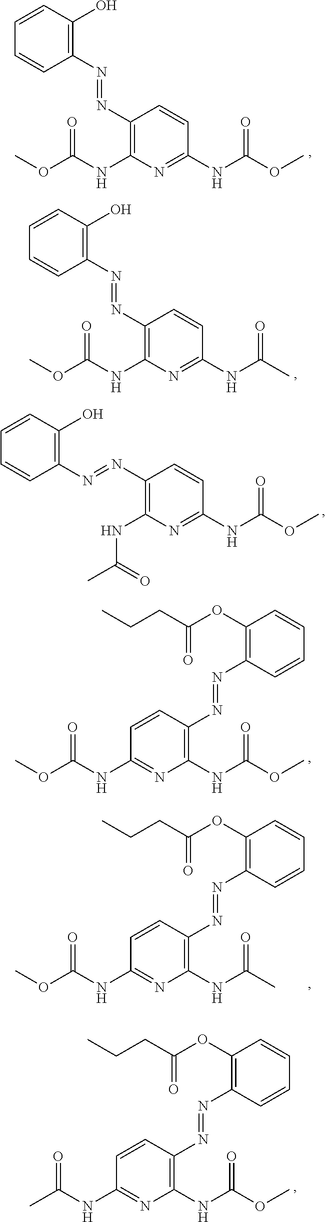

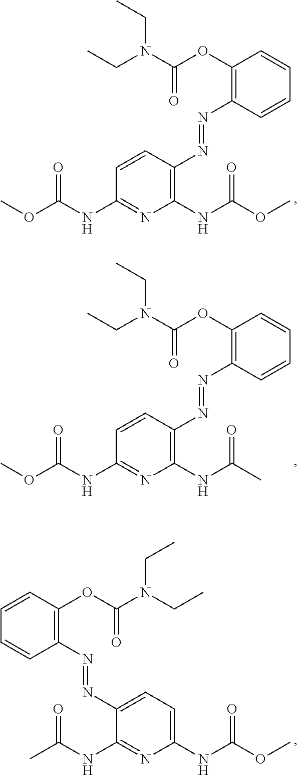

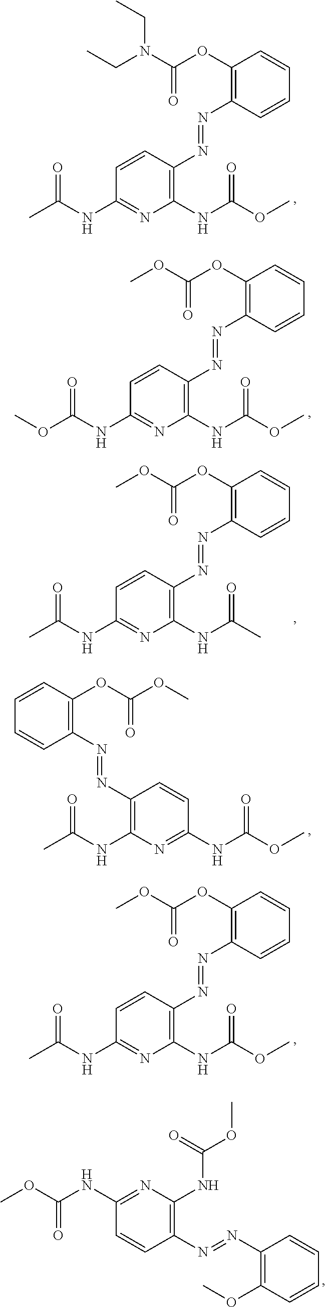

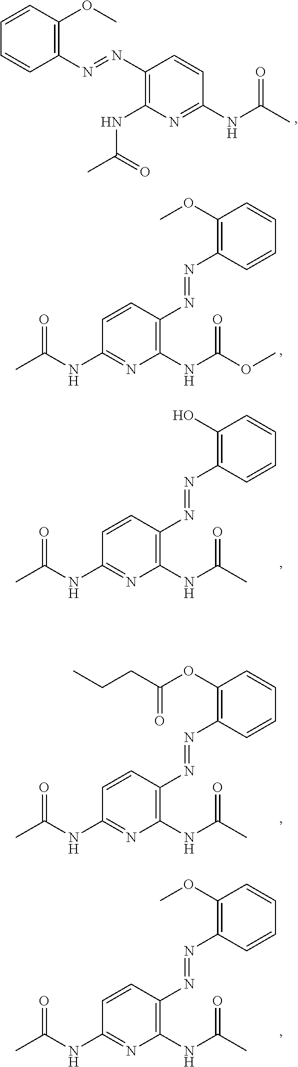

In one embodiment, the present invention relates to compounds of general formula (VIII-A), pharmaceutically acceptable salts or solvates thereof;

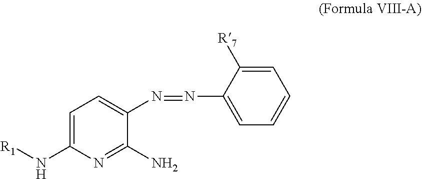

wherein;
R₁ is selected from

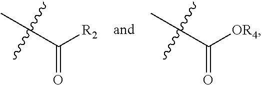

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;

R′₇ is selected from —OH, —OR₁₂,

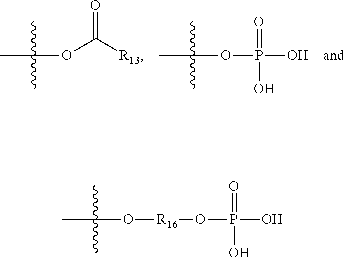

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group. R₁₆ is selected from optionally substituted alkylene(C₁₋₆) group.

In one embodiment the present invention provides the compound selected from the group consisting of:

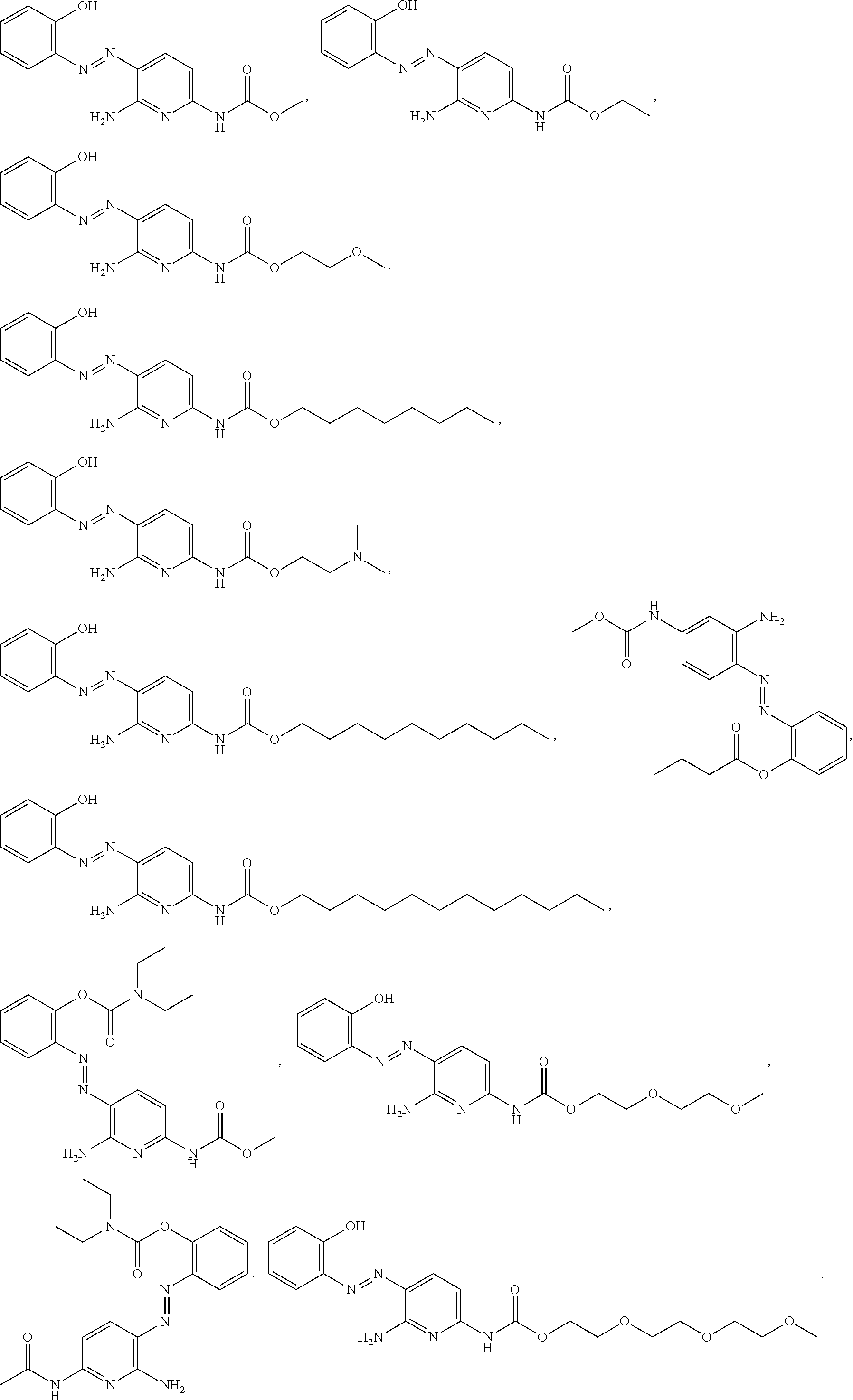

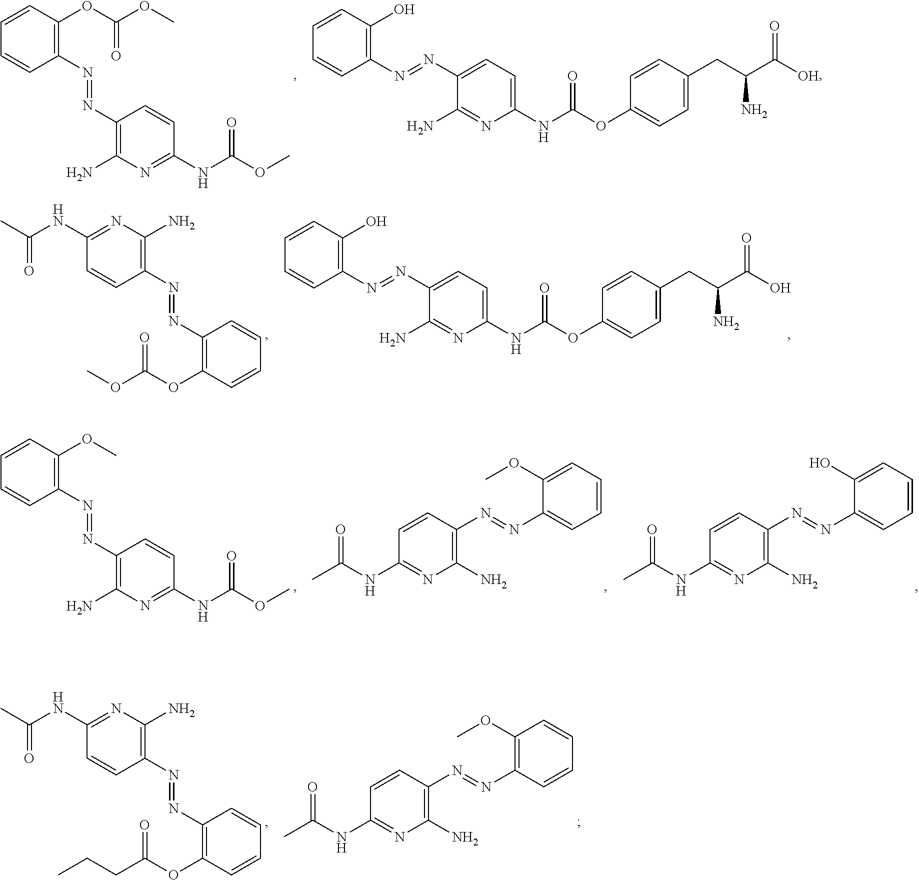

pharmaceutically acceptable salts or solvates thereof;

In one embodiment, the present invention relates to compound of formula (VIII-B), pharmaceutically acceptable salts or solvates thereof;

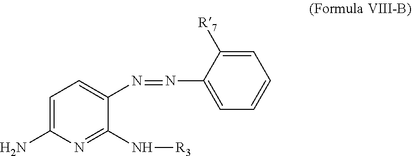

wherein;
R₃ is selected from

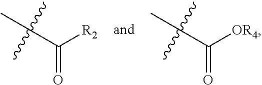

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)n-, cycloalkyl, heterocycloalkyl, aryl, or heteroaryl, where n is from 1-20;
R′₇ is selected from —OH, —OR₁₂,

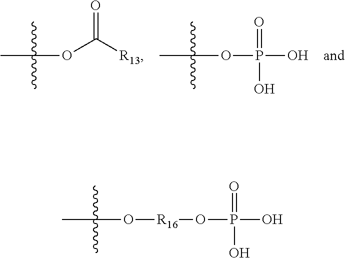

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅; where n is from 1-20; where R₁₄ and R₁₅ are each independently selected from hydrogen, optionally substituted alkyl, heterocycloalkyl, aryl group. R₁₆ is selected from optionally substituted alkylene(C₁₋₆) group.

In one embodiment the present invention provides the compound selected from the group consisting of:

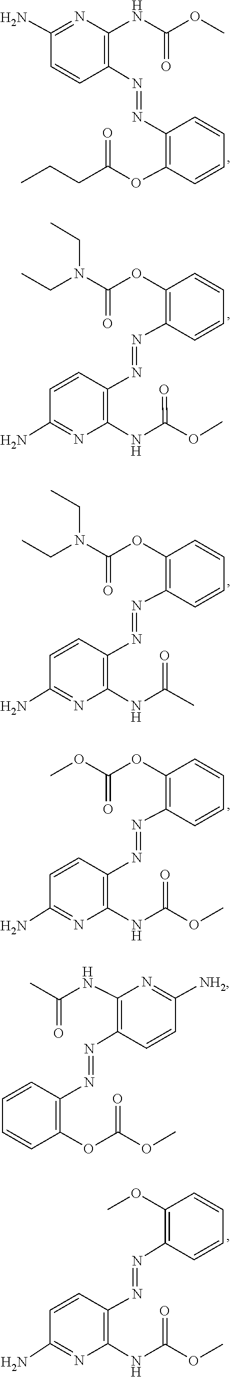

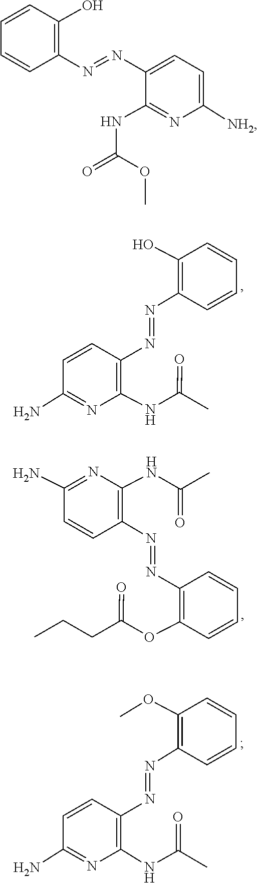

pharmaceutically acceptable salts or solvates thereof.

In one embodiment, the present invention relates to compounds of general formula (VIII-C), pharmaceutically acceptable salts or solvates thereof:

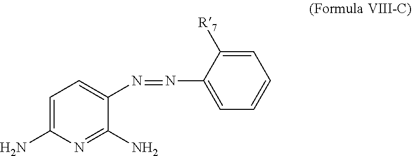

wherein R′₇ is selected from —OR₁₂,

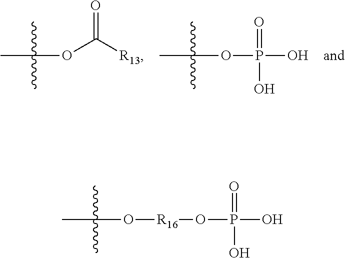

wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group; with proviso that when substitution at R₁, R₃ is hydrogen then R′₇ is not —OH or —O-(alkyl₁₋₃).

In one embodiment the present invention provides the compound selected from the group consisting of:

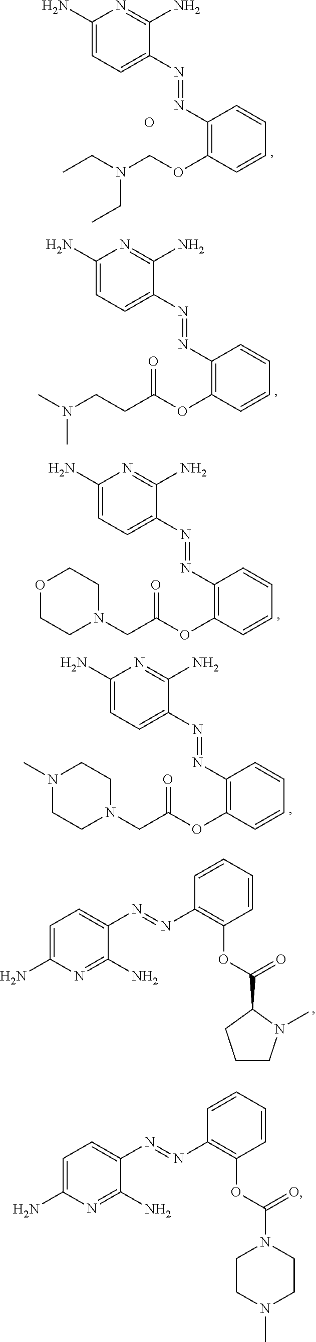

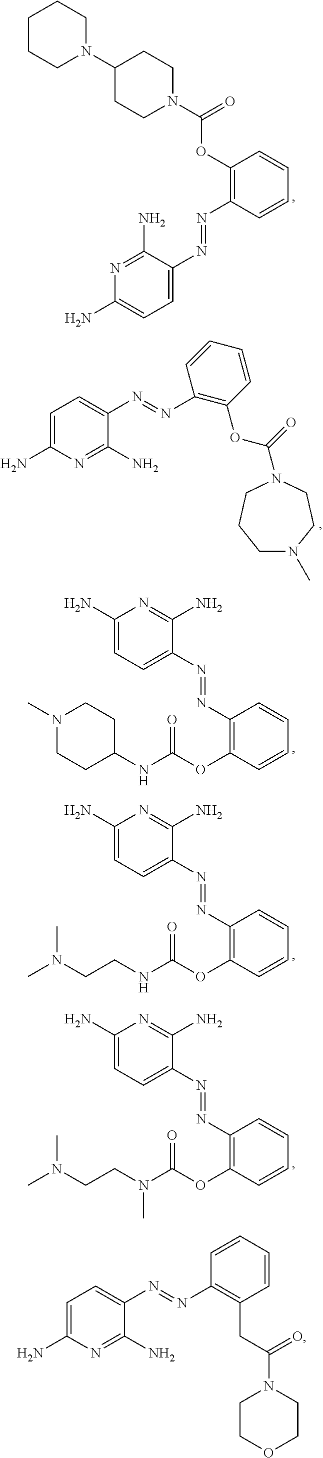

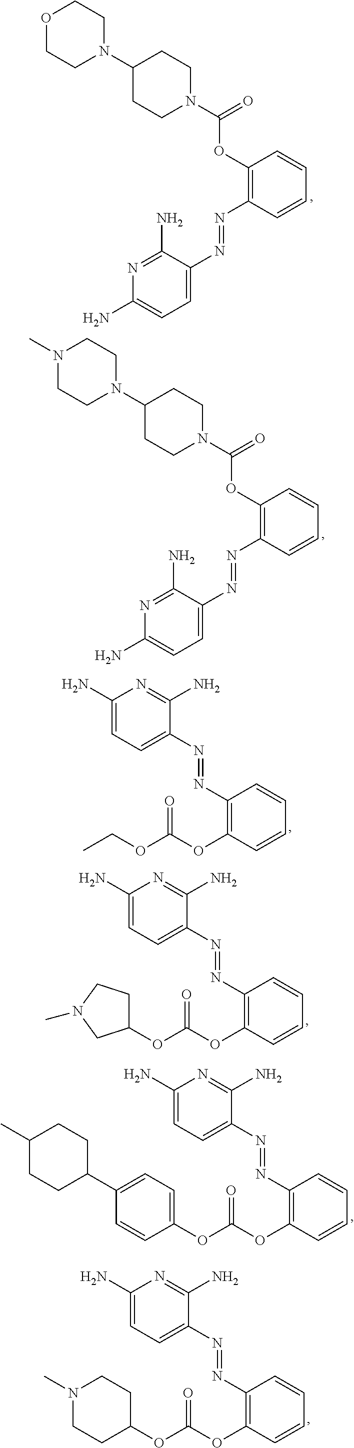

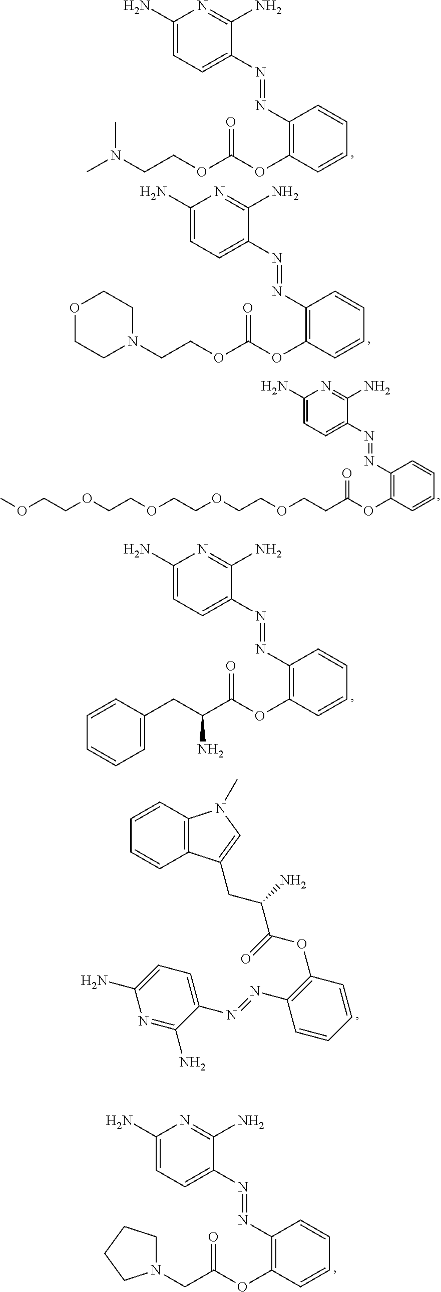

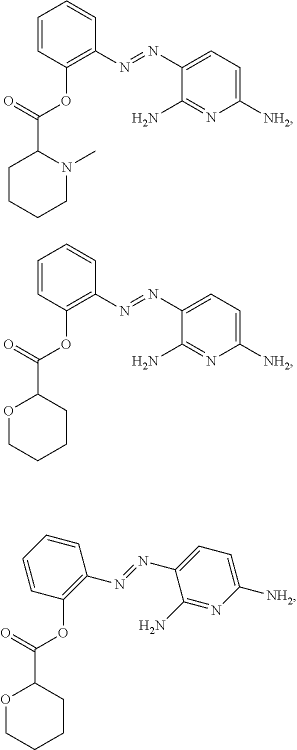

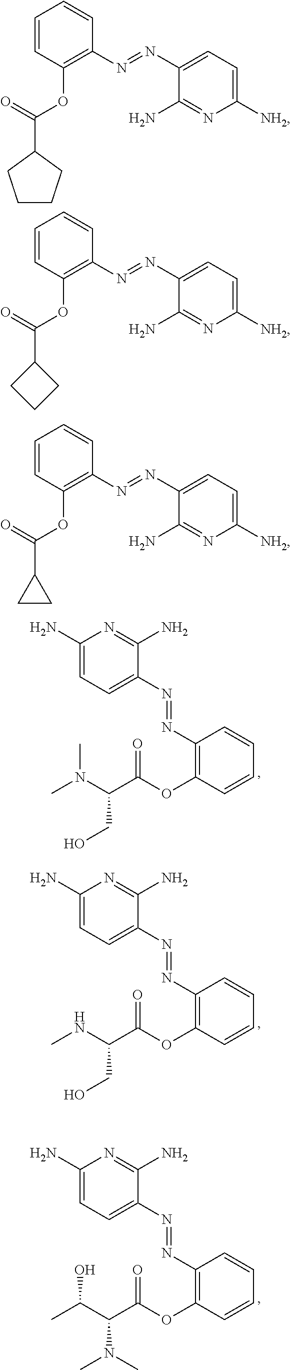

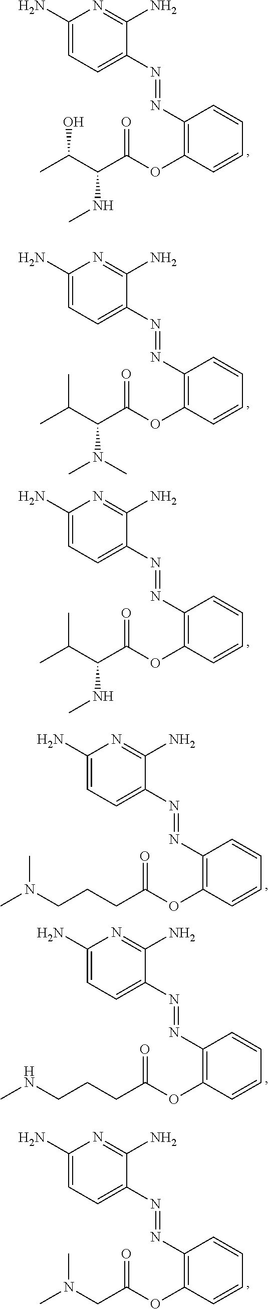

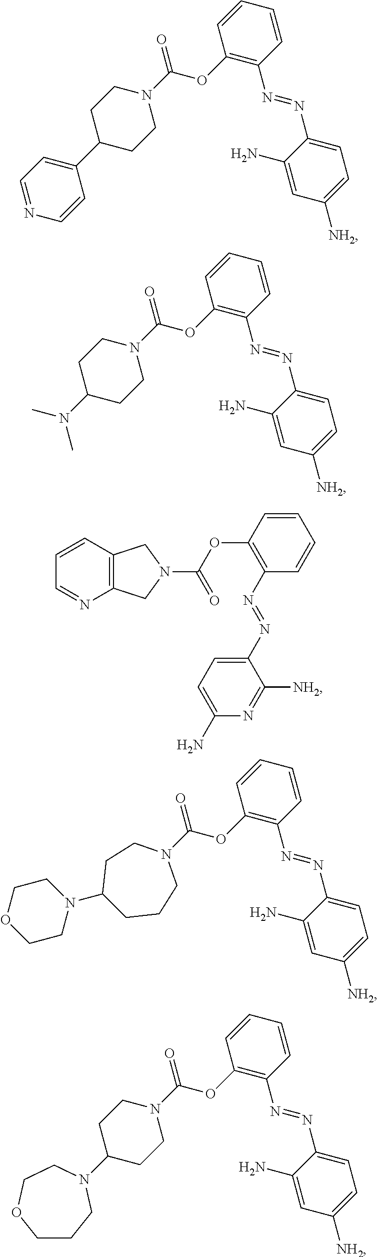

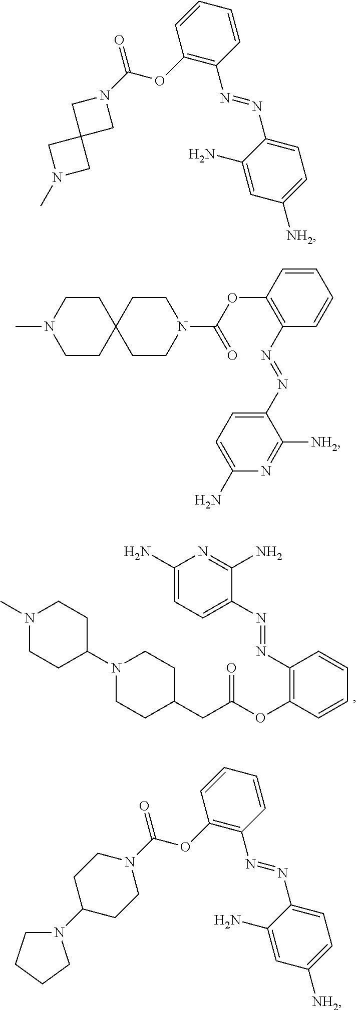

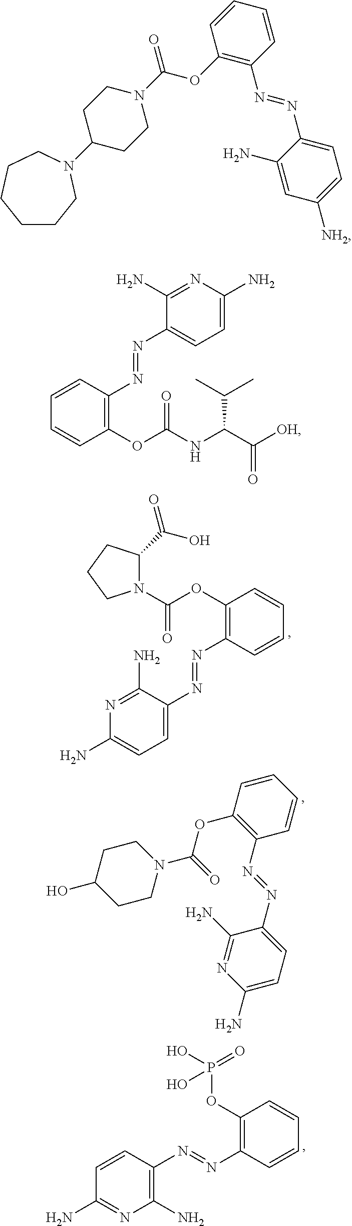

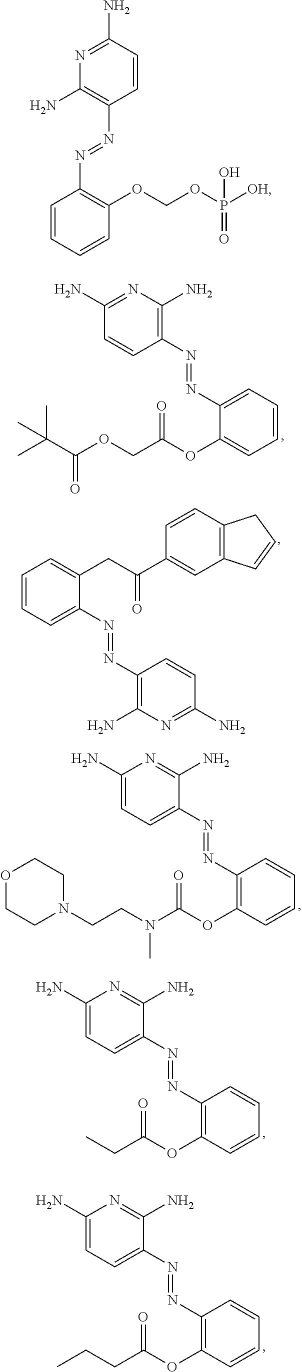

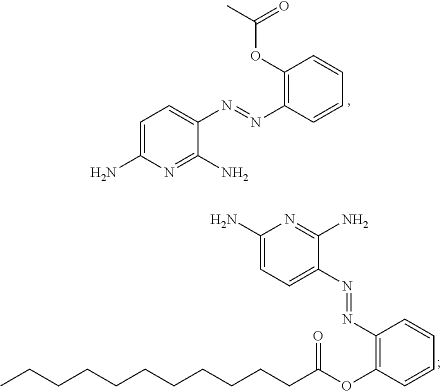 [SMILES: CC(=O)Oc1ccccc1/N=N/c1ccc(N)nc1N ; CCCCCCCCCCCC(=O)Oc1ccccc1/N=N/c1ccc(N)nc1N]

pharmaceutically acceptable salts thereof.

In one embodiment, the present invention provides the compound selected from group of compounds listed in Table 1 pharmaceutically acceptable salts or solvates thereof.

In one embodiment, the present invention provides a method for the treatment of at least one disease condition selected from interstitial cystitis/bladder pain syndrome (IC/BPS), over active bladder

(OAB), chronic cough, endometriosis, neuropathic pain, migraine, pruritus, postherpetic neuralgia, urinary tract infection related pain, osteoarthritis, arthritis and urinary incontinence, comprises administering at least one compound selected from group consisting of compounds listed in Table 1 pharmaceutically acceptable salts or solvates thereof.

In one embodiment, the present invention provides a method of use of at least one compound selected from group consisting of compounds listed in Table 1, pharmaceutically acceptable salts or solvates thereof, for the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence.

In one embodiment, the serial number given in table 1 and 2 is consider as compound number for the reference in the specification.

TABLE 1
Compounds

| Sr. | |
|---|---|
| No | Structures |
| 1 | 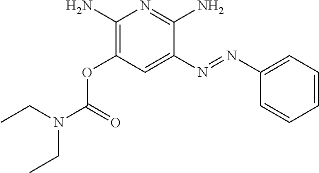 [SMILES: CCN(CC)C(=O)Oc1cc(/N=N/c2ccccc2)c(N)nc1N] |
| 2 | 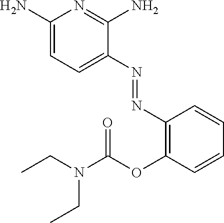 [SMILES: CCN(CC)C(=O)Oc1ccccc1/N=N/c1ccc(N)nc1N] |
| 3 | 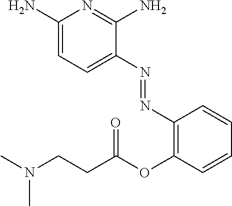 [SMILES: CN(C)CCC(=O)Oc1ccccc1/N=N/c1ccc(N)nc1N] |
| 4 | 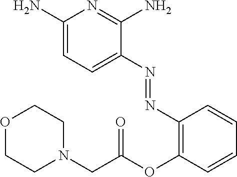 [SMILES: Nc1ccc(/N=N/c2ccccc2OC(=O)CN2CCOCC2)c(N)n1] |
| 5 | 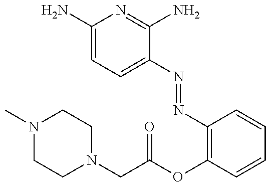 [SMILES: CN1CCN(CC(=O)Oc2ccccc2/N=N/c2ccc(N)nc2N)CC1] |
| 6 | 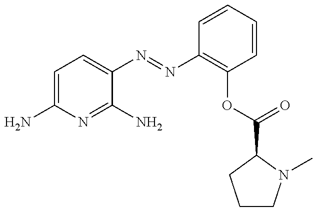 [SMILES: CN1CCC[C@H]1C(=O)Oc1ccccc1/N=N/c1ccc(N)nc1N] |
| 7 | 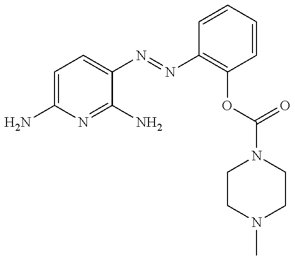 [SMILES: CN1CCN(C(=O)Oc2ccccc2/N=N/c2ccc(N)nc2N)CC1] |
| 8 | 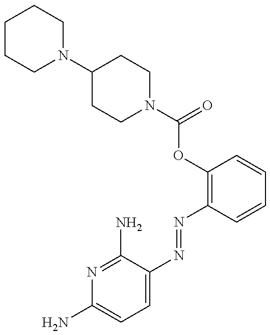 [SMILES: Nc1ccc(/N=N/c2ccccc2OC(=O)N2CCC(N3CCCCC3)CC2)c(N)n1] |
| 9 | 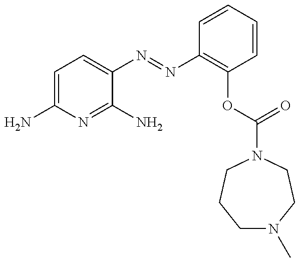 [SMILES: CN1CCCN(C(=O)Oc2ccccc2/N=N/c2ccc(N)nc2N)CC1] |
| 10 | 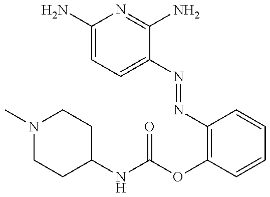 [SMILES: CN1CCC(NC(=O)Oc2ccccc2/N=N/c2ccc(N)nc2N)CC1] |
| 11 | 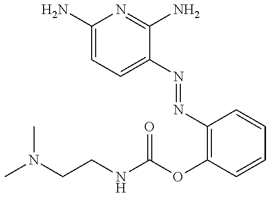 [SMILES: CN(C)CCNC(=O)Oc1ccccc1/N=N/c1ccc(N)nc1N] |
| 12 | 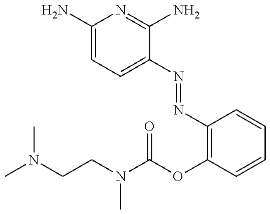 [SMILES: CN(C)CCN(C)C(=O)Oc1ccccc1/N=N/c1ccc(N)nc1N] |
| 13 | 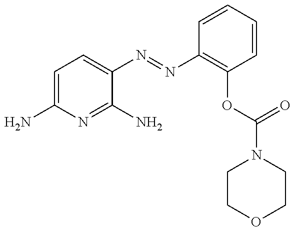 [SMILES: Nc1ccc(/N=N/c2ccccc2OC(=O)N2CCOCC2)c(N)n1] |
| 14 | 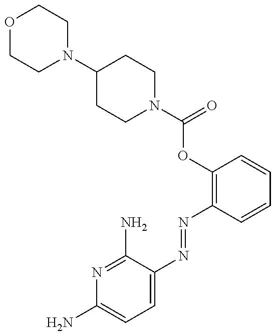 [SMILES: Nc1ccc(/N=N/c2ccccc2OC(=O)N2CCC(N3CCOCC3)CC2)c(N)n1] |
| 15 | 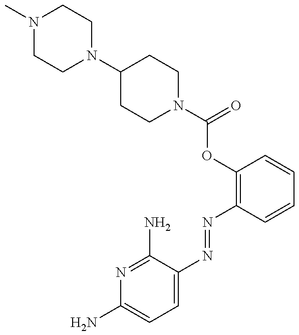 [SMILES: CN1CCN(C2CCN(C(=O)Oc3ccccc3/N=N/c3ccc(N)nc3N)CC2)CC1] |
| 16 | 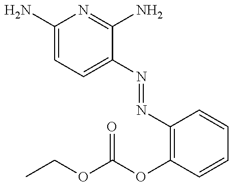 [SMILES: CCOC(=O)Oc1ccccc1/N=N/c1ccc(N)nc1N] |
| 17 | 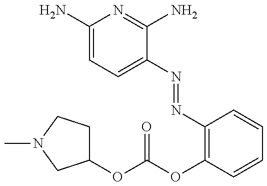 [SMILES: CN1CCC(OC(=O)Oc2ccccc2/N=N/c2ccc(N)nc2N)C1] |
| 18 | 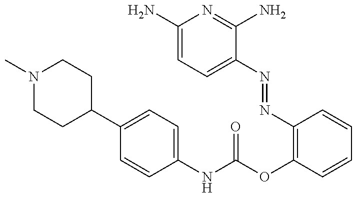 [SMILES: CN1CCC(c2ccc(NC(=O)Oc3ccccc3/N=N/c3ccc(N)nc3N)cc2)CC1] |
| 19 | 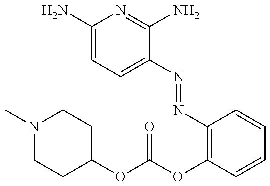 [SMILES: CN1CCC(OC(=O)Oc2ccccc2/N=N/c2ccc(N)nc2N)CC1] |
| 20 | 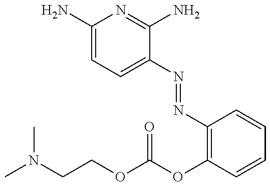 [SMILES: CN(C)CCOC(=O)Oc1ccccc1/N=N/c1ccc(N)nc1N] |
| 21 | 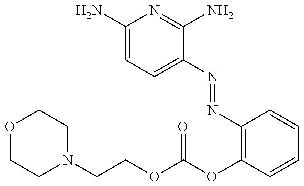 [SMILES: Nc1ccc(/N=N/c2ccccc2OC(=O)OCCN2CCOCC2)c(N)n1] |
| 22 | 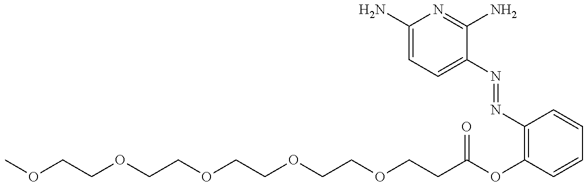 [SMILES: COCCOCCOCCOCCOCCC(=O)Oc1ccccc1/N=N/c1ccc(N)nc1N] |
| 23 | 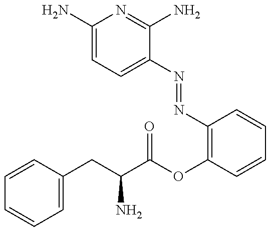 [SMILES: Nc1ccc(/N=N/c2ccccc2OC(=O)[C@@H](N)Cc2ccccc2)c(N)n1] |
| 24 | 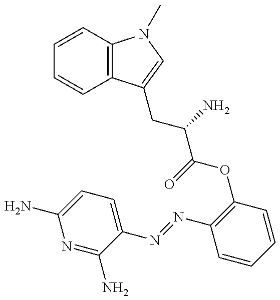 [SMILES: Cn1cc(C[C@H](N)C(=O)Oc2ccccc2/N=N/c2ccc(N)nc2N)c2ccccc21] |
| 25 | 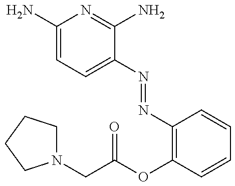 [SMILES: Nc1ccc(/N=N/c2ccccc2OC(=O)CN2CCCC2)c(N)n1] |
| 26 | 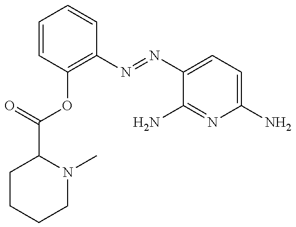 [SMILES: CN1CCCCC1C(=O)Oc1ccccc1/N=N/c1ccc(N)nc1N] |
| 27 | 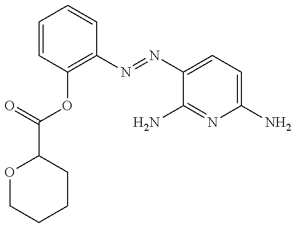 [SMILES: Nc1ccc(/N=N/c2ccccc2OC(=O)C2CCCCO2)c(N)n1] |
| 28 | 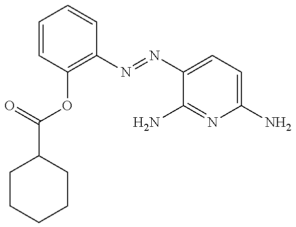 [SMILES: Nc1ccc(/N=N/c2ccccc2OC(=O)C2CCCCC2)c(N)n1] |
| 29 | 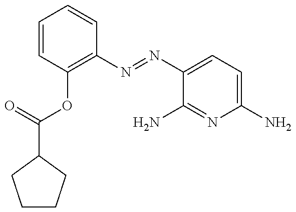 [SMILES: Nc1ccc(/N=N/c2ccccc2OC(=O)C2CCCC2)c(N)n1] |
| 30 | 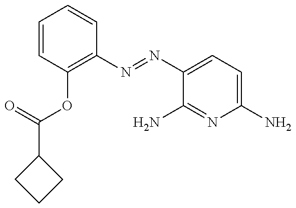 [SMILES: Nc1ccc(/N=N/c2ccccc2OC(=O)C2CCC2)c(N)n1] |
| 31 | 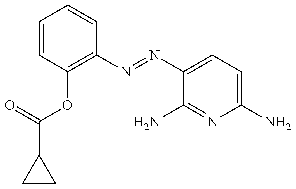 [SMILES: Nc1ccc(/N=N/c2ccccc2OC(=O)C2CC2)c(N)n1] |
| 32 | 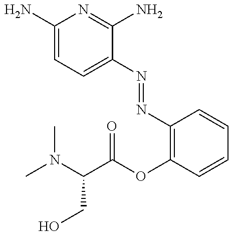 [SMILES: CN(C)[C@@H](CO)C(=O)Oc1ccccc1/N=N/c1ccc(N)nc1N] |
| 33 | 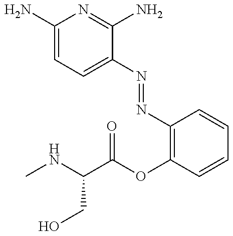 [SMILES: CN[C@@H](CO)C(=O)Oc1ccccc1/N=N/c1ccc(N)nc1N] |
| 34 | 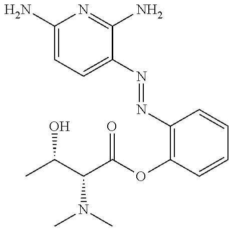 [SMILES: C[C@H](O)[C@H](C(=O)Oc1ccccc1/N=N/c1ccc(N)nc1N)N(C)C] |
| 35 | 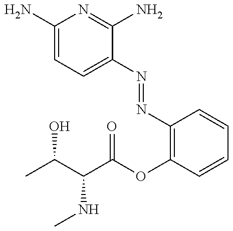 [SMILES: CN[C@@H](C(=O)Oc1ccccc1/N=N/c1ccc(N)nc1N)[C@H](C)O] |
| 36 | 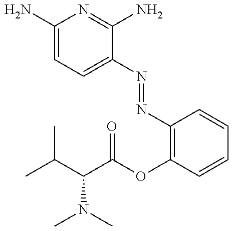 [SMILES: CC(C)[C@H](C(=O)Oc1ccccc1/N=N/c1ccc(N)nc1N)N(C)C] |
| 37 | 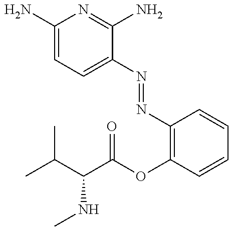 [SMILES: CN[C@@H](C(=O)Oc1ccccc1/N=N/c1ccc(N)nc1N)C(C)C] |
| 38 | 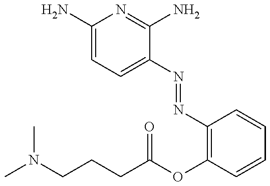 [SMILES: CN(C)CCCC(=O)Oc1ccccc1/N=N/c1ccc(N)nc1N] |
| 39 | 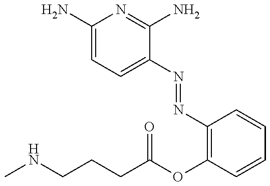 [SMILES: CNCCCC(=O)Oc1ccccc1/N=N/c1ccc(N)nc1N] |
| 40 | 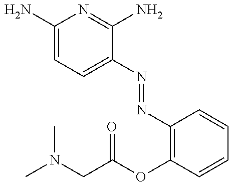 [SMILES: CN(C)CC(=O)Oc1ccccc1/N=N/c1ccc(N)nc1N] |
| 41 | 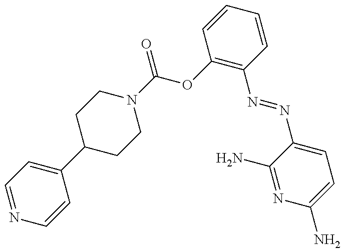 [SMILES: Nc1ccc(/N=N/c2ccccc2OC(=O)N2CCC(c3ccncc3)CC2)c(N)n1] |
| 42 | 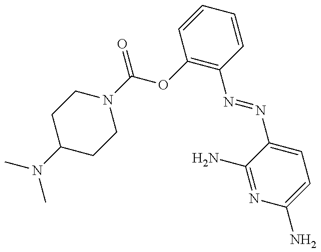 [SMILES: CN(C)C1CCN(C(=O)Oc2ccccc2/N=N/c2ccc(N)nc2N)CC1] |
| 43 | 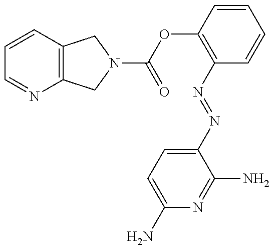 [SMILES: Nc1ccc(/N=N/c2ccccc2OC(=O)N2Cc3cccnc3C2)c(N)n1] |
| 44 | 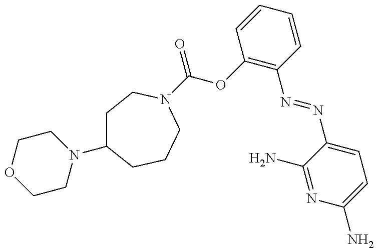 [SMILES: Nc1ccc(/N=N/c2ccccc2OC(=O)N2CCCC(N3CCOCC3)CC2)c(N)n1] |
| 45 | 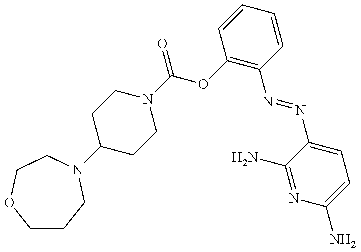 [SMILES: Nc1ccc(/N=N/c2ccccc2OC(=O)N2CCC(N3CCCOCC3)CC2)c(N)n1] |
| 46 | 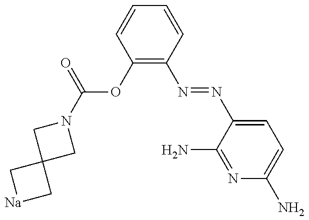 |
| 47 | 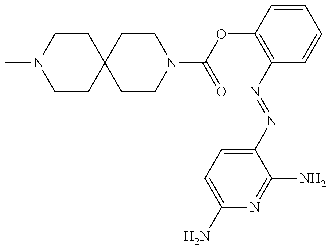 [SMILES: CN1CCC2(CC1)CCN(C(=O)Oc1ccccc1/N=N/c1ccc(N)nc1N)CC2] |
| 48 | 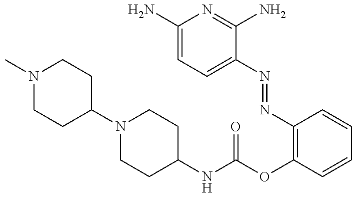 [SMILES: CN1CCC(N2CCC(NC(=O)Oc3ccccc3/N=N/c3ccc(N)nc3N)CC2)CC1] |
| 49 | 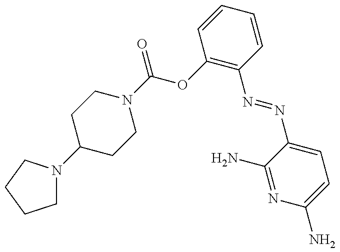 [SMILES: Nc1ccc(/N=N/c2ccccc2OC(=O)N2CCC(N3CCCC3)CC2)c(N)n1] |
| 50 | 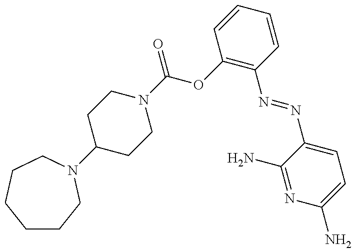 [SMILES: Nc1ccc(/N=N/c2ccccc2OC(=O)N2CCC(N3CCCCCC3)CC2)c(N)n1] |
| 51 | 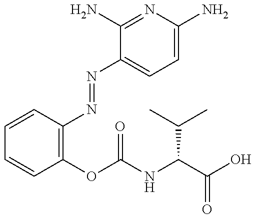 [SMILES: CC(C)[C@@H](NC(=O)Oc1ccccc1/N=N/c1ccc(N)nc1N)C(=O)O] |
| 52 | 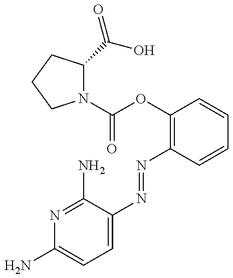 [SMILES: Nc1ccc(/N=N/c2ccccc2OC(=O)N2CCC[C@@H]2C(=O)O)c(N)n1] |
| 53 | 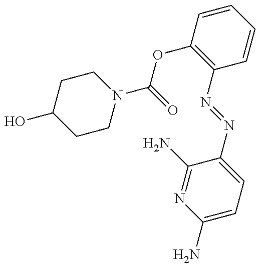 [SMILES: Nc1ccc(/N=N/c2ccccc2OC(=O)N2CCC(O)CC2)c(N)n1] |
| 54 | 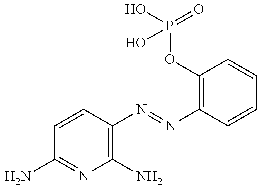 [SMILES: Nc1ccc(/N=N/c2ccccc2OP(=O)(O)O)c(N)n1] |
| 55 | 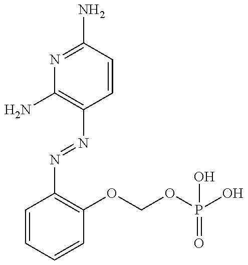 [SMILES: Nc1ccc(/N=N/c2ccccc2OCOP(=O)(O)O)c(N)n1] |
| 56 | 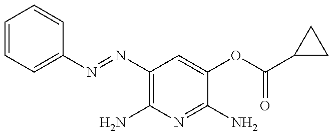 [SMILES: Nc1nc(N)c(OC(=O)C2CC2)cc1/N=N/c1ccccc1] |
| 57 | 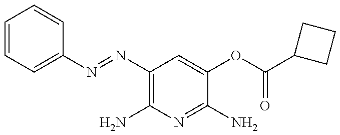 [SMILES: Nc1nc(N)c(OC(=O)C2CCC2)cc1/N=N/c1ccccc1] |
| 58 | 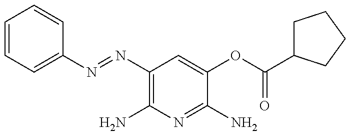 [SMILES: Nc1nc(N)c(OC(=O)C2CCCC2)cc1/N=N/c1ccccc1] |
| 59 | 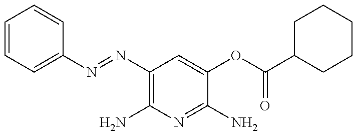 [SMILES: Nc1nc(N)c(OC(=O)C2CCCCC2)cc1/N=N/c1ccccc1] |
| 60 | 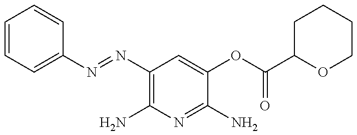 [SMILES: Nc1nc(N)c(OC(=O)C2CCCCO2)cc1/N=N/c1ccccc1] |
| 61 | 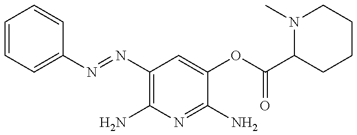 [SMILES: CN1CCCCC1C(=O)Oc1cc(/N=N/c2ccccc2)c(N)nc1N] |
| 62 | 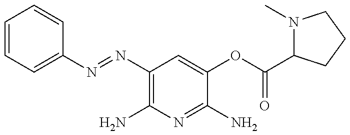 [SMILES: CN1CCCC1C(=O)Oc1cc(/N=N/c2ccccc2)c(N)nc1N] |
| 63 | 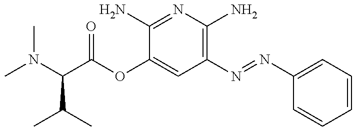 [SMILES: CC(C)[C@H](C(=O)Oc1cc(/N=N/c2ccccc2)c(N)nc1N)N(C)C] |
| 64 | 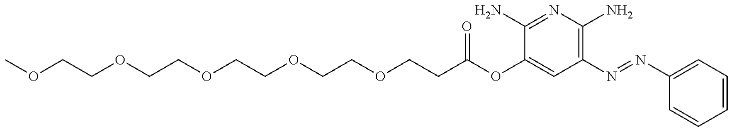 [SMILES: COCCOCCOCCOCCOCCC(=O)Oc1cc(/N=N/c2ccccc2)c(N)nc1N] |
| 65 | 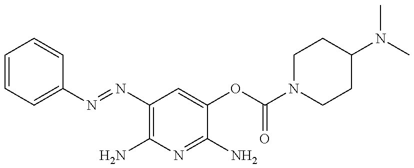 [SMILES: CN(C)C1CCN(C(=O)Oc2cc(/N=N/c3ccccc3)c(N)nc2N)CC1] |
| 66 | 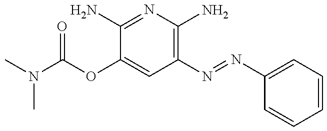 [SMILES: CN(C)C(=O)Oc1cc(/N=N/c2ccccc2)c(N)nc1N] |
| 67 | 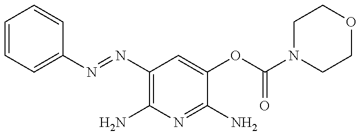 [SMILES: Nc1nc(N)c(OC(=O)N2CCOCC2)cc1/N=N/c1ccccc1] |
| 68 | 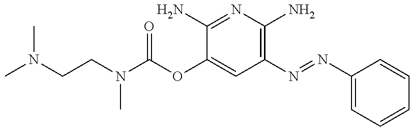 [SMILES: CN(C)CCN(C)C(=O)Oc1cc(/N=N/c2ccccc2)c(N)nc1N] |
| 69 | 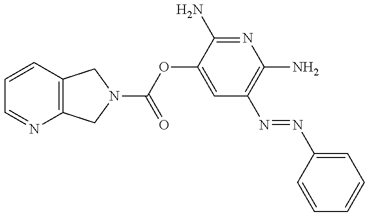 [SMILES: Nc1nc(N)c(OC(=O)N2Cc3cccnc3C2)cc1/N=N/c1ccccc1] |
| 70 | 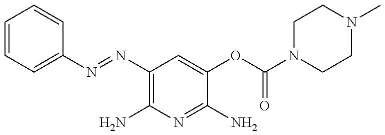 [SMILES: CN1CCN(C(=O)Oc2cc(/N=N/c3ccccc3)c(N)nc2N)CC1] |
| 71 | 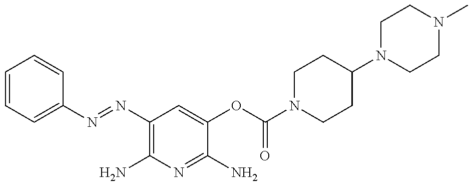 [SMILES: CN1CCN(C2CCN(C(=O)Oc3cc(/N=N/c4ccccc4)c(N)nc3N)CC2)CC1] |
| 72 | 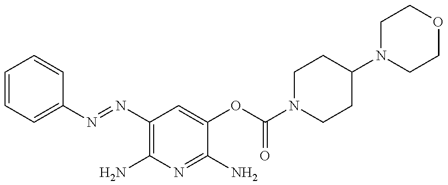 [SMILES: Nc1nc(N)c(OC(=O)N2CCC(N3CCOCC3)CC2)cc1/N=N/c1ccccc1] |
| 73 | 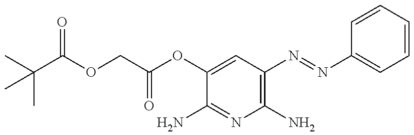 [SMILES: CC(C)(C)C(=O)OCC(=O)Oc1cc(/N=N/c2ccccc2)c(N)nc1N] |
| 74 | 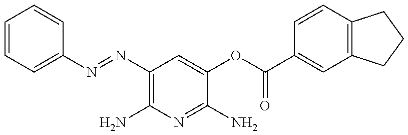 [SMILES: Nc1nc(N)c(OC(=O)c2ccc3c(c2)CCC3)cc1/N=N/c1ccccc1] |
| 75 | 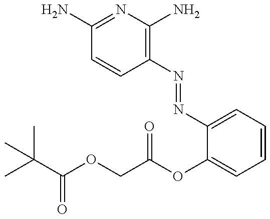 [SMILES: CC(C)(C)C(=O)OCC(=O)Oc1ccccc1/N=N/c1ccc(N)nc1N] |
| 76 | 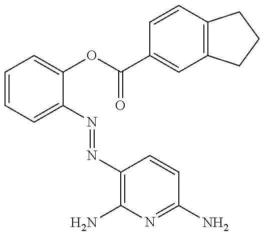 [SMILES: Nc1ccc(/N=N/c2ccccc2OC(=O)c2ccc3c(c2)CCC3)c(N)n1] |
| 77 | 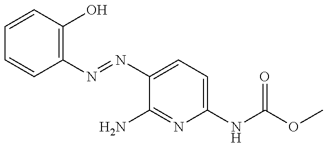 [SMILES: COC(=O)Nc1ccc(/N=N/c2ccccc2O)c(N)n1] |
| 78 | 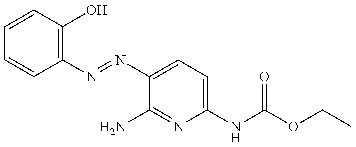 [SMILES: CCOC(=O)Nc1ccc(/N=N/c2ccccc2O)c(N)n1] |
| 79 | 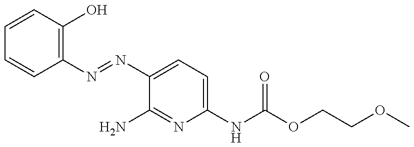 [SMILES: COCCOC(=O)Nc1ccc(/N=N/c2ccccc2O)c(N)n1] |
| 80 | 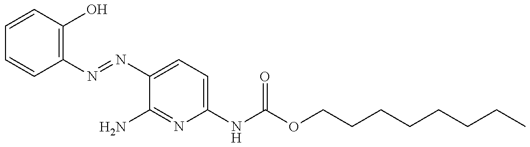 [SMILES: CCCCCCCCOC(=O)Nc1ccc(/N=N/c2ccccc2O)c(N)n1] |
| 81 | 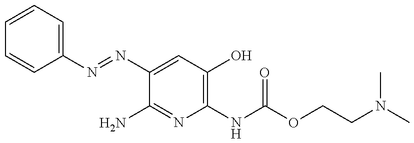 [SMILES: CN(C)CCOC(=O)Nc1nc(N)c(/N=N/c2ccccc2)cc1O] |
| 82 | 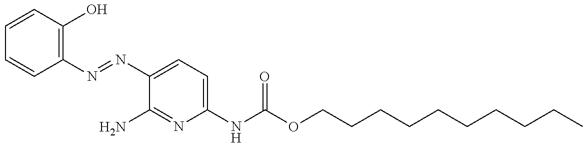 [SMILES: CCCCCCCCCCOC(=O)Nc1ccc(/N=N/c2ccccc2O)c(N)n1] |
| 83 | 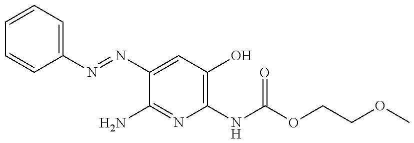 [SMILES: COCCOC(=O)Nc1nc(N)c(/N=N/c2ccccc2)cc1O] |
| 84 | 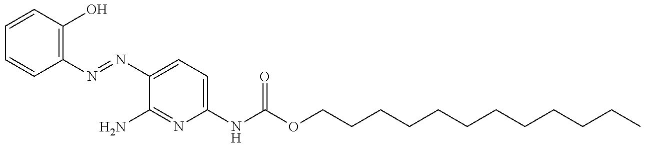 [SMILES: CCCCCCCCCCCCOC(=O)Nc1ccc(/N=N/c2ccccc2O)c(N)n1] |
| 85 | 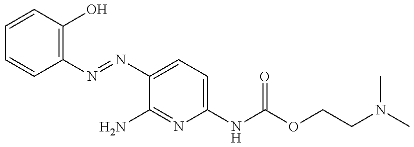 [SMILES: CN(C)CCOC(=O)Nc1ccc(/N=N/c2ccccc2O)c(N)n1] |
| 86 | 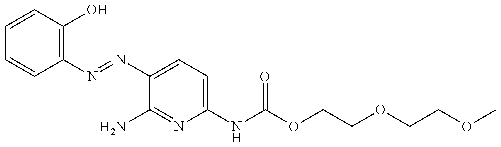 [SMILES: COCCOCCOC(=O)Nc1ccc(/N=N/c2ccccc2O)c(N)n1] |
| 87 | 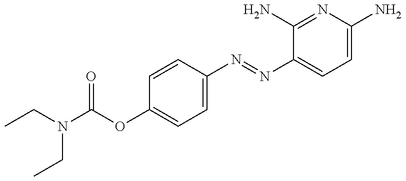 [SMILES: CCN(CC)C(=O)Oc1ccc(/N=N/c2ccc(N)nc2N)cc1] |
| 88 | 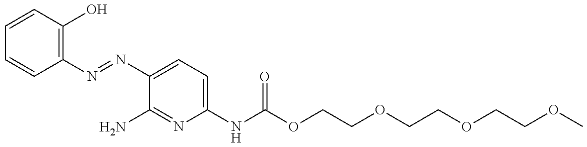 [SMILES: COCCOCCOCCOC(=O)Nc1ccc(/N=N/c2ccccc2O)c(N)n1] |
| 89 | 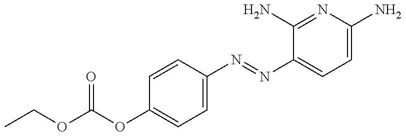 [SMILES: CCOC(=O)Oc1ccc(/N=N/c2ccc(N)nc2N)cc1] |
| 90 | 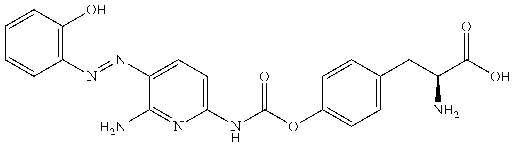 [SMILES: Nc1nc(NC(=O)Oc2ccc(C[C@H](N)C(=O)O)cc2)ccc1/N=N/c1ccccc1O] |
| 91 | 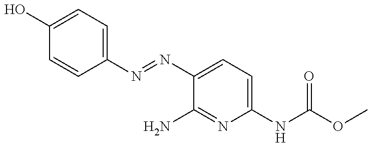 [SMILES: COC(=O)Nc1ccc(/N=N/c2ccc(O)cc2)c(N)n1] |
| 92 | 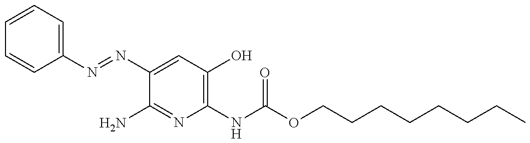 [SMILES: CCCCCCCCOC(=O)Nc1nc(N)c(/N=N/c2ccccc2)cc1O] |
| 93 | 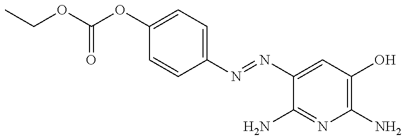 [SMILES: CCOC(=O)Oc1ccc(/N=N/c2cc(O)c(N)nc2N)cc1] |
| 94 | 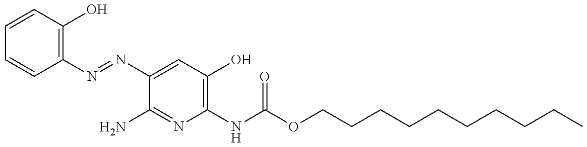 [SMILES: CCCCCCCCCCOC(=O)Nc1nc(N)c(/N=N/c2ccccc2O)cc1O] |
| 95 | 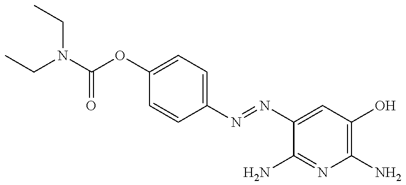 [SMILES: CCN(CC)C(=O)Oc1ccc(/N=N/c2cc(O)c(N)nc2N)cc1] |
| 96 | 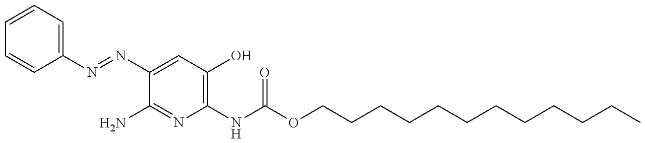 [SMILES: CCCCCCCCCCCCOC(=O)Nc1nc(N)c(/N=N/c2ccccc2)cc1O] |
| 97 | 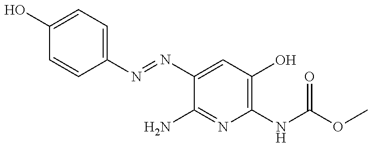 [SMILES: COC(=O)Nc1nc(N)c(/N=N/c2ccc(O)cc2)cc1O] |
| 98 | 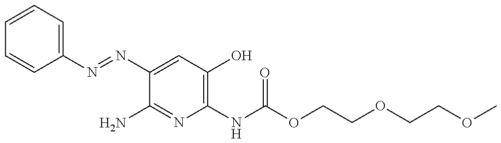 [SMILES: COCCOCCOC(=O)Nc1nc(N)c(/N=N/c2ccccc2)cc1O] |
| 99 | 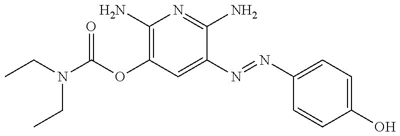 [SMILES: CCN(CC)C(=O)Oc1cc(/N=N/c2ccc(O)cc2)c(N)nc1N] |
| 100 | 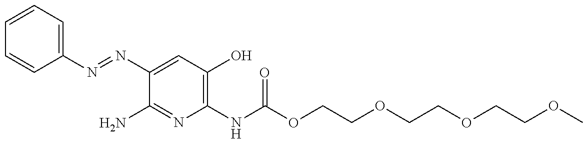 [SMILES: COCCOCCOCCOC(=O)Nc1nc(N)c(/N=N/c2ccccc2)cc1O] |
| 101 | 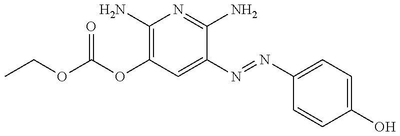 [SMILES: CCOC(=O)Oc1cc(/N=N/c2ccc(O)cc2)c(N)nc1N] |
| 102 | 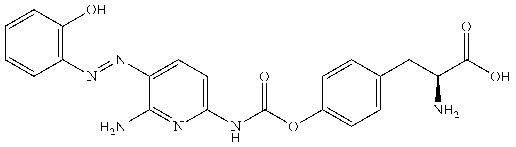 [SMILES: Nc1nc(NC(=O)Oc2ccc(C[C@H](N)C(=O)O)cc2)ccc1/N=N/c1ccccc1O] |
| 103 | 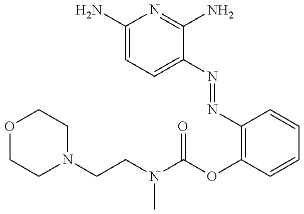 [SMILES: CN(CCN1CCOCC1)C(=O)Oc1ccccc1/N=N/c1ccc(N)nc1N] |
| 104 | 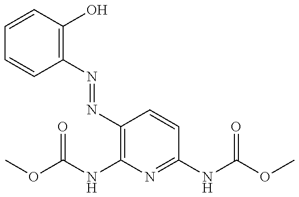 [SMILES: COC(=O)Nc1ccc(/N=N/c2ccccc2O)c(NC(=O)OC)n1] |
| 105 | 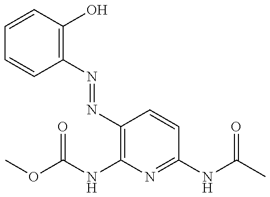 [SMILES: COC(=O)Nc1nc(NC(C)=O)ccc1/N=N/c1ccccc1O] |
| 106 | 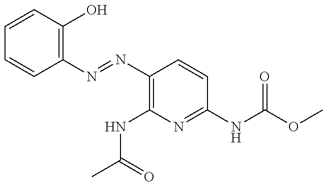 [SMILES: COC(=O)Nc1ccc(/N=N/c2ccccc2O)c(NC(C)=O)n1] |
| 107 | 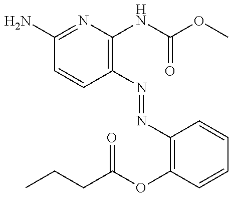 [SMILES: CCCC(=O)Oc1ccccc1/N=N/c1ccc(N)nc1NC(=O)OC] |
| 108 | 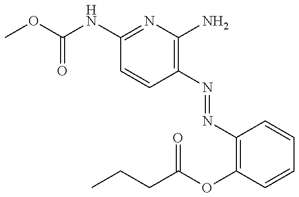 [SMILES: CCCC(=O)Oc1ccccc1/N=N/c1ccc(NC(=O)OC)nc1N] |
| 109 | 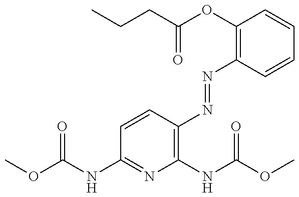 [SMILES: CCCC(=O)Oc1ccccc1/N=N/c1ccc(NC(=O)OC)nc1NC(=O)OC] |
| 110 | 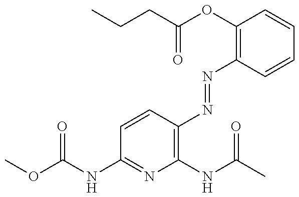 [SMILES: CCCC(=O)Oc1ccccc1/N=N/c1ccc(NC(=O)OC)nc1NC(C)=O] |
| 111 | 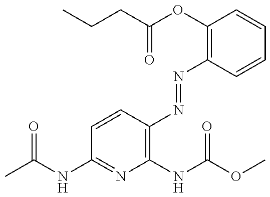 [SMILES: CCCC(=O)Oc1ccccc1/N=N/c1ccc(NC(C)=O)nc1NC(=O)OC] |
| 112 | 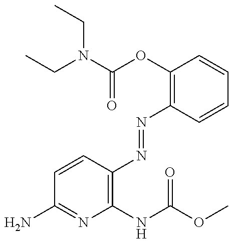 [SMILES: CCN(CC)C(=O)Oc1ccccc1/N=N/c1ccc(N)nc1NC(=O)OC] |
| 113 | 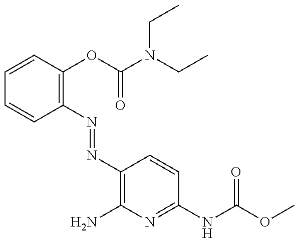 [SMILES: CCN(CC)C(=O)Oc1ccccc1/N=N/c1ccc(NC(=O)OC)nc1N] |
| 114 | 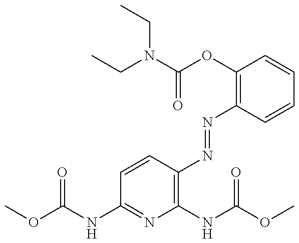 [SMILES: CCN(CC)C(=O)Oc1ccccc1/N=N/c1ccc(NC(=O)OC)nc1NC(=O)OC] |
| 115 | 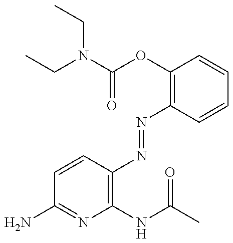 [SMILES: CCN(CC)C(=O)Oc1ccccc1/N=N/c1ccc(N)nc1NC(C)=O] |
| 116 | 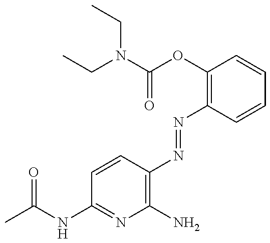 [SMILES: CCN(CC)C(=O)Oc1ccccc1/N=N/c1ccc(NC(C)=O)nc1N] |
| 117 | 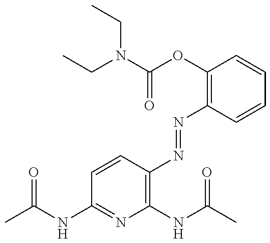 [SMILES: CCN(CC)C(=O)Oc1ccccc1/N=N/c1ccc(NC(C)=O)nc1NC(C)=O] |
| 118 | 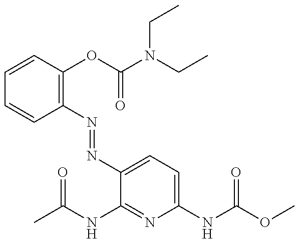 [SMILES: CCN(CC)C(=O)Oc1ccccc1/N=N/c1ccc(NC(=O)OC)nc1NC(C)=O] |
| 119 | 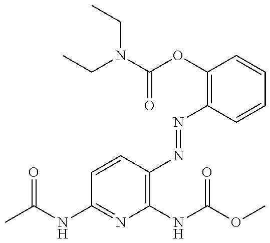 [SMILES: CCN(CC)C(=O)Oc1ccccc1/N=N/c1ccc(NC(C)=O)nc1NC(=O)OC] |
| 120 | 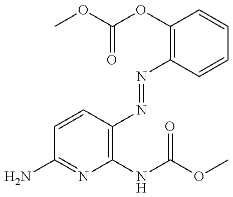 [SMILES: COC(=O)Nc1nc(N)ccc1/N=N/c1ccccc1OC(=O)OC] |
| 121 | 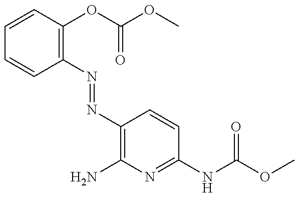 [SMILES: COC(=O)Nc1ccc(/N=N/c2ccccc2OC(=O)OC)c(N)n1] |
| 122 | 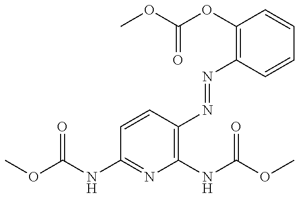 [SMILES: COC(=O)Nc1ccc(/N=N/c2ccccc2OC(=O)OC)c(NC(=O)OC)n1] |
| 123 | 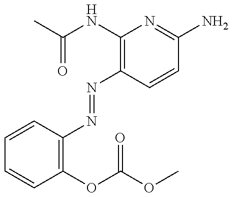 [SMILES: COC(=O)Oc1ccccc1/N=N/c1ccc(N)nc1NC(C)=O] |
| 124 | 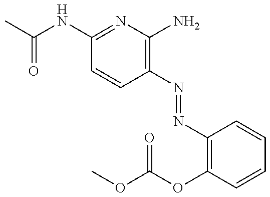 [SMILES: COC(=O)Oc1ccccc1/N=N/c1ccc(NC(C)=O)nc1N] |
| 125 | 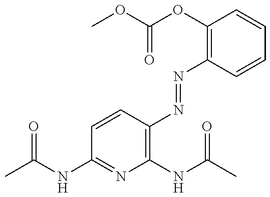 [SMILES: COC(=O)Oc1ccccc1/N=N/c1ccc(NC(C)=O)nc1NC(C)=O] |
| 126 | 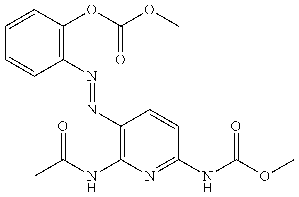 [SMILES: COC(=O)Nc1ccc(/N=N/c2ccccc2OC(=O)OC)c(NC(C)=O)n1] |
| 127 | 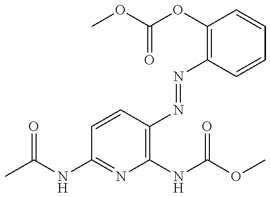 [SMILES: COC(=O)Nc1nc(NC(C)=O)ccc1/N=N/c1ccccc1OC(=O)OC] |
| 128 | 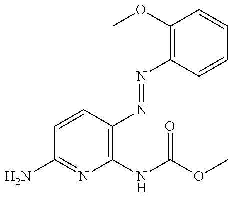 [SMILES: COC(=O)Nc1nc(N)ccc1/N=N/c1ccccc1OC] |
| 129 | 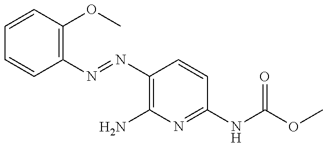 [SMILES: COC(=O)Nc1ccc(/N=N/c2ccccc2OC)c(N)n1] |
| 130 | 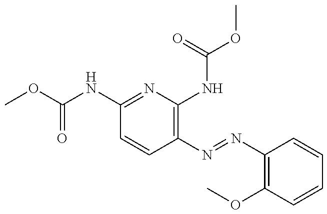 [SMILES: COC(=O)Nc1ccc(/N=N/c2ccccc2OC)c(NC(=O)OC)n1] |
| 131 | 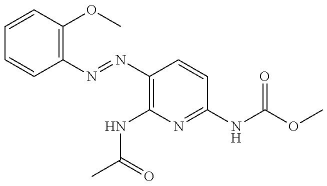 [SMILES: COC(=O)Nc1ccc(/N=N/c2ccccc2OC)c(NC(C)=O)n1] |
| 132 | 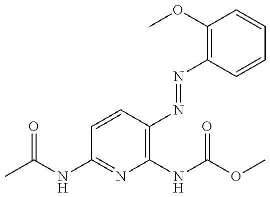 [SMILES: COC(=O)Nc1nc(NC(C)=O)ccc1/N=N/c1ccccc1OC] |
| 133 | 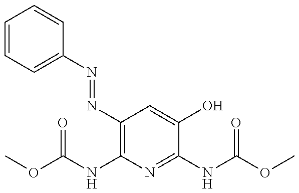 [SMILES: COC(=O)Nc1nc(NC(=O)OC)c(/N=N/c2ccccc2)cc1O] |
| 134 | 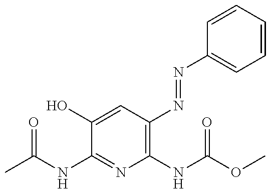 [SMILES: COC(=O)Nc1nc(NC(C)=O)c(O)cc1/N=N/c1ccccc1] |
| 135 | 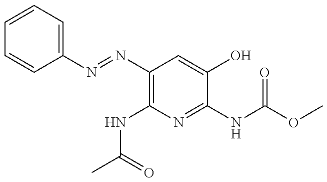 [SMILES: COC(=O)Nc1nc(NC(C)=O)c(/N=N/c2ccccc2)cc1O] |
| 136 | 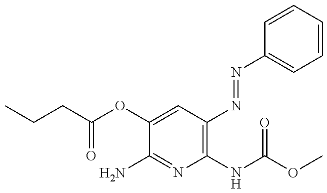 [SMILES: CCCC(=O)Oc1cc(/N=N/c2ccccc2)c(NC(=O)OC)nc1N] |
| 137 | 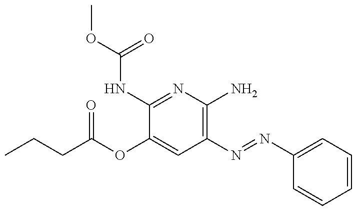 [SMILES: CCCC(=O)Oc1cc(/N=N/c2ccccc2)c(N)nc1NC(=O)OC] |
| 138 | 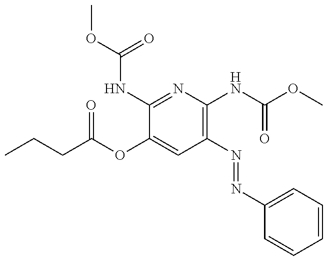 [SMILES: CCCC(=O)Oc1cc(/N=N/c2ccccc2)c(NC(=O)OC)nc1NC(=O)OC] |
| 139 | 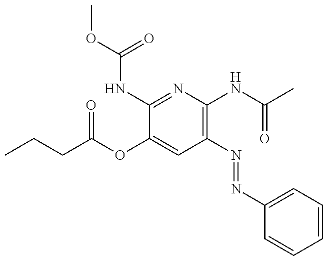 [SMILES: CCCC(=O)Oc1cc(/N=N/c2ccccc2)c(NC(C)=O)nc1NC(=O)OC] |
| 140 | 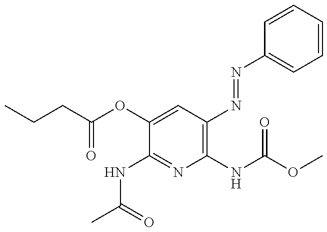 [SMILES: CCCC(=O)Oc1cc(/N=N/c2ccccc2)c(NC(=O)OC)nc1NC(C)=O] |
| 141 | 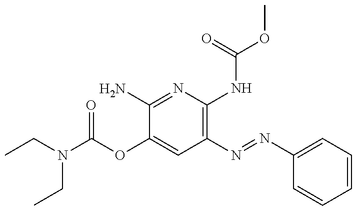 [SMILES: CCN(CC)C(=O)Oc1cc(/N=N/c2ccccc2)c(NC(=O)OC)nc1N] |
| 142 | 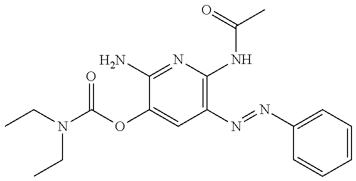 [SMILES: CCN(CC)C(=O)Oc1cc(/N=N/c2ccccc2)c(NC(C)=O)nc1N] |
| 143 | 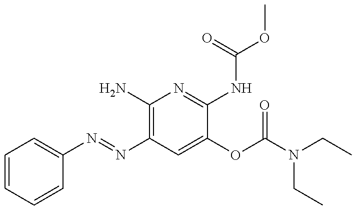 [SMILES: CCN(CC)C(=O)Oc1cc(/N=N/c2ccccc2)c(N)nc1NC(=O)OC] |
| 144 | 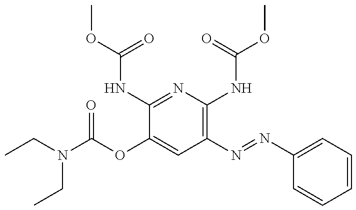 [SMILES: CCN(CC)C(=O)Oc1cc(/N=N/c2ccccc2)c(NC(=O)OC)nc1NC(=O)OC] |
| 145 | 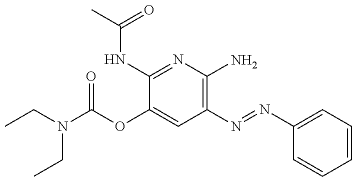 [SMILES: CCN(CC)C(=O)Oc1cc(/N=N/c2ccccc2)c(N)nc1NC(C)=O] |
| 146 | 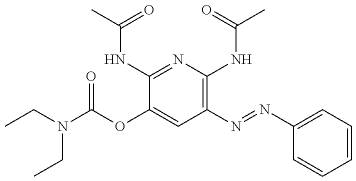 [SMILES: CCN(CC)C(=O)Oc1cc(/N=N/c2ccccc2)c(NC(C)=O)nc1NC(C)=O] |
| 147 | 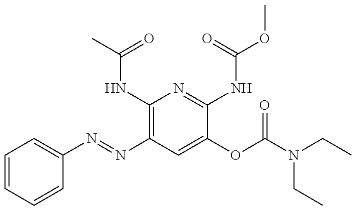 [SMILES: CCN(CC)C(=O)Oc1cc(/N=N/c2ccccc2)c(NC(C)=O)nc1NC(=O)OC] |
| 148 | 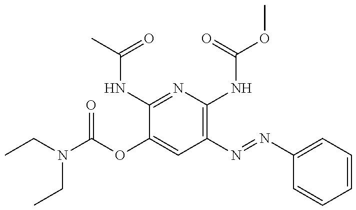 [SMILES: CCN(CC)C(=O)Oc1cc(/N=N/c2ccccc2)c(NC(=O)OC)nc1NC(C)=O] |
| 149 | 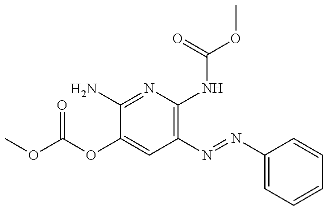 [SMILES: COC(=O)Nc1nc(N)c(OC(=O)OC)cc1/N=N/c1ccccc1] |
| 150 | 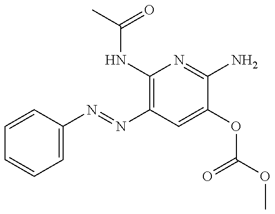 [SMILES: COC(=O)Oc1cc(/N=N/c2ccccc2)c(NC(C)=O)nc1N] |
| 151 | 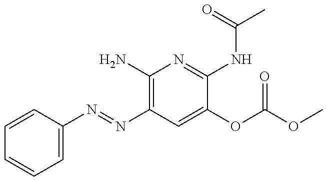 [SMILES: COC(=O)Oc1cc(/N=N/c2ccccc2)c(N)nc1NC(C)=O] |
| 152 | 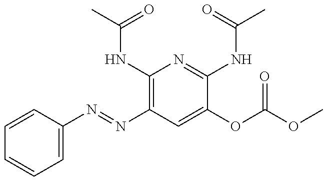 [SMILES: COC(=O)Oc1cc(/N=N/c2ccccc2)c(NC(C)=O)nc1NC(C)=O] |
| 153 | 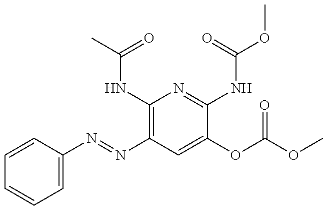 [SMILES: COC(=O)Nc1nc(NC(C)=O)c(/N=N/c2ccccc2)cc1OC(=O)OC] |
| 154 | 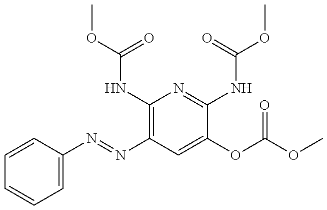 [SMILES: COC(=O)Nc1nc(NC(=O)OC)c(OC(=O)OC)cc1/N=N/c1ccccc1] |
| 155 | 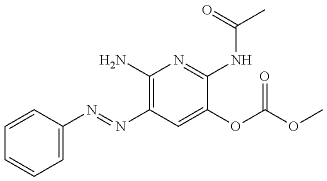 [SMILES: COC(=O)Oc1cc(/N=N/c2ccccc2)c(N)nc1NC(C)=O] |
| 156 | 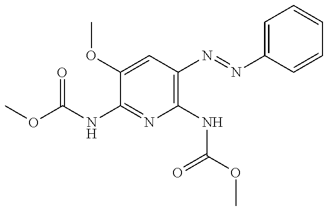 [SMILES: COC(=O)Nc1nc(NC(=O)OC)c(OC)cc1/N=N/c1ccccc1] |
| 157 | 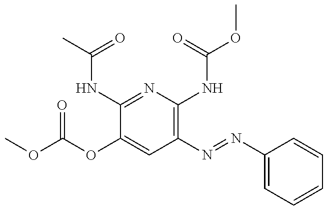 [SMILES: COC(=O)Nc1nc(NC(C)=O)c(OC(=O)OC)cc1/N=N/c1ccccc1] |
| 158 | 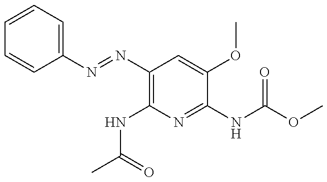 [SMILES: COC(=O)Nc1nc(NC(C)=O)c(/N=N/c2ccccc2)cc1OC] |
| 159 | 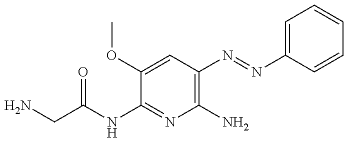 [SMILES: COc1cc(/N=N/c2ccccc2)c(N)nc1NC(=O)CN] |
| 160 | 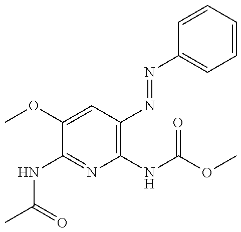 [SMILES: COC(=O)Nc1nc(NC(C)=O)c(OC)cc1/N=N/c1ccccc1] |
| 161 | 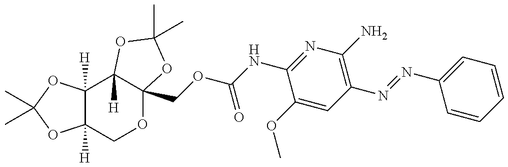 |
| 162 | 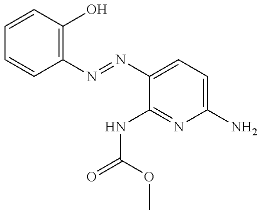 [SMILES: COC(=O)Nc1nc(N)ccc1/N=N/c1ccccc1O] |
| 163 | 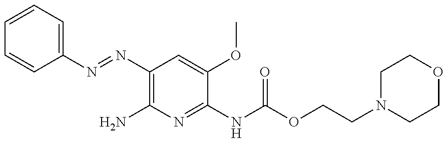 [SMILES: COc1cc(/N=N/c2ccccc2)c(N)nc1NC(=O)OCCN1CCOCC1] |
| 164 | 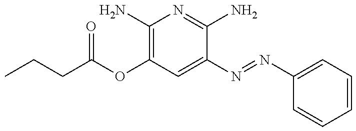 [SMILES: CCCC(=O)Oc1cc(/N=N/c2ccccc2)c(N)nc1N] |
| 165 | 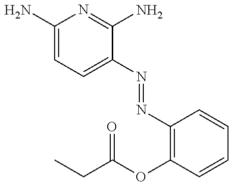 [SMILES: CCC(=O)Oc1ccccc1/N=N/c1ccc(N)nc1N] |
| 166 | 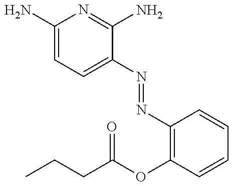 [SMILES: CCCC(=O)Oc1ccccc1/N=N/c1ccc(N)nc1N] |
| 167 | 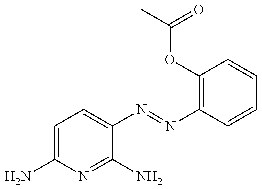 [SMILES: CC(=O)Oc1ccccc1/N=N/c1ccc(N)nc1N] |
| 168 | 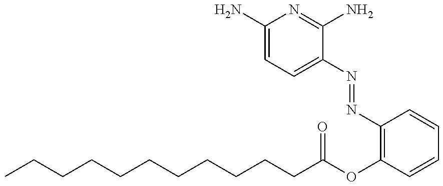 [SMILES: CCCCCCCCCCCC(=O)Oc1ccccc1/N=N/c1ccc(N)nc1N] |
| 169 | 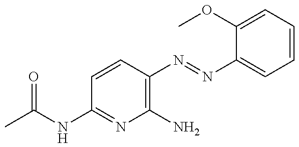 [SMILES: COc1ccccc1/N=N/c1ccc(NC(C)=O)nc1N] |
| 170 | 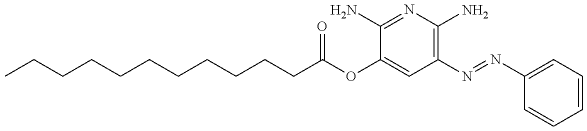 [SMILES: CCCCCCCCCCCC(=O)Oc1cc(/N=N/c2ccccc2)c(N)nc1N] |
| 171 | 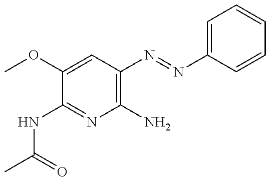 [SMILES: COc1cc(/N=N/c2ccccc2)c(N)nc1NC(C)=O] |
| 172 | 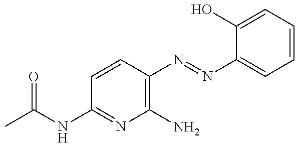 [SMILES: CC(=O)Nc1ccc(/N=N/c2ccccc2O)c(N)n1] |
| 173 | 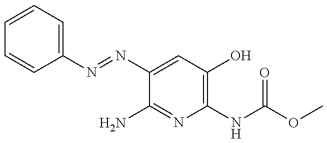 [SMILES: COC(=O)Nc1nc(N)c(/N=N/c2ccccc2)cc1O] |
| 174 | 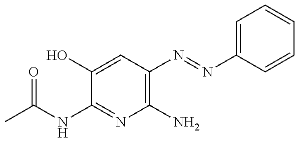 [SMILES: CC(=O)Nc1nc(N)c(/N=N/c2ccccc2)cc1O] |
| 175 | 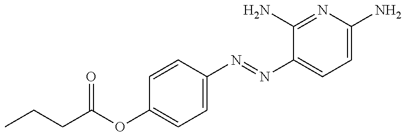 [SMILES: CCCC(=O)Oc1ccc(/N=N/c2ccc(N)nc2N)cc1] |
| 176 | 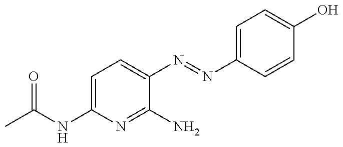 [SMILES: CC(=O)Nc1ccc(/N=N/c2ccc(O)cc2)c(N)n1] |
| 177 | 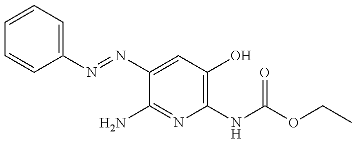 [SMILES: CCOC(=O)Nc1nc(N)c(/N=N/c2ccccc2)cc1O] |
| 178 | 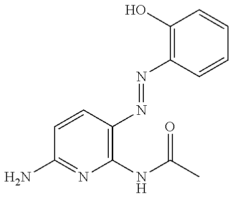 [SMILES: CC(=O)Nc1nc(N)ccc1/N=N/c1ccccc1O] |
| 179 | 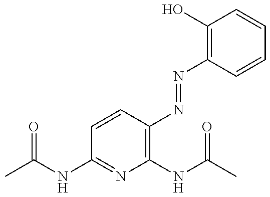 [SMILES: CC(=O)Nc1ccc(/N=N/c2ccccc2O)c(NC(C)=O)n1] |
| 180 | 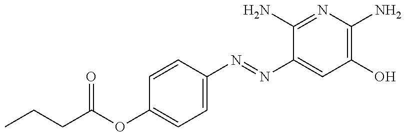 [SMILES: CCCC(=O)Oc1ccc(/N=N/c2cc(O)c(N)nc2N)cc1] |
| 181 | 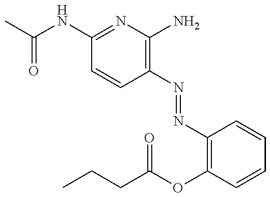 [SMILES: CCCC(=O)Oc1ccccc1/N=N/c1ccc(NC(C)=O)nc1N] |
| 182 | 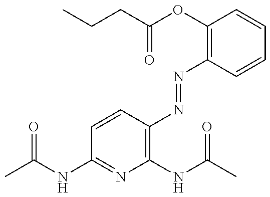 [SMILES: CCCC(=O)Oc1ccccc1/N=N/c1ccc(NC(C)=O)nc1NC(C)=O] |
| 183 | 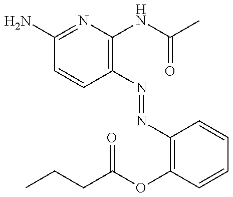 [SMILES: CCCC(=O)Oc1ccccc1/N=N/c1ccc(N)nc1NC(C)=O] |
| 184 | 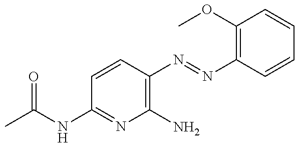 [SMILES: COc1ccccc1/N=N/c1ccc(NC(C)=O)nc1N] |
| 185 | 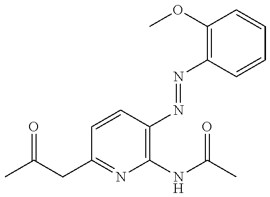 [SMILES: COc1ccccc1/N=N/c1ccc(CC(C)=O)nc1NC(C)=O] |
| 186 | 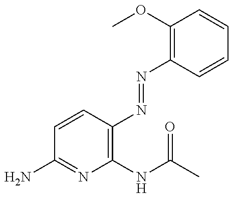 [SMILES: COc1ccccc1/N=N/c1ccc(N)nc1NC(C)=O] |
| 187 | 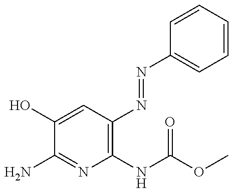 [SMILES: COC(=O)Nc1nc(N)c(O)cc1/N=N/c1ccccc1] |
| 188 | 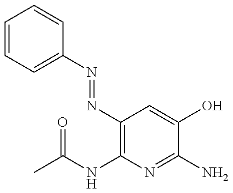 [SMILES: CC(=O)Nc1nc(N)c(O)cc1/N=N/c1ccccc1] |
| 189 | 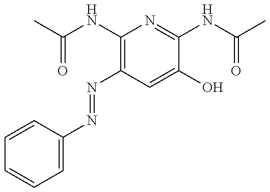 [SMILES: CC(=O)Nc1nc(NC(C)=O)c(/N=N/c2ccccc2)cc1O] |
| 190 | 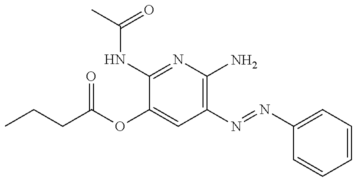 [SMILES: CCCC(=O)Oc1cc(/N=N/c2ccccc2)c(N)nc1NC(C)=O] |
| 191 | 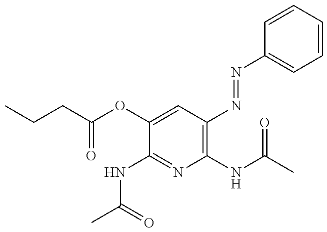 [SMILES: CCCC(=O)Oc1cc(/N=N/c2ccccc2)c(NC(C)=O)nc1NC(C)=O] |
| 192 | 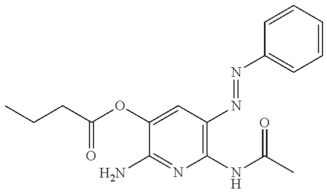 [SMILES: CCCC(=O)Oc1cc(/N=N/c2ccccc2)c(NC(C)=O)nc1N] |
| 193 | 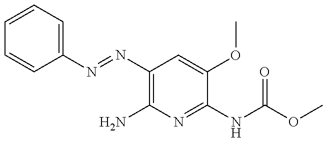 [SMILES: COC(=O)Nc1nc(N)c(/N=N/c2ccccc2)cc1OC] |
| 194 | 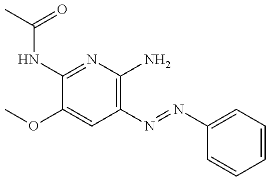 [SMILES: COc1cc(/N=N/c2ccccc2)c(N)nc1NC(C)=O] |
| 195 | 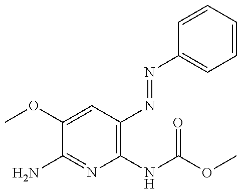 |
| 196 | 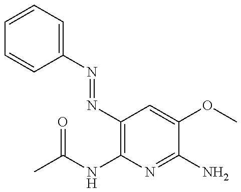 [SMILES: COc1cc(/N=N/c2ccccc2)c(NC(C)=O)nc1N] |
| 197 | 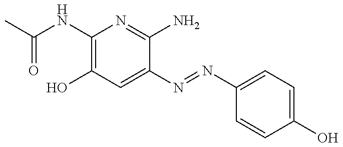 [SMILES: CC(=O)Nc1nc(N)c(/N=N/c2ccc(O)cc2)cc1O] |
| 198 | 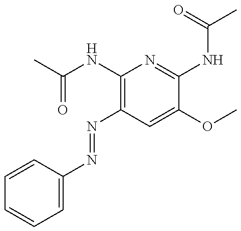 [SMILES: COc1cc(/N=N/c2ccccc2)c(NC(C)=O)nc1NC(C)=O] |
| 199 | 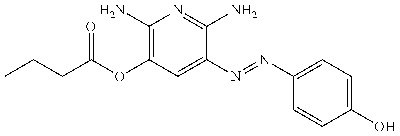 [SMILES: CCCC(=O)Oc1cc(/N=N/c2ccc(O)cc2)c(N)nc1N] |

In one embodiment, the present invention relates to compounds of general formula (IX), pharmaceutically acceptable salts or solvates thereof:

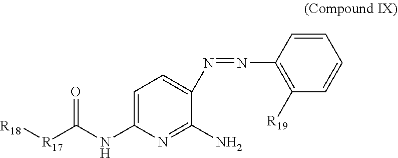

wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₁₉ is selected from —O—R₂₃—O—C(O)—R₂₄, —O—C(O)—R₂₇, and —O—R₂₃—R₂₈, wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group; R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group.

In one embodiment, the present invention relates to compounds of general formula (IX-A), pharmaceutically acceptable salts or solvates thereof:

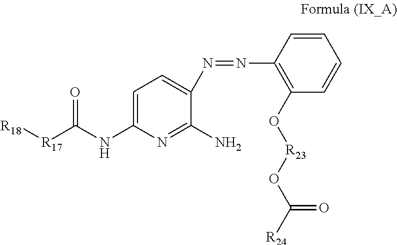

wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
Wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group.

In one embodiment the present invention provides at least one compound selected from the group consisting of:

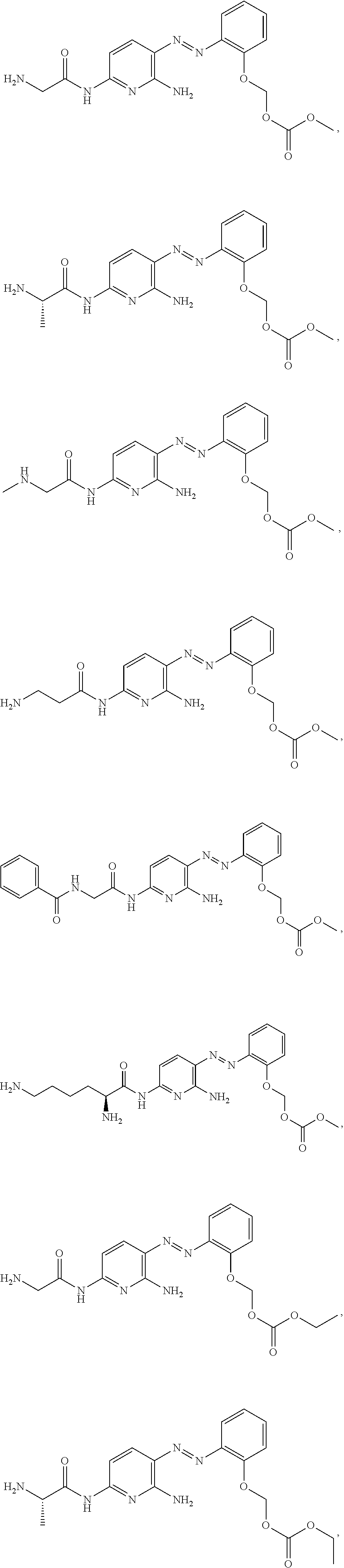

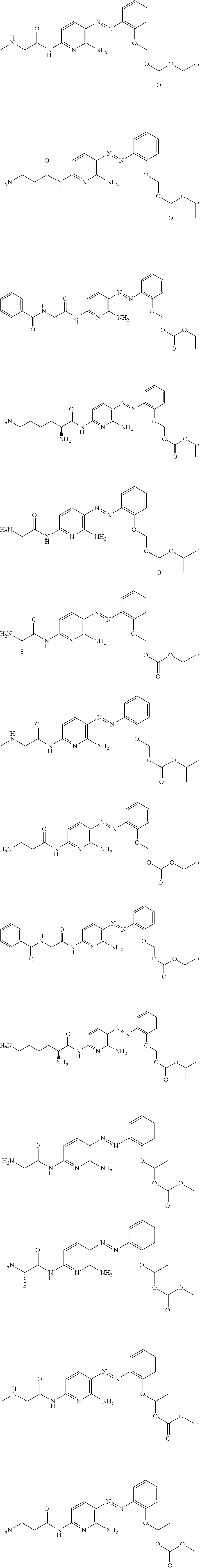

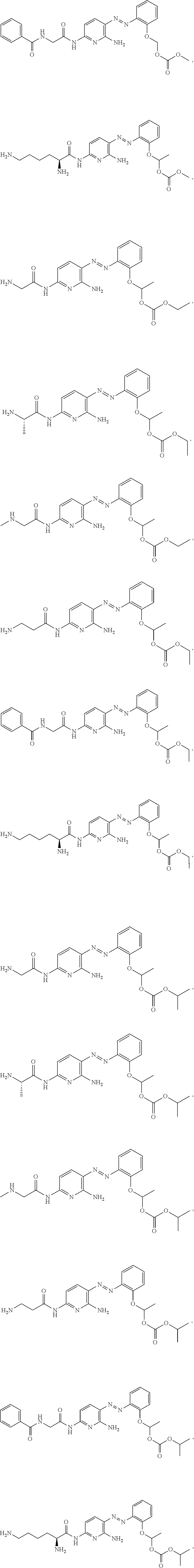

In one embodiment, the present invention relates to compounds of general formula (IX-B), pharmaceutically acceptable salts or solvates thereof:

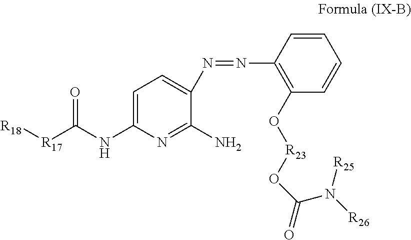

wherein R₁₇ is selected from optionally substituted linear or branched alkylene group, wherein alkylene is optionally substituted by amino, alkylamino and dialkylamino group; Rig is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group; wherein R₂₃ is optionally substituted linear or branched alkylene; wherein R₂₅ and R₂₆ are each independently selected from hydrogen, optionally substituted alkyl and aryl group, wherein alkyl and aryl is optionally substituted with OH, SH, F, Cl, Br, I, and optionally substituted, hydroxyalkyl, amino group; or R₂₅ and R₂₆ is taken together form an optionally substituted heterocycloalkyl ring, wherein the heterocycloalkyl ring is optionally substituted by optionally substituted alkyl, hydroxyalkyl group, OH, SH, F, Cl, Br, I, and optionally substituted amino group.

In one embodiment the present invention provides at least one compound selected from the group consisting of:

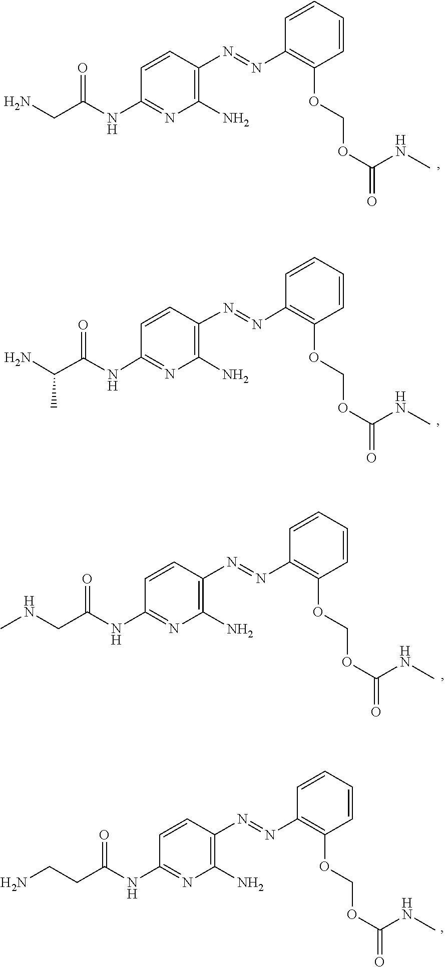

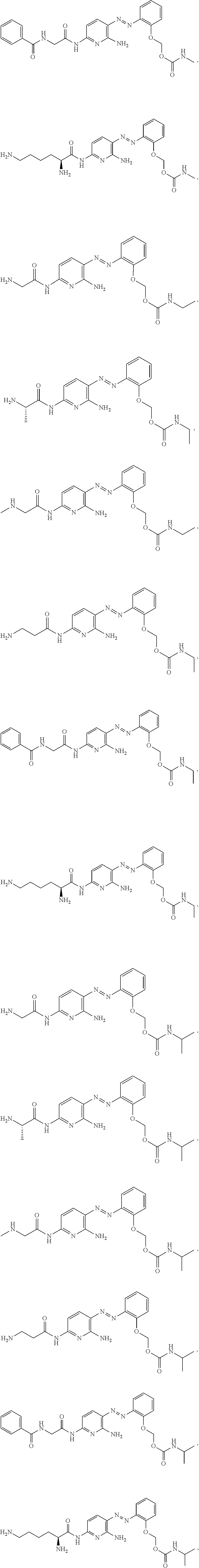

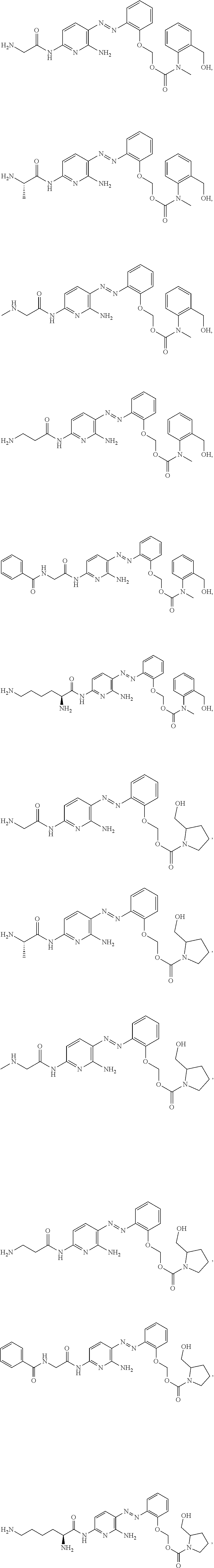

In one embodiment, the present invention relates to compounds of general formula (IX-C), pharmaceutically acceptable salts or solvates thereof:

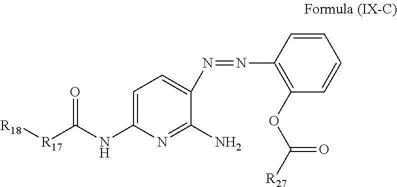

wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted
alkyl, —C(O)—R₂₂, wherein R₂₂ is selected from optionally substituted alkyl, cycloalkyl, heterocycloalkyl, heteroaryl, and aryl group;

R₂₇ is selected from optionally substituted alkyl, cycloalkyl, heterocycloalkyl, heteroaryl, aryl and —N(R₂₅R₂₆) group; wherein R₂₅ and R₂₆ are each independently selected from hydrogen, optionally substituted alkyl and aryl group, wherein alkyl and aryl is optionally substituted with OH, SH, F, Cl, Br, I, and optionally substituted, hydroxyalkyl, amino group; or R₂₅ and R₂₆ is taken together form an optionally substituted heterocycloalkyl ring, wherein the heterocycloalkyl ring is optionally substituted by optionally substituted alkyl, hydroxyalkyl group, OH, SH, F, Cl, Br, I, and optionally substituted amino group.

In one embodiment the present invention provides at least one compound selected from the group consisting of:

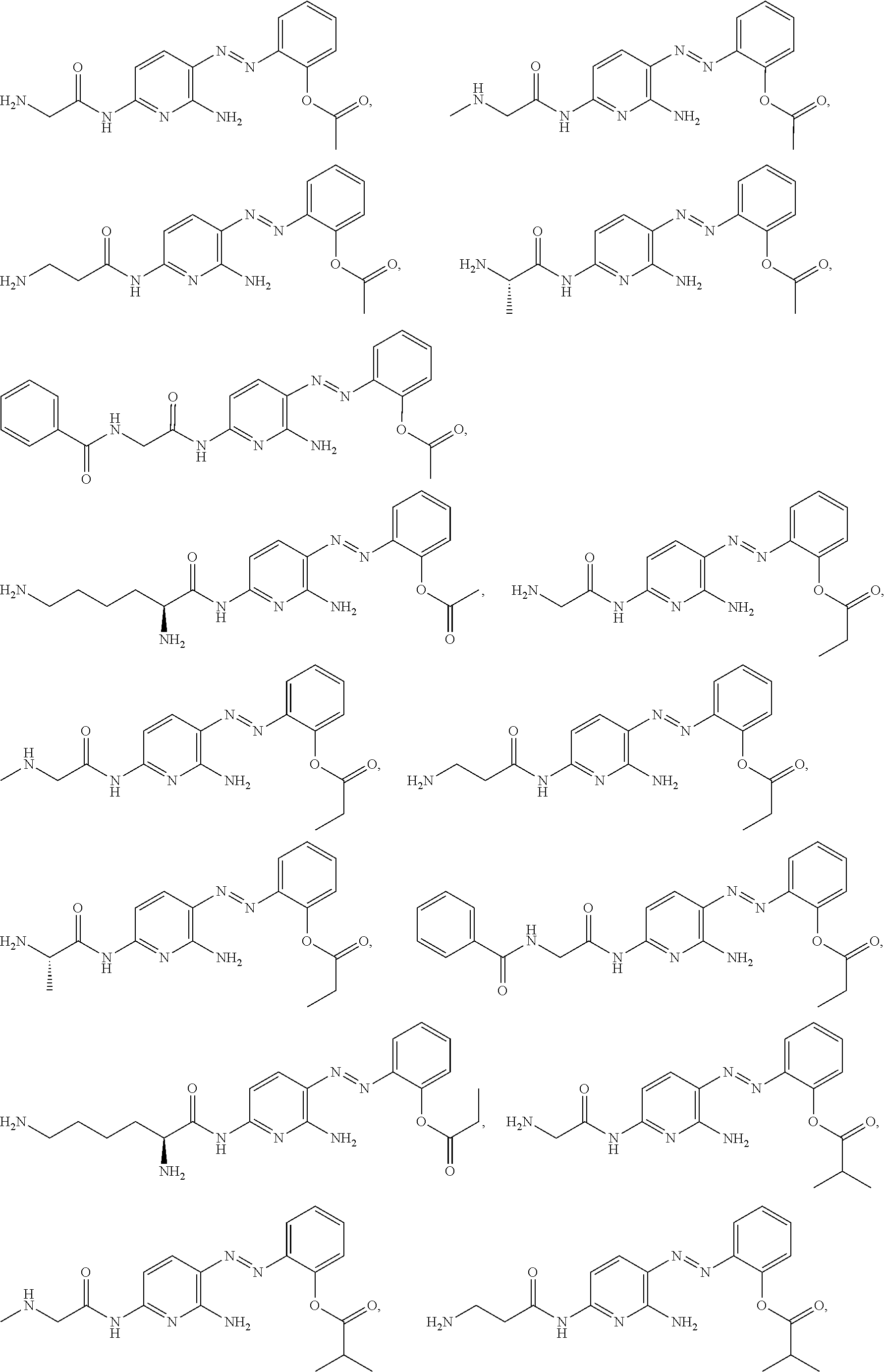

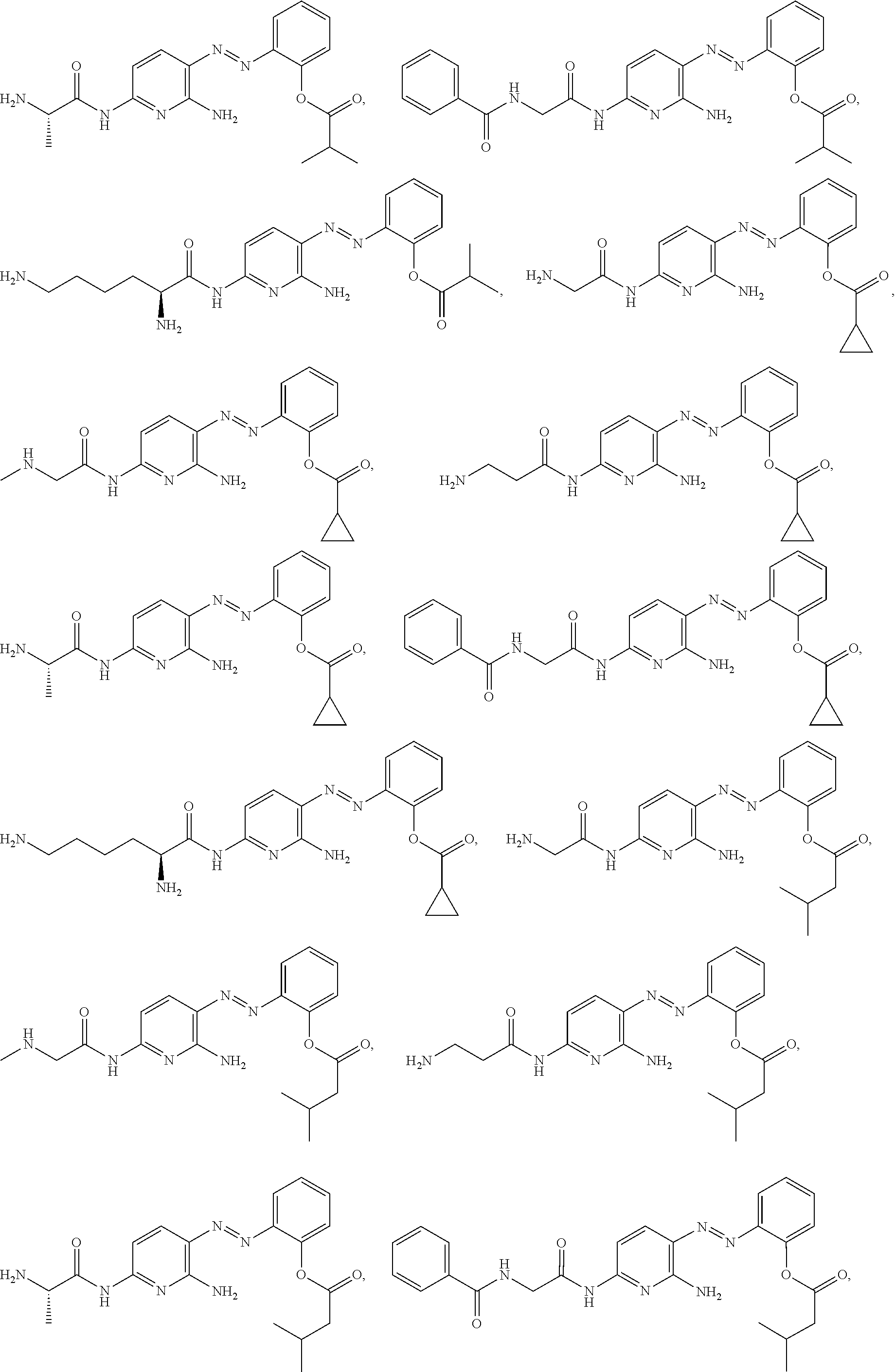

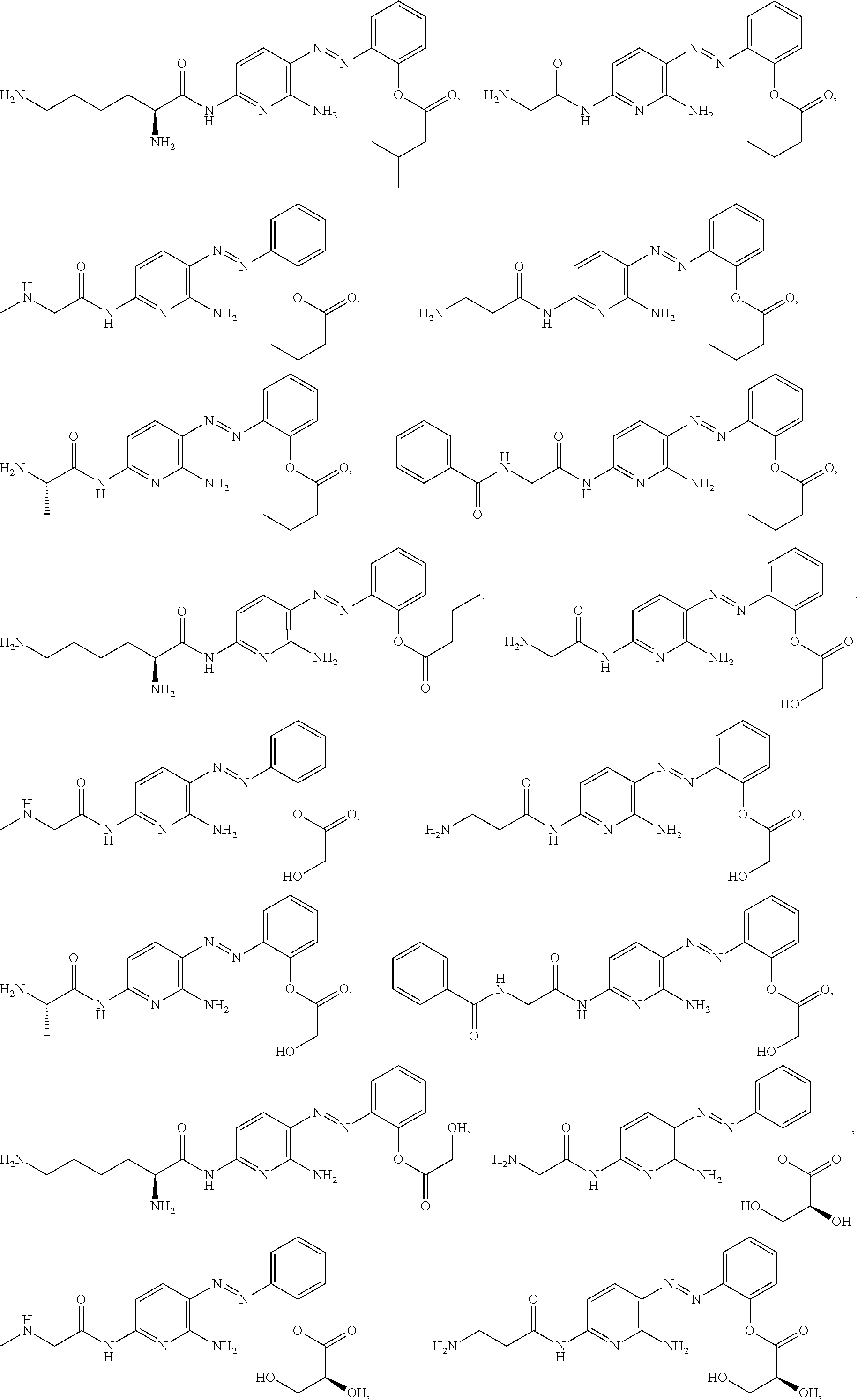

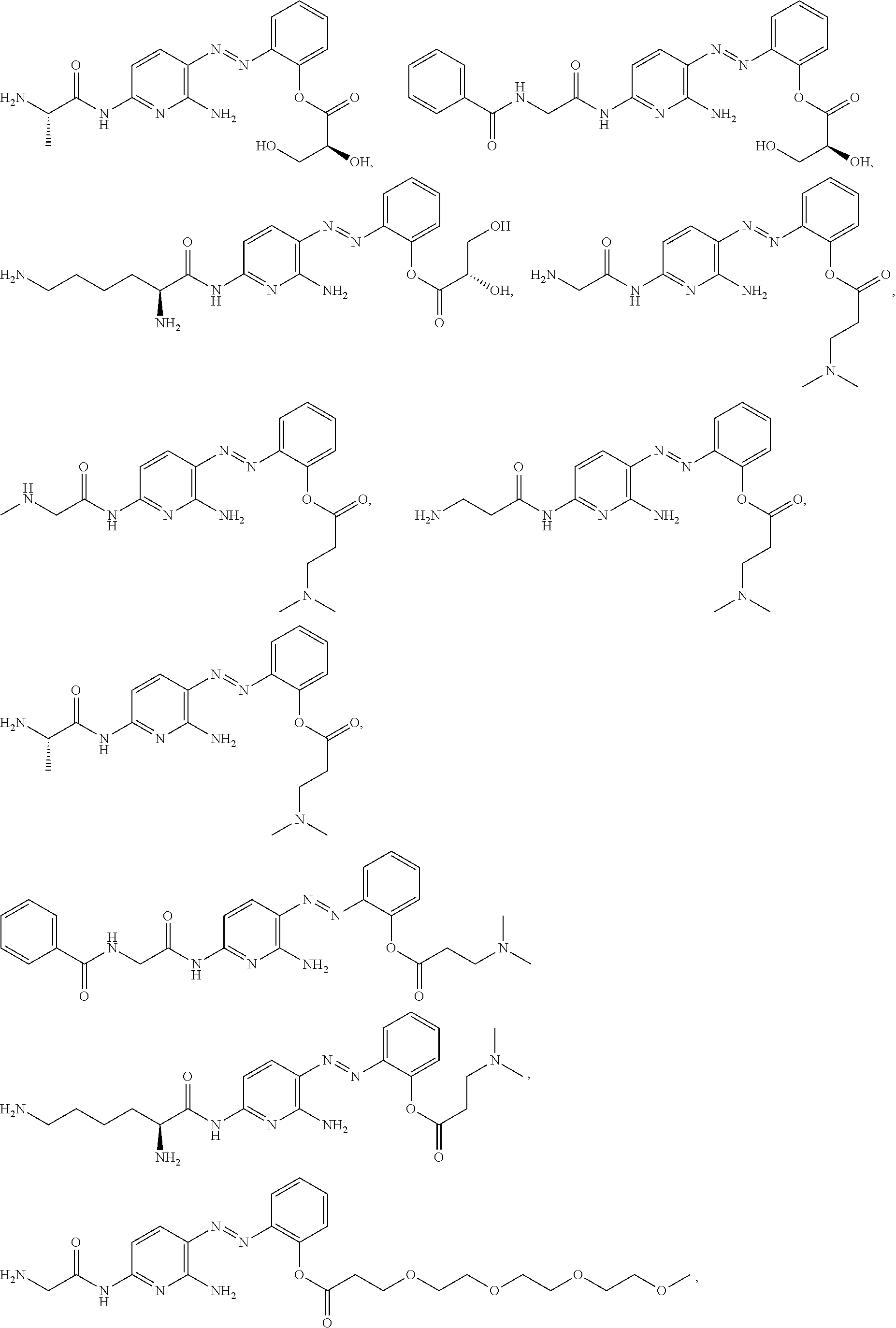

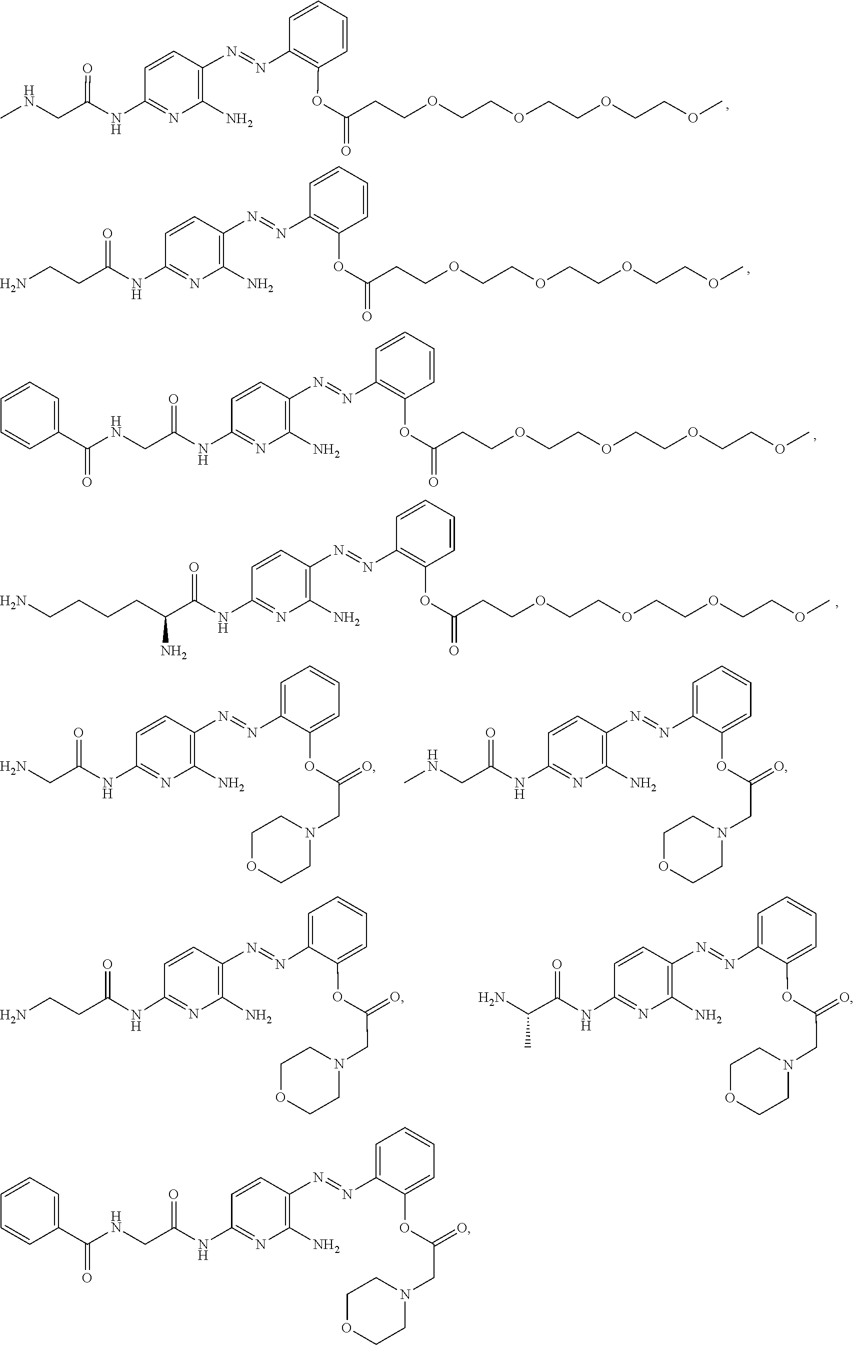

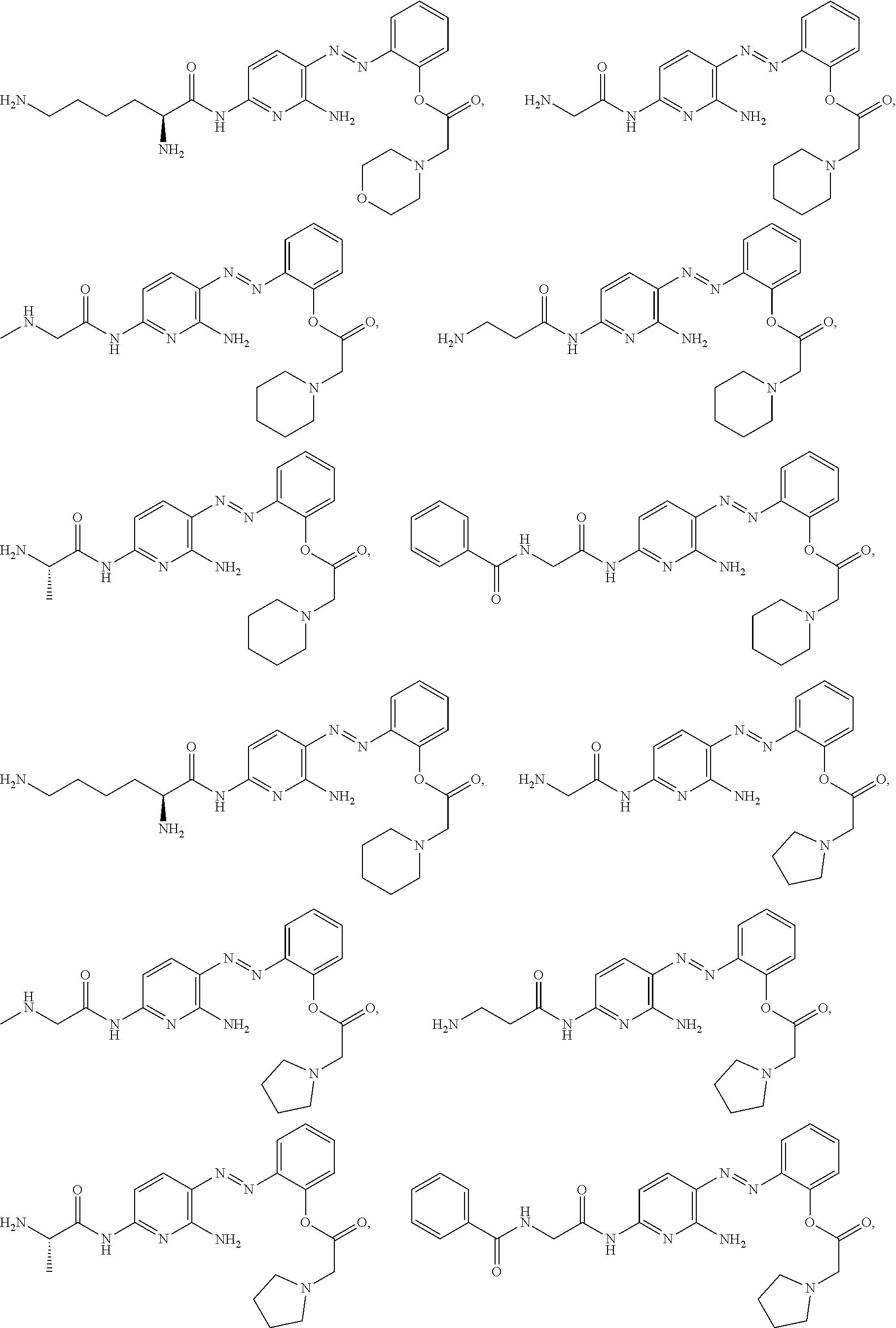

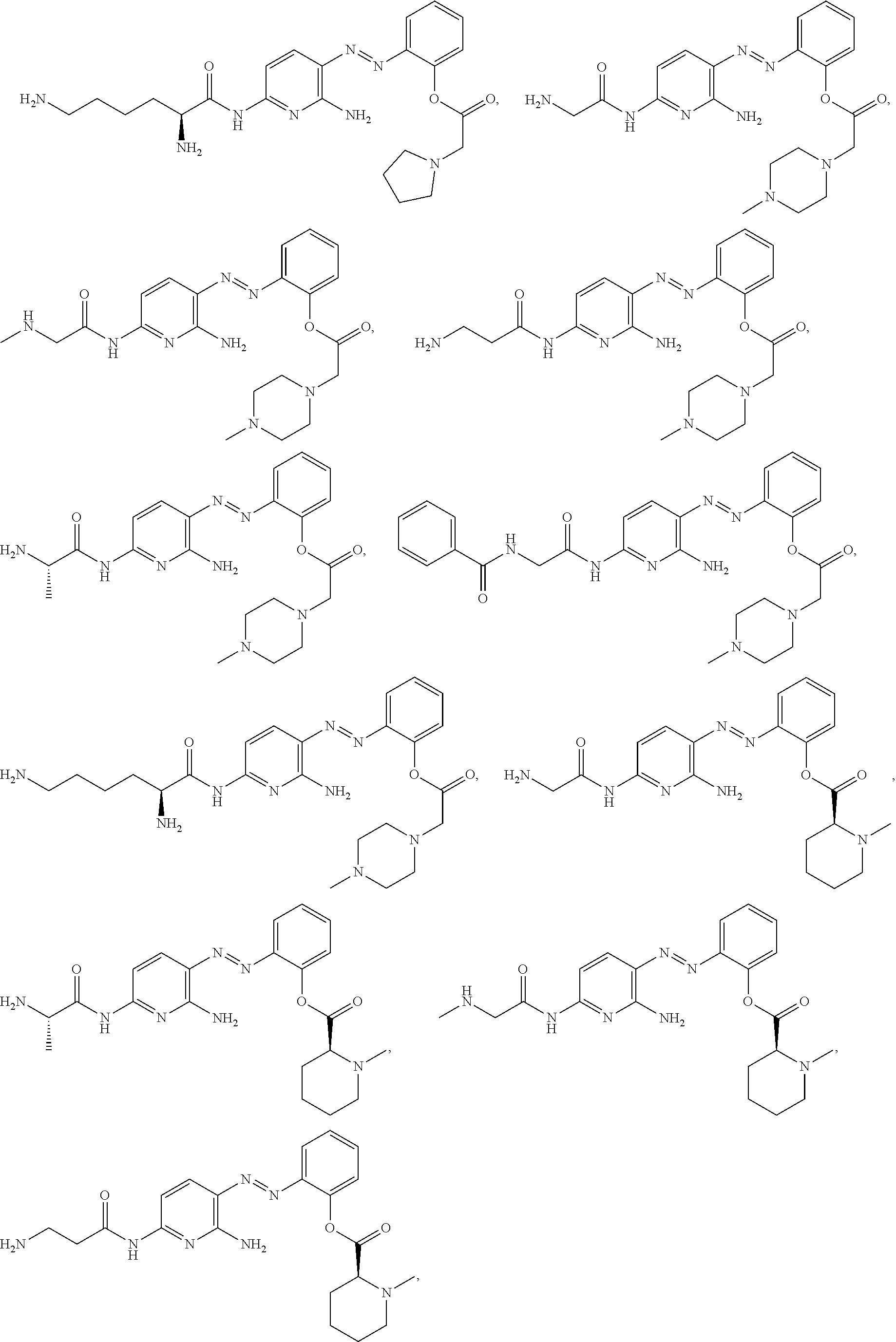

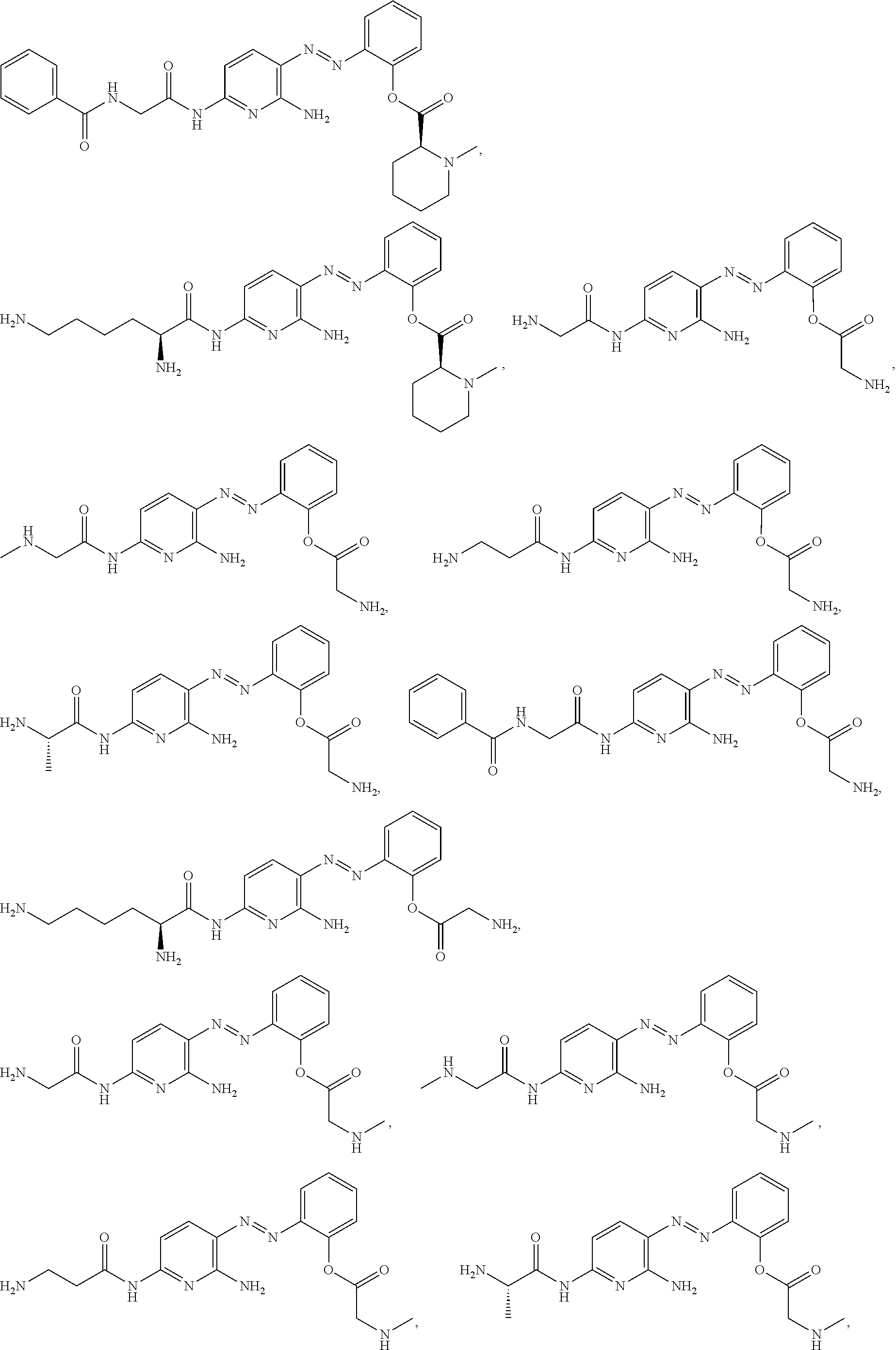

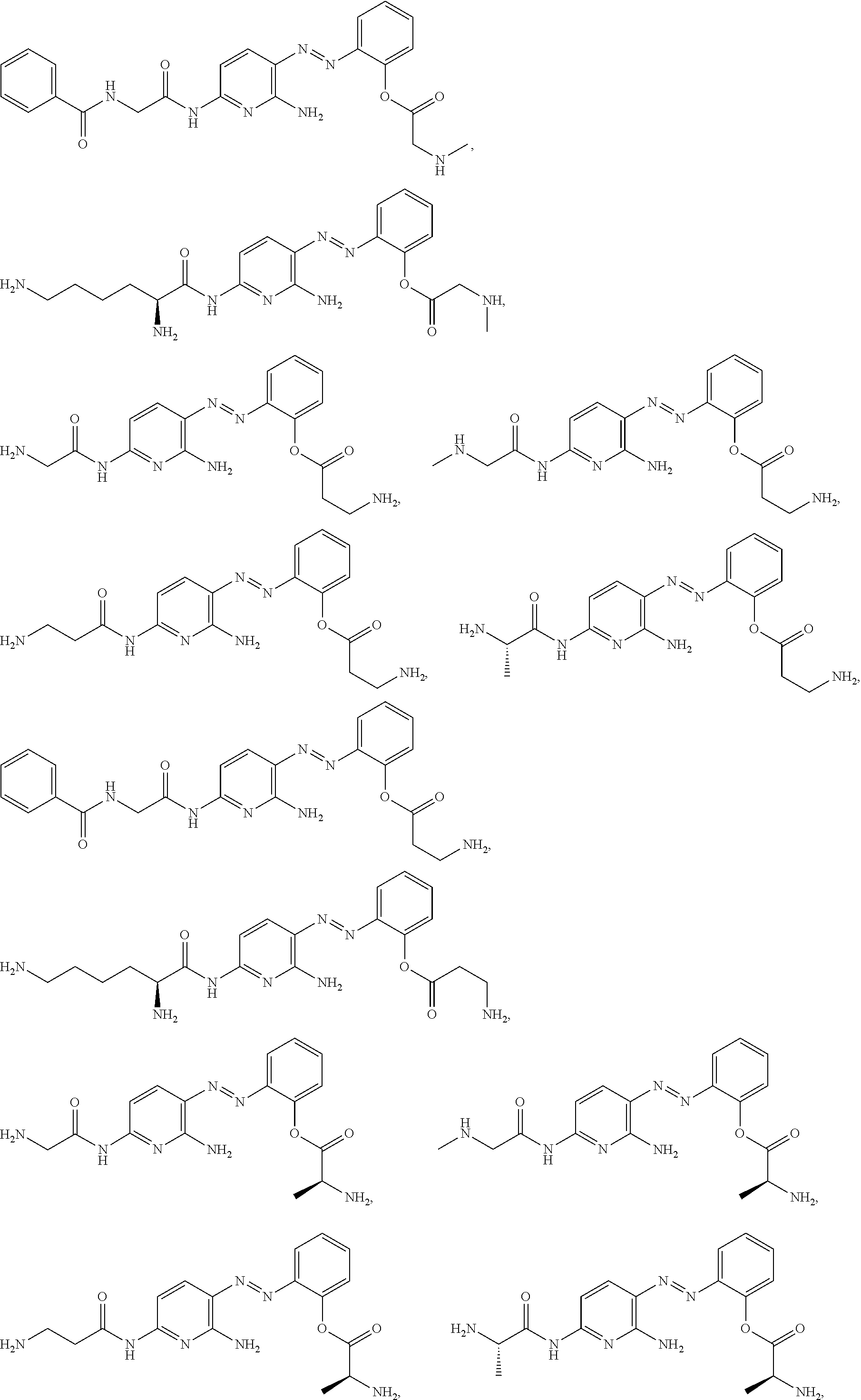

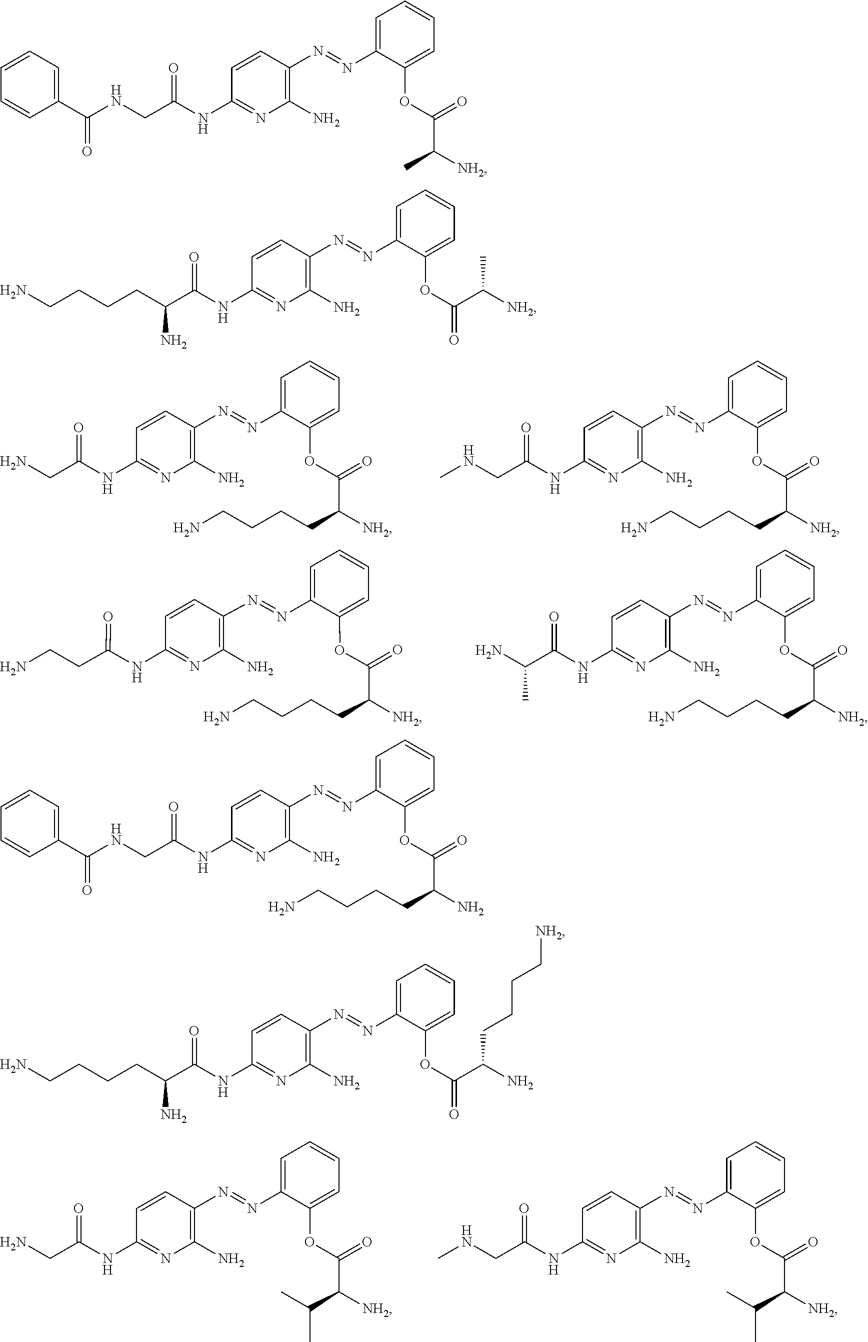

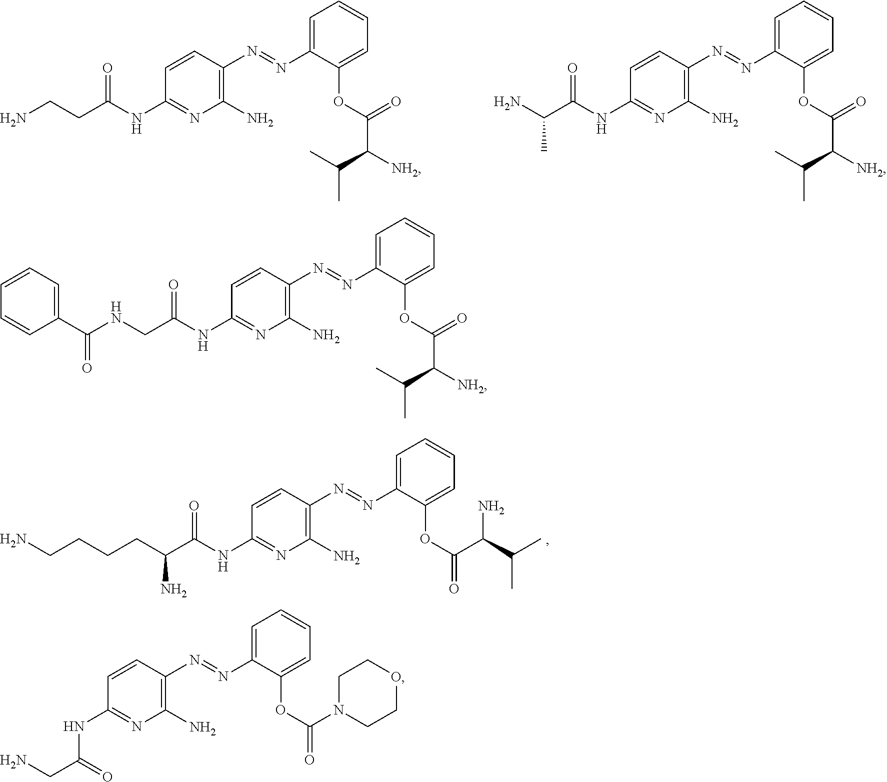

pharmaceutically acceptable salts or solvates thereof:

In one embodiment, the present invention relates to compounds of general formula (IX-D) pharmaceutically acceptable salts or solvates thereof:

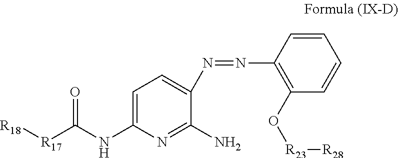

wherein R₁₇ is selected from optionally substituted linear or branched alkylene group, wherein alkylene is optionally substituted by amino, alkylamino and dialkylamino group; Rig is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group; R₂₃ is optionally substituted linear or branched alkylene;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group.

In one embodiment, the present invention provides the compound

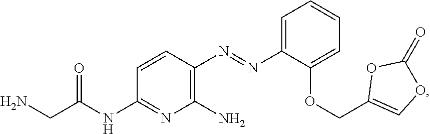 [SMILES: NCC(=O)Nc1ccc(/N=N/c2ccccc2OCc2coc(=O)o2)c(N)n1]

pharmaceutically acceptable salts or solvates thereof.

In one embodiment, the present invention relates to compounds of general formula (X), pharmaceutically acceptable salts or solvates thereof:

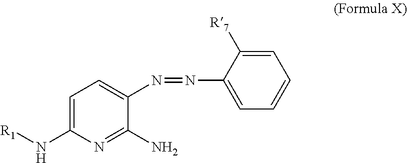

wherein;
R₁ is selected from

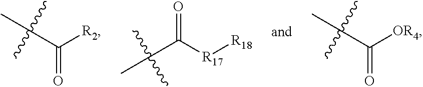

wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20; Wherein R₁₇ is selected from optionally substituted linear or branched alkylene group, wherein alkylene is optionally substituted by amino, alkylamino and dialkylamino group; R₁₈ is selected from NH₂, —NR₂₀R₂₁ wherein R₂₀ and R₂₁ is each independently selected from hydrogen, optionally substituted alkyl and —C(O)—R₂₂ wherein R₂₂ is selected from optionally substituted alkyl, cycloalkyl, heterocycloalkyl, heteroaryl, and aryl group;
R′₇ is selected from —OH, —OR₁₂,

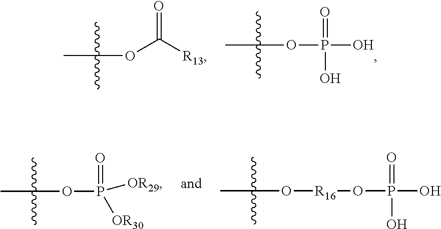

and R₁₉, wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group; R₁₆ is selected from optionally substituted alkylene_{(C1-6)} group; R₁₉ is selected from —O—R₂₃—O—C(O)—R₂₄, —O—C(O)—R₂₃—R₂₇, and —O—R₂₃—R₂₈, wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group;
R₂₉ and R₃₀ are each independently selected from hydrogen, and —R₂₃—O—C(O)—O—R₃₁; wherein R₃₁ is selected from optionally substituted alkyl, cycloalkyl, heterocycloalkyl, aryl, and heteroaryl group.

In one embodiment the present invention provides at least one compound selected from the group consisting of:

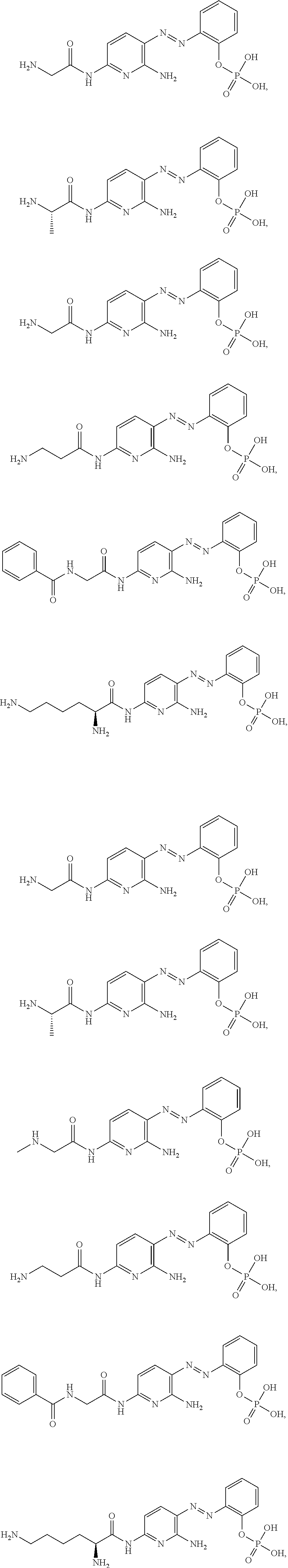

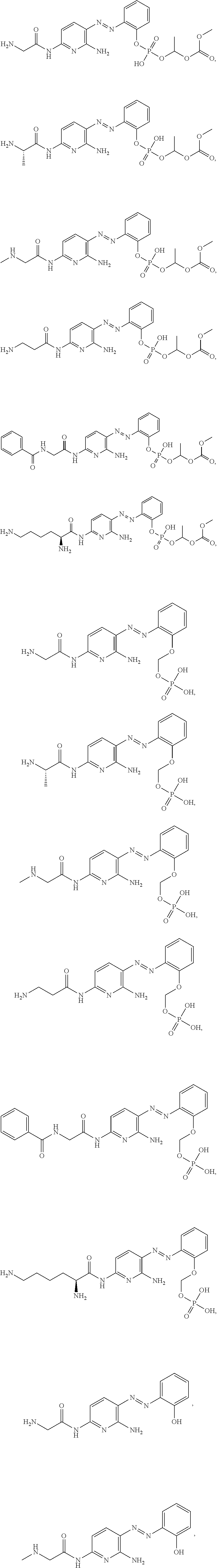

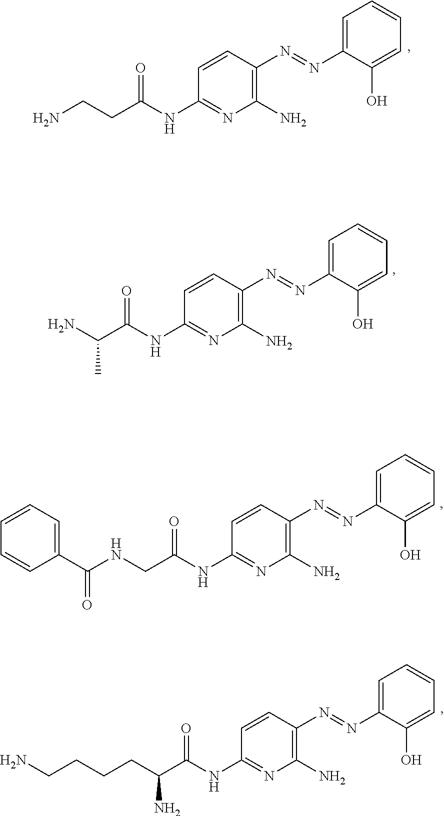

pharmaceutically acceptable salts or solvates thereof.

In one embodiment, the present invention provides at least one compound selected from group consisting of compounds listed in Table 2 pharmaceutically acceptable salts or solvates thereof.

In one embodiment, the present invention provides a method for the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, comprises administering at least one compound selected from group consisting of compounds listed in Table 2, pharmaceutically acceptable salts or solvates thereof.

In one embodiment, the present invention provides a method of use of at least one compound selected from group consisting of compounds listed in Table 2, pharmaceutically acceptable salts or solvates thereof, for treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence.

TABLE 2
Compounds

| Sr. | |
|---|---|
| No. | Structures |
| 200 | 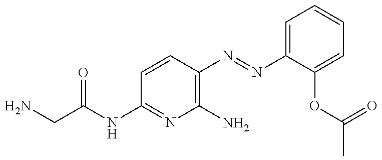 [SMILES: CC(=O)Oc1ccccc1/N=N/c1ccc(NC(=O)CN)nc1N] |
| 201 | 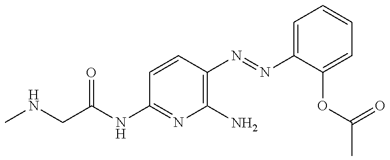 [SMILES: CNCC(=O)Nc1ccc(/N=N/c2ccccc2OC(C)=O)c(N)n1] |
| 202 | 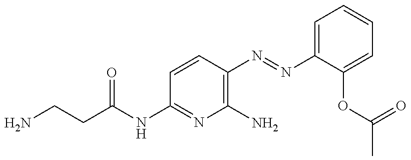 [SMILES: CC(=O)Oc1ccccc1/N=N/c1ccc(NC(=O)CCN)nc1N] |
| 203 | 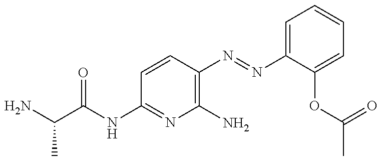 [SMILES: CC(=O)Oc1ccccc1/N=N/c1ccc(NC(=O)[C@H](C)N)nc1N] |
| 204 | 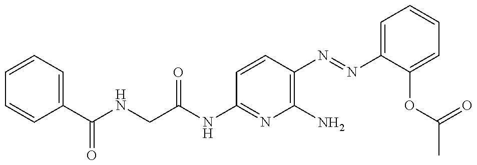 [SMILES: CC(=O)Oc1ccccc1/N=N/c1ccc(NC(=O)CNC(=O)c2ccccc2)nc1N] |
| 205 | 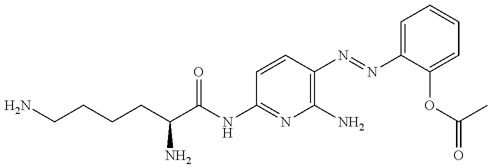 [SMILES: CC(=O)Oc1ccccc1/N=N/c1ccc(NC(=O)[C@@H](N)CCCCN)nc1N] |
| 206 | 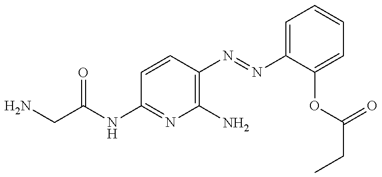 [SMILES: CCC(=O)Oc1ccccc1/N=N/c1ccc(NC(=O)CN)nc1N] |
| 207 | 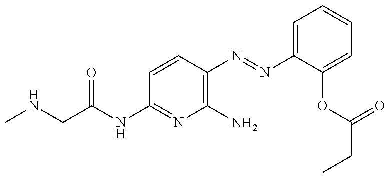 [SMILES: CCC(=O)Oc1ccccc1/N=N/c1ccc(NC(=O)CNC)nc1N] |
| 208 | 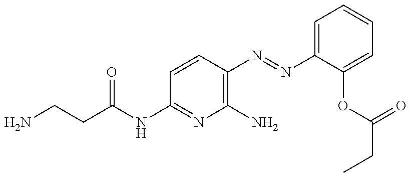 [SMILES: CCC(=O)Oc1ccccc1/N=N/c1ccc(NC(=O)CCN)nc1N] |
| 209 | 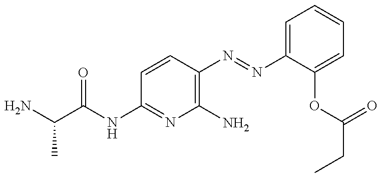 [SMILES: CCC(=O)Oc1ccccc1/N=N/c1ccc(NC(=O)[C@H](C)N)nc1N] |
| 210 | 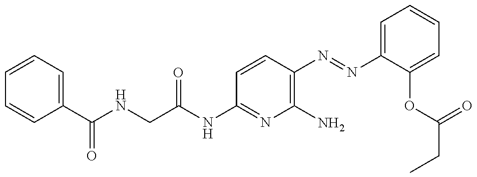 [SMILES: CCC(=O)Oc1ccccc1/N=N/c1ccc(NC(=O)CNC(=O)c2ccccc2)nc1N] |
| 211 | 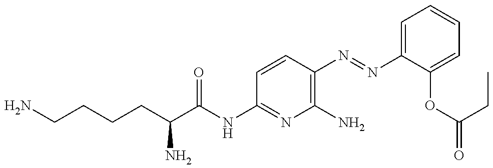 [SMILES: CCC(=O)Oc1ccccc1/N=N/c1ccc(NC(=O)[C@@H](N)CCCCN)nc1N] |
| 212 | 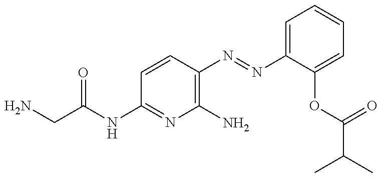 [SMILES: CC(C)C(=O)Oc1ccccc1/N=N/c1ccc(NC(=O)CN)nc1N] |
| 213 | 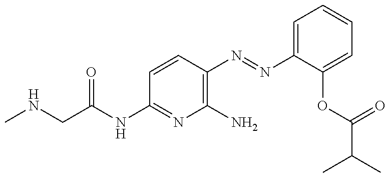 [SMILES: CNCC(=O)Nc1ccc(/N=N/c2ccccc2OC(=O)C(C)C)c(N)n1] |
| 214 | 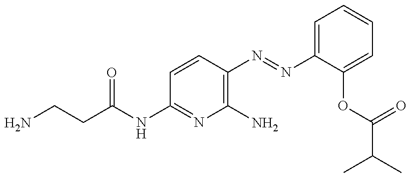 [SMILES: CC(C)C(=O)Oc1ccccc1/N=N/c1ccc(NC(=O)CCN)nc1N] |
| 215 | 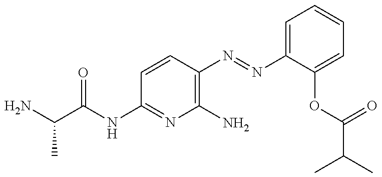 [SMILES: CC(C)C(=O)Oc1ccccc1/N=N/c1ccc(NC(=O)[C@H](C)N)nc1N] |
| 216 | 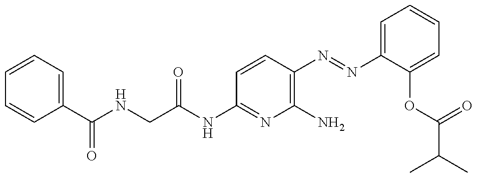 [SMILES: CC(C)C(=O)Oc1ccccc1/N=N/c1ccc(NC(=O)CNC(=O)c2ccccc2)nc1N] |
| 217 | 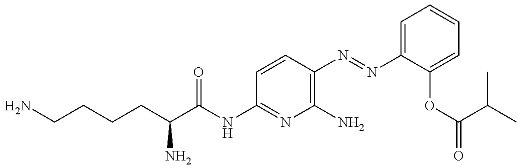 [SMILES: CC(C)C(=O)Oc1ccccc1/N=N/c1ccc(NC(=O)[C@@H](N)CCCCN)nc1N] |
| 218 | 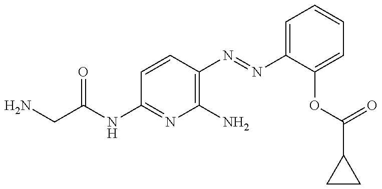 [SMILES: NCC(=O)Nc1ccc(/N=N/c2ccccc2OC(=O)C2CC2)c(N)n1] |
| 219 | 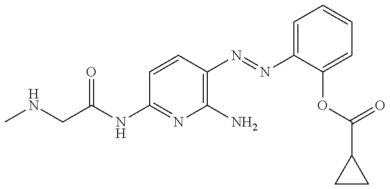 [SMILES: CNCC(=O)Nc1ccc(/N=N/c2ccccc2OC(=O)C2CC2)c(N)n1] |
| 220 | 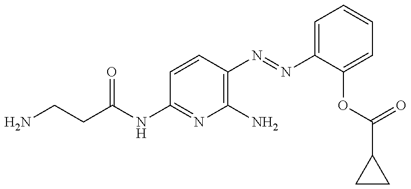 [SMILES: NCCC(=O)Nc1ccc(/N=N/c2ccccc2OC(=O)C2CC2)c(N)n1] |
| 221 | 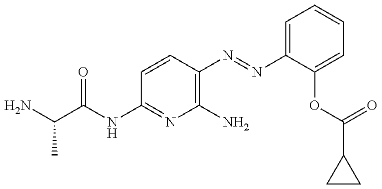 [SMILES: C[C@H](N)C(=O)Nc1ccc(/N=N/c2ccccc2OC(=O)C2CC2)c(N)n1] |
| 222 | 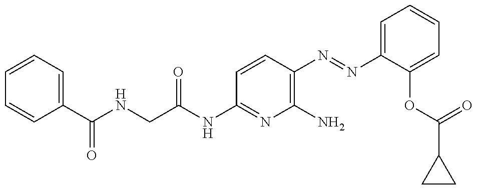 [SMILES: Nc1nc(NC(=O)CNC(=O)c2ccccc2)ccc1/N=N/c1ccccc1OC(=O)C1CC1] |
| 223 | 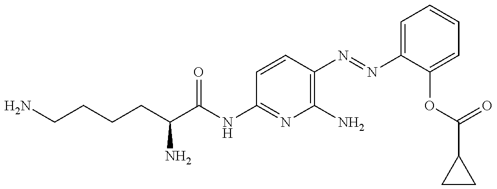 [SMILES: NCCCC[C@H](N)C(=O)Nc1ccc(/N=N/c2ccccc2OC(=O)C2CC2)c(N)n1] |
| 224 | 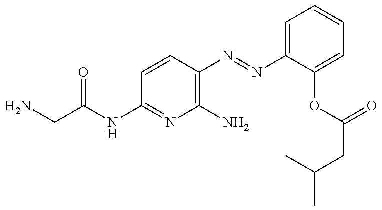 [SMILES: CC(C)CC(=O)Oc1ccccc1/N=N/c1ccc(NC(=O)CN)nc1N] |
| 225 | 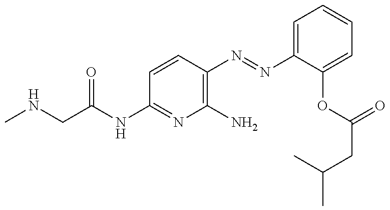 [SMILES: CNCC(=O)Nc1ccc(/N=N/c2ccccc2OC(=O)CC(C)C)c(N)n1] |
| 226 | 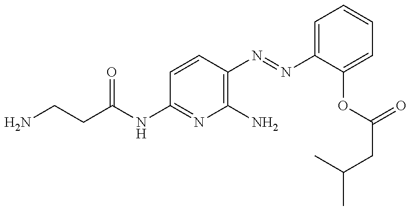 [SMILES: CC(C)CC(=O)Oc1ccccc1/N=N/c1ccc(NC(=O)CCN)nc1N] |
| 227 | 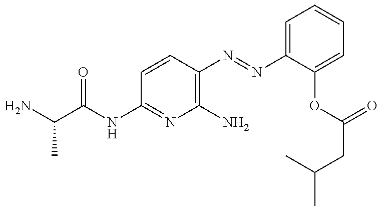 [SMILES: CC(C)CC(=O)Oc1ccccc1/N=N/c1ccc(NC(=O)[C@H](C)N)nc1N] |
| 228 | 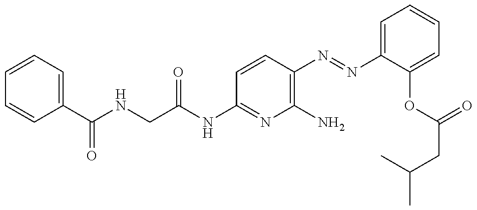 [SMILES: CC(C)CC(=O)Oc1ccccc1/N=N/c1ccc(NC(=O)CNC(=O)c2ccccc2)nc1N] |
| 229 | 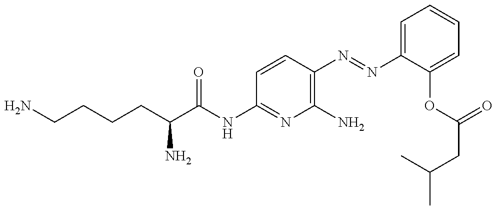 [SMILES: CC(C)CC(=O)Oc1ccccc1/N=N/c1ccc(NC(=O)[C@@H](N)CCCCN)nc1N] |
| 230 | 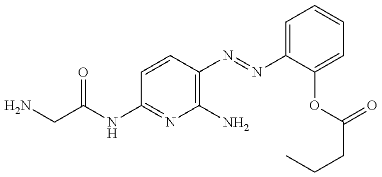 [SMILES: CCCC(=O)Oc1ccccc1/N=N/c1ccc(NC(=O)CN)nc1N] |
| 231 | 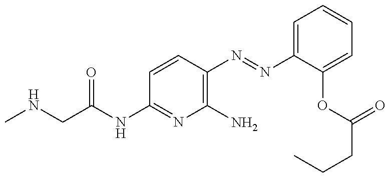 [SMILES: CCCC(=O)Oc1ccccc1/N=N/c1ccc(NC(=O)CNC)nc1N] |
| 232 | 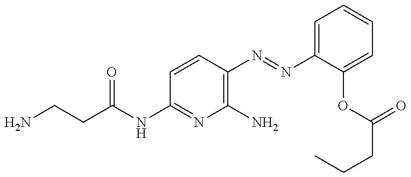 [SMILES: CCCC(=O)Oc1ccccc1/N=N/c1ccc(NC(=O)CCN)nc1N] |
| 233 | 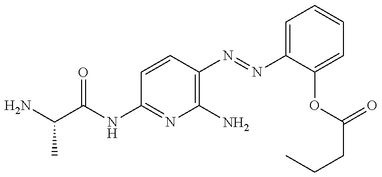 [SMILES: CCCC(=O)Oc1ccccc1/N=N/c1ccc(NC(=O)[C@H](C)N)nc1N] |
| 234 | 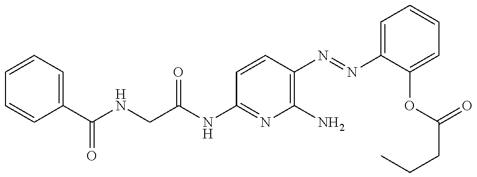 [SMILES: CCCC(=O)Oc1ccccc1/N=N/c1ccc(NC(=O)CNC(=O)c2ccccc2)nc1N] |
| 235 | 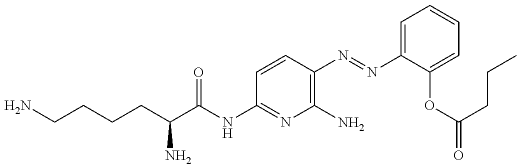 [SMILES: CCCC(=O)Oc1ccccc1/N=N/c1ccc(NC(=O)[C@@H](N)CCCCN)nc1N] |
| 236 | 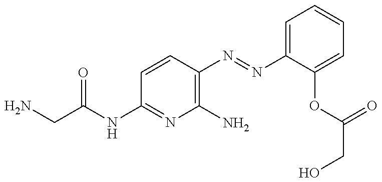 [SMILES: NCC(=O)Nc1ccc(/N=N/c2ccccc2OC(=O)CO)c(N)n1] |
| 237 | 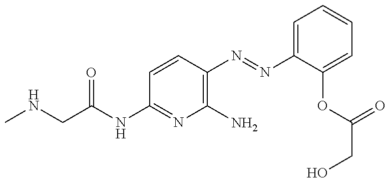 [SMILES: CNCC(=O)Nc1ccc(/N=N/c2ccccc2OC(=O)CO)c(N)n1] |
| 238 | 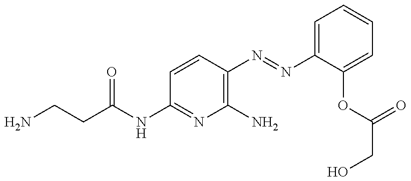 [SMILES: NCCC(=O)Nc1ccc(/N=N/c2ccccc2OC(=O)CO)c(N)n1] |
| 239 | 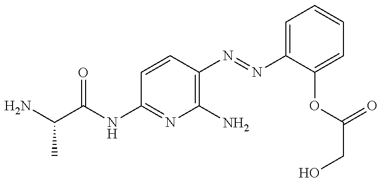 [SMILES: C[C@H](N)C(=O)Nc1ccc(/N=N/c2ccccc2OC(=O)CO)c(N)n1] |
| 240 | 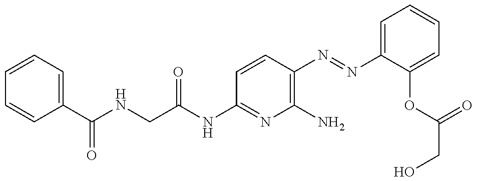 [SMILES: Nc1nc(NC(=O)CNC(=O)c2ccccc2)ccc1/N=N/c1ccccc1OC(=O)CO] |
| 241 | 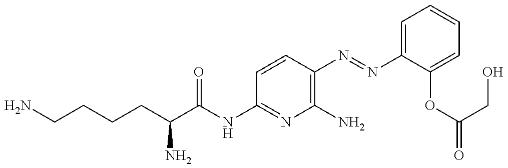 [SMILES: NCCCC[C@H](N)C(=O)Nc1ccc(/N=N/c2ccccc2OC(=O)CO)c(N)n1] |
| 242 | 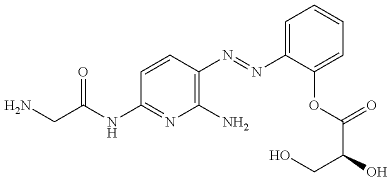 [SMILES: NCC(=O)Nc1ccc(/N=N/c2ccccc2OC(=O)[C@@H](O)CO)c(N)n1] |
| 243 | 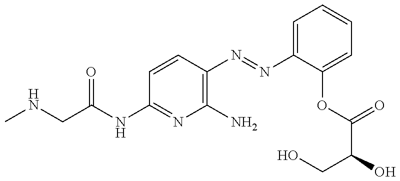 [SMILES: CNCC(=O)Nc1ccc(/N=N/c2ccccc2OC(=O)[C@@H](O)CO)c(N)n1] |
| 244 | 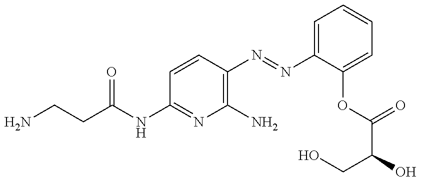 [SMILES: NCCC(=O)Nc1ccc(/N=N/c2ccccc2OC(=O)[C@@H](O)CO)c(N)n1] |
| 245 | 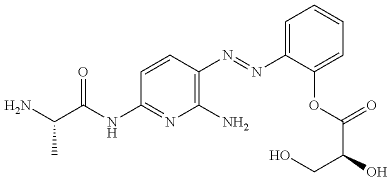 [SMILES: C[C@H](N)C(=O)Nc1ccc(/N=N/c2ccccc2OC(=O)[C@@H](O)CO)c(N)n1] |
| 246 | 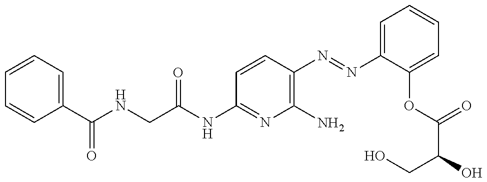 [SMILES: Nc1nc(NC(=O)CNC(=O)c2ccccc2)ccc1/N=N/c1ccccc1OC(=O)[C@@H](O)CO] |
| 247 | 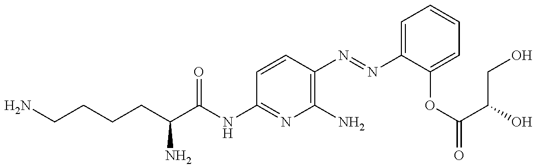 [SMILES: NCCCC[C@H](N)C(=O)Nc1ccc(/N=N/c2ccccc2OC(=O)[C@@H](O)CO)c(N)n1] |
| 248 | 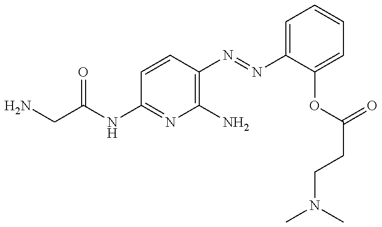 [SMILES: CN(C)CCC(=O)Oc1ccccc1/N=N/c1ccc(NC(=O)CN)nc1N] |
| 249 | 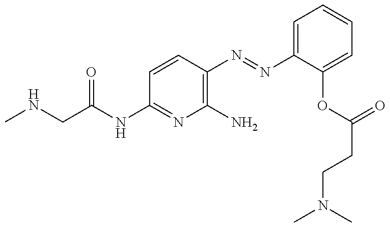 [SMILES: CNCC(=O)Nc1ccc(/N=N/c2ccccc2OC(=O)CCN(C)C)c(N)n1] |
| 250 | 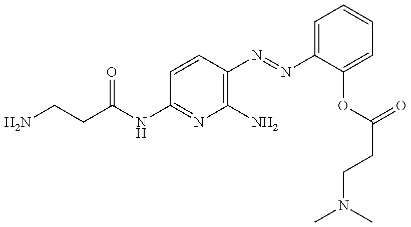 [SMILES: CN(C)CCC(=O)Oc1ccccc1/N=N/c1ccc(NC(=O)CCN)nc1N] |
| 251 | 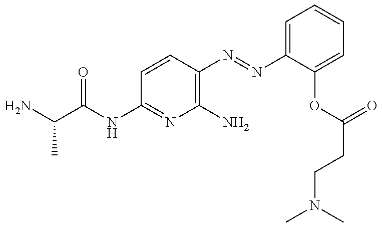 [SMILES: C[C@H](N)C(=O)Nc1ccc(/N=N/c2ccccc2OC(=O)CCN(C)C)c(N)n1] |
| 252 | 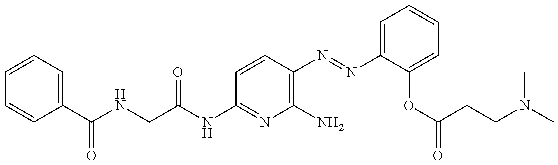 [SMILES: CN(C)CCC(=O)Oc1ccccc1/N=N/c1ccc(NC(=O)CNC(=O)c2ccccc2)nc1N] |
| 253 | 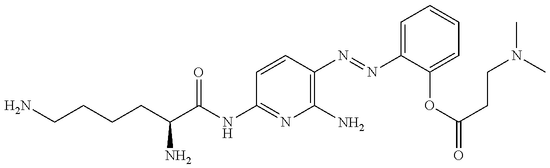 [SMILES: CN(C)CCC(=O)Oc1ccccc1/N=N/c1ccc(NC(=O)[C@@H](N)CCCCN)nc1N] |
| 254 | 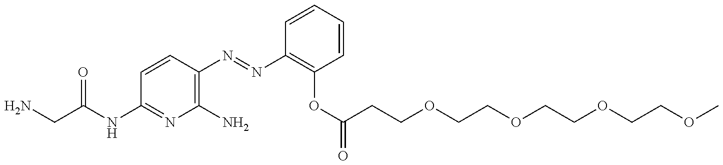 [SMILES: COCCOCCOCCOCCC(=O)Oc1ccccc1/N=N/c1ccc(NC(=O)CN)nc1N] |
| 255 | 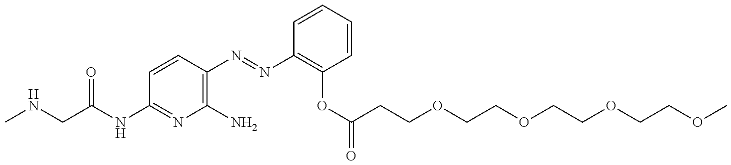 |
| 256 | 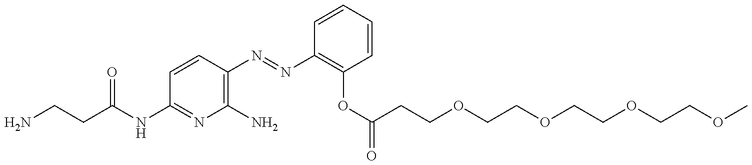 [SMILES: COCCOCCOCCOCCC(=O)Oc1ccccc1/N=N/c1ccc(NC(=O)CCN)nc1N] |
| 257 | 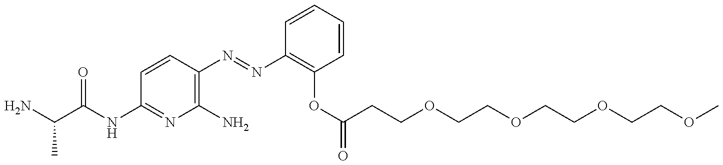 [SMILES: COCCOCCOCCOCCC(=O)Oc1ccccc1/N=N/c1ccc(NC(=O)[C@H](C)N)nc1N] |
| 258 | 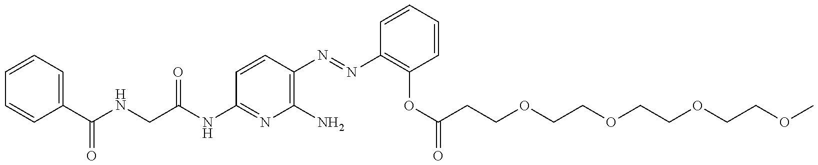 [SMILES: COCCOCCOCCOCCC(=O)Oc1ccccc1/N=N/c1ccc(NC(=O)CNC(=O)c2ccccc2)nc1N] |
| 259 | 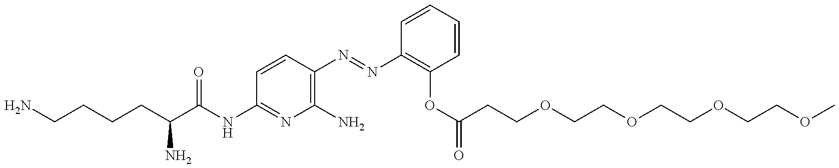 [SMILES: COCCOCCOCCOCCC(=O)Oc1ccccc1/N=N/c1ccc(NC(=O)[C@@H](N)CCCCN)nc1N] |
| 260 | 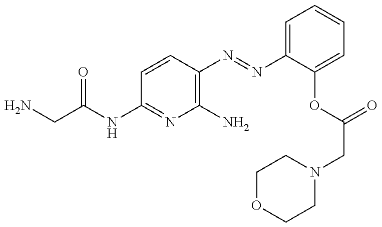 [SMILES: NCC(=O)Nc1ccc(/N=N/c2ccccc2OC(=O)CN2CCOCC2)c(N)n1] |
| 261 | 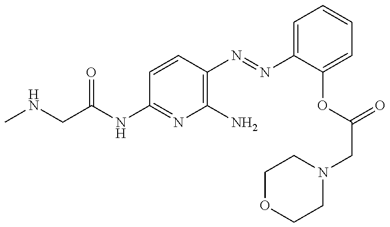 [SMILES: CNCC(=O)Nc1ccc(/N=N/c2ccccc2OC(=O)CN2CCOCC2)c(N)n1] |
| 262 | 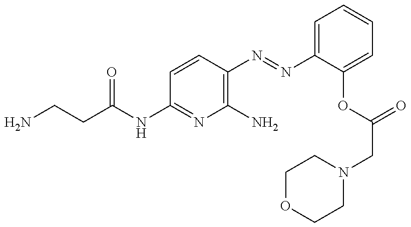 [SMILES: NCCC(=O)Nc1ccc(/N=N/c2ccccc2OC(=O)CN2CCOCC2)c(N)n1] |
| 263 | 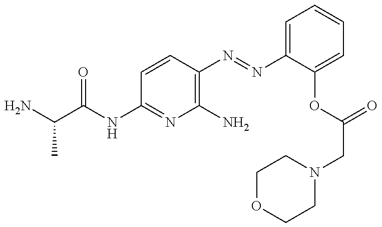 [SMILES: C[C@H](N)C(=O)Nc1ccc(/N=N/c2ccccc2OC(=O)CN2CCOCC2)c(N)n1] |
| 264 | 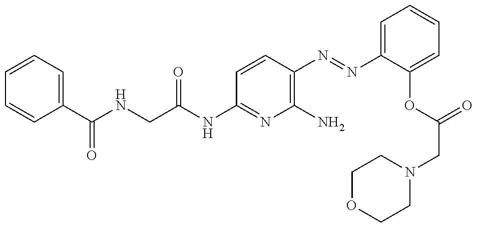 [SMILES: Nc1nc(NC(=O)CNC(=O)c2ccccc2)ccc1/N=N/c1ccccc1OC(=O)CN1CCOCC1] |
| 265 | 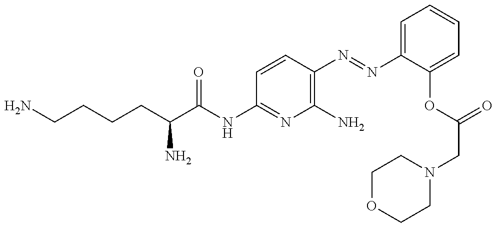 [SMILES: NCCCC[C@H](N)C(=O)Nc1ccc(/N=N/c2ccccc2OC(=O)CN2CCOCC2)c(N)n1] |
| 266 | 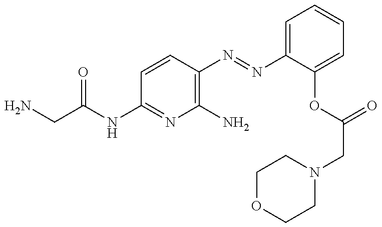 [SMILES: NCC(=O)Nc1ccc(/N=N/c2ccccc2OC(=O)CN2CCOCC2)c(N)n1] |
| 267 | 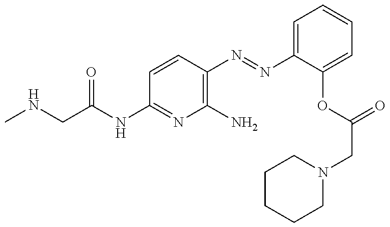 [SMILES: CNCC(=O)Nc1ccc(/N=N/c2ccccc2OC(=O)CN2CCCCC2)c(N)n1] |
| 268 | 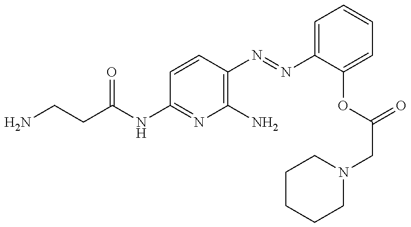 [SMILES: NCCC(=O)Nc1ccc(/N=N/c2ccccc2OC(=O)CN2CCCCC2)c(N)n1] |
| 269 | 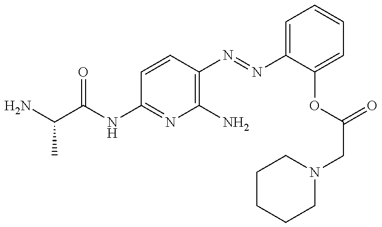 [SMILES: C[C@H](N)C(=O)Nc1ccc(/N=N/c2ccccc2OC(=O)CN2CCCCC2)c(N)n1] |
| 270 | 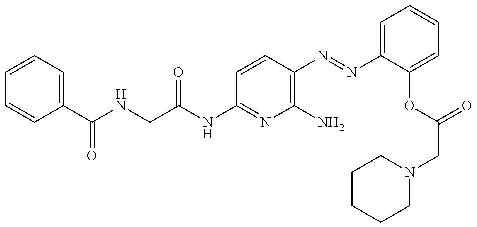 [SMILES: Nc1nc(NC(=O)CNC(=O)c2ccccc2)ccc1/N=N/c1ccccc1OC(=O)CN1CCCCC1] |
| 271 | 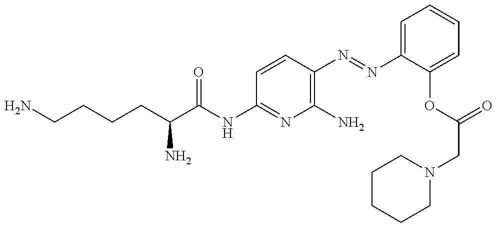 [SMILES: NCCCC[C@H](N)C(=O)Nc1ccc(/N=N/c2ccccc2OC(=O)CN2CCCCC2)c(N)n1] |
| 272 | 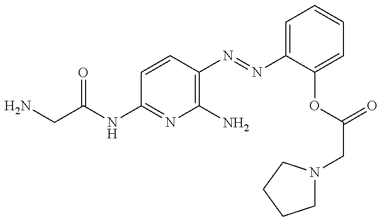 [SMILES: NCC(=O)Nc1ccc(/N=N/c2ccccc2OC(=O)CN2CCCC2)c(N)n1] |
| 273 | 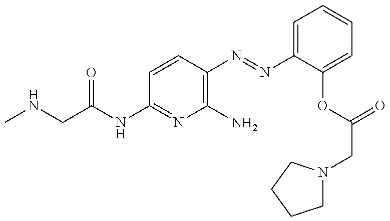 [SMILES: CNCC(=O)Nc1ccc(/N=N/c2ccccc2OC(=O)CN2CCCC2)c(N)n1] |
| 274 | 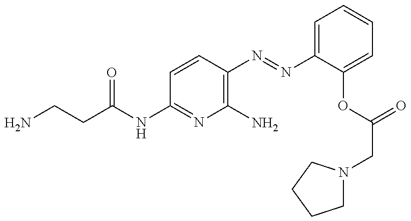 [SMILES: NCCC(=O)Nc1ccc(/N=N/c2ccccc2OC(=O)CN2CCCC2)c(N)n1] |
| 275 | 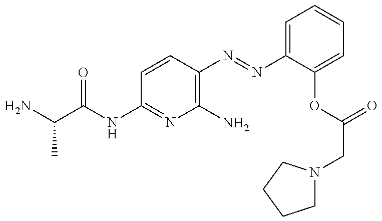 [SMILES: C[C@H](N)C(=O)Nc1ccc(/N=N/c2ccccc2OC(=O)CN2CCCC2)c(N)n1] |
| 276 | 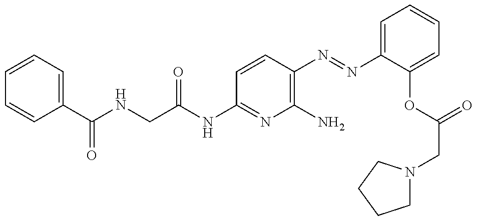 [SMILES: Nc1nc(NC(=O)CNC(=O)c2ccccc2)ccc1/N=N/c1ccccc1OC(=O)CN1CCCC1] |
| 277 | 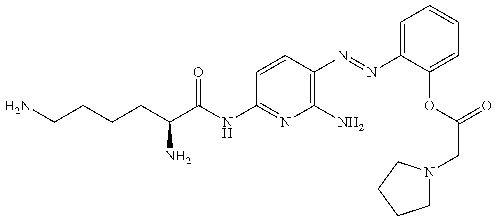 [SMILES: NCCCC[C@H](N)C(=O)Nc1ccc(/N=N/c2ccccc2OC(=O)CN2CCCC2)c(N)n1] |
| 278 | 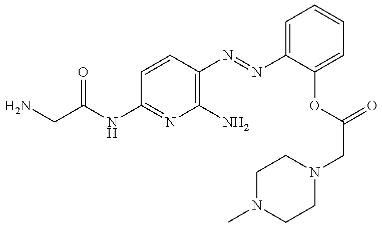 [SMILES: CN1CCN(CC(=O)Oc2ccccc2/N=N/c2ccc(NC(=O)CN)nc2N)CC1] |
| 279 | 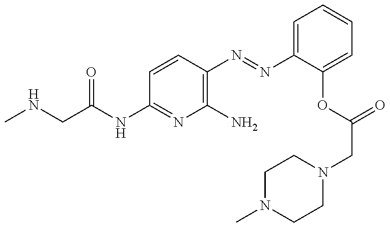 [SMILES: CNCC(=O)Nc1ccc(/N=N/c2ccccc2OC(=O)CN2CCN(C)CC2)c(N)n1] |
| 280 | 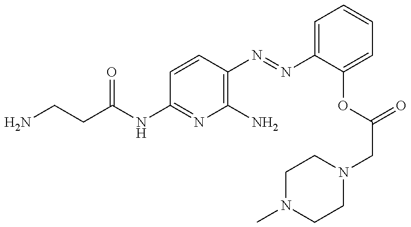 [SMILES: CN1CCN(CC(=O)Oc2ccccc2/N=N/c2ccc(NC(=O)CCN)nc2N)CC1] |
| 281 | 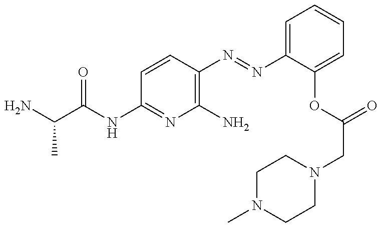 [SMILES: C[C@H](N)C(=O)Nc1ccc(/N=N/c2ccccc2OC(=O)CN2CCN(C)CC2)c(N)n1] |
| 282 | 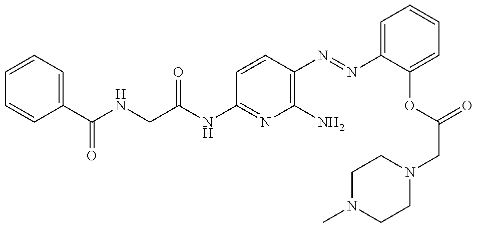 [SMILES: CN1CCN(CC(=O)Oc2ccccc2/N=N/c2ccc(NC(=O)CNC(=O)c3ccccc3)nc2N)CC1] |
| 283 | 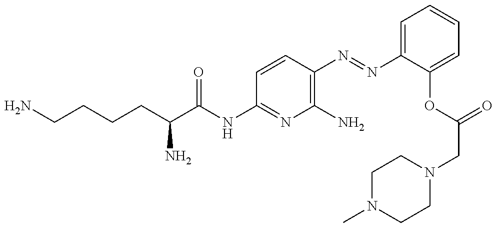 [SMILES: CN1CCN(CC(=O)Oc2ccccc2/N=N/c2ccc(NC(=O)[C@@H](N)CCCCN)nc2N)CC1] |
| 284 | 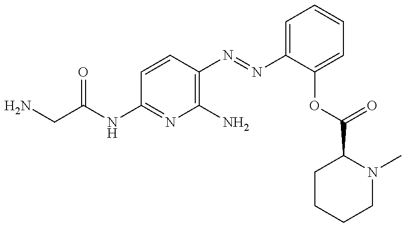 [SMILES: CN1CCCC[C@H]1C(=O)Oc1ccccc1/N=N/c1ccc(NC(=O)CN)nc1N] |
| 285 | 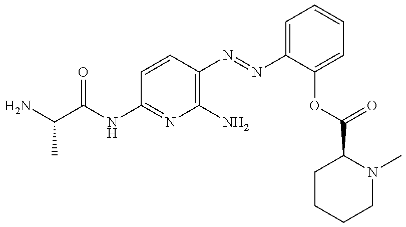 [SMILES: C[C@H](N)C(=O)Nc1ccc(/N=N/c2ccccc2OC(=O)[C@@H]2CCCCN2C)c(N)n1] |
| 286 | 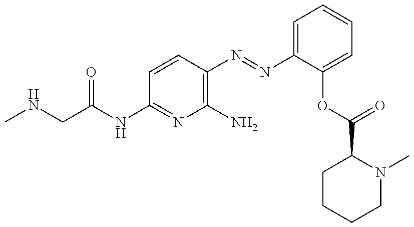 |
| 287 | 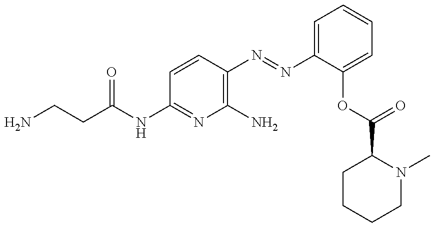 [SMILES: CN1CCCC[C@H]1C(=O)Oc1ccccc1/N=N/c1ccc(NC(=O)CCN)nc1N] |
| 288 | 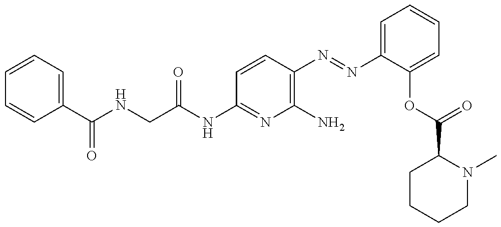 [SMILES: CN1CCCC[C@H]1C(=O)Oc1ccccc1/N=N/c1ccc(NC(=O)CNC(=O)c2ccccc2)nc1N] |
| 289 | 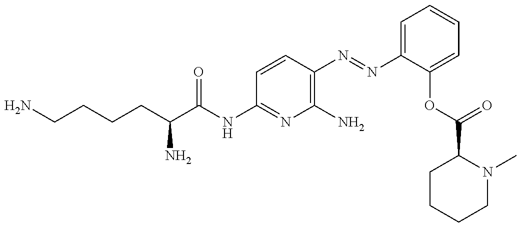 [SMILES: CN1CCCC[C@H]1C(=O)Oc1ccccc1/N=N/c1ccc(NC(=O)[C@@H](N)CCCCN)nc1N] |
| 290 | 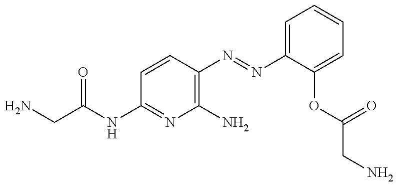 [SMILES: NCC(=O)Nc1ccc(/N=N/c2ccccc2OC(=O)CN)c(N)n1] |
| 291 | 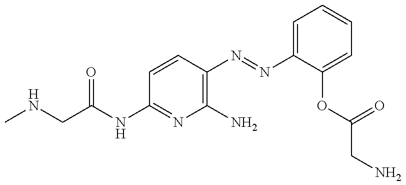 [SMILES: CNCC(=O)Nc1ccc(/N=N/c2ccccc2OC(=O)CN)c(N)n1] |
| 292 | 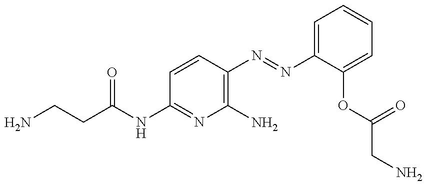 [SMILES: NCCC(=O)Nc1ccc(/N=N/c2ccccc2OC(=O)CN)c(N)n1] |
| 293 | 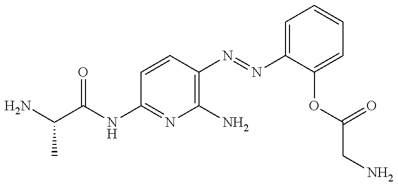 [SMILES: C[C@H](N)C(=O)Nc1ccc(/N=N/c2ccccc2OC(=O)CN)c(N)n1] |
| 294 | 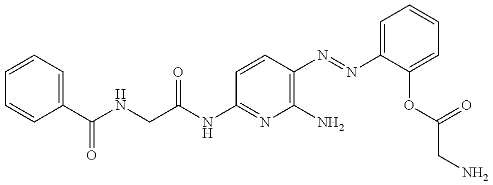 [SMILES: NCC(=O)Oc1ccccc1/N=N/c1ccc(NC(=O)CNC(=O)c2ccccc2)nc1N] |
| 295 | 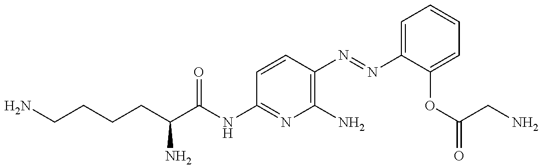 [SMILES: NCCCC[C@H](N)C(=O)Nc1ccc(/N=N/c2ccccc2OC(=O)CN)c(N)n1] |
| 296 | 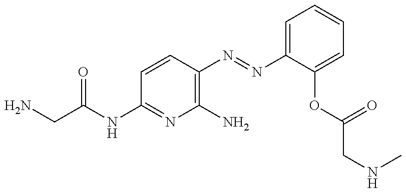 [SMILES: CNCC(=O)Oc1ccccc1/N=N/c1ccc(NC(=O)CN)nc1N] |
| 297 | 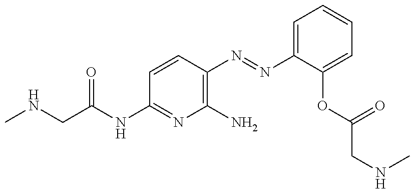 |
| 298 | 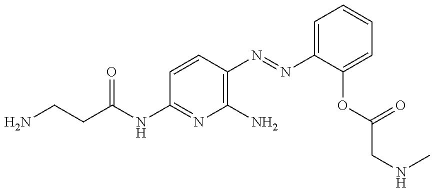 [SMILES: CNCC(=O)Oc1ccccc1/N=N/c1ccc(NC(=O)CCN)nc1N] |
| 299 | 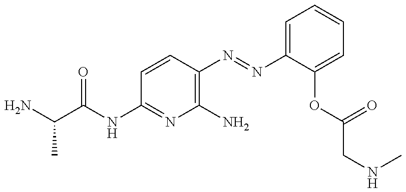 [SMILES: CNCC(=O)Oc1ccccc1/N=N/c1ccc(NC(=O)[C@H](C)N)nc1N] |
| 300 | 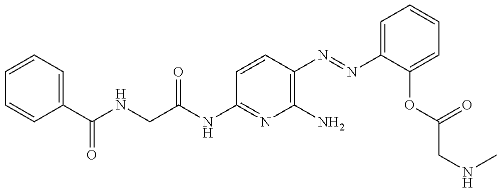 [SMILES: CNCC(=O)Oc1ccccc1/N=N/c1ccc(NC(=O)CNC(=O)c2ccccc2)nc1N] |
| 301 | 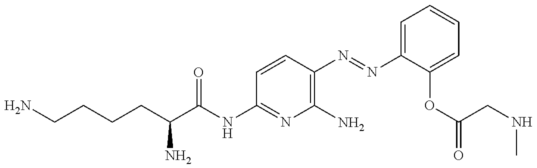 [SMILES: CNCC(=O)Oc1ccccc1/N=N/c1ccc(NC(=O)[C@@H](N)CCCCN)nc1N] |
| 302 | 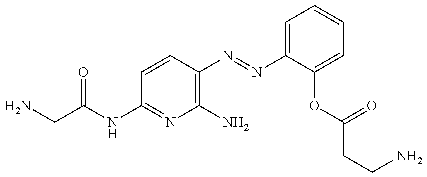 [SMILES: NCCC(=O)Oc1ccccc1/N=N/c1ccc(NC(=O)CN)nc1N] |
| 303 | 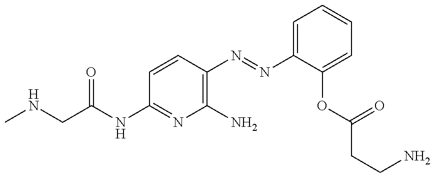 [SMILES: CNCC(=O)Nc1ccc(/N=N/c2ccccc2OC(=O)CCN)c(N)n1] |
| 304 | 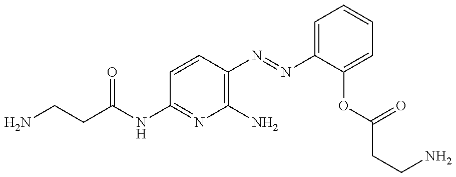 [SMILES: NCCC(=O)Nc1ccc(/N=N/c2ccccc2OC(=O)CCN)c(N)n1] |
| 305 | 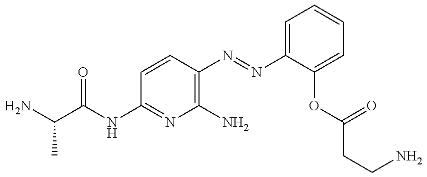 [SMILES: C[C@H](N)C(=O)Nc1ccc(/N=N/c2ccccc2OC(=O)CCN)c(N)n1] |
| 306 | 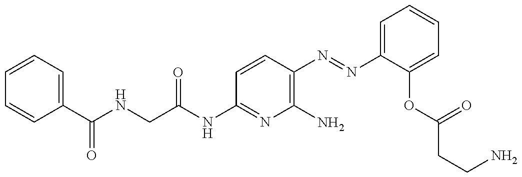 [SMILES: NCCC(=O)Oc1ccccc1/N=N/c1ccc(NC(=O)CNC(=O)c2ccccc2)nc1N] |
| 307 | 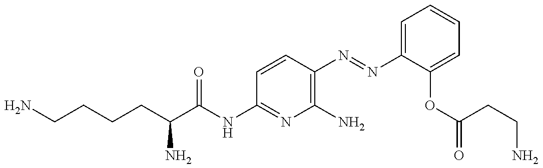 [SMILES: NCCCC[C@H](N)C(=O)Nc1ccc(/N=N/c2ccccc2OC(=O)CCN)c(N)n1] |
| 308 | 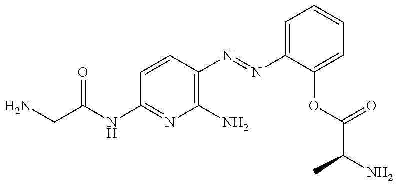 [SMILES: C[C@H](N)C(=O)Oc1ccccc1/N=N/c1ccc(NC(=O)CN)nc1N] |
| 309 | 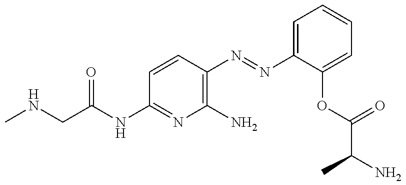 [SMILES: CNCC(=O)Nc1ccc(/N=N/c2ccccc2OC(=O)[C@H](C)N)c(N)n1] |
| 310 | 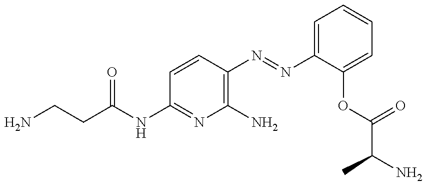 [SMILES: C[C@H](N)C(=O)Oc1ccccc1/N=N/c1ccc(NC(=O)CCN)nc1N] |
| 311 | 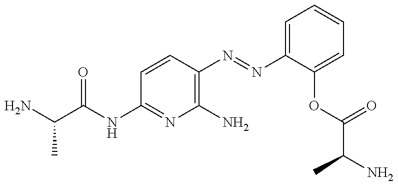 [SMILES: C[C@H](N)C(=O)Nc1ccc(/N=N/c2ccccc2OC(=O)[C@H](C)N)c(N)n1] |
| 312 | 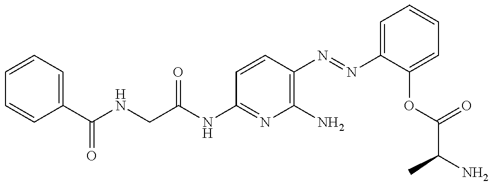 [SMILES: C[C@H](N)C(=O)Oc1ccccc1/N=N/c1ccc(NC(=O)CNC(=O)c2ccccc2)nc1N] |
| 313 | 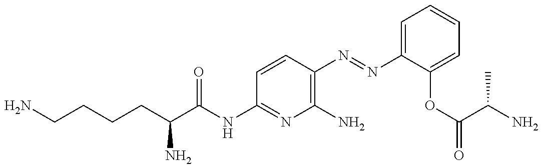 [SMILES: C[C@H](N)C(=O)Oc1ccccc1/N=N/c1ccc(NC(=O)[C@@H](N)CCCCN)nc1N] |
| 314 | 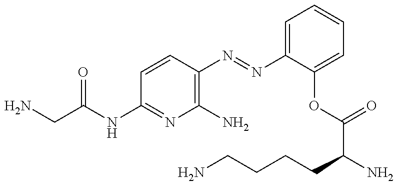 [SMILES: NCCCC[C@H](N)C(=O)Oc1ccccc1/N=N/c1ccc(NC(=O)CN)nc1N] |
| 315 | 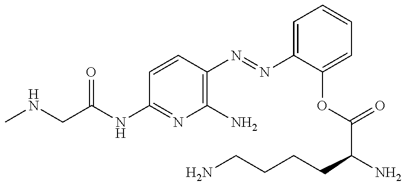 [SMILES: CNCC(=O)Nc1ccc(/N=N/c2ccccc2OC(=O)[C@@H](N)CCCCN)c(N)n1] |
| 316 | 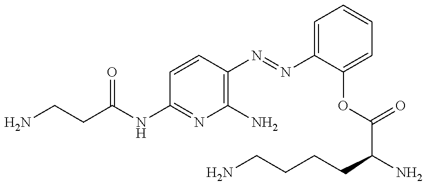 [SMILES: NCCCC[C@H](N)C(=O)Oc1ccccc1/N=N/c1ccc(NC(=O)CCN)nc1N] |
| 317 | 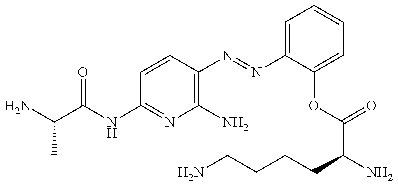 [SMILES: C[C@H](N)C(=O)Nc1ccc(/N=N/c2ccccc2OC(=O)[C@@H](N)CCCCN)c(N)n1] |
| 318 | 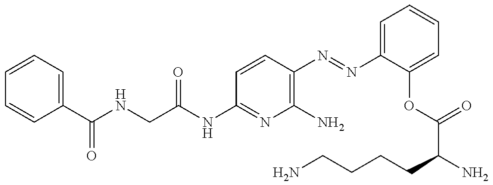 [SMILES: NCCCC[C@H](N)C(=O)Oc1ccccc1/N=N/c1ccc(NC(=O)CNC(=O)c2ccccc2)nc1N] |
| 319 | 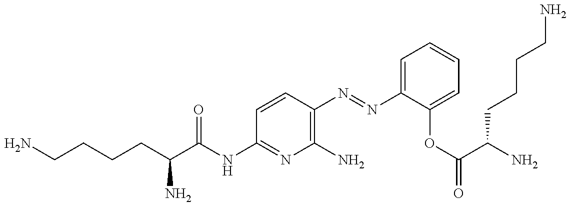 [SMILES: NCCCC[C@H](N)C(=O)Nc1ccc(/N=N/c2ccccc2OC(=O)[C@@H](N)CCCCN)c(N)n1] |
| 320 | 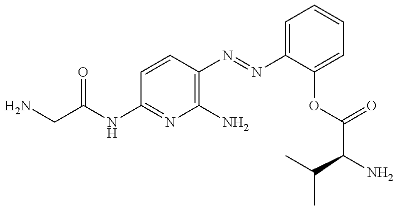 [SMILES: CC(C)[C@H](N)C(=O)Oc1ccccc1/N=N/c1ccc(NC(=O)CN)nc1N] |
| 321 | 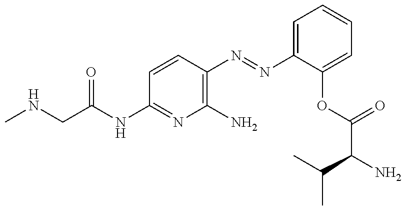 [SMILES: CNCC(=O)Nc1ccc(/N=N/c2ccccc2OC(=O)[C@@H](N)C(C)C)c(N)n1] |
| 322 | 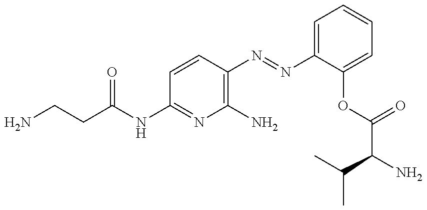 [SMILES: CC(C)[C@H](N)C(=O)Oc1ccccc1/N=N/c1ccc(NC(=O)CCN)nc1N] |
| 323 | 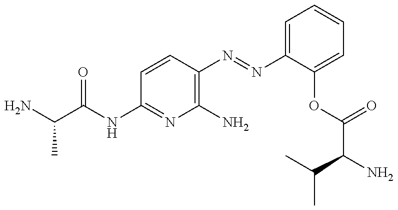 [SMILES: CC(C)[C@H](N)C(=O)Oc1ccccc1/N=N/c1ccc(NC(=O)[C@H](C)N)nc1N] |
| 324 | 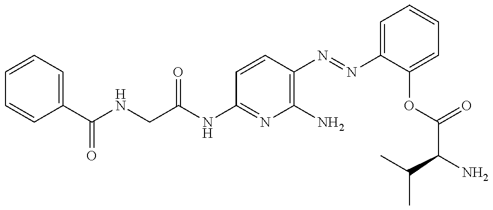 [SMILES: CC(C)[C@H](N)C(=O)Oc1ccccc1/N=N/c1ccc(NC(=O)CNC(=O)c2ccccc2)nc1N] |
| 325 | 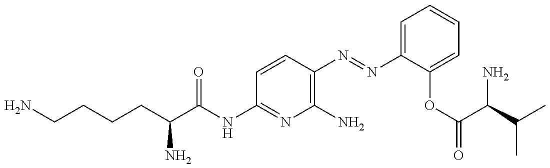 [SMILES: CC(C)[C@H](N)C(=O)Oc1ccccc1/N=N/c1ccc(NC(=O)[C@@H](N)CCCCN)nc1N] |
| 326 | 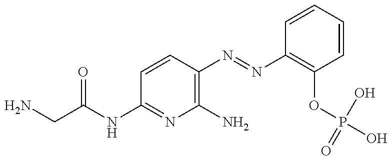 [SMILES: NCC(=O)Nc1ccc(/N=N/c2ccccc2OP(=O)(O)O)c(N)n1] |
| 327 | 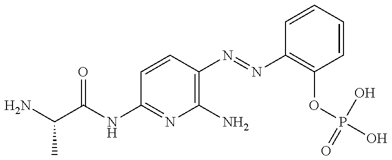 [SMILES: C[C@H](N)C(=O)Nc1ccc(/N=N/c2ccccc2OP(=O)(O)O)c(N)n1] |
| 328 | 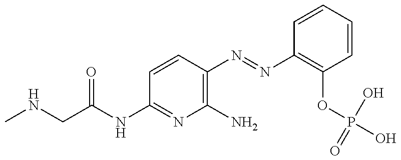 [SMILES: CNCC(=O)Nc1ccc(/N=N/c2ccccc2OP(=O)(O)O)c(N)n1] |
| 329 | 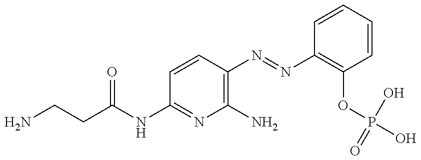 [SMILES: NCCC(=O)Nc1ccc(/N=N/c2ccccc2OP(=O)(O)O)c(N)n1] |
| 330 | 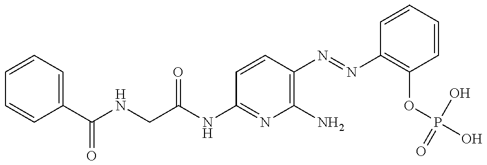 [SMILES: Nc1nc(NC(=O)CNC(=O)c2ccccc2)ccc1/N=N/c1ccccc1OP(=O)(O)O] |
| 331 | 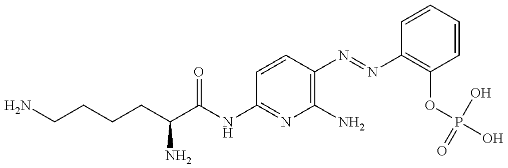 [SMILES: NCCCC[C@H](N)C(=O)Nc1ccc(/N=N/c2ccccc2OP(=O)(O)O)c(N)n1] |
| 332 | 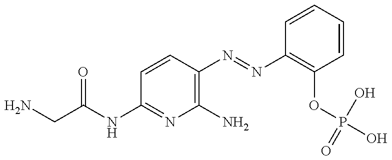 [SMILES: NCC(=O)Nc1ccc(/N=N/c2ccccc2OP(=O)(O)O)c(N)n1] |
| 333 | 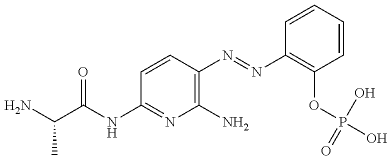 [SMILES: C[C@H](N)C(=O)Nc1ccc(/N=N/c2ccccc2OP(=O)(O)O)c(N)n1] |
| 334 | 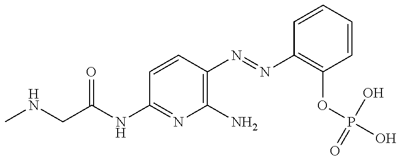 [SMILES: CNCC(=O)Nc1ccc(/N=N/c2ccccc2OP(=O)(O)O)c(N)n1] |
| 335 | 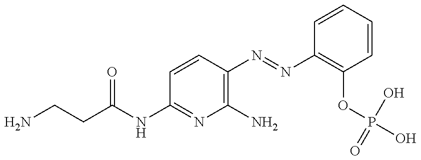 [SMILES: NCCC(=O)Nc1ccc(/N=N/c2ccccc2OP(=O)(O)O)c(N)n1] |
| 336 | 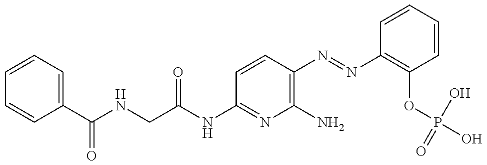 [SMILES: Nc1nc(NC(=O)CNC(=O)c2ccccc2)ccc1/N=N/c1ccccc1OP(=O)(O)O] |
| 337 | 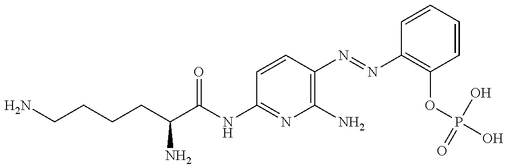 [SMILES: NCCCC[C@H](N)C(=O)Nc1ccc(/N=N/c2ccccc2OP(=O)(O)O)c(N)n1] |
| 338 | 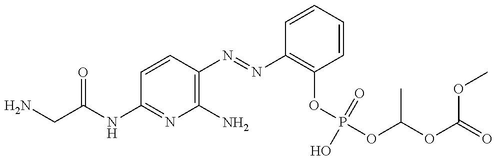 [SMILES: COC(=O)OC(C)OP(=O)(O)Oc1ccccc1/N=N/c1ccc(NC(=O)CN)nc1N] |
| 339 | 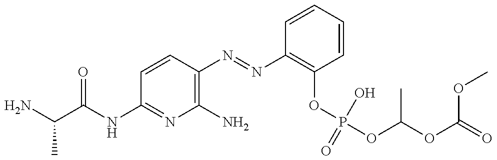 [SMILES: COC(=O)OC(C)OP(=O)(O)Oc1ccccc1/N=N/c1ccc(NC(=O)[C@H](C)N)nc1N] |
| 340 | 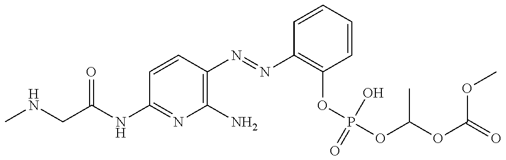 |
| 341 | 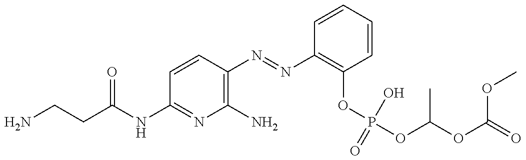 [SMILES: COC(=O)OC(C)OP(=O)(O)Oc1ccccc1/N=N/c1ccc(NC(=O)CCN)nc1N] |
| 342 | 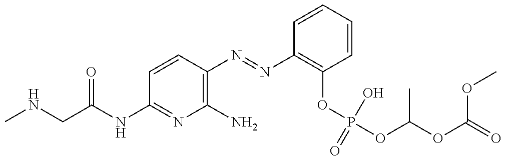 |
| 343 | 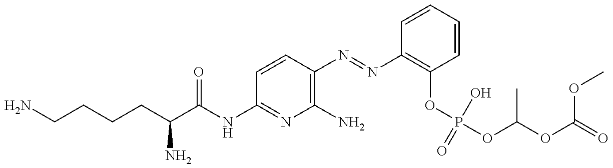 [SMILES: COC(=O)OC(C)OP(=O)(O)Oc1ccccc1/N=N/c1ccc(NC(=O)[C@@H](N)CCCCN)nc1N] |
| 344 | 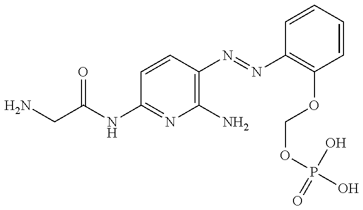 [SMILES: NCC(=O)Nc1ccc(/N=N/c2ccccc2OCOP(=O)(O)O)c(N)n1] |
| 345 | 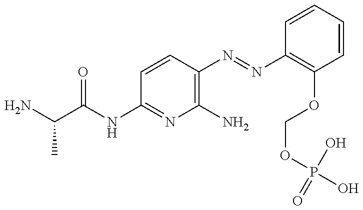 [SMILES: C[C@H](N)C(=O)Nc1ccc(/N=N/c2ccccc2OCOP(=O)(O)O)c(N)n1] |
| 346 | 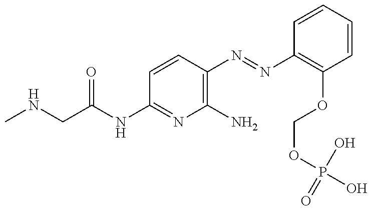 [SMILES: CNCC(=O)Nc1ccc(/N=N/c2ccccc2OCOP(=O)(O)O)c(N)n1] |
| 347 | 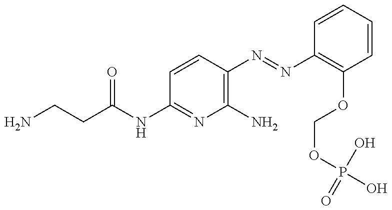 [SMILES: NCCC(=O)Nc1ccc(/N=N/c2ccccc2OCOP(=O)(O)O)c(N)n1] |
| 348 | 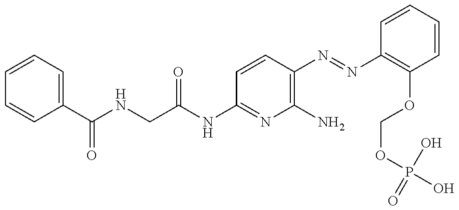 [SMILES: Nc1nc(NC(=O)CNC(=O)c2ccccc2)ccc1/N=N/c1ccccc1OCOP(=O)(O)O] |
| 349 | 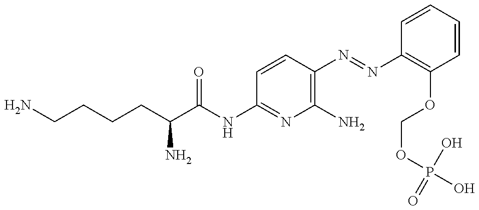 [SMILES: NCCCC[C@H](N)C(=O)Nc1ccc(/N=N/c2ccccc2OCOP(=O)(O)O)c(N)n1] |
| 350 | 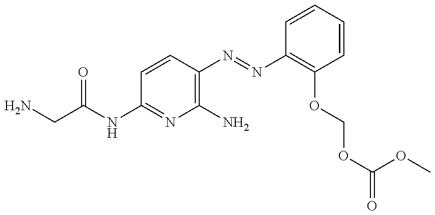 [SMILES: COC(=O)OCOc1ccccc1/N=N/c1ccc(NC(=O)CN)nc1N] |
| 351 | 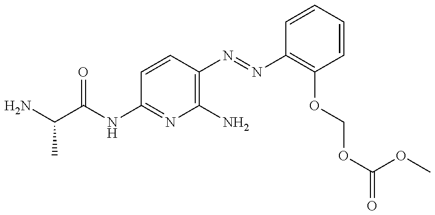 [SMILES: COC(=O)OCOc1ccccc1/N=N/c1ccc(NC(=O)[C@H](C)N)nc1N] |
| 352 | 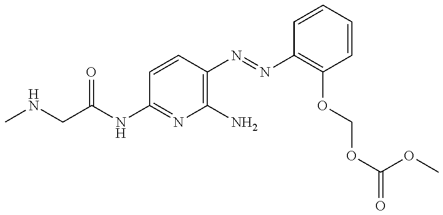 |
| 353 | 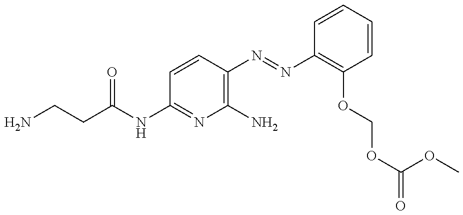 [SMILES: COC(=O)OCOc1ccccc1/N=N/c1ccc(NC(=O)CCN)nc1N] |
| 354 | 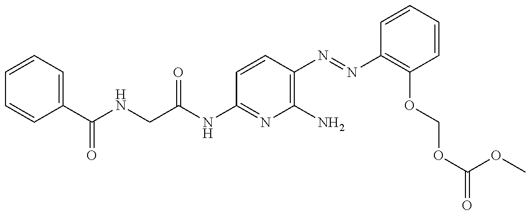 [SMILES: COC(=O)OCOc1ccccc1/N=N/c1ccc(NC(=O)CNC(=O)c2ccccc2)nc1N] |
| 355 | 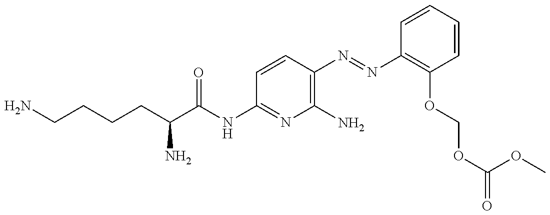 [SMILES: COC(=O)OCOc1ccccc1/N=N/c1ccc(NC(=O)[C@@H](N)CCCCN)nc1N] |
| 356 | 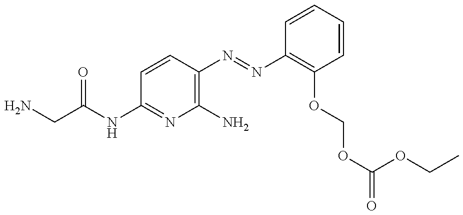 [SMILES: CCOC(=O)OCOc1ccccc1/N=N/c1ccc(NC(=O)CN)nc1N] |
| 357 | 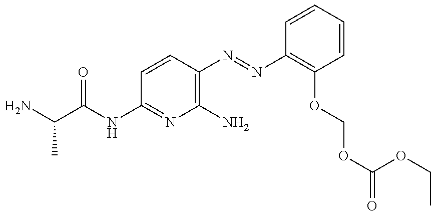 [SMILES: CCOC(=O)OCOc1ccccc1/N=N/c1ccc(NC(=O)[C@H](C)N)nc1N] |
| 358 | 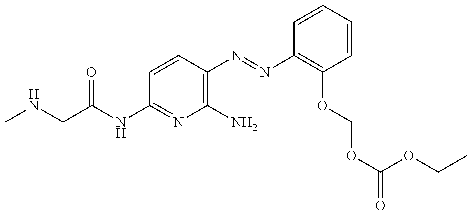 [SMILES: CCOC(=O)OCOc1ccccc1/N=N/c1ccc(NC(=O)CNC)nc1N] |
| 359 | 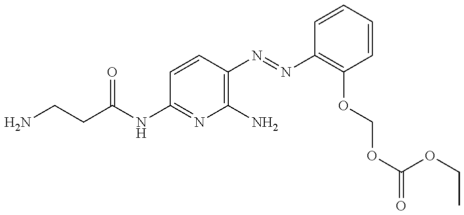 [SMILES: CCOC(=O)OCOc1ccccc1/N=N/c1ccc(NC(=O)CCN)nc1N] |
| 360 | 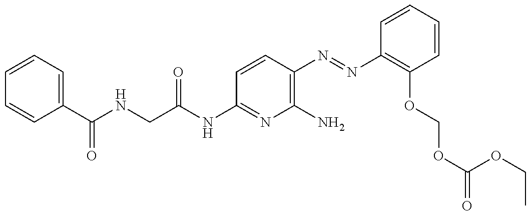 [SMILES: CCOC(=O)OCOc1ccccc1/N=N/c1ccc(NC(=O)CNC(=O)c2ccccc2)nc1N] |
| 361 | 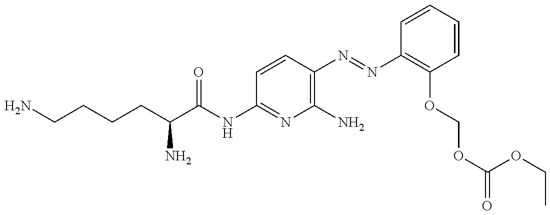 [SMILES: CCOC(=O)OCOc1ccccc1/N=N/c1ccc(NC(=O)[C@@H](N)CCCCN)nc1N] |
| 362 | 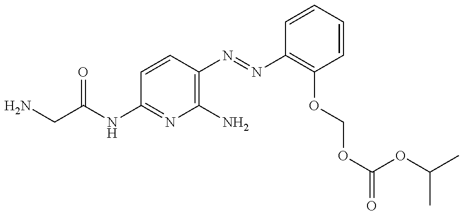 [SMILES: CC(C)OC(=O)OCOc1ccccc1/N=N/c1ccc(NC(=O)CN)nc1N] |
| 363 | 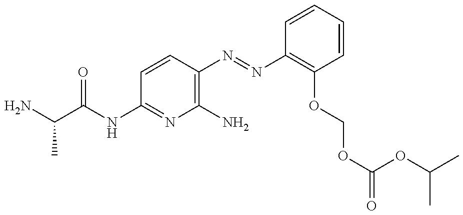 [SMILES: CC(C)OC(=O)OCOc1ccccc1/N=N/c1ccc(NC(=O)[C@H](C)N)nc1N] |
| 364 | 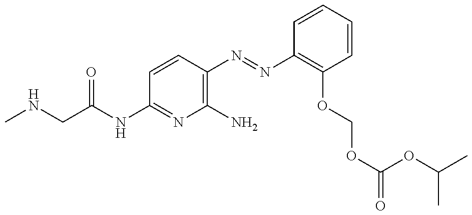 [SMILES: CNCC(=O)Nc1ccc(/N=N/c2ccccc2OCOC(=O)OC(C)C)c(N)n1] |
| 365 | 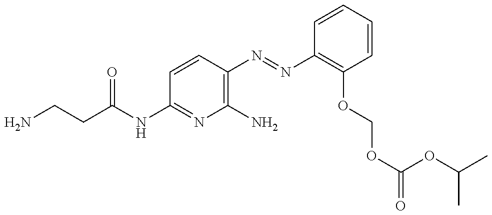 [SMILES: CC(C)OC(=O)OCOc1ccccc1/N=N/c1ccc(NC(=O)CCN)nc1N] |
| 366 | 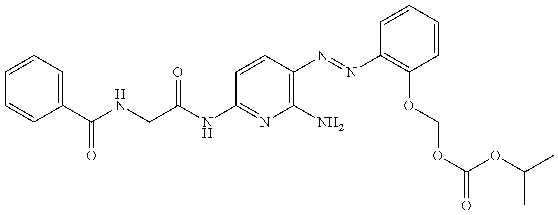 [SMILES: CC(C)OC(=O)OCOc1ccccc1/N=N/c1ccc(NC(=O)CNC(=O)c2ccccc2)nc1N] |
| 367 | 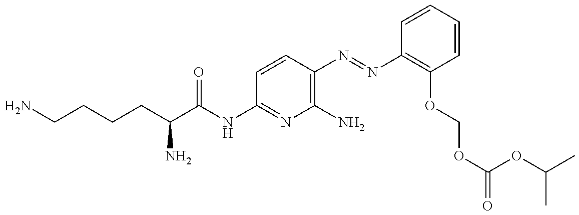 [SMILES: CC(C)OC(=O)OCOc1ccccc1/N=N/c1ccc(NC(=O)[C@@H](N)CCCCN)nc1N] |
| 368 | 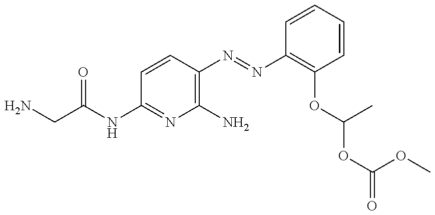 [SMILES: COC(=O)OC(C)Oc1ccccc1/N=N/c1ccc(NC(=O)CN)nc1N] |
| 369 | 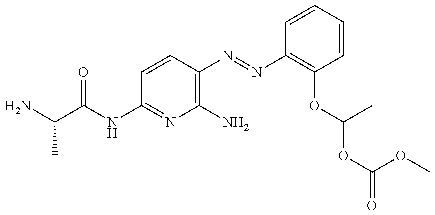 [SMILES: COC(=O)OC(C)Oc1ccccc1/N=N/c1ccc(NC(=O)[C@H](C)N)nc1N] |
| 370 | 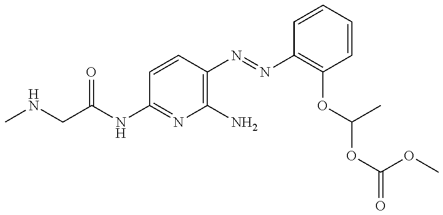 |
| 371 | 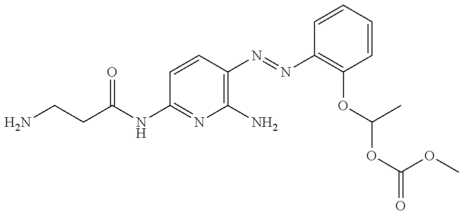 [SMILES: COC(=O)OC(C)Oc1ccccc1/N=N/c1ccc(NC(=O)CCN)nc1N] |
| 372 | 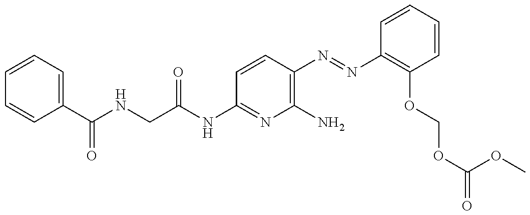 [SMILES: COC(=O)OCOc1ccccc1/N=N/c1ccc(NC(=O)CNC(=O)c2ccccc2)nc1N] |
| 373 | 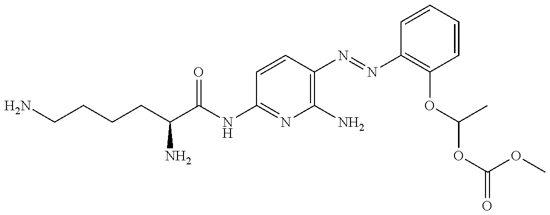 [SMILES: COC(=O)OC(C)Oc1ccccc1/N=N/c1ccc(NC(=O)[C@@H](N)CCCCN)nc1N] |
| 374 | 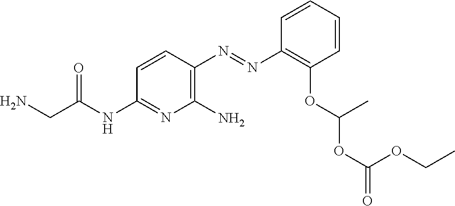 [SMILES: CCOC(=O)OC(C)Oc1ccccc1/N=N/c1ccc(NC(=O)CN)nc1N] |
| 375 | 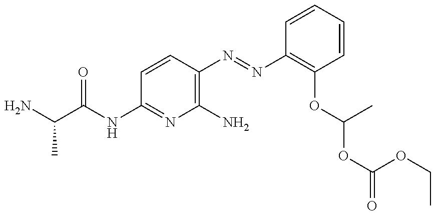 [SMILES: CCOC(=O)OC(C)Oc1ccccc1/N=N/c1ccc(NC(=O)[C@H](C)N)nc1N] |
| 376 | 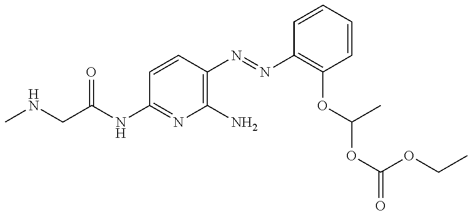 [SMILES: CCOC(=O)OC(C)Oc1ccccc1/N=N/c1ccc(NC(=O)CNC)nc1N] |
| 377 | 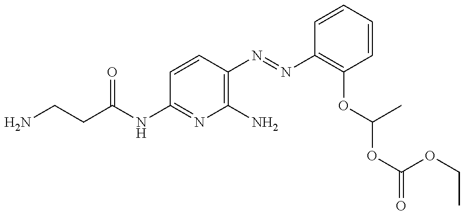 [SMILES: CCOC(=O)OC(C)Oc1ccccc1/N=N/c1ccc(NC(=O)CCN)nc1N] |
| 378 | 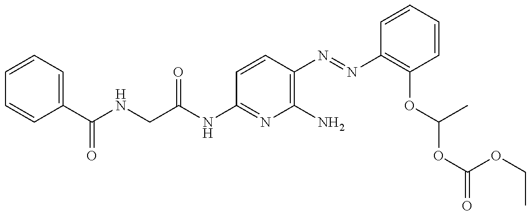 [SMILES: CCOC(=O)OC(C)Oc1ccccc1/N=N/c1ccc(NC(=O)CNC(=O)c2ccccc2)nc1N] |
| 379 | 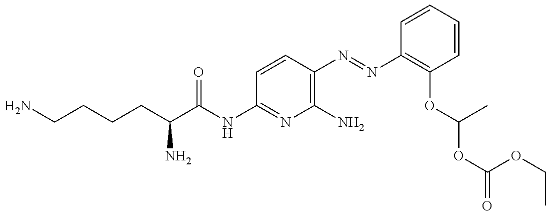 [SMILES: CCOC(=O)OC(C)Oc1ccccc1/N=N/c1ccc(NC(=O)[C@@H](N)CCCCN)nc1N] |
| 380 | 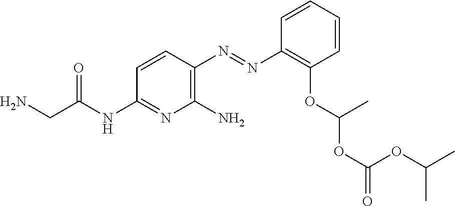 [SMILES: CC(C)OC(=O)OC(C)Oc1ccccc1/N=N/c1ccc(NC(=O)CN)nc1N] |
| 381 | 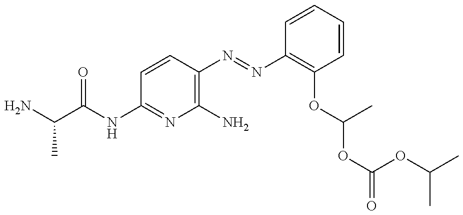 [SMILES: CC(C)OC(=O)OC(C)Oc1ccccc1/N=N/c1ccc(NC(=O)[C@H](C)N)nc1N] |
| 382 | 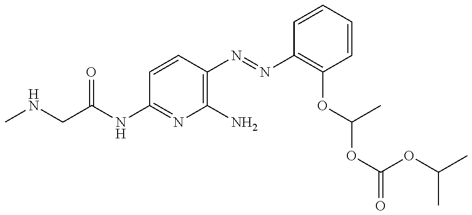 [SMILES: CNCC(=O)Nc1ccc(/N=N/c2ccccc2OC(C)OC(=O)OC(C)C)c(N)n1] |
| 383 | 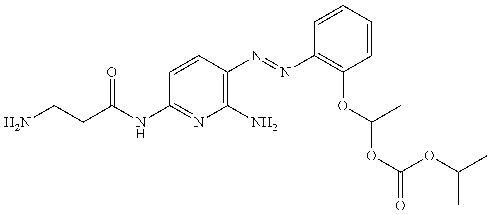 [SMILES: CC(C)OC(=O)OC(C)Oc1ccccc1/N=N/c1ccc(NC(=O)CCN)nc1N] |
| 384 | 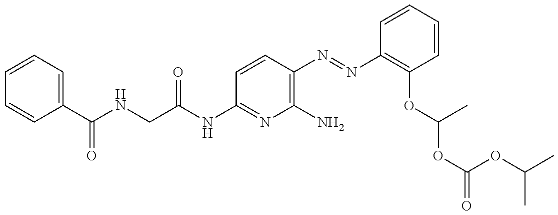 [SMILES: CC(C)OC(=O)OC(C)Oc1ccccc1/N=N/c1ccc(NC(=O)CNC(=O)c2ccccc2)nc1N] |
| 385 | 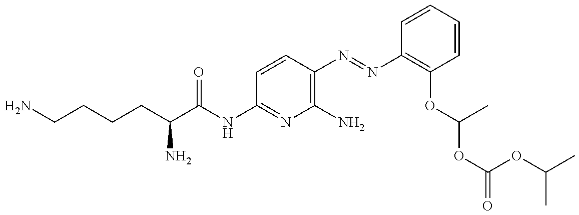 [SMILES: CC(C)OC(=O)OC(C)Oc1ccccc1/N=N/c1ccc(NC(=O)[C@@H](N)CCCCN)nc1N] |
| 386 | 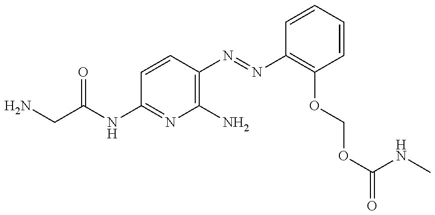 [SMILES: CNC(=O)OCOc1ccccc1/N=N/c1ccc(NC(=O)CN)nc1N] |
| 387 | 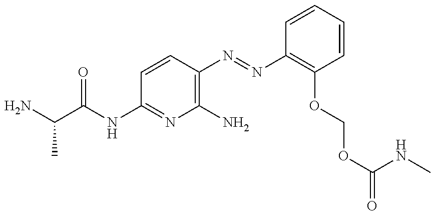 [SMILES: CNC(=O)OCOc1ccccc1/N=N/c1ccc(NC(=O)[C@H](C)N)nc1N] |
| 388 | 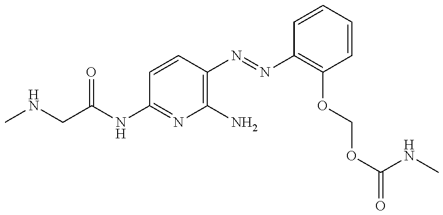 |
| 389 | 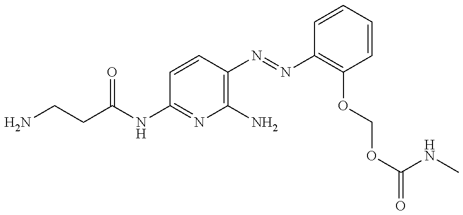 [SMILES: CNC(=O)OCOc1ccccc1/N=N/c1ccc(NC(=O)CCN)nc1N] |
| 390 | 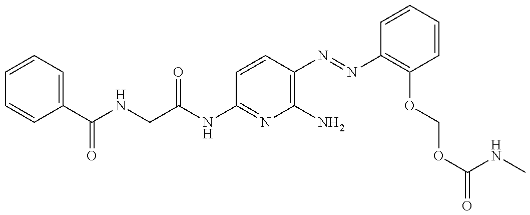 [SMILES: CNC(=O)OCOc1ccccc1/N=N/c1ccc(NC(=O)CNC(=O)c2ccccc2)nc1N] |
| 391 | 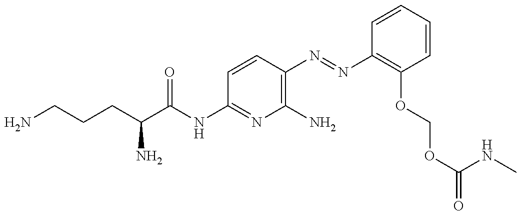 [SMILES: CNC(=O)OCOc1ccccc1/N=N/c1ccc(NC(=O)[C@@H](N)CCCN)nc1N] |
| 392 | 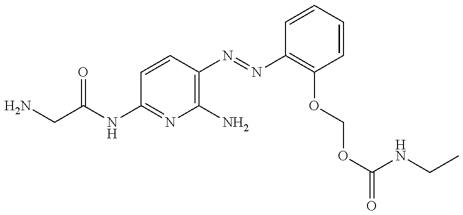 [SMILES: CCNC(=O)OCOc1ccccc1/N=N/c1ccc(NC(=O)CN)nc1N] |
| 393 | 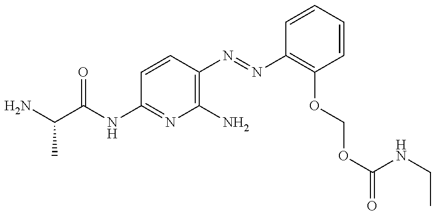 [SMILES: CCNC(=O)OCOc1ccccc1/N=N/c1ccc(NC(=O)[C@H](C)N)nc1N] |
| 394 | 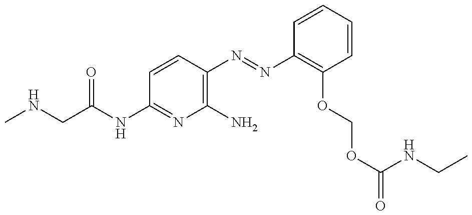 [SMILES: CCNC(=O)OCOc1ccccc1/N=N/c1ccc(NC(=O)CNC)nc1N] |
| 395 | 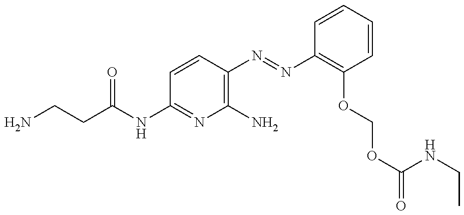 [SMILES: CCNC(=O)OCOc1ccccc1/N=N/c1ccc(NC(=O)CCN)nc1N] |
| 396 | 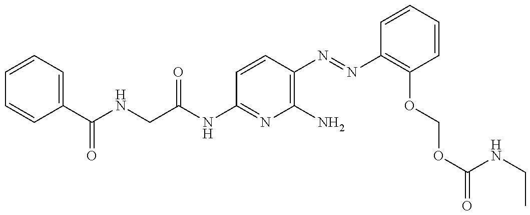 [SMILES: CCNC(=O)OCOc1ccccc1/N=N/c1ccc(NC(=O)CNC(=O)c2ccccc2)nc1N] |
| 397 | 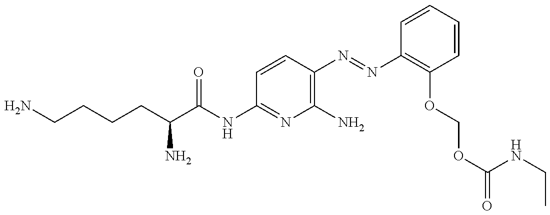 [SMILES: CCNC(=O)OCOc1ccccc1/N=N/c1ccc(NC(=O)[C@@H](N)CCCCN)nc1N] |
| 398 | 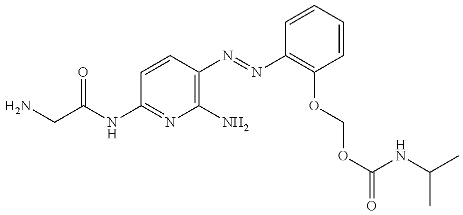 [SMILES: CC(C)NC(=O)OCOc1ccccc1/N=N/c1ccc(NC(=O)CN)nc1N] |
| 399 | 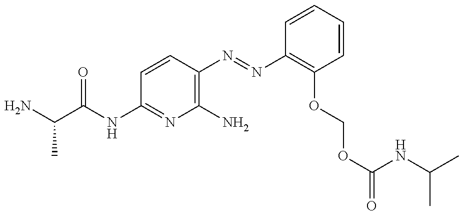 [SMILES: CC(C)NC(=O)OCOc1ccccc1/N=N/c1ccc(NC(=O)[C@H](C)N)nc1N] |
| 400 | 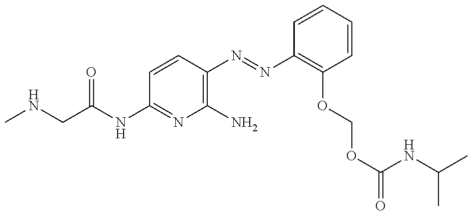 [SMILES: CNCC(=O)Nc1ccc(/N=N/c2ccccc2OCOC(=O)NC(C)C)c(N)n1] |
| 401 | 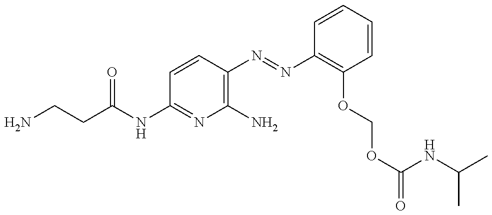 [SMILES: CC(C)NC(=O)OCOc1ccccc1/N=N/c1ccc(NC(=O)CCN)nc1N] |
| 402 | 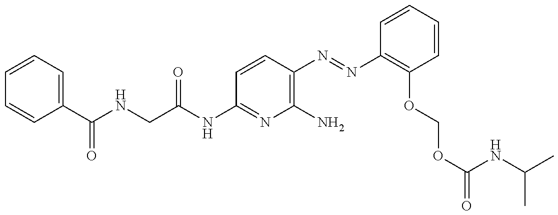 [SMILES: CC(C)NC(=O)OCOc1ccccc1/N=N/c1ccc(NC(=O)CNC(=O)c2ccccc2)nc1N] |
| 403 | 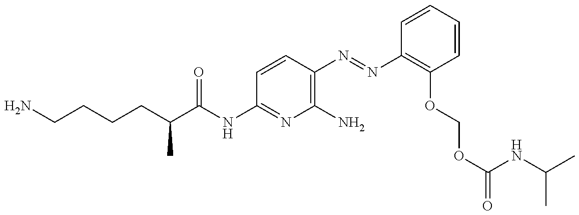 [SMILES: CC(C)NC(=O)OCOc1ccccc1/N=N/c1ccc(NC(=O)[C@@H](C)CCCCN)nc1N] |
| 404 | 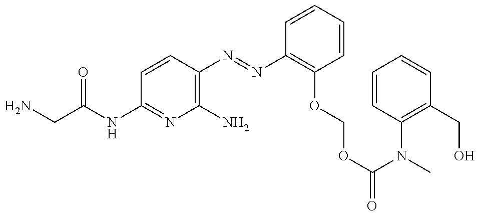 [SMILES: CN(C(=O)OCOc1ccccc1/N=N/c1ccc(NC(=O)CN)nc1N)c1ccccc1CO] |
| 405 | 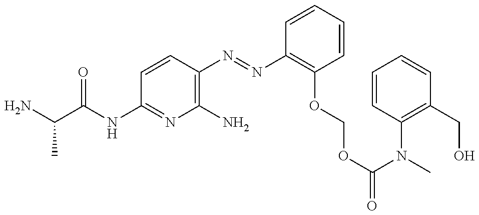 [SMILES: C[C@H](N)C(=O)Nc1ccc(/N=N/c2ccccc2OCOC(=O)N(C)c2ccccc2CO)c(N)n1] |
| 406 | 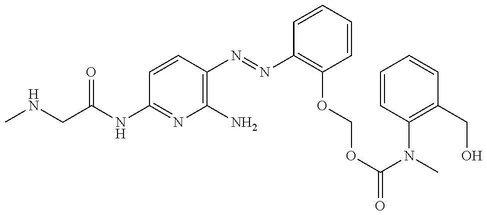 [SMILES: CNCC(=O)Nc1ccc(/N=N/c2ccccc2OCOC(=O)N(C)c2ccccc2CO)c(N)n1] |
| 407 | 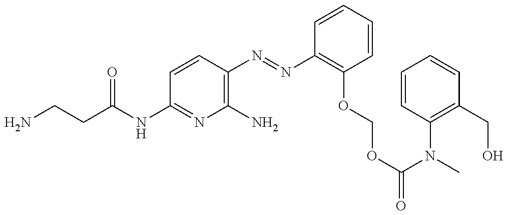 [SMILES: CN(C(=O)OCOc1ccccc1/N=N/c1ccc(NC(=O)CCN)nc1N)c1ccccc1CO] |
| 408 | 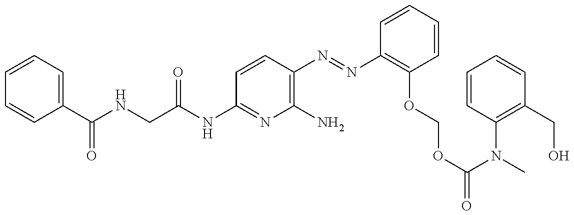 [SMILES: CN(C(=O)OCOc1ccccc1/N=N/c1ccc(NC(=O)CNC(=O)c2ccccc2)nc1N)c1ccccc1CO] |
| 409 | 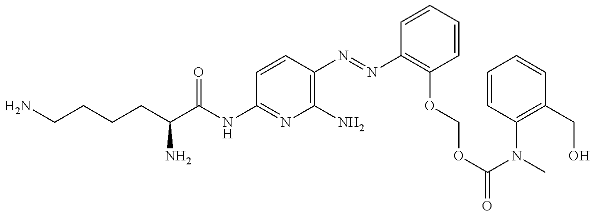 [SMILES: CN(C(=O)OCOc1ccccc1/N=N/c1ccc(NC(=O)[C@@H](N)CCCCN)nc1N)c1ccccc1CO] |
| 410 | 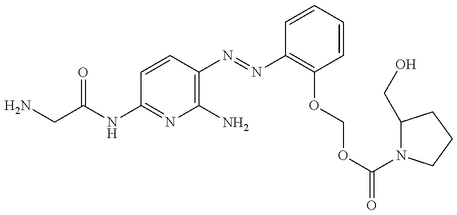 [SMILES: NCC(=O)Nc1ccc(/N=N/c2ccccc2OCOC(=O)N2CCCC2CO)c(N)n1] |
| 411 | 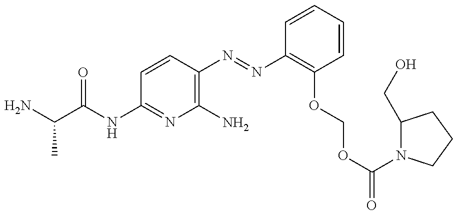 [SMILES: C[C@H](N)C(=O)Nc1ccc(/N=N/c2ccccc2OCOC(=O)N2CCCC2CO)c(N)n1] |
| 412 | 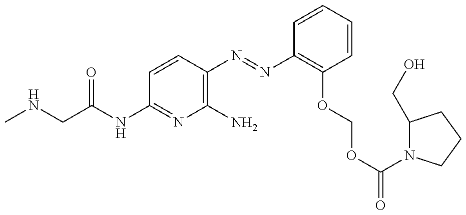 [SMILES: CNCC(=O)Nc1ccc(/N=N/c2ccccc2OCOC(=O)N2CCCC2CO)c(N)n1] |
| 413 | 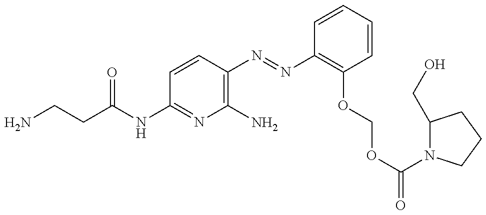 [SMILES: NCCC(=O)Nc1ccc(/N=N/c2ccccc2OCOC(=O)N2CCCC2CO)c(N)n1] |
| 414 | 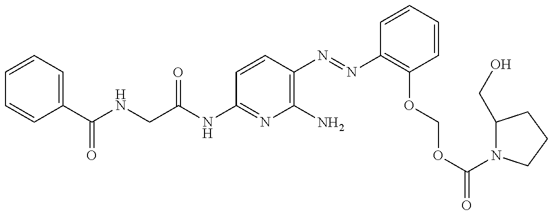 [SMILES: Nc1nc(NC(=O)CNC(=O)c2ccccc2)ccc1/N=N/c1ccccc1OCOC(=O)N1CCCC1CO] |
| 415 | 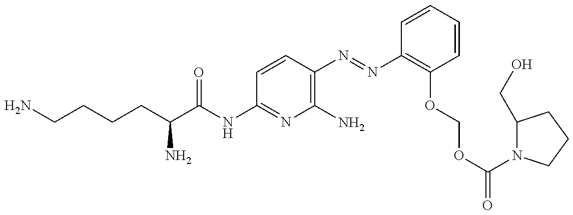 [SMILES: NCCCC[C@H](N)C(=O)Nc1ccc(/N=N/c2ccccc2OCOC(=O)N2CCCC2CO)c(N)n1] |
| 416 | 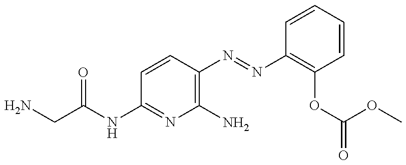 [SMILES: COC(=O)Oc1ccccc1/N=N/c1ccc(NC(=O)CN)nc1N] |
| 417 | 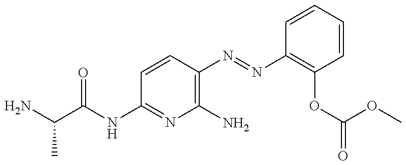 [SMILES: COC(=O)Oc1ccccc1/N=N/c1ccc(NC(=O)[C@H](C)N)nc1N] |
| 418 | 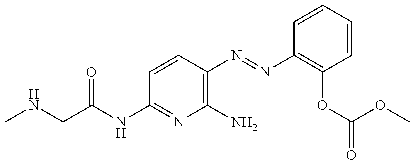 |
| 419 | 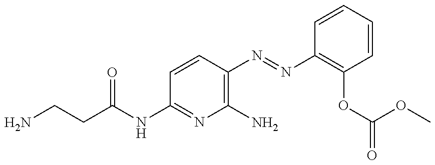 [SMILES: COC(=O)Oc1ccccc1/N=N/c1ccc(NC(=O)CCN)nc1N] |
| 420 | 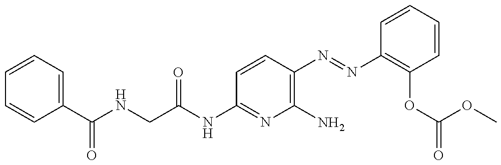 [SMILES: COC(=O)Oc1ccccc1/N=N/c1ccc(NC(=O)CNC(=O)c2ccccc2)nc1N] |
| 421 | 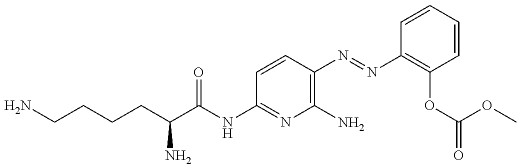 [SMILES: COC(=O)Oc1ccccc1/N=N/c1ccc(NC(=O)[C@@H](N)CCCCN)nc1N] |
| 422 | 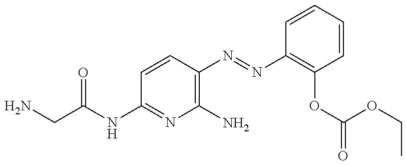 [SMILES: CCOC(=O)Oc1ccccc1/N=N/c1ccc(NC(=O)CN)nc1N] |
| 423 | 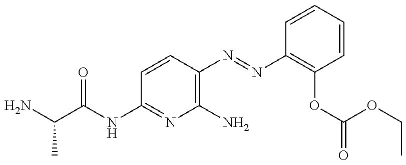 [SMILES: CCOC(=O)Oc1ccccc1/N=N/c1ccc(NC(=O)[C@H](C)N)nc1N] |
| 424 | 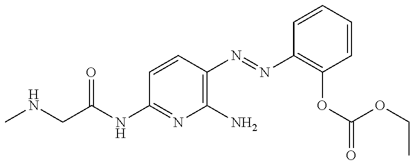 [SMILES: CCOC(=O)Oc1ccccc1/N=N/c1ccc(NC(=O)CNC)nc1N] |
| 425 | 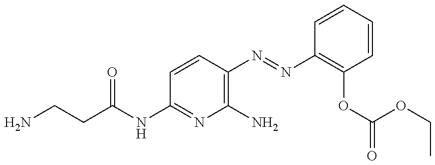 [SMILES: CCOC(=O)Oc1ccccc1/N=N/c1ccc(NC(=O)CCN)nc1N] |
| 426 | 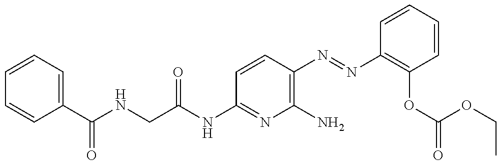 [SMILES: CCOC(=O)Oc1ccccc1/N=N/c1ccc(NC(=O)CNC(=O)c2ccccc2)nc1N] |
| 427 | 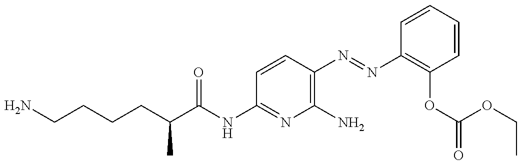 [SMILES: CCOC(=O)Oc1ccccc1/N=N/c1ccc(NC(=O)[C@@H](C)CCCCN)nc1N] |
| 428 | 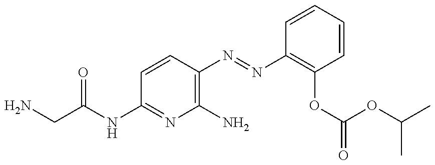 [SMILES: CC(C)OC(=O)Oc1ccccc1/N=N/c1ccc(NC(=O)CN)nc1N] |
| 429 | 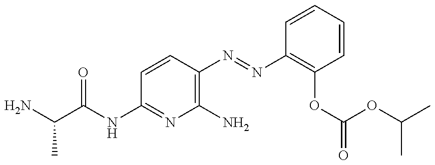 [SMILES: CC(C)OC(=O)Oc1ccccc1/N=N/c1ccc(NC(=O)[C@H](C)N)nc1N] |
| 430 | 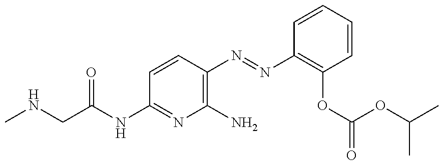 [SMILES: CNCC(=O)Nc1ccc(/N=N/c2ccccc2OC(=O)OC(C)C)c(N)n1] |
| 431 | 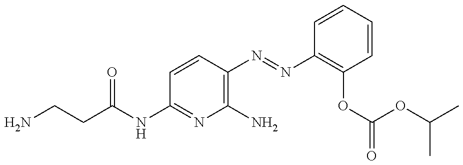 [SMILES: CC(C)OC(=O)Oc1ccccc1/N=N/c1ccc(NC(=O)CCN)nc1N] |
| 432 | 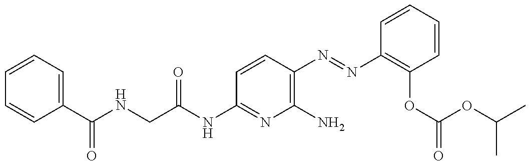 [SMILES: CC(C)OC(=O)Oc1ccccc1/N=N/c1ccc(NC(=O)CNC(=O)c2ccccc2)nc1N] |
| 433 | 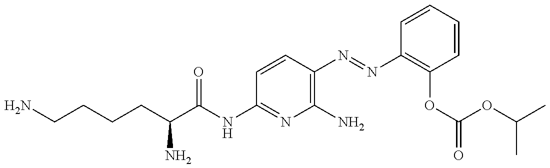 [SMILES: CC(C)OC(=O)Oc1ccccc1/N=N/c1ccc(NC(=O)[C@@H](N)CCCCN)nc1N] |
| 434 | 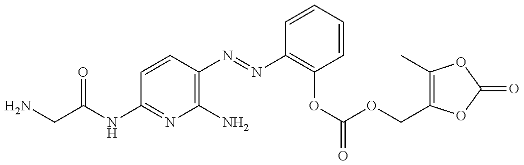 [SMILES: Cc1oc(=O)oc1COC(=O)Oc1ccccc1/N=N/c1ccc(NC(=O)CN)nc1N] |
| 435 | 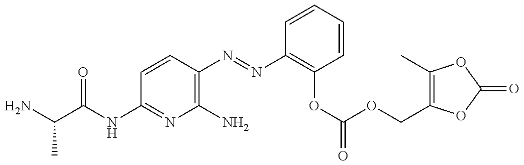 [SMILES: Cc1oc(=O)oc1COC(=O)Oc1ccccc1/N=N/c1ccc(NC(=O)[C@H](C)N)nc1N] |
| 436 | 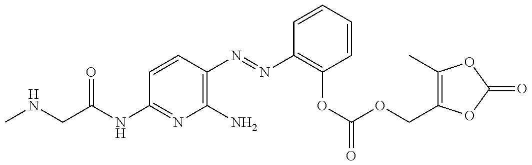 [SMILES: CNCC(=O)Nc1ccc(/N=N/c2ccccc2OC(=O)OCc2oc(=O)oc2C)c(N)n1] |
| 437 | 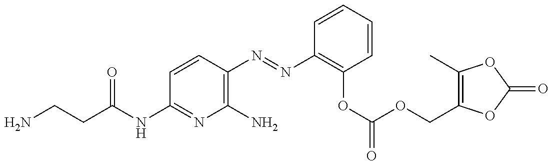 [SMILES: Cc1oc(=O)oc1COC(=O)Oc1ccccc1/N=N/c1ccc(NC(=O)CCN)nc1N] |
| 438 | 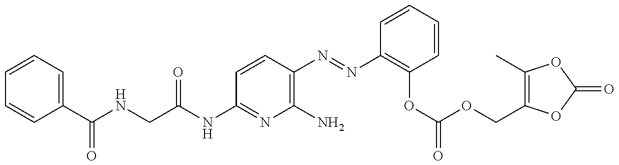 [SMILES: Cc1oc(=O)oc1COC(=O)Oc1ccccc1/N=N/c1ccc(NC(=O)CNC(=O)c2ccccc2)nc1N] |
| 439 | 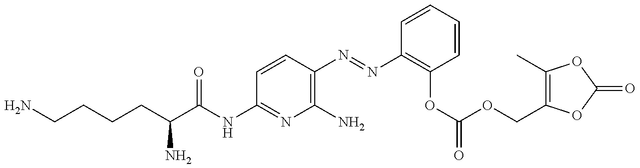 [SMILES: Cc1oc(=O)oc1COC(=O)Oc1ccccc1/N=N/c1ccc(NC(=O)[C@@H](N)CCCCN)nc1N] |
| 440 | 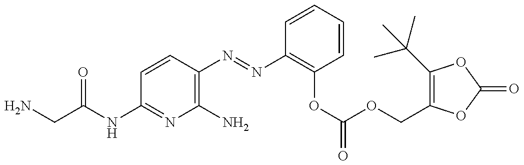 [SMILES: CC(C)(C)c1oc(=O)oc1COC(=O)Oc1ccccc1/N=N/c1ccc(NC(=O)CN)nc1N] |
| 441 | 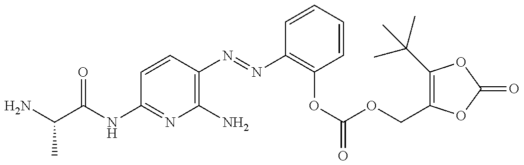 [SMILES: C[C@H](N)C(=O)Nc1ccc(/N=N/c2ccccc2OC(=O)OCc2oc(=O)oc2C(C)(C)C)c(N)n1] |
| 442 | 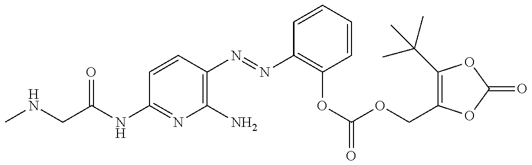 [SMILES: CNCC(=O)Nc1ccc(/N=N/c2ccccc2OC(=O)OCc2oc(=O)oc2C(C)(C)C)c(N)n1] |
| 443 | 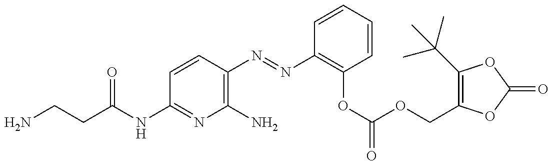 [SMILES: CC(C)(C)c1oc(=O)oc1COC(=O)Oc1ccccc1/N=N/c1ccc(NC(=O)CCN)nc1N] |
| 444 | 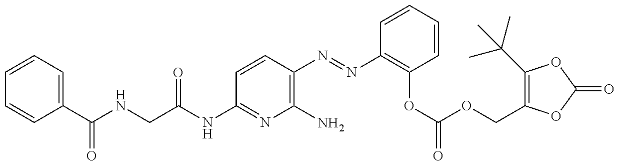 [SMILES: CC(C)(C)c1oc(=O)oc1COC(=O)Oc1ccccc1/N=N/c1ccc(NC(=O)CNC(=O)c2ccccc2)nc1N] |
| 445 | 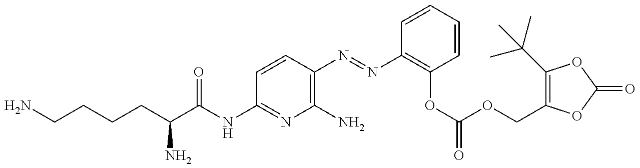 [SMILES: CC(C)(C)c1oc(=O)oc1COC(=O)Oc1ccccc1/N=N/c1ccc(NC(=O)[C@@H](N)CCCCN)nc1N] |
| 446 | 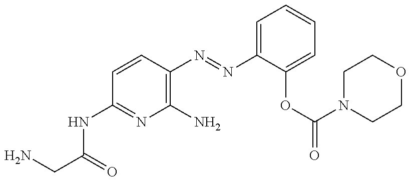 [SMILES: NCC(=O)Nc1ccc(/N=N/c2ccccc2OC(=O)N2CCOCC2)c(N)n1] |
| 447 | 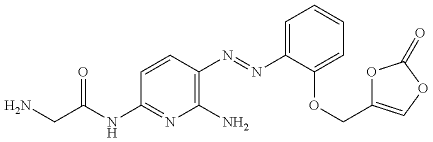 [SMILES: NCC(=O)Nc1ccc(/N=N/c2ccccc2OCc2coc(=O)o2)c(N)n1] |
| 448 | 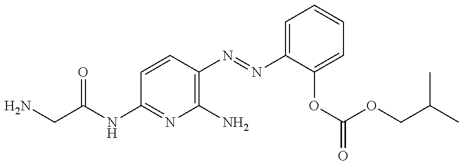 [SMILES: CC(C)COC(=O)Oc1ccccc1/N=N/c1ccc(NC(=O)CN)nc1N] |
| 449 | 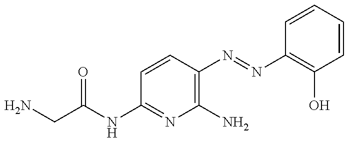 [SMILES: NCC(=O)Nc1ccc(/N=N/c2ccccc2O)c(N)n1] |
| 450 | 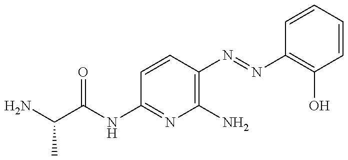 [SMILES: C[C@H](N)C(=O)Nc1ccc(/N=N/c2ccccc2O)c(N)n1] |
| 451 | 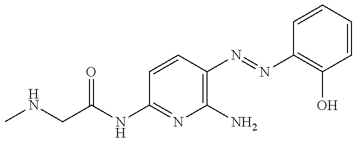 [SMILES: CNCC(=O)Nc1ccc(/N=N/c2ccccc2O)c(N)n1] |
| 452 | 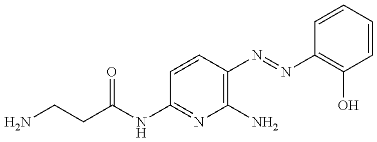 [SMILES: NCCC(=O)Nc1ccc(/N=N/c2ccccc2O)c(N)n1] |
| 453 | 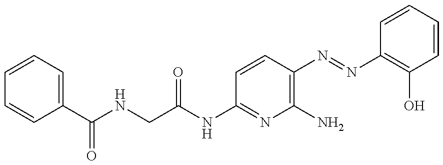 [SMILES: Nc1nc(NC(=O)CNC(=O)c2ccccc2)ccc1/N=N/c1ccccc1O] |
| 454 | 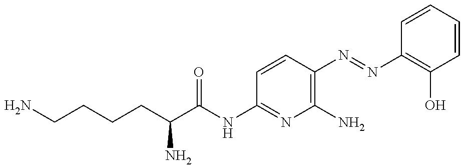 [SMILES: NCCCC[C@H](N)C(=O)Nc1ccc(/N=N/c2ccccc2O)c(N)n1] |

In another aspect, the present invention provides a method for the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, comprises administering a compound selected from general formula (I) or (I-A), pharmaceutically acceptable salts or solvates thereof.

In another aspect, the present invention provides a method for the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, comprises administering a compound selected from compounds of general formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), formula (X) pharmaceutically acceptable salts or solvates thereof.

In another aspect, the present invention provides a method for the symptomatic relief of pain, burning, urgency, frequency, and other discomforts arising from irritation of the lower urinary tract mucosa caused by infection, trauma, surgery, endoscopic procedures, or the passage of sounds or catheters comprises administering a therapeutically effective amount of compound of formula (I) or formula (I-A); pharmaceutically acceptable salts or solvates thereof.

In another aspect, the present invention provides a method for the symptomatic relief of pain, burning, urgency, frequency, and other discomforts arising from irritation of the lower urinary tract mucosa caused by infection, trauma, surgery, endoscopic procedures, or the passage of sounds or catheters comprises administering a compound selected from compounds of general formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), formula (X) pharmaceutically acceptable salts or solvates thereof.

In yet another aspect, the present invention relates to pharmaceutical composition comprising at least one compound selected from compounds of general formula (I-A), formula (I), formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), Formula (X) pharmaceutically acceptable salts or solvates thereof and a pharmaceutically acceptable excipient.

In another aspect, the present invention provides pharmaceutical composition for use in the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, comprises administering a compound selected from compounds of general formula (I-A), formula (I), formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), formula (X) pharmaceutically acceptable salts or solvates thereof.

In another aspect, the present invention provides a method for the treatment of at least one disease condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, which comprises administering a compound selected from:

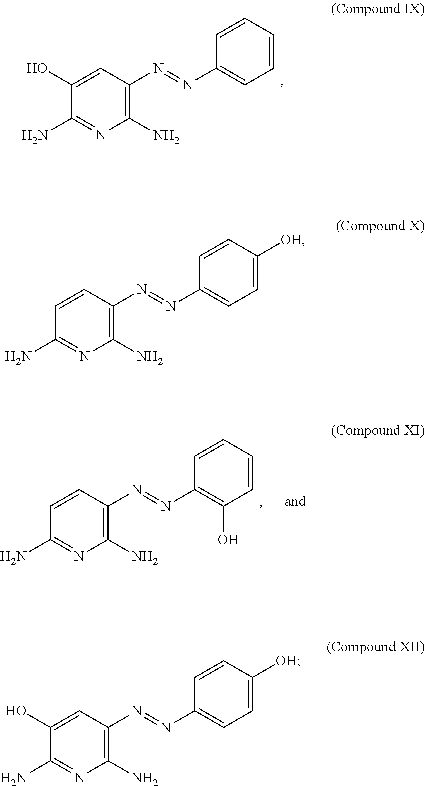 [SMILES: Nc1nc(N)c(/N=N/c2ccccc2)cc1O ; Nc1ccc(/N=N/c2ccc(O)cc2)c(N)n1 ; Nc1ccc(/N=N/c2ccccc2O)c(N)n1 ; Nc1nc(N)c(/N=N/c2ccc(O)cc2)cc1O]

pharmaceutically acceptable salts or solvates thereof.

In another aspect, the present invention provides a method for the treatment of at least one disease condition selected from over active bladder (OAB), chronic cough, endometriosis, neuropathic pain, migraine, pruritus, postherpetic neuralgia, urinary tract infection related pain, osteoarthritis, arthritis and urinary incontinence; which comprises administering a compound selected from:

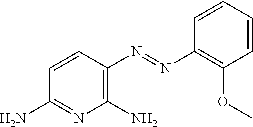 [SMILES: COc1ccccc1/N=N/c1ccc(N)nc1N]

(Compound XIII); pharmaceutically acceptable salts or solvates thereof.

The following examples are given for the purpose of illustrating the present invention and should not be considered as limiting the scope of the invention.

### Example-01: Preparation of (E)-2-((2,6-diaminopyridin-3-yl)diazenyl)phenyl 1-methyl-L-tryptophanate

### Step-01: Preparation of N-(tert-butoxycarbonyl)-1-methyl-L-tryptophan

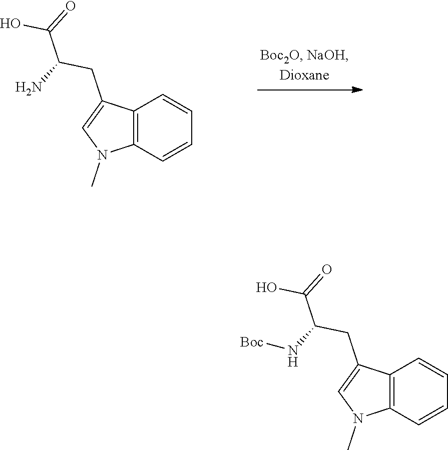 [SMILES: Cn1cc(C[C@H](N)C(=O)O)c2ccccc21 ; Cn1cc(C[C@H](NC(=O)OC(C)(C)C)C(=O)O)c2ccccc21]

To a stirred solution of 1-methyl-L-tryptophan (2.0 g) in 1,4-dioxane (40 mL) were added sodium hydroxide (364 mg/20 mL water) at 0° C. temperature, followed by addition of di-tert-butyl dicarbonate (2.7 mL) at same temperature. The reaction mixture was stirred at 0° C. for 4 h and then continued stirring at room temperature for additional 16 h. After completion of reaction (monitored by TLC: 20% methanol in dichloromethane), excess of solvent was evaporated under reduced pressure, followed by acidification of the reaction mixture using 1N hydrochloric acid. This mixture was extracted with ethyl acetate (3×1500 mL) followed by washings with water (3×500 mL). The combined organic layer was dried over anhydrous sodium sulfate and evaporated under reduced pressure to obtain a crude. The crude was washed with n-pentane and dried under reduced pressure to give N-(tert-butoxycarbonyl)-1-methyl-L-tryptophan (1.4 g, 48% yield) as an off-white solid.

LCMS: 96.83% (m/z: 317.39), [M−1]⁺, 1.48 min (10 min run), 214 nm).

¹H-NMR (400 MHz, DMSO-d₆): δ 12.51 (s, 1H), 7.53 (d, 1H), 7.37 (d, 1H), 7.15-7.11 (m, 2H), 7.02 (t, 1H), 6.93 (d, 1H), 4.15-4.12 (m, 1H), 3.72 (s, 3H), 3.12 (dd, 1H), 3.01-2.95 (m, 1H), 1.33 (s, 9H).

### Step-02: Preparation of (E)-2-((2,6-diaminopyridin-3-yl)diazenyl)phenyl(S)-3-(112-indol-3-yl)-2-((tert-butoxycarbonyl)amino)propanoate

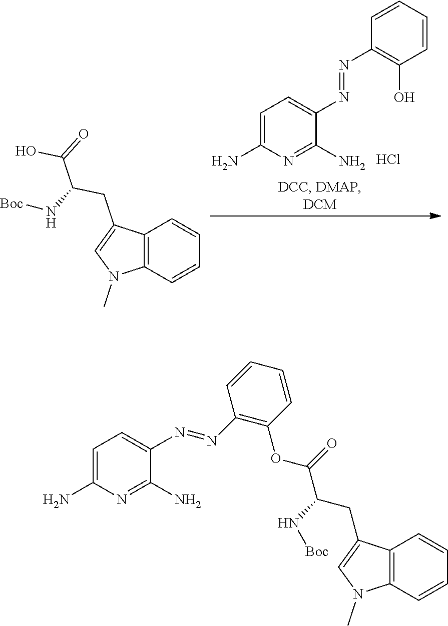 [SMILES: Cn1cc(C[C@H](NC(=O)OC(C)(C)C)C(=O)O)c2ccccc21 ; Nc1ccc(/N=N/c2ccccc2O)c(N)n1 ; Cl ; Cn1cc(C[C@H](NC(=O)OC(C)(C)C)C(=O)Oc2ccccc2/N=N/c2ccc(N)nc2N)c2ccccc21]

To a stirred solution of (E)-2-((2,6-diaminopyridin-3-yl)diazenyl)phenol (0.500 g) in dichloromethane (6.0 mL) were added dicyclohexylcarbodiimide (1.16 g), 4-(dimethylamino)pyridine (0.12 g) and N-(tert-butoxycarbonyl)-1-methyl-L-tryptophan (1.2 g). The reaction mixture was stirred at room temperature for 16 h. Completion of the reaction was monitored by TLC (mobile phase: 50% ethyl acetate in heptane). After completion of the reaction, the reaction mixture was quenched with demineralized water and extracted with 5% methanol in dichloromethane (3×100 mL). The combined organic layer was dried over anhydrous sodium sulfate and evaporated under reduced pressure to obtain crude. The crude was washed with n-pentane and dried under reduced pressure to give of (E)-2-((2,6-diaminopyridin-3-yl)diazenyl)phenyl (S)-3-(112-indol-3-yl)-2-((tert-butoxycarbonyl)amino)propanoate (0.1 g, 50% purity) as a yellow solid.

LCMS: 50.45% (m/z: 530.14), [M+1]⁺, 2.43 min (10 min run), 214 nm).

### Step-03: Preparation of (E)-2-((2,6-diaminopyridin-3-yl)diazenyl)phenyl 1-methyl-L-tryptophanate

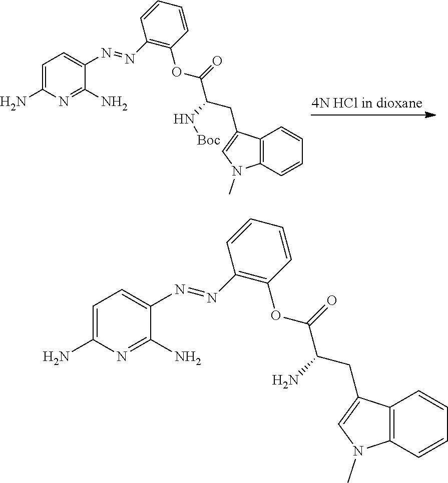

To a stirred solution of (E)-2-((2,6-diaminopyridin-3-yl)diazenyl)phenyl (S)-3-(1H-indol-3-yl)-2-((tert-butoxycarbonyl)amino)propanoate (1 g) in dichloromethane (20 mL) was added 4M hydrochloric acid in 1,4-dioxane (2 mL) at 0° C. temperature and then reaction mixture stirred at room temperature for additional 16 h. After completion of the reaction (monitored by TLC: mobile phase: 50% ethyl acetate in heptane), excess of the solvent was evaporated under reduced pressure to obtain crude material. The crude was purified by prep-HPLC (T3 column, triflluoroacetic acid:acetonitrile, 1:1) to give (E)-2-((2,6-diaminopyridin-3-yl)diazenyl)phenyl 1-methyl-L-tryptophanate (0.09 g, 11%) as a yellow solid.

LCMS: 95.22% (m/z: 430.15 [M+1]⁺, 4.74 min (10 min run), 214 nm).

¹H-NMR (400 MHz, DMSO-d₆): δ 7.80-7.78 (m, 2H), 7.59 (d, 1H), 7.42-7.36 (m, 3H), 7.26-7.11 (m, 3H), 7.04 (t, 1H), 6.00 (d, 1H), 4.59 (t, 1H), 3.69 (s, 3H), 3.52 (dd, 1H), 3.40 (dd, 1H).

As per step-02 reaction condition of example-01 (DCC, DMAP, DCM); following ester prodrugs have been synthesized where respective acids were procured commercially.

| Examples | Structure | Analytical details |
|---|---|---|
| Example-02 | 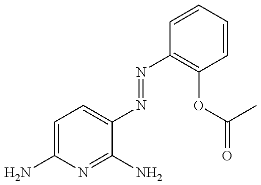 [SMILES: CC(=O)Oc1ccccc1/N=N/c1ccc(N)nc1N] | LCMS: 99.76% (m/z: 272.29, [M + 1]⁺, 4.93 min (10 min run), 214 nm). ¹H NMR (400 MHz, DMSO-d₆): δ ppm 7.72-7.70 (m, 1H), 7.65 (d, 1H), 7.32-7.29 (m, 2H), 7.22-7.20 (m, 1H), 6.87 (br s, 2H), 6.01 (d, 1H), 2.30 (s, 3H) |
| Example-03 | 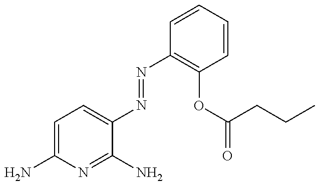 [SMILES: CCCC(=O)Oc1ccccc1/N=N/c1ccc(N)nc1N] | LCMS: 99.57% (m/z: 300.14, [M + 1]⁺, 5.20 min (10 min run), 214 nm) ¹H NMR (400 MHz, DMSO-d₆): δ ppm 7.71- 7.69 (m, 1H), 7.64 (d, 1H), 7.32-7.28 (m, 2H), 7.20-7.18 (m, 1H), 6.84 (br s, 2H), 5.99 (d, 1H), 2.59 (t, 2H), 1.69-1.63 (m, 2H), 0.90 (t, 3H). |
| Example-04 | 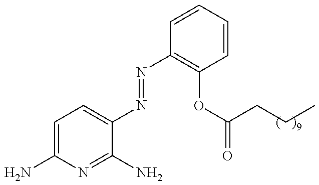 | LCMS: 99.50% (m/z: 412.27, [M + 1]⁺, 7.02 min (10 min run), 214 nm). ¹H NMR (400 MHz, DMSO-d₆): δ ppm 7.71- 7.69 (m, 1H), 7.63 (d, 1H) 7.32-7.27 (m, 2H), 7.20-7.17 (m, 1H), 6.83 (br s, 2H), 5.98 (d, 1H), 2.59 (t, 2H), 1.65-1.61 (m, 2H), 1.36-1.24 (m, 16H), 0.85 (t, 3H). |
| Example-05 | 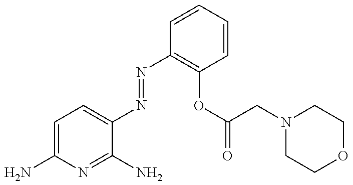 [SMILES: Nc1ccc(/N=N/c2ccccc2OC(=O)CN2CCOCC2)c(N)n1] | LCMS: 95.38% (m/z: 357.18, [M + 1]⁺, 3.90 min (10 min run), 214 nm). ¹H NMR (400 MHz, DMSO-d₆): δ ppm 7.74- 7.71 (m, 1H), 7.65 (d, 1H) 7.32-7.29 (m, 2H), 7.24-7.05 (m, 1H), 6.86 (br s, 2H), 5.99 (d, 1H), 3.60-3.58 (m, 6H), 2.61 (br s, 4H) |
| Example-06 | 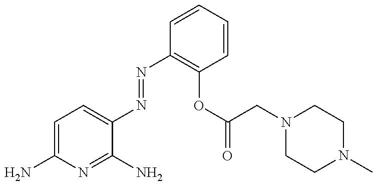 [SMILES: CN1CCN(CC(=O)Oc2ccccc2/N=N/c2ccc(N)nc2N)CC1] | LCMS: 89.02% (m/z: 370.27, [M + 1]⁺, 4.04 min (10 min run), 214 nm). |
| Example-07 | 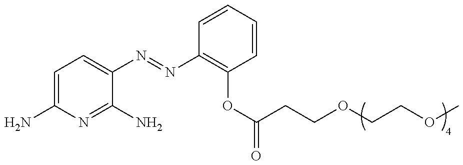 | LCMS: 98.03% (m/z: 492.25, [M + 1]⁺, 5.05 min (10 min run), 214 nm). ¹H-NMR (400 MHz, DMSO-d₆): δ ppm 7.72- 7.70 (m, 1H), 7.65 (d, 1H), 7.32-7.30 (m, 2H), 7.20-7.17 (m, 1H), 6.82 (br s, 2H), 6.00 (d, 1H), 3.76 (t, 2H), 3.56-3.48 (m, 14H), 3.42-3.41 (m, 2H), 3.23 (s, 3H), 2.86 (t, 2H). |
| Example-08 | 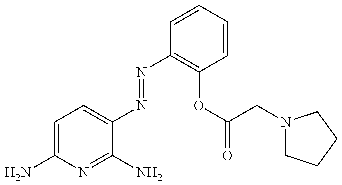 [SMILES: Nc1ccc(/N=N/c2ccccc2OC(=O)CN2CCCC2)c(N)n1] | LCMS: 96.72% (m/z: 341.21, [M + 1]⁺, 5.18 min (10 min run), 214 nm. ¹H-NMR (400 MHz, DMSO-d₆): δ ppm 10.50 (br s, 1H), 7.89 (d, 1H), 7.80 (d, 1H), 7.59-7.21 (m, 5H), 6.04 (d, 1H), 4.70 (s, 2H), 3.63 (br s, 2H), 3.18 (br s, 2H), 2.06 (br s, 2H), 1.92 (br s, 2H). |
| Example-09 | 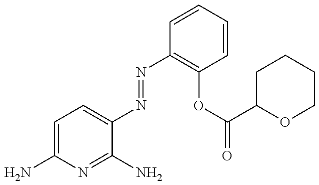 [SMILES: Nc1ccc(/N=N/c2ccccc2OC(=O)C2CCCCO2)c(N)n1] | LCMS: 99.81% (m/z: 342.25, [M + 1]⁺, 5.09 min (10 min run), 214 nm). ¹H-NMR (400 MHz, DMSO-d₆): δ ppm 8.23 (br s, 1H), 7.81 (t, 2H) 7.60 (br s, 1H), 7.42- 7.33 (m, 2H), 7.27-7.25 (m, 1H), 6.10 (d, 1H), 4.38-4.35 (m, 1H), 3.99-3.96 (m, 1H), 3.58-3.52 (m, 1H), 2.03-1.99 (m, 1H), 1.85-1.82 (m, 1H), 1.75-1.62 (m, 2H), 1.60-1.54 (m, 2H). |
| Example-10 | 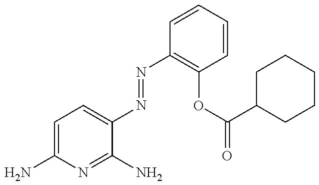 [SMILES: Nc1ccc(/N=N/c2ccccc2OC(=O)C2CCCCC2)c(N)n1] | LCMS: 99.87% (m/z: 340.24, [M + 1]+, 5.80 min (10 min run), 214 nm). ¹H-NMR (400 MHz, DMSO-d₆): δ ppm 8.33- 8.18 (m, 1H), 7.82-7.77 (m, 2H), 7.59-7.51 (m, 1H), 7.40-7.30 (m, 2H), 7.21 (d, 1H), 6.09 (d, 1H), 2.69-2.63 (m, 1H), 2.02-1.99 (m, 2H), 1.76-1.73 (m, 2H), 1.65-1.62 (m, 1H), 1.55-1.46 (m, 2H), 1.41-1.20 (m, 3H). |
| Example-11 | 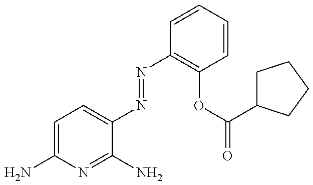 [SMILES: Nc1ccc(/N=N/c2ccccc2OC(=O)C2CCCC2)c(N)n1] | LCMS: 99.55% (m/z: 326.20, [M + 1]⁺, 5.57 min (10 min run), 214 nm). ¹H-NMR (400 MHz, DMSO-d₆): δ ppm 8.16 (br s, 1H), 7.80-7.77 (m, 2H), 7.70-7.57 (m, 1H), 7.43-7.31 (m, 2H), 7.23 (d, 1H), 6.09 (d, 1H), 3.13-3.05 (m, 1H), 2.05-1.97 (m, 2H), 1.91-1.83 (m, 2H), 1.70-1.57 (m, 4H). |
| Example-12 | 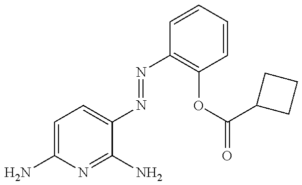 [SMILES: Nc1ccc(/N=N/c2ccccc2OC(=O)C2CCC2)c(N)n1] | LCMS: 99.11% (m/z: 312.16 [M + 1]⁺, 5.33 min (10 min run), 214 nm). ¹H NMR (400 MHz, DMSO-d₆): δ ppm 8.03- 8.17 (m, 1H), 7.81-7.79 (m, 2H), 7.72-7.46 (m, 1H), 7.41-7.31 (m, 2H), 7.25 (d, 1H), 6.09 (d, 1H), 3.55-3.48 (m, 1H), 2.35-2.26 (m, 4H), 2.07-1.96 (m, 1H), 1.94-1.88 (m, 1H). |
| Example-13 | 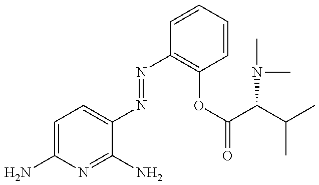 [SMILES: CC(C)[C@H](C(=O)Oc1ccccc1/N=N/c1ccc(N)nc1N)N(C)C] | LCMS: 98.59% (m/z: 357.15, [M + 1]⁺, 6.06 min (10 min run), 214 nm). ¹H-NMR (400 MHz, DMSO-d₆): δ ppm 7.71- 7.43 (m, 3H), 7.33-7.28 (m, 2H), 7.17-7.15 (m, 1H), 6.79 (br s, 2H), 5.93 (d, 1H), 3.03 (d, 1H), 2.37 (s, 6H), 2.04-1.98 (m, 1H), 1.00-0.95 (m, 6H). |
| Example-14 | 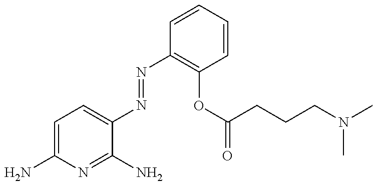 [SMILES: CN(C)CCCC(=O)Oc1ccccc1/N=N/c1ccc(N)nc1N] | LCMS: 96.39% (m/z: 343.26 [M + 1]⁺, 4.71 min (10 min run), 214 nm). ¹H-NMR (400 MHz, DMSO-d₆): δ ppm 8.18 (s, 1H), 8.11 (d, 1H), 7.73-7.60 (m, 3H), 7.55 (d, 1H), 7.31-7.27 (m, 1H), 7.02 (d, 1H), 6.94-6.88 (m, 1H), 2.43 (t, 2H), 2.38 (t, 2H), 2.25 (s, 6H), 1.78-1.70 (m, 2H). |
| Example-15 | 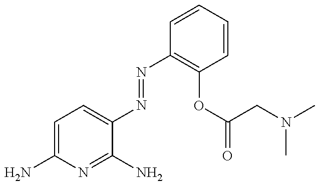 [SMILES: CN(C)CC(=O)Oc1ccccc1/N=N/c1ccc(N)nc1N] | LCMS: 97.88% (m/z: 315.22 [M + 1]⁺, 4.74 min (10 min run), 214 nm). ¹H-NMR (400 MHz, DMSO-d₆): δ ppm 11.05 (s, 1H), 10.50 (br s, 1H), 8.17 (d, 1H), 7.83 (br s, 2H), 7.74 (d, 1H), 7.49 (br s, 1H), 7.30 (t, 1H), 7.04 (d, 1H), 6.90 (t, 1H), 3.71 (br s, 2H), 2.63 (s, 6H). |
| Example-16 | 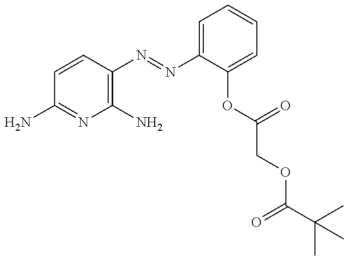 [SMILES: CC(C)(C)C(=O)OCC(=O)Oc1ccccc1/N=N/c1ccc(N)nc1N] | LCMS: 97.03% (m/z: 372.13, [M + 1]⁺, 7.69 min (15 min run), 214 nm). ¹H-NMR (400 MHz, DMSO-d₆): δ ppm 7.79- 7.77 (m, 1H), 7.69 (d, 1H), 7.34-7.30 (m, 2H), 7.24-7.13 (m, 1H), 6.78 (br s, 2H), 5.99 (d, 1H), 5.00 (s, 2H), 1.20 (s, 9H). |
| Example-17 | 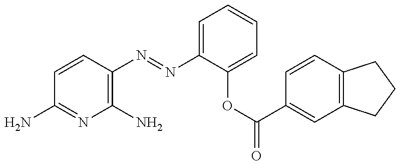 [SMILES: Nc1ccc(/N=N/c2ccccc2OC(=O)c2ccc3c(c2)CCC3)c(N)n1] | LCMS: 99.89% (m/z: 374.14, [M + 1]⁺, 6.13 min (10 min run), 214 nm). ¹H NMR (400 MHz, DMSO-d₆): δ ppm 7.97 (s, 1H), 7.92-7.90 (d, 1H), 7.74-7.72 (m, 1H), 7.52 (d, 1H), 7.44 (d, 1H), 7.38-7.13 (m, 3H), 6.75 (br s, 2H), 5.91 (d, 1H), 2.97 (t, 4H), 2.13-2.04 (m, 2H). |

### Example-18: Preparation of (E)-2-((2,6-diaminopyridin-3-yl)diazenyl)phenyl propionate

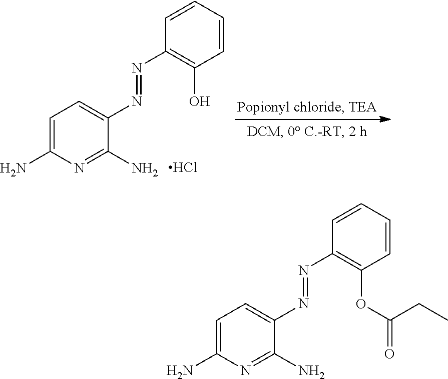 [SMILES: Nc1ccc(/N=N/c2ccccc2O)c(N)n1 ; Cl ; CCC(=O)Oc1ccccc1/N=N/c1ccc(N)nc1N]

To a solution of (E)-2-((2,6-diaminopyridin-3-yl)diazenyl)phenol hydrochloride (0.2 g) in dichloromethane (8 mL) were added triethylamine (0.25 mL) and propionyl chloride (0.1 g) at 0° C. temperature and the reaction mixture was stirred at room temperature for 3 h. After completion of reaction (monitored by TLC), the reaction mass was quenched with cold water (10 mL) and extracted with dichloromethane (3×30 mL). The combined organic layer was washed with water (2×100 mL), dried over anhydrous sodium sulfate, and concentrated under reduced pressure to obtained crude material which was purified by Prep HPLC [(60% Acetonitrile:Formic acid) to afford (E)-2-((2,6-diaminopyridin-3-yl)diazenyl)phenyl propionate. (0.042 g, 19.5%)

LCMS: 99.52% (m/z: 286.2, [M+1]⁺, 4.85 min (10 min run), 214 nm).

¹H NMR (400 MHz, DMSO-d₆): δ ppm 7.71-7.69 (m, 1H), 7.64 (d, 1H), 7.31-7.28 (m, 2H), 7.22-7.19 (m, 1H), 6.84 (br s, 2H), 6.00 (d, 1H), 2.64 (q, 2H), 1.15 (t, 3H).

As per reaction condition of example-18 (Triethylamine, DCM); following ester prodrugs have been synthesized where respective acyl chlorides were procured commercially.

| Examples | Structure | Analytical details |
|---|---|---|
| Example-19 | 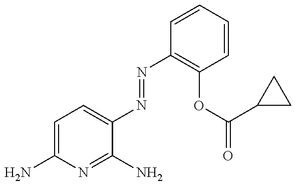 [SMILES: Nc1ccc(/N=N/c2ccccc2OC(=O)C2CC2)c(N)n1] | LCMS: 97.89% (m/z: 298.16 [M + 1]⁺, 5.61 min (10 min run), 214 nm). ¹H-NMR (400 MHz, DMSO-d₆): δ ppm 8.10 (br s, 1H), 7.70-7.68 (m, 1H), 7.65 (d, 1H), 7.31-7.28 (m, 2H), 7.22-7.20 (m, 1H), 6.82 (br s, 2H), 6.01 (d, 1H), 1.95-1.89 (m, 1H), 1.15-1.08 (m, 2H), 1.00-0.98 (m, 2H). |

### Example-20: Preparation of (E)-2-((2,6-diaminopyridin-3-yl)diazenyl)phenyl 1-methylpiperidine-2-carboxylate

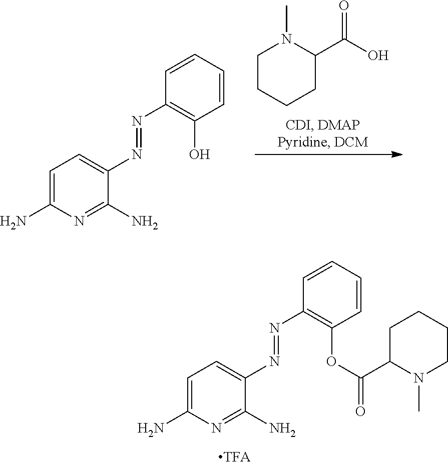

To a stirred solution of (E)-2-((2,6-diaminopyridin-3-yl)diazenyl)phenol (0.3 g) in dichloromethane (3 mL) was added pyridine (3 mL), 1,1′-carbonyl diimidazole (0.68 g) and 4-(dimethylamino)pyridine (0.067 g). The resulting reaction mixture was stirred at 0° C. for 30 min. To this above stirring solution, 1-methylpiperidine-2-carboxylic acid (0.592 g) was added and resulting solution was stirred at room temperature for 16 h. Reaction mixture was monitored by TLC (mobile phase: 70% ethyl acetate in hexane). After completion of reaction, the reaction mixture was quenched with water, extracted with dichloromethane (3×100 mL), dried over anhydrous sodium sulfate, evaporated under reduced pressure to obtain crude material. The crude was purified by prep HPLC [method: (A) 0.5% TFA in water (B) 100% Acetonitrile] and the product fractions were lyophilized to give (E)-2-((2,6-diaminopyridin-3-yl)diazenyl)phenyl 1-methylpiperidine-2-carboxylate (0.037 g, 9%) as yellow solid.

LCMS: 95.00% (m/z: 355.25, [M+1]⁺, 4.02 min (10 min run), 214 nm).

¹H-NMR (400 MHz, DMSO-d₆): δ 7.80 (dd, 1H), 7.73 (d, 1H), 7.43-7.36 (m, 3H), 6.05 (d, 1H), 4.41-4.55 (m, 1H), 3.47-3.44 (m, 1H), 3.12-3.07 (m, 1H), 2.87 (s, 3H), 2.45-2.25 (m, 2H), 1.83-1.59 (m, 4H).

### Example-21: Preparation of (E)-2-((2,6-diaminopyridin-3-yl)diazenyl)phenyl diethylcarbamate

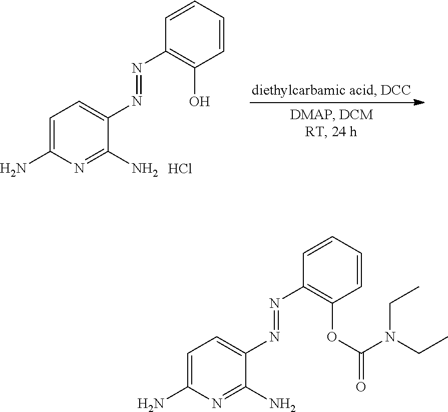 [SMILES: Nc1ccc(/N=N/c2ccccc2O)c(N)n1 ; Cl ; CCN(CC)C(=O)Oc1ccccc1/N=N/c1ccc(N)nc1N]

To a solution of (E)-2-((2,6-diaminopyridin-3-yl)diazenyl) phenol hydrochloride (0.200 g) in Dichloromethane (10 mL) were added dicyclohexylcarbodiimide (0.466 g) and 4-(dimethylamino)pyridine (0.046 g) and stirred at room temperature for 15 min. After that, diethylcarbamic acid (0.114 g) was added and the reaction mixture was stirred at room temperature for 24 h. After completion of reaction, (monitored by TLC), the reaction mass was quenched with cold water (10 mL) and extracted with dichloromethane (3×30 mL), washed with water (2×100 mL), dried over anhydrous sodium sulfate, and concentrated under vacuum to obtain the crude material. The crude material was purified by prep HPLC [70% Acetonitrile/30% TFA] to afford (E)-2-((2,6-diaminopyridin-3-yl)diazenyl)phenyl diethylcarbamate (0.025 g, 10.1%) as yellow solid.

LCMS: 99.70% (m/z: 329.20, [M+1]⁺, 5.58 min (10 min run), 214 nm).

¹H NMR (400 MHz, DMSO-d₆): δ ppm 8.16-7.87 (m, 2H), 7.68-7.63 (m, 2H), 7.30-7.17 (m, 3H), 6.76 (br s, 2H), 5.98 (d, 1H), 3.46-3.41 (m, 2H), 3.28-3.25 (m, 2H), 1.21 (t, 3H), 1.09 (t, 3H).

### Example-22: Preparation of (E)-2-((2,6-diaminopyridin-3-yl) diazenyl) phenyl 4-methyl-1,4-diazepane-1-carboxylate

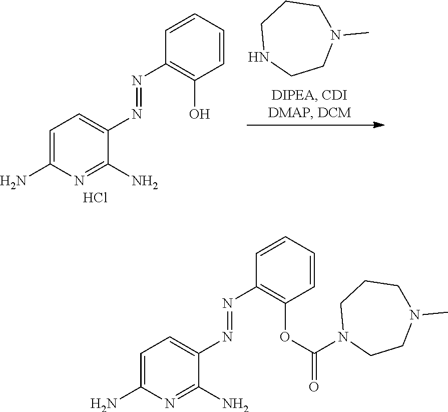

To a stirred solution of (E)-2-((2,6-diaminopyridin-3-yl) diazenyl) phenol hydrogen chloride (0.500 g) in 10 mL dichloromethane at 0° C. under nitrogen atmosphere were added N,N-diisopropylethylamine (2.43 g) and 1,1′-carbonyldiimidazole (0.918 g) followed by 4-(dimethylamino)pyridine (0.116 g). Stirred the reaction mixture for 10 min at 0° C. temperature and then added 1-methyl 1,4-diazepane (0.861 g). The reaction mixture was stirred at room temperature for 26 h. After completion of the reaction, the mixture was quenched with water and extracted with dichloromethane (3×50 mL). The combined organic layer was dried over anhydrous sodium sulfate and evaporated under reduced pressure to obtained crude material. The crude was enriched by Flash chromatography and then purified by prep HPLC [method: TFA buffer] and the product fractions were lyophilized to give 0.080 g of (E)-2-((2,6-diaminopyridin-3-yl) diazenyl) phenyl 4-methyl-1,4-diazepane-1-carboxylate HCl salt as red solid.

LCMS: 97.21% (m/z: 370.22 [M+1]⁺, 4.39 min (10 min run), 214 nm).

¹H NMR (400 MHz, DMSO-d₆): δ ppm 7.78 (dd, 1H), 7.72 (d, 1H), 7.39-7.25 (m, 3H), 6.10 (d, 1H), 4.08-3.80 (m, 1H), 3.70-3.68 (m, 2H), 3.62-3.54 (m, 3H), 3.31-3.16 (m, 2H), 2.88 (s, 3H), 2.20-2.01 (m, 2H).

### Example-23: Preparation of (E)-2-((2,6-diaminopyridin-3-yl)diazenyl)phenyl morpholine-4-carboxylate

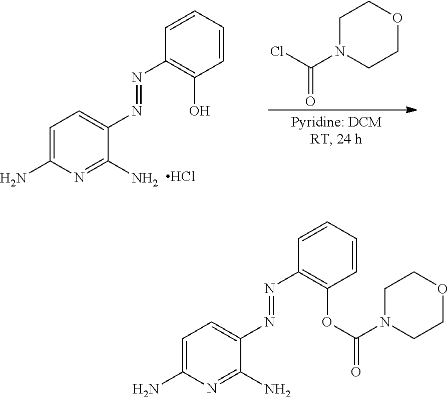 [SMILES: Nc1ccc(/N=N/c2ccccc2O)c(N)n1 ; Cl ; O=C(Cl)N1CCOCC1 ; Nc1ccc(/N=N/c2ccccc2OC(=O)N2CCOCC2)c(N)n1]

To a solution of (E)-2-((2,6-diaminopyridin-3-yl)diazenyl)phenol hydrochloride (0.2 g) in dichloromethane:pyridine (1:1, 5 mL) were added morpholine 4-carbonyl chloride (0.279 g) and stirred at room temperature for 24 h. After completion of the reaction (monitored by TLC), the reaction mass was quenched with cold water (10 mL) and extracted with dichloromethane (3×30 mL). The organic layer was washed with water (2×100 mL), dried over anhydrous sodium sulfate, and concentrated under reduced pressure to obtain crude material. The crude was purified by using flash chromatography with 9% methanol in dichloromethane as eluent to afford (E)-2-((2,6-diaminopyridin-3-yl)diazenyl)phenyl 4-methylpiperazine-1-carboxylate (0.035 g, 13.5%) as a yellow solid.

LCMS: 98.18% (m/z: 341.19 [M+1]⁺, 4.97 min (10 min run), 214 nm).

¹H NMR (400 MHz, DMSO-d₆): δ ppm 8.40 (br s, 1H), 7.68-7.65 (m, 2H), 7.31-7.21 (m, 3H), 6.81 (br s, 2H), 6.01 (d, 1H), 3.65 (br s, 6H), 3.41 (br s, 2H).

### Example-24: Preparation of (E)-2-((2,6-diaminopyridin-3-yl)diazenyl)phenyl 4-methylpiperazine-1-carboxylate

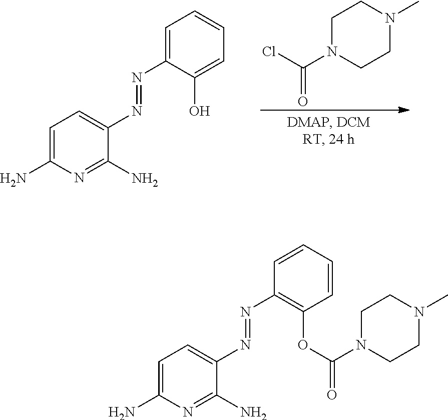

To a solution of (E)-2-((2,6-diaminopyridin-3-yl)diazenyl)phenol hydrochloride (0.2 g) in dichloromethane (10 mL) were added 4-methylpiperazine-1-carbonyl chloride (0.305 g) and 4-(dimethylamino)pyridine (0.045 g) and stirred at room temperature for 24 h. After completion of the reaction (monitored by TLC), the reaction mass was quenched with cold water (10 mL) and extracted with dichloromethane (3×30 mL). The organic layer was washed with water (2×100 mL), washed with brine, dried over anhydrous sodium sulfate, and concentrated under reduced pressure to obtain the crude material. The crude was purified by using flash chromatography with 10% methanol:dichloromethane as eluent to afford (E)-2-((2,6-diaminopyridin-3-yl)diazenyl)phenyl 4-methylpiperazine-1-carboxylate (0.051 g, 19.1%) as yellow solid.

LCMS: 96.78% (m/z: 356.20, [M+1]⁺, 4.75 min (10 min run), 214 nm).

¹H NMR (400 MHz, DMSO-d₆): δ ppm 7.68-7.65 (m, 2H), 7.30-7.25 (m, 2H), 7.24-7.19 (m, 1H), 6.79 (br s, 2H), 6.00 (d, 1H), 3.63 (br s, 2H), 3.42 (br s, 2H), 2.41 (br s, 4H), 2.27 (br s, 3H).

As per reaction condition of example-24 (Acyl chloride, DMAP, DCM); following carbamate prodrugs have been synthesized where respective acyl chlorides were procured commercially.

| Examples | Structure | Analytical details |
|---|---|---|
| Example-25 | 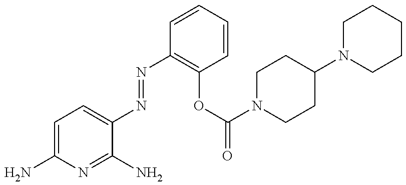 [SMILES: Nc1ccc(/N=N/c2ccccc2OC(=O)N2CCC(N3CCCCC3)CC2)c(N)n1] | LCMS: 97.70% (m/z: 424.4), [M + 1]⁺, 4.16 min (10 min run), 214 nm). ¹H NMR (400 MHz, DMSO-d₆): δ ppm 7.67-7.63 (m, 2H), 7.30-7.19 (m, 3H), 6.78 (br s, 1H), 5.99 (d, 1H), 4.22- 4.20 (m, 1H), 4.01-3.98 (m, 1H), 3.05- 3.01 (m, 1H), 2.88-2.83 (m, 1H), 2.49- 2.44 (m, 5H), 1.79 (br s, 2H), 1.50-1.38 (m, 8H) |

### Example-26: Preparation of (E)-2-((2,6-diaminopyridin-3-yl)diazenyl)phenyl (2-(dimethylamino)ethyl)(methyl) carbamate hydrochloride

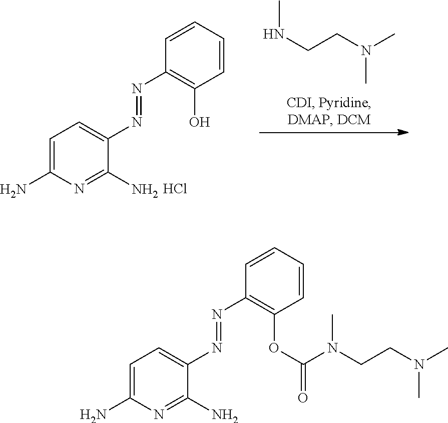

To a stirred solution of (E)-2-((2,6-diaminopyridin-3-yl)diazenyl)phenol hydrochloride (0.25 g) in dichloromethane (2.5 mL) and pyridine (2.5 mL) at 0° C. was added 1,1′-carbonyldiimidaziole (0.456 g) and 4-(dimethylamino)pyridine (0.057 g) and the resulting mixture was stirred at 0° C. for 30 min followed by the addition of N¹,N¹,N²-trimethylethane-1,2-diamine (0.385 g). The reaction mixture was stirred at room temperature for 16 h. The completion of the reaction was monitored by TLC (mobile phase: 10% methanol in dichloromethane). After completion of reaction, the reaction mixture was quenched with DM water and extracted with dichloromethane (3×100 mL). The combined organic layer was dried over anhydrous sodium sulfate and evaporated under reduced pressure to obtain crude material. The crude was purified by prep HPLC [method: (A) 0.5% TFA in water (B) 100% Acetonitrile] and the product fractions were lyophilized to give 0.135 g of (E)-2-((2,6-diaminopyridin-3-yl)diazenyl)phenyl(2-(dimethylamino)ethyl)(methyl)carbamate hydrochloride as a red solid.

LCMS: 98.96% (m/z: 358.25 [M+1]⁺, 4.33 min (10 min run), 214 nm).

¹H NMR (400 MHz, DMSO-d₆): δ ppm 9.54-9.46 (m, 1H), 8.20 (br s, 1H), 7.75-7.72 (m, 2H), 7.36-7.25 (m, 4H), 6.07 (d, 1H), 3.78 (t, 1H), 3.81-3.62 (m, 2H), 3.39-3.30 (m, 2H), 3.11-2.94 (m, 3H), 2.87-2.83 (m, 6H)

As per reaction condition of example-26 (CDI, Pyridine, DMAP, DCM); following carbamate prodrugs have been synthesized where respective secondary amines were procured commercially.

| Examples | Structure | Analytical details |
|---|---|---|
| Example-27 | 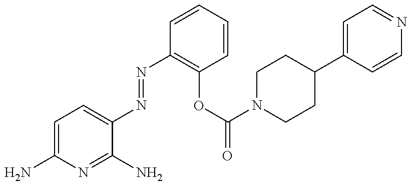 [SMILES: Nc1ccc(/N=N/c2ccccc2OC(=O)N2CCC(c3ccncc3)CC2)c(N)n1] | LCMS: 98.82% (m/z: 418.23, [M + 1]⁺, 4.31 min (10 min run), 214 nm. ¹H-NMR (400 MHz, DMSO-d₆): δ ppm 8.76 (d, 2H), 8.30 (br s, 2H), 7.7-7.77 (m, 3H), 7.73 (d, 1H), 7.73-7.25 (m, 4H), 6.06 (d, 1H), 4.36-4.05 (m, 2H), 3.20-3.02 (m, 3H), 1.91 (br s, 2H), 1.72 (m, 2H). |
| Example-28 | 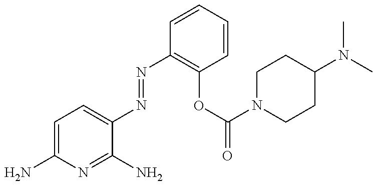 [SMILES: CN(C)C1CCN(C(=O)Oc2ccccc2/N=N/c2ccc(N)nc2N)CC1] | LCMS: 99.07% (m/z: 384.27, [M + 1]⁺, 4.10 min (10 min run), 214 nm). ¹H NMR (400 MHz, DMSO-d₆): δ ppm 9.56 (br s, 1H), 8.22 (br s, 1H), 7.74-7.70 (m, 2H), 7.35-7.27 (m, 3H), 7.23-7.21 (dd, 1H), 7.10 (br s, 1H), 6.06 (d, 1H), 4.35 (br s, 1H), 4.11-4.05 (m, 1H), 3.43-3.37 (m, 2H), 3.09 (br s, 1H), 2.92 (br s, 1H), 2.81- 2.80 (m, 6H), 2.06 (br s, 2H), ), 1.61 (br s, 2H). |
| Example-29 | 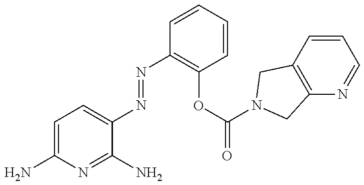 [SMILES: Nc1ccc(/N=N/c2ccccc2OC(=O)N2Cc3cccnc3C2)c(N)n1] | LCMS: 99.56 %, (m/z: 376.10, [M + 1]⁺, 4.47 min (10 min run), 214 nm). ¹H-NMR (400 MHz, DMSO-d₆): δ ppm 8.51 (d, 1H), 7.82 (d, 1H), 7.69 (d, 1H), 7.58 (d, 1H), 7.37-7.29 (m, 4H), 6.75 (br s, 2H), 5.90 (d, 1H), 4.95 (s, 1H), 4.88 (s, 1H), 4.75 (s, 1H), 4.69 (s, 1H). |
| Example-30 | 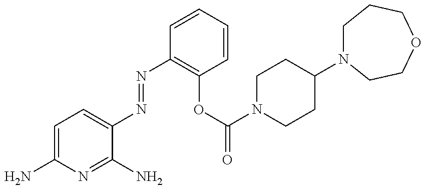 [SMILES: Nc1ccc(/N=N/c2ccccc2OC(=O)N2CCC(N3CCCOCC3)CC2)c(N)n1] | LCMS: 98.03% (m/z: 440.23 [M + 1]⁺, 4.72 min (10 min run), 214 nm). ¹H-NMR (400 MHz, DMSO-d₆): δ ppm 8.17 (s, 1H), 7.67-7.63 (m, 2H), 7.29-7.19 (m, 3H), 6.78 (br s, 2H), 5.98 (d, 1H), 4.23- 4.20 (m, 1H), 4.01-3.98 (m, 1H), 3.68 (t, 2H), 3.60-3.58 (t, 2H), 3.07-3.02 (m, 1H), 2.90-2.87 (m, 1H), 2.75-2.70 (m, 5H), 1.78- 1.73 (m, 4H), 1.43 (br s, 2H). |
| Example-31 | 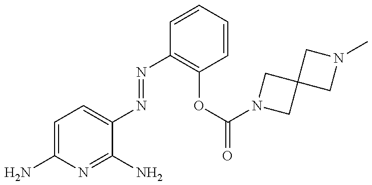 [SMILES: CN1CC2(C1)CN(C(=O)Oc1ccccc1/N=N/c1ccc(N)nc1N)C2] | LCMS: 93.37% (m/z: 368.26, [M + 1]⁺, 3.98 min (10 min run), 214 nm). ¹H-NMR (400 MHz, DMSO-d₆): δ ppm 9.60 (br s, 1H), 8.37 (br s, 1H), 7.78-769 (d, 2H), 7.34-6.96 (m, 5H), 6.07 (d, 1H), 4.46- 4.42 (m, 2H), 4.36-4.32 (m, 2H), 4.19-4.15 (m, 4H), 2.82 (d, 3H). |
| Example-32 | 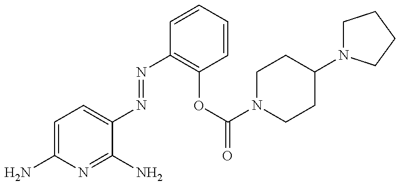 [SMILES: Nc1ccc(/N=N/c2ccccc2OC(=O)N2CCC(N3CCCC3)CC2)c(N)n1] | LCMS: 98.18% (m/z: 410.31, [M + 1]⁺, 4.41 min (10 min run), 214 nm). ¹H-NMR (400 MHz, DMSO-d₆): δ ppm 9.70 (br s, 1H), 8.22 (br s, 1H), 7.76-7.72 (m, 2H), 7.36-7.28 (m, 2H), 7.24-6.97 (m, 2H), 6.08 (d, 1H), 4.31 (br s, 1H), 4.07 (br s, 1H), 3.58-3.57 (m, 2H), 3.38-3.36 (m, 2H), 3.13-3.10 (m, 3H), 2.92 (br s, 1H), 2.16-2.05 (m, 4H), 1.90-1.87 (m, 2H), 1.57 (br s, 2H). |
| Example-33 | 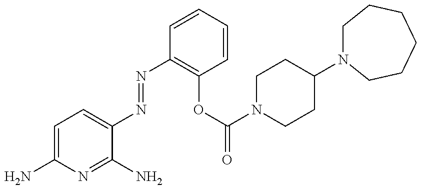 [SMILES: Nc1ccc(/N=N/c2ccccc2OC(=O)N2CCC(N3CCCCCC3)CC2)c(N)n1] | LCMS: 97.53% (m/z: 438.26 [M + 1]⁺, 4.47 min (10 min run), 214 nm). ¹H NMR (400 MHz, DMSO): δ ppm 9.20 (br s, 1H), 7.71-7.67 (m, 2H), 7.34-7.26 (m, 3H), 7.22 (dd, 1H), 7.01 (br s, 2H), 6.04 (d, 1H), 4.33-4.10 (m, 2H), 3.54-3.51 (m, 1H), 3.38-3.33 (m, 2H), 3.28-3.17 (m, 2H), 3.09 (br s, 1H), 2.93 (br s, 1H), 2.08 (br s, 2H), 1.86-1.63 (m, 10H). |
| Example-34 | 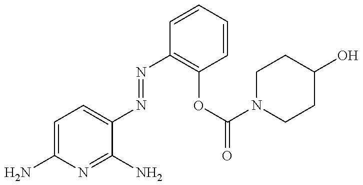 [SMILES: Nc1ccc(/N=N/c2ccccc2OC(=O)N2CCC(O)CC2)c(N)n1] | LCMS: 95.69% (m/z: 357.11 [M + 1]⁺, 4.52 min (10 min run), 214 nm). ¹H-NMR (400 MHz, DMSO-d₆): δ ppm 8.07 (br s, 2H), 7.79-7.72 (m, 2H), 7.44- 7.08 (m, 5H), 6.07 (d, 1H), 4.75 (br s, 1H), 3.94 (br s, 1H), 3.75-3.71 (m, 2H), 3.31- 3.12 (m, 2H), 1.80 (br s, 2H), 1.41-1.36 (m, 2H). |

### Example-35: Preparation of (E)-2-((2,6-diaminopyridin-3-yl)diazenyl)phenyl 4-morpholinopiperidine-1-carboxylate

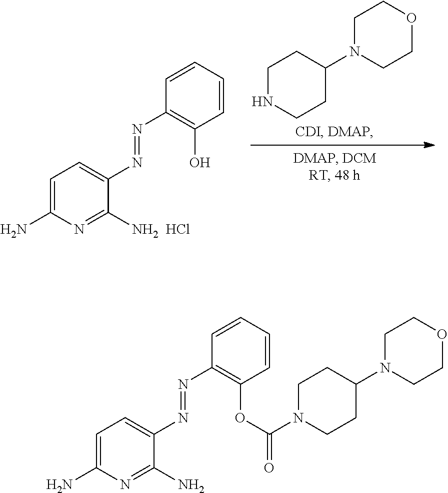

To a solution of (E)-2-((2,6-diaminopyridin-3-yl)diazenyl)phenol hydrochloride (0.2 g) in dichloromethane (10 mL) were added pyridine (0.288 g), 1,1′-carbonyldiimidazole (0.6 g) and 4-(dimethylamino)pyridine (0.046 g) at 0° C. and stirred at room temperature for 30 min. After that, 4-(piperidin-4-yl)morpholine (0.32 g) was added and the reaction mixture was stirred at room temperature for 48 h. After completion of reaction (monitored by TLC), the reaction mass was quenched with cold water (10 mL) and extracted with dichloromethane (3×30 mL). The organic layer was washed with water (2×100 mL), dried over anhydrous sodium sulfate, and concentrated under reduced pressure to obtain the crude material. The crude material was purified by prep HPLC (using TFA method) to afford (E)-2-((2,6-diaminopyridin-3-yl)diazenyl)phenyl 4-morpholinopiperidine-1-carboxylate (0.094 g, 28.9%) as a yellow solid.

LCMS: 99.74% (m/z: 426.24, [M+1]⁺, 4.10 min (10 min run), 214 nm).

¹H NMR (400 MHz, DMSO-d₆): δ ppm 9.80 (s, 1H), 8.20 (br s, 1H), 7.74-7.70 (m, 2H), 7.36-7.27 (m, 2H), 7.24-7.21 (m, 1H), 7.14 (br s, 1H), 6.06 (d, 1H), 4.35-4.11 (m, 2H), 4.07-4.04 (m, 2H), 3.73-3.67 (m, 2H), 3.48-3.45 (m, 3H), 3.19-3.09 (m, 3H), 2.93 (br s, 1H), 2.14 (br s, 2H), 1.62 (br s, 2H).

As per reaction condition of example-35 (CDI, DMAP, DCM); following carbamate prodrugs have been synthesized where respective secondary amines were procured commercially.

| Examples | Structure | Analytical details |
|---|---|---|
| Example-36 | 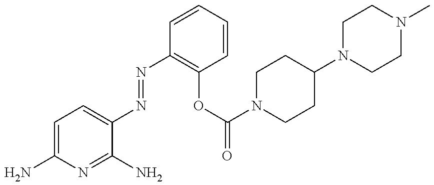 [SMILES: CN1CCN(C2CCN(C(=O)Oc3ccccc3/N=N/c3ccc(N)nc3N)CC2)CC1] | LCMS: 94.17% (m/z: 439., [M + 1]⁺, 4.38 min (10 min run), 214 nm). ¹H NMR (400 MHz, DMSO-d₆): δ ppm 8.37-8.19 (m, 1H), 7.77 (d, 1H) 7.73 (d, 1H), 7.36-7.10 (m, 5H), 6.07 (d, 1H), 4.25- 3.70 (m, 3H), 3.51-2.91 (m, 12H), 2.79 (s, 3H), 1.92 (br s, 2H), 1.49 (br s, 2H). |
| Example-37 | 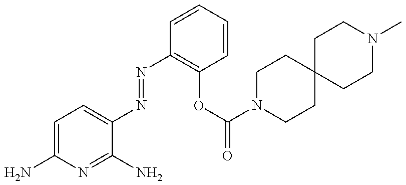 [SMILES: CN1CCC2(CC1)CCN(C(=O)Oc1ccccc1/N=N/c1ccc(N)nc1N)CC2] | LCMS: 98.42% (m/z: 424.52, [M + 1]⁺, 4.20 min (10 min run), 214 nm). ¹H-NMR (400 MHz, DMSO-d₆): δ ppm 8.59 (br s, 1H), 8.22 (s, 1H), 7.92 (br s, 1H), 7.68-7.64 (m, 2H), 7.30-7.20 (m, 2H), 7.18 (dd, 1H), 6.76 (br s, 2H), 5.99 (d, 1H), 3.59- 3.39 (m, 2H), 2.42 (br s, 4H), 2.52 (s, 3H), 1.55-1.44 (m, 8H). |
| Example-38 | 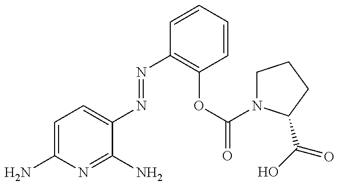 [SMILES: Nc1ccc(/N=N/c2ccccc2OC(=O)N2CCC[C@@H]2C(=O)O)c(N)n1] | LCMS: 91.22 % (m/z: 371.21 [M + 1]⁺, 4.49 min (10 min run), 214 nm). ¹H NMR (400 MHz, DMSO-d₆): δ ppm 12.81 (br s, 1H), 8.25 (br s, 1H), 7.86-7.84 (m, 1H), 7.75 (d, 1H), 7.53 (br s, 1H), 7.36- 7.24 (m, 3H), 7.14 (d, 1H), 6.09 (d, 1H), 4.46-4.20 (m, 1H), 3.68-3.64 (m, 1H), 3.47- 3.43 (m, 1H), 2.40-2.26 (m, 1H), 2.02-1.88 (m, 3H). |

### Example-39: Preparation of (E)-2-((2,6-diaminopyridin-3-yl)diazenyl)phenyl ethyl carbonat

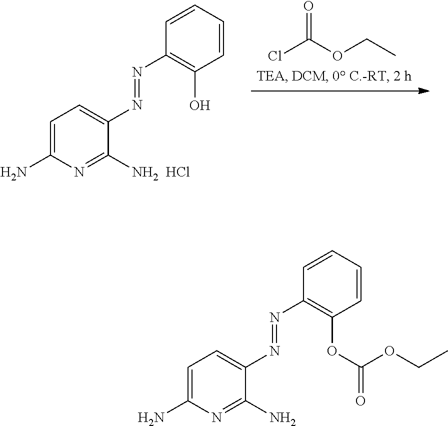

To a solution of (E)-2-((2,6-diaminopyridin-3-yl)diazenyl)phenol hydrochloride (0.3 g) in dichloromethane (8 mL) were added triethylamine (0.4 mL) and ethyl carbonochloridate (0.183 g) at 0° C. temperature. Resulting reaction mixture was stirred at room temperature for 2 h. After completion of reaction (monitored by TLC), the reaction mass was quenched with cold water (10 mL) and extracted with dichloromethane (3×30 mL). The combined organic layer was washed with water (2×100 mL), dried over anhydrous sodium sulfate, and concentrated under reduced pressure to obtain crude. Crude was purified by flash chromatography (70% ethyl acetate in hexane) to afford (E)-2-((2,6-diaminopyridin-3-yl)diazenyl)phenyl ethyl carbonate (0.095 g, 28%) as orange color solid.

LCMS: 97.86% (m/z: 302.07, [M+1]⁺, 4.96 min (10 min run), 214 nm).

¹H-NMR (400 MHz, DMSO-d₆): δ 7.70-7.67 (m, 1H), 7.65 (d, 1H), 7.34-7.26 (m, 3H), 6.02 (d, 1H), 4.19 (q, 2H), 1.23 (t, 3H).

### Example-40: Preparation of (E)-N-(6-amino-5-((2-((tert-butyldimethylsilyl)oxy)phenyl) diazenyl)pyridin-2-yl)acetamide

### Step-01: Preparation of (E)-3-((2-((tert-butyldimethylsilyl)oxy)phenyl)diazenyl)pyridine-2,6-diamine

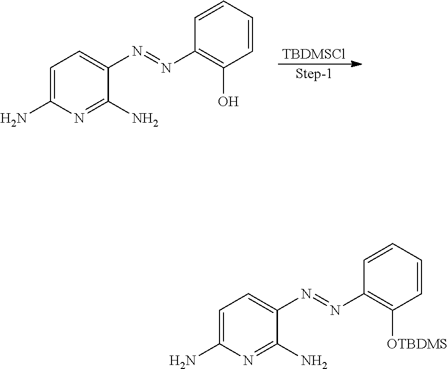

To a stirred solution of ((E)-2-((2,6-diaminopyridin-3-yl)diazenyl)phenol (2.0 g) in dichloromethane (25 mL) were added triethylamine (1.98 mL) and tert-butyldimethylsilyl chloride (2.2 g) at 0° C. temperature. The resulting reaction mixture was stirred at room temperature for additional 4 h. After completion of the reaction (monitored by TLC), the mixture was diluted with water (20 mL) and extracted with dichloromethane (3×70 mL). The combined organic layer was dried over anhydrous sodium sulfate and concentrated under reduced pressure to obtain a crude. The crude was purified by combi-flash chromatography (using 23% ethyl acetate in heptane) to afford (E)-3-((2-((tert-butyldimethylsilyl)oxy)phenyl)diazenyl)pyridine-2,6-diamine (2.6 g, 99%) as a red solid.

LCMS: 99.69% (m/z: 344.46), [M+1]⁺, 2.47 min (4 min run), 214 nm).

¹H-NMR (400 MHz, DMSO-d⁶): δ 7.66 (d, 1H), 7.56 (d, 1H), 7.18 (t, 1H), 6.98 (t, 2H), 6.65 (s, 2H), 5.96 (d, 1H), 0.96 (s, 9H), 0.20 (s, 6H).

### Step-02: Preparation of (E)-N-(6-amino-54(2-((tert-butyldimethylsilyl)oxy)phenyl)diazenyl)pyridin-2-yl)acetamide

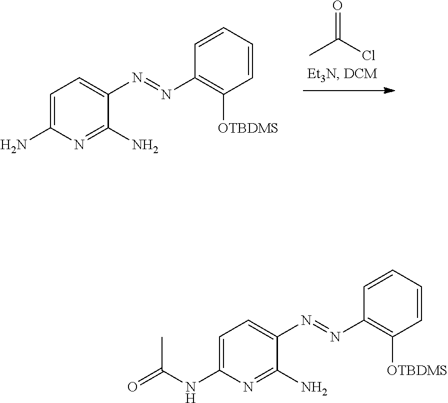

To a stirred solution of (E)-3-((2-((tert-butyldimethylsilyl)oxy)phenyl)diazenyl)pyridine-2,6-diamine (0.4 g) in dichloromethane (6 mL) were added triethylamine (0.32 mL) and acetyl chloride (0.16 mL) at 0° C. temperature. The resulting reaction mixture was stirred at room temperature for 3 h. After completion of the reaction (monitored by TLC), the mixture was diluted with water (20 mL) and extracted with dichloromethane (3×70 mL). The combined organic layer was dried over anhydrous sodium sulfate and concentrated under reduced pressure to obtain a crude. The crude was purified by combi-flash chromatography (18% ethyl acetate in n-heptane) to afford (E)-N-(6-amino-5-((2-((tert-butyldimethylsilyl)oxy)phenyl)diazenyl)pyridin-2-yl)acetamide (0.350 g, 89%) as a yellow solid.

Analytical Details:

LCMS: 95.04% (m/z: 385.19), [M+1]⁺, 2.47 min (4 min run), 214 nm).

### Step-03: Preparation of (E)-N-(6-amino-5-((2-((tert-butyldimethylsilyl)oxy)phenyl) diazenyl)pyridin-2-yl)acetamide

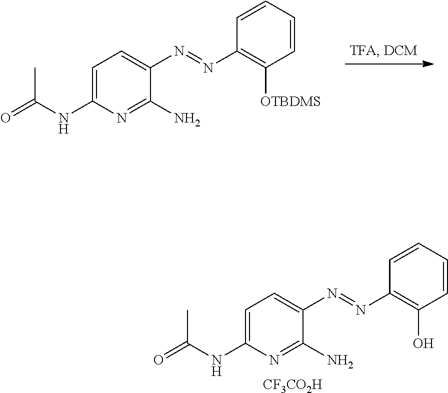 [SMILES: CC(=O)Nc1ccc(/N=N/c2ccccc2O[Si](C)(C)C(C)(C)C)c(N)n1 ; CC(=O)Nc1ccc(/N=N/c2ccccc2O)c(N)n1 ; "CF3CO2H"]

To a stirred solution (E)-N-(6-amino-54(2-((tert-butyldimethylsilypoxy)phenyl)diazenyl)pyridin-2-yl)acetamide (0.35 g) in dichloromethane (5 mL) was added trifluoroacetic acid (4 mL) at 0° C. and then reaction mixture was stirred at room temperature for 3 h. After completion of the reaction (monitored by TLC), the reaction mass was extracted with dichloromethane (2×20 mL) followed by washings with water (2×50 mL). The combined organic layer was dried over anhydrous sodium sulfate and concentrated under reduced pressure to obtain a crude. The crude was purified by ether washings to furnish (E)-N-(6-amino-5-((2-hydroxyphenyl)diazenyl)pyridin-2-yl)acetamide (67 mg, 27.9%) as a yellow solid.

LCMS: 98.69% (m/z: 271.11), [M+1]⁺, 5.14 min (10 min run), 214 nm).

¹H-NMR (400 MHz, DMSO-d₆): δ 8.13 (d, 1H), 7.71 (dd, 1H), 7.40 (d, 1H), 7.29 (t, 1H), 7.01 (d, 1H), 6.93 (t, 1H), 2.10 (s, 3H).

### Example-41: Preparation of methyl (E)-(6-amino-5-((2-hydroxyphenyl)diazenyl)pyridin-2-yl)carbamate

### Step-01: Preparation of (E)-3-((2-((tert-butyldimethylsilyl)oxy)phenyl)diazenyl)pyridine-2,6-diamine

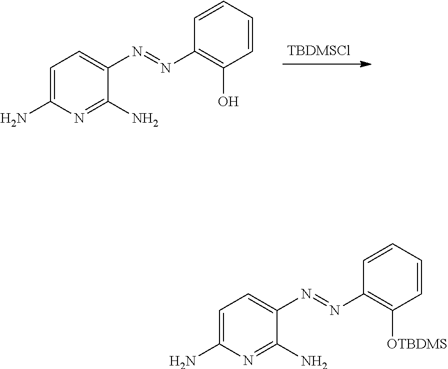

To a stirred solution of ((E)-2-((2,6-diaminopyridin-3-yl)diazenyl)phenol (2 g) in dichloromethane (25 mL) were added triethylamine (1.98 mL) and tert-butyldimethylsilylchloride (2.2 g) at 0° C. temperature. Resulting reaction mixture was stirred at room temperature for additional 4 h. After completion of reaction (monitored by TLC), the mixture was diluted with water (20 mL) and extracted with dichloromethane (3×70 mL). Collected organics were dried over anhydrous sodium sulfate and concentrated under reduced pressure to obtain a crude. The crude was purified by combi-flash chromatography (using 23% ethyl acetate in heptane) affording (E)-3-((2-((tert-butyldimethylsilyl)oxy)phenyl)diazenyl)pyridine-2,6-diamine (2.6 g, 99%) as a red solid.

LCMS: 99.69% (m/z: 344.46), [M+1]⁺, 2.47 min (4 min run), 214 nm).

¹H-NMR (400 MHz, DMSO-d₆): δ 7.66 (d, 1H), 7.56 (d, 1H), 7.18 (t, 1H), 6.98 (t, 2H), 6.65 (s, 2H), 5.96 (d, 1H), 0.96 (s, 9H), 0.20 (s, 6H).

### Step-02: Preparation of Methyl butyldimethylsilyl)oxy)phenyl)diazenyl)pyridin-2-yl)carbamate

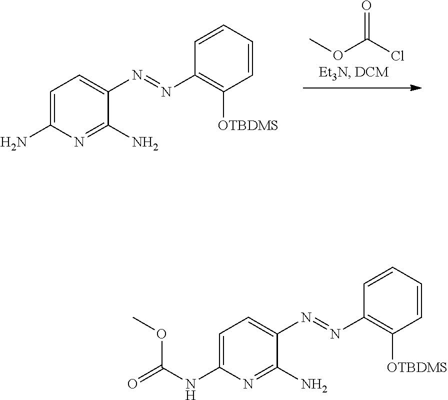

To a stirred solution of (E)-3-((2-((tert-butyldimethylsilyl)oxy)phenyl)diazenyl)pyridine-2,6-diamine (0.3 g) in dichloromethane (6 mL) were added triethylamine (0.2 mL) and methyl chloroformate (0.165 mL) at 0° C. temperature. The resulting reaction mixture was stirred at room temperature for 4 h. After completion of the reaction (monitored by TLC), the mixture was diluted with water (20 mL) and extracted with dichloromethane (3×70 mL). Collected organics were dried the over anhydrous sodium sulfate and concentrated under reduced pressure to obtain the crude. The crude was purified by combi-flash chromatography (18% ethyl acetate in n-heptane) to afford methyl (E)-(6-amino-5-((2-((tert-butyldimethylsilyl)oxy)phenyl)diazenyl)pyridin-2-yl)carbamate (0.16 g, 50.7%) as a yellow solid.

Analytical Details:

LCMS: 80.58% (m/z: 402.47), [M+1]⁺, 2.70 min (4 min run), 214 nm).

### Step-03: Preparation of methyl (E)-(6-amino-5-((2-hydroxyphenyl)diazenyl)pyridin-2-yl)carbamate

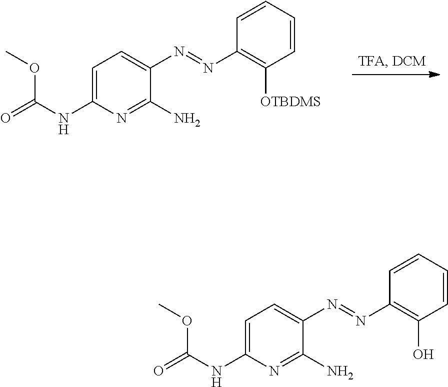 [SMILES: COC(=O)Nc1ccc(/N=N/c2ccccc2O[Si](C)(C)C(C)(C)C)c(N)n1 ; COC(=O)Nc1ccc(/N=N/c2ccccc2O)c(N)n1]

To a stirred solution methyl (E)-(6-amino-54(2-((tert-butyldimethylsilyfloxy)phenyl)diazenyl)pyridin-2-yl)carbamate (0.16 g) in dichloromethane (5 mL) was added trifluoroacetic acid (2 mL) at 0° C. temperature and then reaction mixture was stirred at room temperature for 3 h. After completion of the reaction (monitored by TLC), reaction mass was extracted with dichloromethane (2×20 mL) followed by washings with water (2×50 mL). Collected organics were dried over anhydrous sodium sulfate and concentrated under reduced pressure to obtain a crude. The crude was purified by combi-flash chromatography (using 25% ethyl acetate in n-hexane) to furnish methyl (E)-(6-amino-5-((2-hydroxyphenyl)diazenyl)pyridin-2-yl)carbamate (40 mg, 36.3%) as a yellow solid.

LCMS: 98.84% (m/z: 288.13), [M+1]⁺, 5.77 min (10 min run), 214 nm).

¹H NMR (400 MHz, DMSO-d₆): δ 8.09 (d, 1H), 7.69 (dd, 1H), 7.30-7.25 (m, 2H), 6.99 (d, 1H), 6.93 (t, 1H), 3.67 (s, 3H).

As per reaction condition of example-41 (chloroformate, TEA, DCM); following carbamate prodrugs have been synthesized where respective chloroformate derivatives were procured commercially.

| Examples | Structure | Analytical details |
|---|---|---|
| Example-42 | 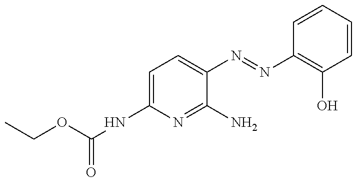 [SMILES: CCOC(=O)Nc1ccc(/N=N/c2ccccc2O)c(N)n1] | LCMS: 98.83% (m/z: 302.05), [M + 1]⁺, 6.07 min (10 min run), 214 nm). ¹H NMR (400 MHz, DMSO-d₆): δ ppm 10.53 (s, 1H), 10.04 (s, 1H), 8.09 (d, 1H), 7.76-7.70 (m, 3H), 7.32- 7.25 (m, 2H), 7.01 (d, 1H), 6.93-6.89 (m, 1H), 4.16 (q, 2H), 1.25 (t, 3H). |
| Example-43 | 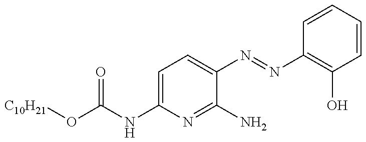 | LCMS: 99.22% (m/z: 414.14), [M + 1]⁺, 4.95 min (10 min run), 214 nm). ¹H-NMR (400 MHz, DMSO-d₆): δ ppm 10.53 (s, 1H), 10.05 (s, 1H), 8.10 (d, 1H), 7.75-7.70 (m, 3H), 7.31- 7.25 (m, 2H), 7.01 (d, 1H), 6.93-6.89 (m, 1H), 4.09 (t, 2H), 1.65-1.58 (m, 2H), 1.34-1.25 (m, 14H), 0.85 (t, 3H). |
| Example-44 | 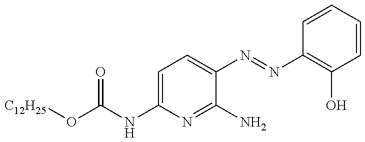 | LCMS: 98.18% (m/z: 442.14 [M + 1]⁺, 6.09 min (10 min run), 214 nm). ¹H-NMR (400 MHz, DMSO-d₆): δ ppm 10.53 (s, 1H), 10.04 (s, 1H), 8.10 (d, 1H), 7.80-7.70 (m, 3H), 7.31- 7.25 (m, 2H), 7.01 (d, 1H), 6.93-6.89 (m, 1H), 4.09 (t, 2H), 1.63-1.58 (m, 2H), 1.34-1.24 (m, 18H), 0.84 (t, 3H). |
| Example-45 | 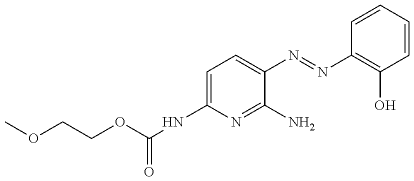 [SMILES: COCCOC(=O)Nc1ccc(/N=N/c2ccccc2O)c(N)n1] | LCMS: 99.21% (m/z: 332.04 [M + 1]⁺, 5.80 min (10 min run), 214 nm). ¹H-NMR (400 MHz, DMSO-d₆): δ ppm 10.52 (br s, 1H), 10.17 (s, 1H), 8.11 (d, 1H), 7.78-7.76 (br s, 2H), 7.72 (dd, 1H), 7.30-7.25 (m, 1H), 7.01 (d, 1H), 6.93-6.89 (m, 1H), 4.24 (t, 2H), 3.57 (t, 2H), 3.29 (s, 3H). |

### Example-46: Preparation of octyl (E)-(6-amino-5-((2-hydroxyphenyl)diazenyl)pyridin-2-yl)carbamate

### Step-01: Preparation of octyl (E)-(6-amino-54(2-((tert-butyldimethylsilyl)oxy)phenyl)diazenyl)pyridin-2-yl)carbamate

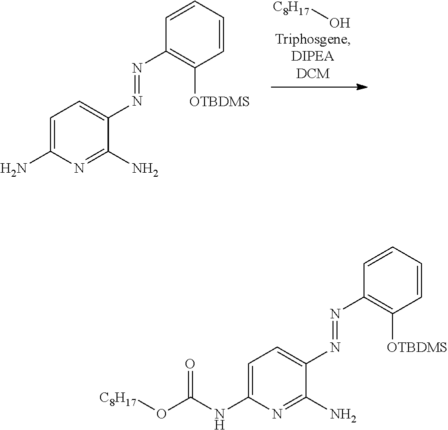

To a stirred solution of (E)-3-((2-((tert-butyldimethylsilyl)oxy)phenyl)diazenyl)pyridine-2,6-diamine (0.3 g) in dichloromethane (12 mL) were added triphosgene (0.128 g) and N, N′-diisopropylethylamine (0.25 mL) at 0° C. temperature. This reaction mixture was stirred at same temperature for additional 15 min. After that, octanol (0.12 g) was added to above mixture, and it was continued stirring at room temperature for additional 16 h. After completion of the reaction (monitored by TLC: 30% ethyl acetate in heptane), the mixture was diluted with water (25 mL) followed by extraction with dichloromethane (3×50 mL). Collected organics layer was dried over anhydrous sodium sulfate and concentrated under reduced pressure to obtain crude. The crude was purified by flash chromatography (using 15% ethyl acetate in n-heptane) to afford octyl (E)-(6-amino-5-((2-((tert-butyldimethylsilyl)oxy)phenyl)diazenyl)pyridin-2-yl)carbamate (0.35 g, 85%) as a brown solid.

Analytical Details:

LCMS: 97.04% (m/z: 500.17), [M+1]⁺, 3.09 min (4 min run), 214 nm).

### Step-02: Preparation of octyl (E)-(6-amino-54(2-hydroxyphenyl)diazenyl)pyridin-2-yl)carbamate

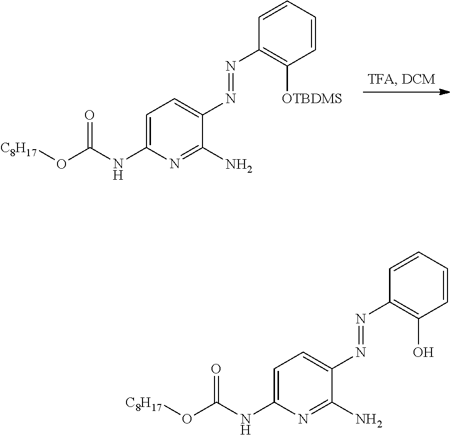

A stirred solution octyl (E)-(6-amino-54(2-((tert-butyldimethylsilyl)oxy)phenyl)diazenyl) pyridin-2-yl)carbamate (0.3 g) in dichloromethane (10 mL) was added trifluoroacetic acid (1.1 mL) at 0° C. temperature and resulting solution was stirred for 2 h at the room temperature. Completion of the reaction was monitored by TLC (70% ethyl acetate in heptane). After completion of the reaction, excess of solvent was evaporated under reduced pressure to obtain crude. The crude was purified by washing with n-pentane followed by washings with diethyl ether to give octyl (E)-(6-amino-5-((2-hydroxyphenyl)diazenyl)pyridin-2-yl)carbamate (0.14 g, 31%) as a red color solid.

LCMS: 99.05% (m/z: 386.09), [M+1]⁺, 4.69 min (10 min run), 214 nm).

¹H-NMR (400 MHz, DMSO-d₆): δ 8.09 (d, 1H), 7.70 (d, 1H), 7.26-7.27 (m, 2H), 7.01-6.93 (m, 2H), 4.18-4.00 (m, 2H), 1.65-1.53 (m, 2H), 1.33-1.12 (m, 10H), 0.93-0.53 (m, 3H).

As per reaction condition of example-46 (Triphosgene, DIPEA, DCM); following carbamate prodrugs have been synthesized where respective alcohol derivatives were procured commercially.

| Examples | Structure | Analytical details |
|---|---|---|
| Example-47 | 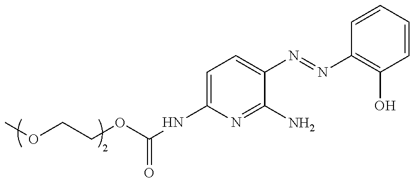 | LCMS: 96.48% (m/z: 376.04, [M + 1]⁺, 5.80 min (10 min run), 214 nm). ¹H-NMR (400 MHz, DMSO-d₆): ppm δ 10.53 (s, 1H), 10.17 (s, 1H), 8.10 (d, 1H), 7.76 (br s, 2H), 7.73- 7.71 (m, 1H), 7.30-7.26 (m, 2H), 7.01 (d, 1H), 6.93-6.89 (m, 1H), 4.23 (br s, 2H), 3.65 (br s, 2H), 3.57-3.56 (m, 2H), 3.46-3.45 (m, 2H), 3.25 (s, 3H). |
| Example-48 | 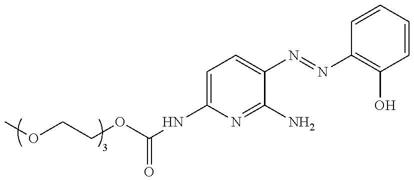 | LCMS: 96.00% (m/z: 420.03), [M + 1]⁺, 5.54 min (10 min run), 214 nm). ¹H-NMR (400 MHz, DMSO-d₆): ppm δ 10.53 (s, 1H), 10.16 (s, 1H), 8.11 (d, 1H), 7.76 (br s, 2H), 7.72 (dd, 1H), 7.31-7.25 (m, 2H), 7.01 (d, 1H), 6.93-6.90 (m, 1H), 4.24 (t, 2H), 3.66 (t, 2H), 3.58-3.56 (m, 2H), 3.54-3.51 (m, 4H), 3.44-3.41 (m, 2H), 3.23 (s, 3H). |

### Example-49: Preparation of (E)-2-((2,6-diaminopyridin-3-yl)diazenyl)phenyl dihydrogen phosphate

### Step-01: Preparation of (E)-dibenzyl (2-((2,6-diaminopyridin-3-yl)diazenyl)phenyl) phosphate

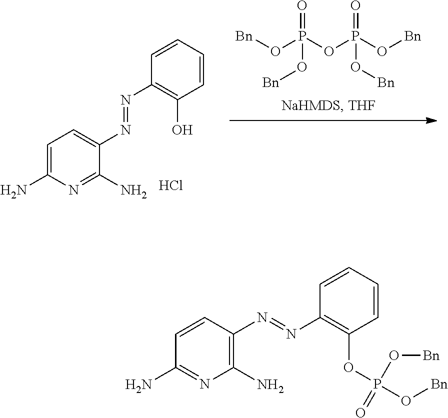 [SMILES: Nc1ccc(/N=N/c2ccccc2O)c(N)n1 ; Cl ; O=P(OCc1ccccc1)(OCc1ccccc1)OP(=O)(OCc1ccccc1)OCc1ccccc1 ; Nc1ccc(/N=N/c2ccccc2OP(=O)(OCc2ccccc2)OCc2ccccc2)c(N)n1]

To a stirred solution of tert-butyl (E)-2-((2,6-diaminopyridin-3-yl)diazenyl)phenol hydrochloride (1 g) in tetrahydrofuran (20 mL) was added sodium bis(trimethylsilyl)amide (1M in THF) at 0° C. temperature and the resulting mixture was stirred at same temperature for 15 min. After that, tetra benzyl diphosphate (4.6 g) was added at same temperature and the reaction mixture continued stirring at room temperature for 30 min. After completion of reaction (monitored by TLC: 50% ethyl acetate in heptane), the mixture was quenched with ammonium chloride followed by extraction with ethyl acetate. Collected organics were dried over anhydrous sodium sulfate and concentrated under reduced pressure to obtain the crude (2 g, crude). The crude proceeded next step without further purification.

LCMS: 60.69% (m/z: 491.20, [M+1]⁺, 2.25 min (10 min run), 214 nm).

### Step-02: Preparation of (E)-2-((2,6-diaminopyridin-3-yl)diazenyl)phenyl dihydrogen phosphate

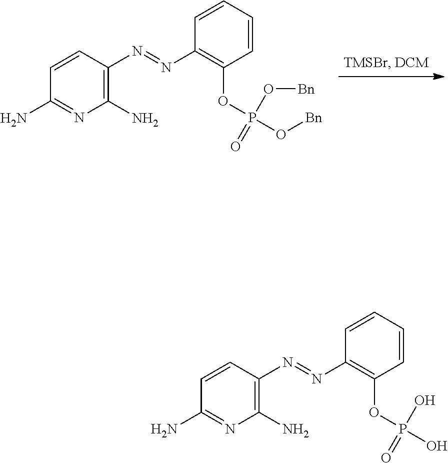 [SMILES: Nc1ccc(/N=N/c2ccccc2OP(=O)(OCc2ccccc2)OCc2ccccc2)c(N)n1 ; Nc1ccc(/N=N/c2ccccc2OP(=O)(O)O)c(N)n1]

To a stirred solution of (E)-dibenzyl (2((2,6-diaminopyridin-3-yl)diazenyl)phenyl) phosphate (1 g) in dichloromethane was added bromotrimethylsilane (0.86 mL) at 0° C. temperature and the resulting reaction mixture was stirred for 15 min at the room temperature. After completion of the reaction (monitored by TLC: 50% ethyl acetate in heptane), excess of solvent was evaporated under reduced pressure to obtain crude material. The crude was purified by prep-HPLC [(NH₄HCO₃ 65%, ACN 35%), X-bridge column] to give of (E)-2-((2,6-diaminopyridin-3-yl)diazenyl)phenyl dihydrogen phosphate (0.170 g, 27%) as orange solid.

LCMS: 95.67% (m/z: 310.04, [M+1]⁺, 4.24 min (10 min run), 214 nm).

¹H-NMR (400 MHz, DMSO-d₆): δ 7.84 (d, 1H), 7.60 (d, 1H), 7.33 (d, 1H), 7.27 (t, 1H), 7.11 (t, 1H), 6.08 (d, 1H).

### Example-50: Preparation of (E)-(2-((2,6-diaminopyridin-3-yl)diazenyl)phenoxy)methyl dihydrogen phosphate

### Step-01: Preparation of (E)-di-tert-butyl ((2-((2,6-diaminopyridin-3-yl)diazenyl)phenoxy)methyl) phosphate

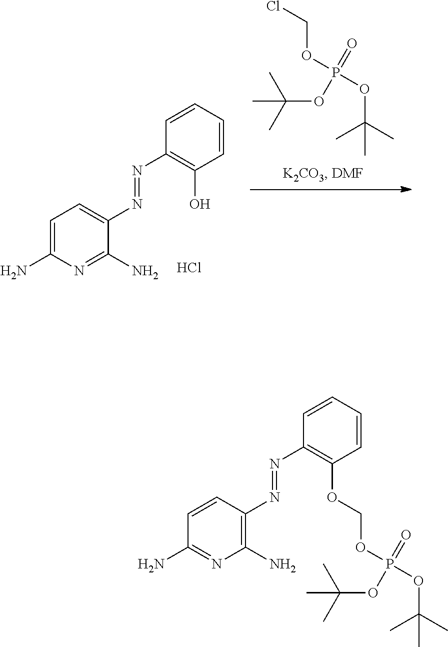

To a stirred solution of (E)-2-((2,6-diaminopyridin-3-yl)diazenyl)phenol hydrogen chloride (0.3 g) in N,N′-dimethylformamide (6 mL) was added potassium carbonate (0.937 g) followed by addition of di-tert-butyl (chloromethyl) phosphate (1.16 g). This solution was stirred at 60° C. temperature for additional 6 h. After completion of the reaction (monitored by LCMS), the mixture was quenched with water (25 mL) and extracted with ethyl acetate (2×50 mL). The combined organic layer was washed with chilled water, dried over anhydrous sodium sulfate, and evaporated under reduced pressure to obtain (E)-di-tert-butyl ((2-((2,6-diaminopyridin-3-yl)diazenyl)phenoxy)methyl) phosphate (0.500 g, crude). The crude was carried forward to next step without further purification.

LCMS: 37.14% (m/z: 452.51), [M+1]⁺, 2.14 min (4 min run), 214 nm).

### Step-02: Preparation of (E)-(2-((2,6-diaminopyridin-3-yl)diazenyl)phenoxy)methyl dihydrogen phosphate

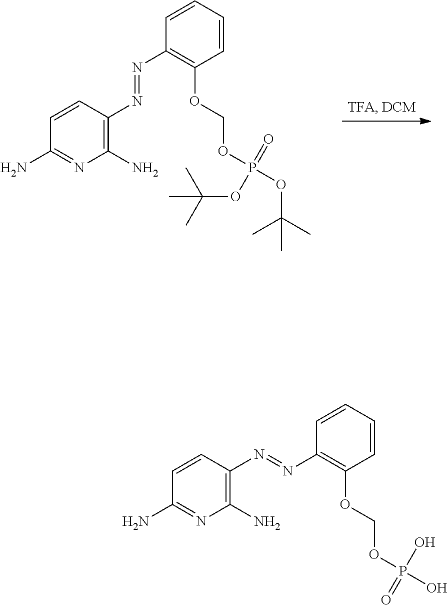 [SMILES: CC(C)(C)OP(=O)(OCOc1ccccc1/N=N/c1ccc(N)nc1N)OC(C)(C)C ; Nc1ccc(/N=N/c2ccccc2OCOP(=O)(O)O)c(N)n1]

To a stirred solution of (E)-di-tert-butyl ((2-((2,6-diaminopyridin-3-yl)diazenyl)phenoxy)methyl) phosphate (0.5 g) in dichloromethane (10 mL) was added trifluoroacetic acid (2 mL) at 0° C. temperature. the resulting solution was stirred for additional 30 min at 0° C. temperature. After completion of the reaction (monitored by LCMS), mixture was evaporated under reduced pressure (bellow 25° C.) to obtain a crude. The crude was purified by prep HPLC (using ammonium bicarbonate as a buffer) to give (E)-(2-((2,6-diaminopyridin-3-yl)diazenyl)phenoxy)methyl dihydrogen phosphate (0.012 g, 3.2%) as a yellow solid.

LCMS: 95.81% (m/z: 340.05 [M+1]⁺, 2.67 min (10 min run), 214 nm).

¹H-NMR (400 MHz, DMSO-d₆): δ 7.76 (d, 1H), 7.60 (dd, 1H), 7.33 (d, 1H), 7.26 (t, 1H), 7.05 (t, 1H), 6.02 (d, 1H), 5.55 (d, 2H).

### Example-51: Preparation of (E)-2,6-diamino-5-(phenyldiazenyl)pyridin-3-yl butyrate

### Step-01: Preparation of 2,6-dinitropyridin-3-ol

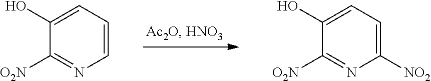 [SMILES: O=[N+]([O-])c1ncccc1O ; O=[N+]([O-])c1ccc(O)c([N+](=O)[O-])n1]

To a pre-mixture solution of acetic anhydride (14 mL) and nitric acid (6.2 mL) was added a solution of 2-nitropyridin-3-ol (5 g) drop wisely at 0° C. temperature. Resulting solution was stirred at 0° C. for 2 h followed by stirring at room temperature for additional 2 h. After completion of the reaction (monitored by TLC), the reaction mass was quenched with ice water and extracted with dichloromethane (3×150 mL). The combined organic layer was dried over anhydrous sodium sulfate, evaporated under reduced pressure to obtain a crude. The crude was triturated with n-pentane followed by drying under reduced pressure to afford 2,6-dinitropyridin-3-ol (3.4 g, 51%) as a yellow solid.

¹H NMR (400 MHz, DMSO-d₆): δ 10.72 (b s, 1H), 8.53 (d, 1H), 7.89-7.85 (m, 1H).

### Step-02: Preparation of 2,6-diaminopyridin-3-yl butyrate

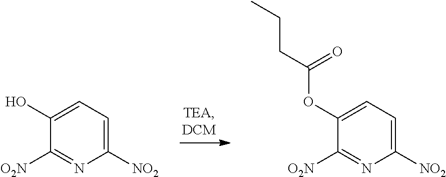 [SMILES: O=[N+]([O-])c1ccc(O)c([N+](=O)[O-])n1 ; CCCC(=O)Oc1ccc([N+](=O)[O-])nc1[N+](=O)[O-]]

To a stirred solution of 2,6-dinitropyridin-3-ol (1 g) in dichloromethane (100 mL) were added triethylamine (1.7 mL) and butyryl chloride (1.09 g) at 0° C. temperature. Then, resulting mixture was stirred at 50° C. for 2.5 h. After completion of the reaction (monitored by TLC), reaction mass was cooled to room temperature and then poured onto ice water (300 mL). Reaction mass was extracted with ethyl acetate (3×150 mL), followed by washings with water (2×100 mL). The combined organic layer was dried over anhydrous sodium sulfate, evaporated under reduced pressure to afford 3-((4-methoxybenzyl)oxy)-2,6-dinitropyridine (1 g, crude) as a brown oil. Crude product was carried forward to next step without purification.

¹H NMR (400 MHz, DMSO-d₆): δ 8.47 (d, 1H), 7.75 (d, 1H), 2.17 (t, 2H), 1.55-1.46 (m, 2H), 0.88 (t, 3H)

### Step-03: Preparation of 2,6-diaminopyridin-3-yl butyrate

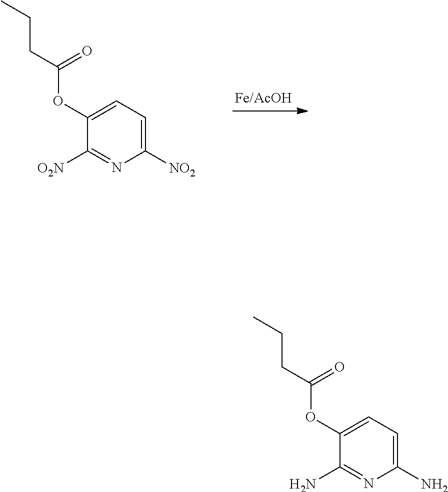 [SMILES: CCCC(=O)Oc1ccc([N+](=O)[O-])nc1[N+](=O)[O-] ; CCCC(=O)Oc1ccc(N)nc1N]

To a stirred solution of 3-((4-methoxybenzyl)oxy)-2,6-dinitropyridine (1 g) in acetic acid (20 mL) was added iron powder (1.5 g) and resulting mixture was stirred at room temperature for 1 h. After completion of the reaction (monitored by TLC), reaction mass was diluted with ethyl acetate (100 mL). Reaction mass was passed through celite bed to remove inorganics followed by several washings with ethyl acetate. The combined organics were washed with water (2×200 mL), dried over anhydrous sodium sulfate, and concentrated under reduced pressure to obtain 3-((4-methoxybenzyl)oxy)pyridine-2,6-diamine as brown oil (0.80 g, crude). Product was carried forward to next step without further purification.

LCMS: 42.03% (m/z: 195.22), [M+1]⁺, 1.24 min (4 min run), 214 nm).

¹H NMR (400 MHz, DMSO-d₆): δ 6.81 (d, 1H), 5.61 (d, 1H), 5.34 (br s, 4H), 2.54 (t, 2H), 1.65-1.58 (m, 2H), 0.95 (m, 3H)

### Step-04: Preparation of (E)-2,6-diamino-5-(phenyldiazenyl)pyridin-3-yl butyrate

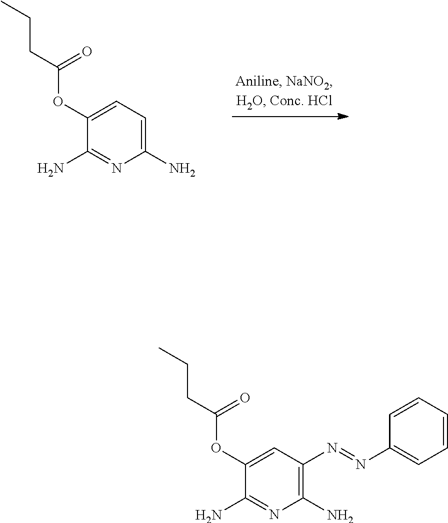 [SMILES: CCCC(=O)Oc1ccc(N)nc1N ; CCCC(=O)Oc1cc(/N=N/c2ccccc2)c(N)nc1N]

To a stirred solution of sodium nitrite (0.28 g) in water (1.5 mL) was added aniline (0.38 g) and 6N hydrochloric acid (0.56 ml). Resulting mixture was stirred at room temperature for 30 min. After that, 3-((4-methoxybenzyl) oxy)pyridine-2,6-diamine (0.8 g; dissolved in water: 5.0 mL) was added to reaction mass and then it was stirred at room temperature for additional 1 h. After completion of reaction (monitored by TLC), it was quenched with sodium acetate and stirred for 15 min. The reaction mixture was extracted with ethyl acetate (3×70 mL), washed with water (2×200 mL), collected organics were dried over anhydrous sodium sulfate, evaporated under reduced pressure to obtain the crude. Crude was purified by flash chromatography (50% ethyl acetate in hexane) to afford (E)-2,6-diamino-5-(phenyldiazenyl)pyridin-3-yl butyrate (0.063 g, 5.16%) as a red solid.

LCMS: 99.14% (m/z: 300.07, [M+1]⁺, 5.49 min (10 min run), 214 nm).

¹H NMR (400 MHz, DMSO-d₆): δ 10.38 (s, 1H), 9.38 (s, 1H), 7.91 (d, 2H), 7.55-7.52 (m, 3H), 7.48-7.44 (m, 1H), 6.95 (br s, 2H), 2.52 (t, 2H), 1.67-1.58 (m, 2H), 0.93 (t, 3H)

### Procedure:

| Example # | Structure | Analytical details |
|---|---|---|
| Example-52 | 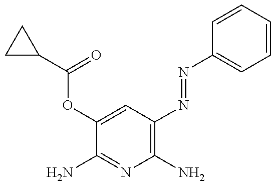 [SMILES: Nc1nc(N)c(OC(=O)C2CC2)cc1/N=N/c1ccccc1] | LCMS: (93.09)% (m/z: 298.11 [M + 1]⁺, 5.33min (10 min run), 214 nm). ¹H-NMR (400 MHz, DMSO-d₆): δ 10.92 (br s, 1H), 9.65 (br s, 1H), 7.92 (d, 2H), 7.58-7.45 (m, 4H), 7.39-6.99 (m, 2H), 2.28-2.26 (m, 1H), 0.98-0.92 (m, 4H). |
| Example-53 | 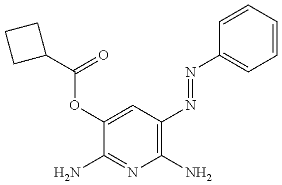 [SMILES: Nc1nc(N)c(OC(=O)C2CCC2)cc1/N=N/c1ccccc1] | LCMS: 97.45% (m/z: 312.07 [M + 1]⁺, 5.60 min (10 min run), 214 nm). ¹H-NMR (400 MHz, DMSO-d₆): δ 10.16 (s, 1H), 9.45 (s, 1H), 7.90 (d, 2H), 7.55-7.52 (m, 3H), 7.49-7.44 (m, 1H), 6.96 (br s, 2H), 3.64-3.56 (m, 1H), 2.28-2.11 (m, 4H), 2.01-1.92 (m, 1H), 1.85-1.81 (m, 1H). |
| Example-54 | 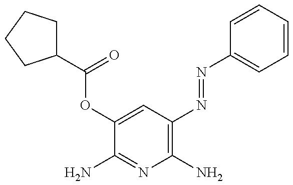 [SMILES: Nc1nc(N)c(OC(=O)C2CCCC2)cc1/N=N/c1ccccc1] | LCMS: (91.84 and 4.01)% (m/z: 326.07 and 326.10), [M + 1]⁺, 5.90 and 5.60 min (10 min run), 214 nm). ¹H-NMR (400 MHz, DMSO-d₆): δ 10.43 (s, 1H), 9.39-9.38 (m, 1H), 7.92- 7.90 (m, 2H), 7.56-7.50 (m, 3H), 7.47- 7.43 (m, 1H), 6.95-6.93 (m, 2H), 3.15- 3.10 (m, 1H), 1.93-1.87 (m, 2H), 2.02- 1.85 (m, 2H), 1.73-1.61 (m, 4H), 1.58- 1.53 (m, 2H). |
| Example-55 | 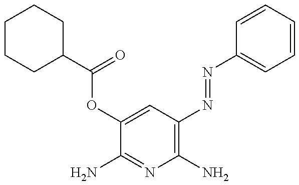 [SMILES: Nc1nc(N)c(OC(=O)C2CCCCC2)cc1/N=N/c1ccccc1] | LCMS: 92.53% (m/z: 340.43 [M + 1]⁺, 6.05 min (10 min run), 214 nm). ¹H-NMR (400 MHz, DMSO-d₆): δ 10.29 (s, 1H), 9.38 (s, 1H), 7.91 (d, 2H), 7.56-7.52 (m, 3H), 7.48-7.46 (m, 1H), 6.96 (br s, 2H), 2.73-2.70 (m, 1H), 1.84-1.81 (m, 2H), 1.77-1.74 (m, 2H), 1.67-1.64 (m, 1H), 1.45-1.36 (m, 2H), 1.32-1.20 (m, 3H). |

### Example-56: Preparation of (E)-2,6-diamino-5-(phenyldiazenyl)pyridin-3-yl diethylcarbamate

### Step-01: Preparation of 2,6-dinitropyridin-3-yl diethylcarbamate

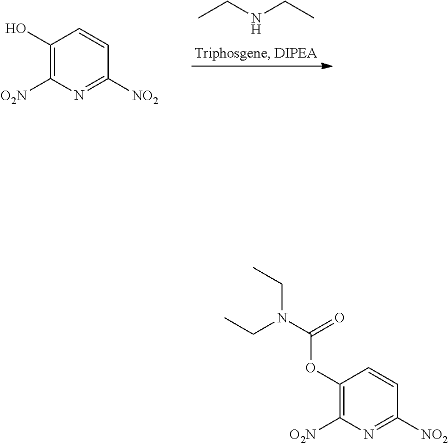

A stirred solution of 2,6-dinitropyridin-3-ol (0.5 g) in dichloromethane (100 mL) was added N,N′-diisopropylethylamine (0.75 mL) followed by addition of triphosgene (0.4 g) at 0° C. temperature. The resulting mixture was stirred at room temperature for 10 min. After that, diethylamine (0.296 g) was added to above mixture and it was stirred at room temperature for additional 16 h. After completion of the reaction (monitored by TLC), water (100 mL) was added to it and reaction mass was extracted with ethyl acetate (3×150 mL). The combined organic layer was dried over anhydrous sodium sulfate, filtered, and evaporated under reduced pressure to afford crude. The crude was purified using flash chromatography to furnish 2,6-dinitropyridin-3-yl diethylcarbamate (330 mg, 46%) as a yellow solid. The compound was carried forward to next step without further purification.

### Step-02: Preparation of 2,6-diaminopyridin-3-yl diethylcarbamate

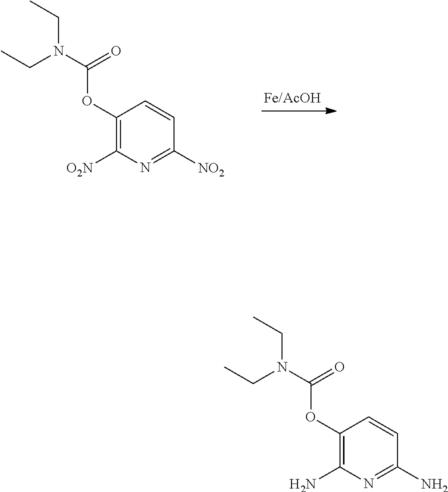 [SMILES: CCN(CC)C(=O)Oc1ccc([N+](=O)[O-])nc1[N+](=O)[O-] ; CCN(CC)C(=O)Oc1ccc(N)nc1N]

To a stirred solution of 2,6-dinitropyridin-3-yl diethylcarbamate (330 mg) in acetic acid (20 mL) was added iron powder (0.063 g) and resulting mixture was stirred at room temperature for 1 h. After completion of the reaction (monitored by TLC), reaction mass was filtered through celite and concentrated under reduced pressure to obtain crude. Crude was further dried to furnish 2,6-diaminopyridin-3-yl diethylcarbamate (300, crude) as a brown oil. Product was carried forward to next step without purification.

LCMS: 76.50% (m/z: 225.05), [M+1]⁺, 1.17 min (4 min run), 214 nm).

### Step-03: Preparation of (E)-2,6-diamino-5-(phenyldiazenyl)pyridin-3-yl diethylcarbamate

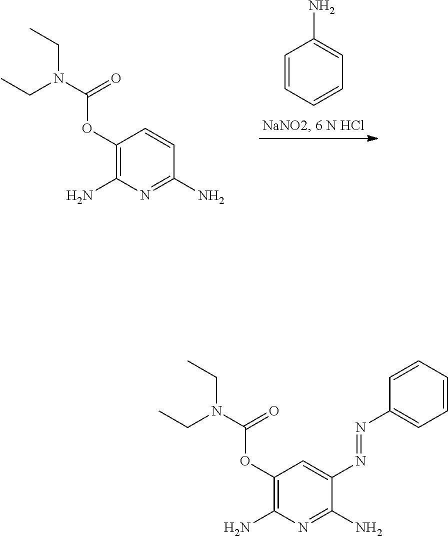 [SMILES: CCN(CC)C(=O)Oc1ccc(N)nc1N ; Nc1ccccc1 ; CCN(CC)C(=O)Oc1cc(/N=N/c2ccccc2)c(N)nc1N]

To a stirred solution of aniline (0.381 g) in water (5 mL) and 6 N hydrochloric acid (1 mL) was added solution of sodium nitrite (0.27 g) in water (1.5 mL) drop-wisely at 0° C. temperature. The resulting mixture was stirred at 0° C. temperature for 30 min. After that, 2,6-diaminopyridin-3-yl diethylcarbamate (0.3 g) was added at 0° C. temperature and resulting reaction mass was stirred at room temperature for additional 1 h. After completion of the reaction (monitored by TLC), reaction mass was quenched with sodium acetate and reaction was stirred for additional 15 min. The reaction mixture was extracted with ethyl acetate (3×70 mL), washed with water (2×50 mL). The collected organic layer was dried over anhydrous sodium sulfate, evaporated under reduced pressure to obtain a crude. The crude was purified by flash chromatography (50% ethyl acetate in hexane) to afford (E)-2,6-diamino-5-(phenyldiazenyl)pyridin-3-yl butyrate (0.063 g, 5.2%) as a red solid.

LCMS: 99.96% (m/z: 329.23), [M+1]⁺, 5.30 min (10 min run), 214 nm).

¹H NMR (400 MHz, DMSO-d₆): δ 8.79 (br s, 1H), 7.75 (d, 2H), 7.46-7.42 (m, 4H), 7.431-7.27 (m, 1H), 6.68 (br s, 2H), 3.46-3.41 (m, 2H), 3.30-3.28 (m, 2H), 1.19 (t, 3H), 1.13 (t, 3H).

| Example | Structure | Analytical details |
|---|---|---|
| Example-57 | 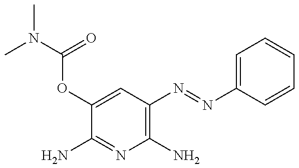 [SMILES: CN(C)C(=O)Oc1cc(/N=N/c2ccccc2)c(N)nc1N] | LCMS: 96.95% (m/z: 301.10 [M + 1] ⁺, 5.53 min (10 min run), 214 nm). ¹H-NMR (400 MHz, DMSO-d₆): δ 7.73 (d, 2H), 7.46-7.28 (m, 6H), 6.72 (br s, 2H), 3.04 (s, 3H), 2.91 (s, 3H) |
| Example-58 | 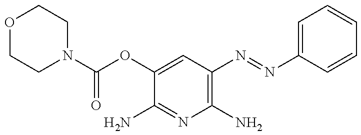 [SMILES: Nc1nc(N)c(OC(=O)N2CCOCC2)cc1/N=N/c1ccccc1] | LCMS: 98.98% (m/z: 343.00, [M + 1]⁺, 5.32 min (10 min run), 214 nm). ¹H-NMR (400 MHz, DMSO-d₆): δ 7.73 (d, 2H), 7.48-7.29 (m, 6H), 6.76 (br s, 2H), 3.65 (br |
| | | s, 4H), 3.59 (br s, 2H), |
| | | 3.40 (br s, 2H). |
| Example-59 | 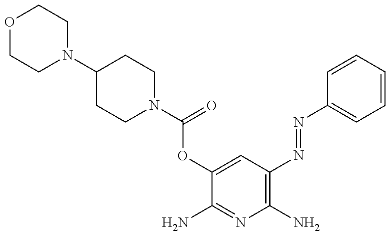 [SMILES: Nc1nc(N)c(OC(=O)N2CCC(N3CCOCC3)CC2)cc1/N=N/c1ccccc1] | LCMS: 98.58% (m/z: 426.15, [M + 1] ⁺, 4.24 min (10 min run), 214 nm). ¹H-NMR (400 MHz, DMSO-d₆): δ 7.73 (d, 2H), 7.46-7.28 (m, 6H), 6.70 (br s, 2H), 4.18- 4.15 (m, 1H), 4.02-3.99 (m, 1H), 3.59-3.57 (m, 4H), 3.05-2.99 (m, 1H), 2.90-2.84 (m, 1H), 2.48-2.40 (m, 4H), 2.37-2.32 (m, 1H), |
| | | 1.82-1.79 (m, 2H), |
| | | 1.53-1.51 (m, 1H), |
| | | 1.42-1.39 (m, 1H). |

### Example 60: Preparation of (E)-2-amino-N-(6-amino-5-((2-hydroxyphenyl)diazenyl) pyridin-2-yl)acetamide

### Step-1: Preparation of tert-butyl (E)-(2-((6-amino-5-((2-hydroxyphenyl)diazenyl)pyridin-2-yl)amino)-2-oxoethyl)carbamate

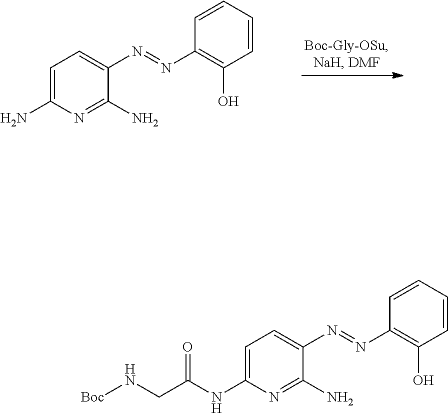 [SMILES: Nc1ccc(/N=N/c2ccccc2O)c(N)n1 ; CC(C)(C)OC(=O)NCC(=O)Nc1ccc(/N=N/c2ccccc2O)c(N)n1]

To a stirred solution of (E)-2-((2,6-diaminopyridin-3-yl)diazenyl)phenol (2 g) in N,N′-dimethyl formamide (20 mL) was added sodium hydride (60% w/w dispersion in mineral oil, 0.87 g) at 0° C. temperature and then reaction mixture was stirred at room temperature for 15 min. After that, N-boc-glycine N-hydroxysuccinimide ester (9.5 g) was added to above solution. The resulting reaction mixture was stirred at room temperature for 4 h. After completion of the reaction (monitored by TLC, 50% ethyl acetate in n-heptane), the mixture was quenched with ice-cold water (50 mL). Reaction mass was extracted by using ethyl acetate (2×50 mL) to afford a crude. The crude was purified by flash chromatography to afford 0.18 g of tert-butyl (E)-(2-((6-amino-5-((2-hydroxyphenyl)diazenyl)pyridin-2-yl)amino)-2-oxoethyl)carbamate as a yellow solid.

LCMS purity: 95.31% (m/z: 387, [M-F1], 0.83 min (4 min run), 214 nm).

### Step-2: Preparation of (E)-2-amino-N-(6-amino-5-((2-hydroxyphenyl)diazenyl) pyridin-2-yl)acetamide

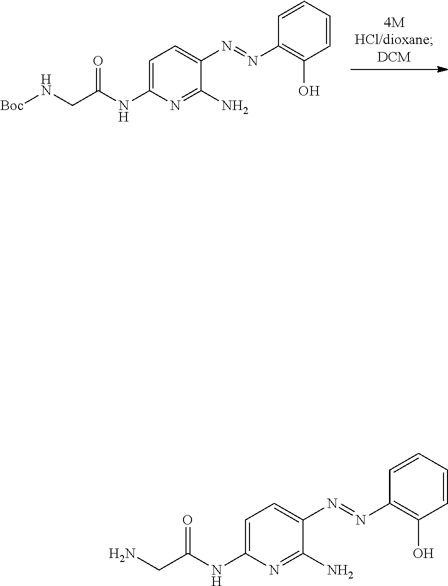 [SMILES: CC(C)(C)OC(=O)NCC(=O)Nc1ccc(/N=N/c2ccccc2O)c(N)n1 ; NCC(=O)Nc1ccc(/N=N/c2ccccc2O)c(N)n1]

To a stirred solution of tert-butyl (E)-(2-((6-amino-5-((2-hydroxyphenyl)diazenyl) pyridin-2-yl)amino)-2-oxoethyl)carbamate (0.25 g) in dichloromethane (5 mL) was added 4 M HCl in 1,4-dioxane (1 mL) at 0° C. temperature under nitrogen atmosphere. Resulting reaction mixture was stirred at room temperature for 2 h. After completion of the reaction (monitored by TLC), the mixture was evaporated under reduced pressure to obtain a crude mass. The crude compound was purified by preparative HPLC using trifluoroacetic acid as a modifier to afford 0.11 g of (E)-2-amino-N-(6-amino-5-((2-hydroxyphenyl) diazenyl)pyridin-2-yl)acetamide mono trifluoroacetate salt as a yellow solid.

LCMS purity: 99.22% (m/z: 287, [M-F1], 4.66 min (10 min run), 214 nm).

¹H-NMR (400 MHz, DMSO-d₆): δ 10.79 (s, 1H), 10.49 (s, 1H), 8.19 (d, 1H), 8.09 (br s, 3H), 7.80 (br s, 2H), 7.73 (d, 1H), 7.50 (br s, 1H), 7.33-7.29 (m, 1H), 7.04 (d, 1H), 6.95-6.91 (m, 1H), 3.87 (br s, 2H).

### Example-61: Preparation of (E)-2-((2-amino-6-(2-aminoacetamido)pyridin-3-yl)diazenyl) phenyl cyclopropanecarboxylate

### Step-1: Preparation of (E)-2-((2-amino-6-(2-((tert-butoxycarbonyl)amino) acetamido)pyridin-3-yl)diazenyl)phenyl cyclopropane carboxylate

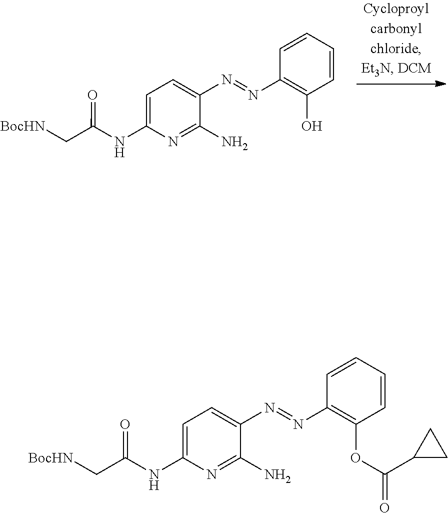 [SMILES: CC(C)(C)OC(=O)NCC(=O)Nc1ccc(/N=N/c2ccccc2O)c(N)n1 ; CC(C)(C)OC(=O)NCC(=O)Nc1ccc(/N=N/c2ccccc2OC(=O)C2CC2)c(N)n1]

To a stirred solution of tert-butyl (E)-(2-((6-amino-5-((2-hydroxyphenyl)diazenyl) pyridin-2-yl) amino)-2-oxoethyl)carbamate (0.5 g, as prepared in example-60, step-1) in dichloromethane (7.5 mL) was added triethylamine (0.45 ml) and resultant mixture was stirred at 0° C. for 5 min. Cyclopropylcarbonyl chloride (0.15 ml) was added to the above reaction mixture and reaction was allowed to stir at room temperature for additional 1 h. After completion of the reaction as monitored by TLC, reaction mixture was quenched with cold water (10 mL). The reaction mass was extracted with dichloromethane (3×15 mL) followed by washings with water (2×30 mL). Collected organics, dried over anhydrous sodium sulfate, and concentrated under reduced pressure to obtain the crude material. The crude was purified by combi-flash chromatography to afford 0.32 g of (E)-2-((2-amino-6-(2-((tert-butoxycarbonyl)amino)acetamido)pyridin-3-yl)diazenyl)phenyl cyclopropane carboxylate as a yellow solid.

LCMS purity: 72.80% (m/z: 455.2, [M+1]⁺, 453.2 [M−1]⁺, 8.11 min (15 min run), 431 nm).

### Step-2: Preparation of (E)-2-((2-amino-6-(2-aminoacetamido)pyridin-3-yl)diazenyl)phenyl cyclopropanecarboxylate

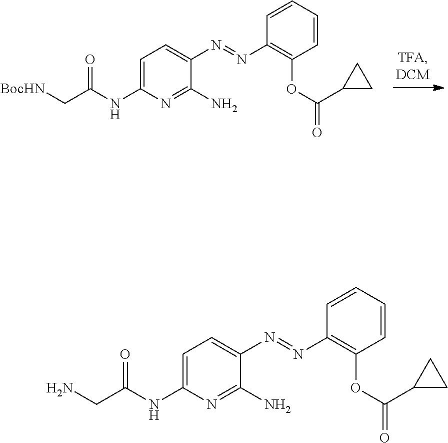 [SMILES: CC(C)(C)OC(=O)NCC(=O)Nc1ccc(/N=N/c2ccccc2OC(=O)C2CC2)c(N)n1 ; NCC(=O)Nc1ccc(/N=N/c2ccccc2OC(=O)C2CC2)c(N)n1]

To a stirred solution of (E)-2-((2-amino-6-(2-((tert-butoxycarbonyl)amino) acetamido)pyridin-3-yl)diazenyl)phenyl cyclopropane carboxylate (0.30 g) in dichloromethane (6 mL) was added trifluoroacetic acid (0.5 mL) and resulting reaction mixture was stirred at room temperature for 1 h. After completion of the reaction as monitored by TLC, solvent was concentrated under reduced pressure. Obtained crude mass was triturated with diethyl ether to obtain 0.15 g of (E)-2-((2-amino-6-(2-aminoacetamido)pyridin-3-yl)diazenyl)phenyl cyclopropane carboxylate as an yellow solid.

LCMS purity: 83.55% (m/z: 355.0 [M+1]⁺, 353.1 [M−1]⁺, 8.16 min (15 min run), 273 nm).

### Example-62: Preparation of (E)-2-((2-amino-6-(2-aminoacetamido)pyridin-3-yl)diazenyl)phenyl cyclopropanecarboxylate

### Step-1: Preparation of (E)-2-((2-amino-6-(2-((tert-butoxycarbonyl)amino) acetamido)pyridin-3-yl)diazenyl)phenyl butyrate

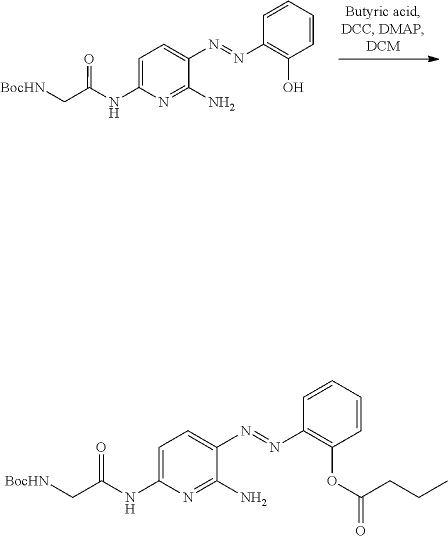 [SMILES: CC(C)(C)OC(=O)NCC(=O)Nc1ccc(/N=N/c2ccccc2O)c(N)n1 ; CCCC(=O)Oc1ccccc1/N=N/c1ccc(NC(=O)CNC(=O)OC(C)(C)C)nc1N]

To a solution of (E)-(2-((6-amino-5-((2-hydroxyphenyl)diazenyl)pyridin-2-yl) amino)-2-oxoethyl) carbamate (0.3 g, as prepared in example-60, step-1) in dichloromethane (10 ml) were added dicylohexylcarbodiimide (0.698 g) and 4-(dimethylamino)pyridine (0.068 g) and stirred the reaction mass at room temperature for 15 min, followed by the addition of butyric acid (0.118 g) and resulting reaction mixture was stirred at room temperature for additional 2 h. After completion of reaction (monitored by TLC), the reaction mass was quenched with cold water (10 ml) and extracted with dichloromethane (3×30 mL). The combined organic layer was washed with water (2×10 mL), dried over anhydrous sodium sulfate, and concentrated under reduced pressure to obtain the crude material which was purified by preparative HPLC to afford 0.21 g of (E)-2-((2-amino-6-(2-((tert-butoxycarbonyl)amino)acetamido) pyridin-3-yl)diazenyl)phenyl as a yellow solid.

LCMS purity: 45.76% (m/z: 457.2, [M+1]⁺, 455.2 [M−1]⁺, 8.38 min (15 min run), 268 nm).

### Step-2: Preparation of ((E)-2-((2-amino-6-(2-aminoacetamido)pyridin-3-yl) diazenyl)phenyl butyrate

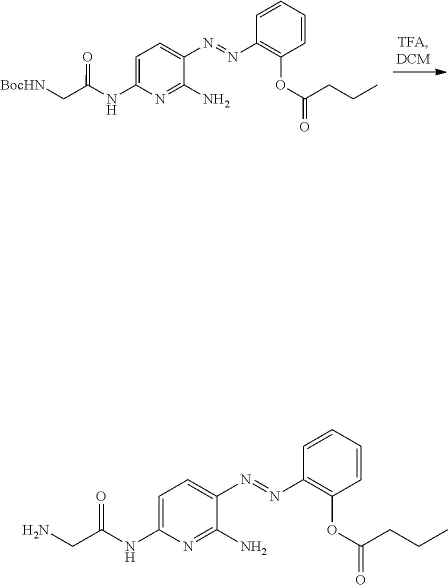 [SMILES: CCCC(=O)Oc1ccccc1/N=N/c1ccc(NC(=O)CNC(=O)OC(C)(C)C)nc1N ; CCCC(=O)Oc1ccccc1/N=N/c1ccc(NC(=O)CN)nc1N]

To a stirred solution of (E)-2-((2-amino-6-(2-((tert-butoxycarbonyl)amino) acetamido)pyridin-3-yl)diazenyl)phenyl butyrate (0.21 g) in dichloromethane (4 mL) was added trifluoroacetic acid (0.3 mL) and resulting reaction mixture was stirred at room temperature for 1 h. After completion of the reaction as monitored by TLC, solvent was concentrated under reduced pressure. Obtained crude mass was triturated with diethyl ether to furnish 0.080 g of ((E)-2-((2-amino-6-(2-amino acetamido)pyridin-3-yl)diazenyl)phenyl butyrate as a light brown solid.

LCMS purity: 94.48% (m/z: 357.2 [M+1]⁺, 355.1 [M−1]⁺, 8.75 min (15 min run), 268 nm).

### Example 63: Preparation of (E)-2-((2-amino-6-(2-aminoacetamido)pyridin-3-yl)diazenyl) phenyl morpholine-4-carboxylate

### Step-1: Preparation of morpholine-4-carbonyl chloride

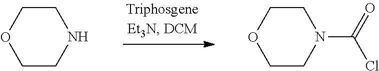 [SMILES: C1COCCN1 ; O=C(Cl)N1CCOCC1]

To a stirred solution of morpholine (4 g) in dichloromethane (40 mL) were added triethylamine (11.2 mL) and triphosgene (20.44 g) at 0° C. temperature under nitrogen atmosphere. Resulting reaction mixture was stirred at room temperature for 3 h. After completion of the reaction (monitored by TLC), reaction mass was diluted with ice cold water followed by extraction with dichloromethane (3×100 mL). The organic layer was washed with water (3×100 mL), dried over anhydrous sodium sulfate, and concentrated under reduced pressure to afford crude. Crude was purified by flash chromatography to obtain 4.52 g of morpholine-4-carbonyl chloride as a light-yellow liquid.

¹H NMR (400 MHz, DMSO-d₆): δ 3.70-3.64 (m, 8H).

### Step-2: Preparation of (E)-2-((2,6-diaminopyridin-3-yl)diazenyl)phenyl morpholine-4-carboxylate

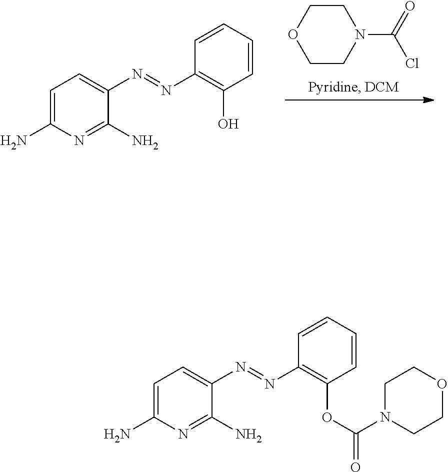

To a stirred solution of (E)-2-((2,6-diaminopyridin-3-yl)diazenyl)phenol (2 g) in pyridine (15 mL) and dichloromethane (15 mL) was added morpholine-4-carbonyl chloride (5.2 g). The resulting reaction mixture was stirred at room temperature for 16 h. After completion of the reaction (monitored by TLC), reaction mass was quenched with 1 M hydrochloric acid solution followed by extraction with dichloromethane (2×50 mL). The combined organic layers were washed with water (50 mL), dried over anhydrous sodium sulfate, and concentrated under reduced pressure to obtain the crude material. Crude was purified by flash chromatography to afford 1.02 g of (E)-2-((2,6-diaminopyridin-3-yl)diazenyl) phenyl morpholine-4-carboxylate as a yellow solid.

LCMS purity: 96.92% (m/z: 343.30, [M+1]⁺, 1.83 min (4 min run), 214 nm).

¹H NMR (400 MHz, DMSO-d₆-D₂O): δ 7.88 (d, 1H), 7.73 (d, 1H), 7.37 (t, 1H), 7.31 (t, 1H), 7.25 (d, 1H), 6.15 (d, 1H), 3.62-3.65 (m, 6H), 3.37-3.39 (m, 2H).

### Step-3: Preparation of (E)-2-((2-amino-6-(2-((tert-butoxycarbonyl)amino) acetamido)pyridin-3-yl)diazenyl)phenyl morpholine-4-carboxylate

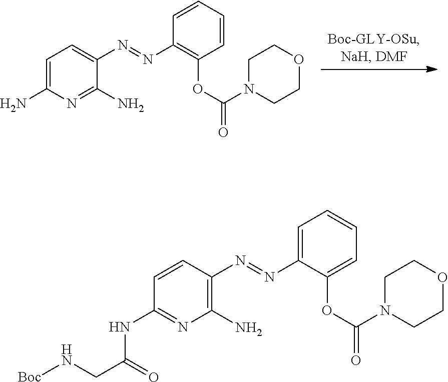 [SMILES: Nc1ccc(/N=N/c2ccccc2OC(=O)N2CCOCC2)c(N)n1 ; CC(C)(C)OC(=O)NCC(=O)Nc1ccc(/N=N/c2ccccc2OC(=O)N2CCOCC2)c(N)n1]

To a stirred solution of (E)-2-((2,6-diaminopyridin-3-yl)diazenyl)phenyl morpholine-4-carboxylate (0.65 g) in N,N-dimethylformamide (7.0 mL) at 0° C. temperature was added sodium hydride (60% w/w dispersion in mineral oil, 0.19 g) in portion wise. Resulting reaction mixture was stirred at 0° C. for 15 min. After that, boc-glycine N-hydroxysuccinimide ester (2.07 g) was added to above solution and reaction mixture is stirred at room temperature for 16 h. After completion of the reaction (monitored by TLC), reaction mass was quenched with ice-water followed by extraction using ethyl acetate (3×50 mL). The combined organic layers were washed with water (50 mL), dried over anhydrous sodium sulfate, and concentrated under reduced pressure to obtain the crude material. Crude was purified by flash chromatography to afford 0.34 g of (E)-24(2-amino-6-(2-((tert-butoxycarbonypamino)acetamido)pyridin-3-yl)diazenyl)phenyl morpholine-4-carboxylate as a yellow solid.

LCMS purity: 93.16% (m/z: 500.13 [M+1]⁺, 498.11 [M−1]⁺, 2.06 min (4 min run), 214 nm).

### Step-4: Preparation of (E)-2-((2-amino-6-(2-aminoacetamido)pyridin-3-yl)diazenyl)phenyl morpholine-4-carboxylate

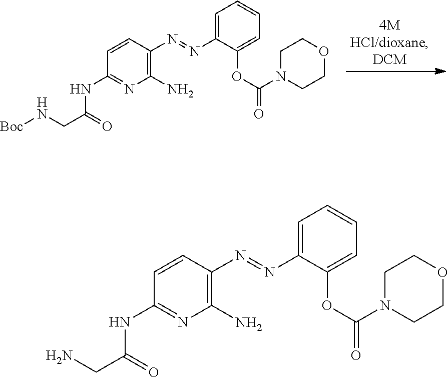 [SMILES: CC(C)(C)OC(=O)NCC(=O)Nc1ccc(/N=N/c2ccccc2OC(=O)N2CCOCC2)c(N)n1 ; NCC(=O)Nc1ccc(/N=N/c2ccccc2OC(=O)N2CCOCC2)c(N)n1]

To a stirred solution of (E)-2-((2-amino-6-(2-((tert-butoxycarbonyl)amino) acetamido)pyridin-3-yl)diazenyl)phenyl morpholine-4-carboxylate (0.3 g) in dichloromethane (2 mL) at 0° C. temperature was added 4M HCl in dioxane (1 mL). Resulting reaction mixture was stirred at room temperature for 2 h. After completion of the reaction (monitored by TLC), reaction mass was evaporated under reduced pressure to obtain the crude material. Crude was triturated with diethyl ether followed by purification using preparative HPLC afforded 0.12 g of (E)-2-((2-amino-6-(2-aminoacetamido) pyridin-3-yl)diazenyl) phenyl morpholine-4-carboxylate as a yellow solid.

LCMS purity: 95.22% (m/z: 400.42, [M+1]⁺, 4.65 min (10 min run), 214 nm).

¹H NMR (400 MHz, DMSO-d₆-D₂O): 8.09 (d, 1H), 7.73 (d, 1H), 7.47 (t, 2H), 7.29-7.36 (m, 2H), 3.76-3.96 (m, 2H), 3.50-3.83 (m, 6H), 3.33-3.40 (m, 2H).

### Example-64: Preparation of (E)-2-((2-amino-6-(2-aminoacetamido)pyridin-3-yl)diazenyl)phenyl acetate

### Step-1: Preparation of (E)-24(2-amino-6-(2-((tert-butoxycarbonyl)amino)acetamido)pyridin-3-yl)diazenyl)phenyl acetate

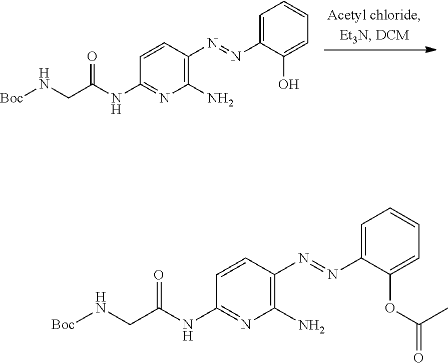 [SMILES: CC(C)(C)OC(=O)NCC(=O)Nc1ccc(/N=N/c2ccccc2O)c(N)n1 ; CC(=O)Oc1ccccc1/N=N/c1ccc(NC(=O)CNC(=O)OC(C)(C)C)nc1N]

To a stirred solution of tert-butyl (E)-(2-((6-amino-54(2-hydroxyphenyl)diazenyl)pyridin-2-yl)amino)-2-oxoethyl)carbamate (0.11 g) in dichloromethane (3 mL) was added triethylamine (0.11 ml) followed by addition of acetyl chloride (0.026 ml) at 0° C. Resulting reaction mixture was allowed to stir at room temperature for 2 h. After completion of reaction by TLC, reaction mass was diluted with ice water (10 mL) and washed with saturated sodium bicarbonate solution. The organic layer was dried over anhydrous sodium sulfate and concentrated under reduced pressure to afford 0.12 g of (E)-24(2-amino-6-(2-((tert-butoxycarbonyl)amino)acetamido)pyridin-3-yl)diazenyl)phenyl acetate as orange color solid.

LCMS Purity: 93.45% (m/z: 427.2, [M−1]⁺, 6.59 min (10 min run), 273 nm).

### Step-2: Preparation of (E)-2-((2-amino-6-(2-aminoacetamido)pyridin-3-yl)diazenyl)phenyl acetate

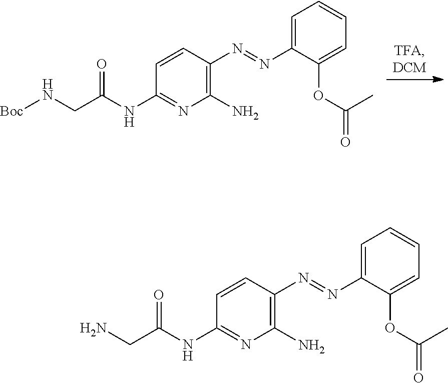 [SMILES: CC(=O)Oc1ccccc1/N=N/c1ccc(NC(=O)CNC(=O)OC(C)(C)C)nc1N ; CC(=O)Oc1ccccc1/N=N/c1ccc(NC(=O)CN)nc1N]

To a stirred solution of (E)-2-((2-amino-6-(2-((tert-butoxycarbonyl)amino) acetamido)pyridin-3-yl)diazenyl)phenyl acetate (0.1 g) in dichloromethane (3 mL) was added trifluoroacetic acid (0.2 mL) at 0° C. temperature under nitrogen atmosphere. Resulting reaction mixture was allowed to stir at room temperature for 1 h. After completion of reaction by TLC, reaction mass was diluted with ice water (10 mL) and washed with saturated sodium bicarbonate solution. The organic layer was dried overanhydrous sodium sulfate and concentrated under reduced pressure to afford 0.075 g of (E)-2-((2-amino-6-(2-aminoacetamido)pyridin-3-yl)diazenyl) phenyl acetate as a yellow solid.

LCMS purity: 94.83% (m/z: 329.0 [M+1]⁺, 327.1 [M−1]⁺, 4.17 min (10 min run), 273 nm).

### Example-65: Preparation of (E)-2-amino-N-(6-amino-5-((24(5-methyl-2-oxo-1,3-dioxol-4-yl)methoxy)phenyl)diazenyl)pyridin-2-yl)acetamide

### Step-1: Preparation of tert-butyl (E)-(2-((6-amino-5-((2-((5-methyl-2-oxo-1,3-dioxol-4-yl)methoxy)phenyl)diazenyl)pyridin-2-yl)amino)-2-oxoethyl)carbamate

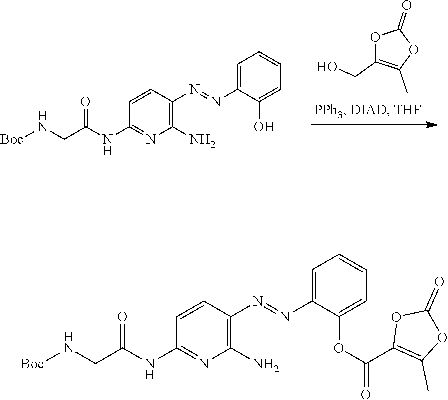

To a stirred solution of (E)-(2-((6-amino-5-((2-hydroxyphenyl)diazenyl)pyridin-2-yl)amino)-2-oxoethyl)carbamate (0.1 g, as prepared in example-60, step-1) in tetrahydrofuran (3 mL) was sequentially added triphenyl phosphine (0.088 g) and diisopropyl azadicarboxylate (0.068 g) at 0° C. temperature under nitrogen atmosphere and the resulting reaction mixture was allowed to stir at room temperature for 10 min followed by addition of 4-(hydroxymethyl)-5-methyl-1,3-dioxol-2-one (0.050 g). After completion of reaction by TLC, reaction mass was diluted with ice water (10 mL) and extracted with ethyl acetate (2×10 mL). The organic layer was dried over anhydrous sodium sulfate and concentrated under reduced pressure to get crude mass which was purified through column chromatography using 10% ethyl acetate in petroleum ether to afford 0.03 g of tert-butyl (E)-(2-((6-amino-5-((2-((5-methyl-2-oxo-1,3-dioxol-4-yl)methoxy)phenyl)diazenyl)pyridin-2-yl)amino)-2-oxoethyl) carbamate as a brown semisolid.

LCMS Purity: 31.28% (m/z: 497.2, [M−1]⁺, 6.91 min (10 min run), 273 nm).

### Step-2: Preparation of (E)-2-amino-N-(6-amino-5-((2-((5-methyl-2-oxo-1,3-dioxol-4-yl)methoxy)phenyl)diazenyl)pyridin-2-yl)acetamide

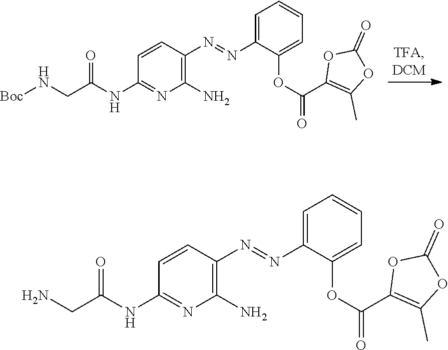 [SMILES: Cc1oc(=O)oc1C(=O)Oc1ccccc1/N=N/c1ccc(NC(=O)CNC(=O)OC(C)(C)C)nc1N ; Cc1oc(=O)oc1C(=O)Oc1ccccc1/N=N/c1ccc(NC(=O)CN)nc1N]

To a stirred solution of tert-butyl (E)-(2-((6-amino-5-((2-(5-methyl-2-oxo-1,3-dioxol-4-yl)methoxy)phenyl)diazenyl)pyridin-2-yl)amino)-2-oxoethyl)carbamate (0.03 g) in dichloromethane (1.5 mL) was added trifluoroacetic acid (0.1 mL) at 0° C. temperature. Resulting reaction mixture was allowed to stir at room temperature for 1 h. After completion of reaction by TLC, reaction mass was diluted with ice water (10 mL) and washed with saturated solution of sodium bicarbonate. The organic layer was dried over anhydrous sodium sulfate and concentrated under reduced pressure to afford 0.015 g of (E)-2-amino-N-(6-amino-5-((2-((5-methyl-2-oxo-1,3-dioxol-4-yl)methoxy)phenyl)diazenyl)pyridin-2-yl)acetamide as an orange color solid.

LCMS purity: 29.11% (m/z: 399.0, [M+1]⁺, 397.1 [M−1]⁺, 4.71 min (10 min run), 273 nm).

### Example-66: Preparation of (E)-2-((2-amino-6-(2-aminoacetamido)pyridin-3-yl)diazenyl)phenyl isobutyl carbonate

### Step-1: Preparation of tert-butyl (E)-(2-((6-amino-5-((2-((isobutoxycarbonyl) oxy)phenyl)diazenyl)pyridin-2-yl)amino)-2-oxoethyl)carbamate

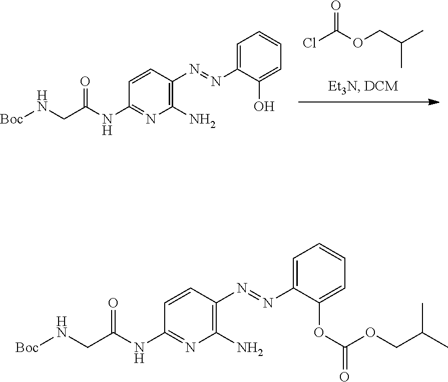

To a stirred solution of (E)-(2-((6-amino-5-((2-hydroxyphenyl)diazenyl)pyridin-2-yl)amino)-2-oxoethyl)carbamate (0.10 g; as prepared in example-60, step-1) in dichloromethane (3 mL) was added triethylamine (0.1 mL) followed by addition of isobutyl chloroformate (0.045 mL) at 0° C. temperature under nitrogen atmosphere. Resulting reaction mixture was allowed to stir at room temperature for 2 h. After completion of reaction by TLC, reaction mass was diluted with ice-cold water (10 mL) and washed with saturated solution of sodium bicarbonate. The organic layer was dried over anhydrous sodium sulfate and concentrated under reduced pressure to afford 0.10 g of tert-butyl (E)-(2-((6-amino-5-((2-((isobutoxy carbonyl)oxy)phenyl)diazenyl)pyridin-2-yl)amino)-2-oxoethyl)carbamate as a light brown solid.

LCMS Purity: 97.15% (m/z: 487.0, [M+1]⁺, 485.2 [M−1]⁺, 7.48 min (10 min run), 268 nm).

### Step-2: Preparation of (E)-2-((2-amino-6-(2-aminoacetamido)pyridin-3-yl) diazenyl)phenyl isobutyl carbonate

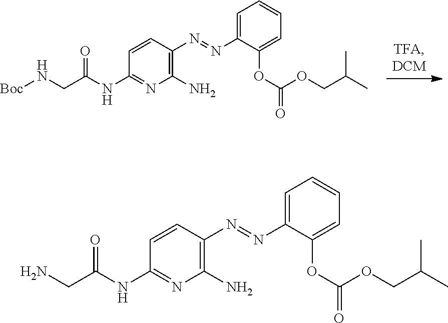 [SMILES: CC(C)COC(=O)Oc1ccccc1/N=N/c1ccc(NC(=O)CNC(=O)OC(C)(C)C)nc1N ; CC(C)COC(=O)Oc1ccccc1/N=N/c1ccc(NC(=O)CN)nc1N]

To a stirred solution of tert-butyl (E)-(2-((6-amino-5-((2-((isobutoxycarbonyl)oxy) phenyl)diazenyl)pyridin-2-yl)amino)-2-oxoethyl)carbamate (0.1 g) in dichloromethane (3 mL) was added trifluoroacetic acid (0.3 mL) at 0° C. temperature under nitrogen atmosphere. Resulting reaction mixture was allowed to stir at room temperature for 1 h. After completion of reaction by TLC, reaction mass was diluted with ice water (10 mL) and washed with saturated sodium bicarbonate solution. The organic layer was dried over anhydrous sodium sulfate and concentrated under reduced pressure to afford 0.07 g of (E)-2-((2-amino-6-(2-aminoacetamido)pyridin-3-yl)diazenyl)phenyl isobutyl carbonate as a yellow solid.

LCMS Purity: 94.27% (m/z: 487.1 [M+1]⁺, 385.1 [M−1]⁺, 5.22 min (10 min run), 273 nm).

### Example-67: Preparation of (E)-2,6-diamino-5-(phenyldiazenyl)pyridin-3-yl methyl carbonate

### Step-1: (E)-2-((2-amino-6-(2-((tert-butoxycarbonyl)amino)acetamido)pyridin-3-yl)diazenyl)phenyl 2-morpholinoacetate

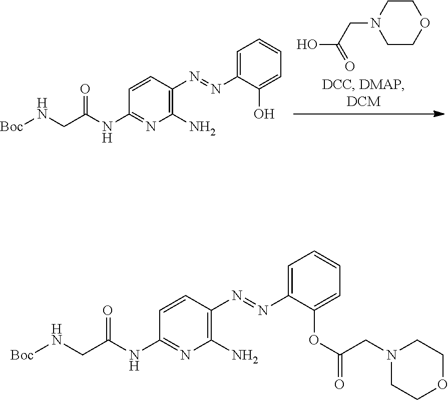

To a stirred solution of tert-butyl (E)-(2-((6-amino-5-((2-hydroxyphenyl) diazenyl)pyridin-2-yl)amino)-2-oxoethyl)carbamate (0.10 g, as prepared in example-60, step-1) in dichloromethane (4 mL) was sequentially added dicyclohexylcarbodiimide (0.13 g) and 4-(dimethylamino)pyridine (0.039 g) at 0° C. temperature. Resulting reaction mixture was allowed to stir at same temperature for 30 min followed by addition of 2-morpholinoacetic acid (0.056 g) and stirred at room temperature for additional 2 h. After completion of reaction by TLC, reaction mass was diluted with ice-cold water (50 mL) and extracted with ethyl acetate (3×200 mL). The combined organic layers dried over anhydrous sodium sulfate and concentrated under reduced pressure which gave crude material which was subjected to column chromatography using 30% ethyl acetate in petroleum ether as mobile phase which afforded 0.11 g of (E)-2-((2-amino-6-(2-((tert-butoxycarbonyl)amino)acetamido)pyridin-3-yl)diazenyl)phenyl 2-morpholinoacetate as a yellow solid.

LCMS purity: 69% (m/z: 514.1 [M+1]⁺, 512.2 [M−1]⁺, 5.23 min (10 min run), 268 nm).

### Step-2: Preparation of (E)-2-((2-amino-6-(2-aminoacetamido)pyridin-3-yl)diazenyl)phenyl 2-morpholinoacetate

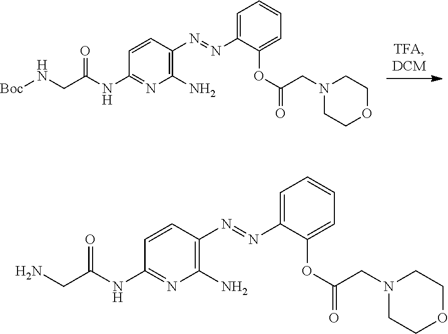 [SMILES: CC(C)(C)OC(=O)NCC(=O)Nc1ccc(/N=N/c2ccccc2OC(=O)CN2CCOCC2)c(N)n1 ; NCC(=O)Nc1ccc(/N=N/c2ccccc2OC(=O)CN2CCOCC2)c(N)n1]

To a stirred solution of (E)-2-((2-amino-6-(2-((tert-butoxycarbonyl)amino) acetamido)pyridin-3-yl)diazenyl)phenyl 2-morpholinoacetate (0.1 g) in dichloromethane (3 mL) was added trifluoroacetic acid (0.3 mL) at 0° C. temperature under nitrogen atmosphere. Resulting reaction mixture was allowed to stir at room temperature for 1 h. After completion of reaction by TLC, reaction mass was diluted with ice-cold water (10 mL) and washed with saturated sodium bicarbonate solution. The organic layer was dried over anhydrous sodium sulfate and concentrated under reduced pressure to afford 0.06 g of (E)-2-((2-amino-6-(2-aminoacetamido)pyridin-3-yl)diazenyl)phenyl 2-morpholinoacetate as a dark orange solid.

LCMS Purity: 35.50% (m/z: 412.2 [M−1]⁺, 3.61 min (10 min run), 273 nm).

### Example-68: Preparation of (E)-2,6-diamino-5-(phenyldiazenyl)pyridin-3-yl methyl carbonate

### Step-1: Synthesis of (E)-3-bromo-5-(phenyldiazenyl)pyridine-2,6-diamine

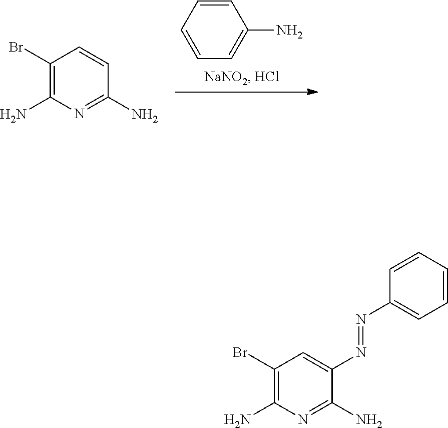 [SMILES: Nc1ccc(Br)c(N)n1 ; Nc1ccccc1 ; Nc1nc(N)c(/N=N/c2ccccc2)cc1Br]

To a stirred solution of aniline (2.9 g) in water (5 mL) was added 6 N hydrochloric acid (3.01 g) followed by addition of sodium nitrite (2.2 g) at 0° C. temperature and the resulting suspension was stirred at 0° C. for additional 30 min followed by the drop wise addition of solution of 3-bromopyridine-2,6-diamine (5 g) in 6 N HCl (25 mL). The resulting reaction mixture was stirred at room temperature for additional 2 h. After completion of the reaction as monitored by TLC, the reaction mass was quenched with sodium acetate (30 mL) and stirred for 15 min. Reaction mass was extracted with ethyl acetate (100 mL×3), followed by washings with water (300 mL×2). The combined organic layer was dried over anhydrous sodium sulfate, and concentrated under reduced pressure to obtain crude. The crude was purified by flash chromatography to afford 2.8 g of (E)-3-bromo-5-(phenyl diazenyl)pyridine-2,6-diamine as a red solid.

LCMS purity: 86.73% (m/z: 293.92, [M+2]⁺, 2.16 min (4 min run), 214 nm).

¹H NMR (400 MHz, DMSO-d₆): δ 7.90 (s, 1H), 7.70 (d, 2H), 7.46 (t, 2H), 7.34 (t, 2H), 6.85 (br s, 2H).

### Step-2: Synthesis of (E)-3-(phenyldiazenyl)-5-(4,4,5,5-tetramethyl-1,3,2-dioxaborolan-2-yl)pyridine-2,6-diamine

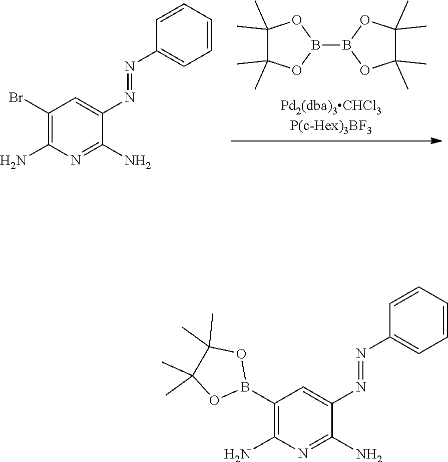

To a stirred solution of (E)-3-bromo-5-(phenyldiazenyl)pyridine-2,6-diamine (2.8 g) in dioxane (30 mL) were added bis(pinacolato)diborane (14.4 g), potassium acetate (1.41 g) and tricyclohexylphosphine tetrafluoroborate (0.353 g) at room temperature. The reaction mixture was degassed using continuous flow of argon gas for next 15 min. After that, Pd₂(dba)₃·CHCl₃ (0.496 g) was added and resulting reaction mixture was stirred at 80° C. for 3 h. After completion of the reaction (monitored by TLC), the reaction mass was cooled to room temperature and diluted with ethyl acetate (100 mL). The organic layers were washed with water (150 mL×2), dried over anhydrous sodium sulfate and concentrated under reduced pressure to obtain the crude. Crude was purified by flash chromatography to afford 2.2 g of (E)-3-(phenyldiazenyl)-5-(4,4,5,5-tetramethyl-1,3,2-dioxaborolan-2-yl)pyridine-2,6-diamine as a yellow solid.

LCMS purity: 75% (m/z: 340.33, [M+1]⁺, 2.44 min (4 min run), 214 nm).

¹H NMR (400 MHz, DMSO-d₆): (δ 8.03 (s, 1H), 7.77 (d, 2H), 7.46 (t, 2H), 7.34 (t, 1H), 6.88 (s, 1H), 6.55 (s, 1H), 6.26 (s, 1H), 1.30 (s, 9H), 1.16 (s, 3H).

### Step-3: Synthesis of (E)-2,6-diamino-5-(phenyldiazenyl)pyridin-3-ol

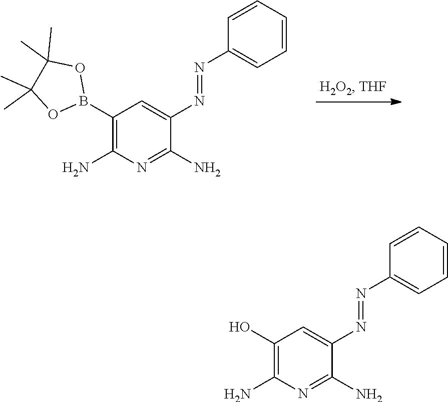 [SMILES: CC1(C)OB(c2cc(/N=N/c3ccccc3)c(N)nc2N)OC1(C)C ; Nc1nc(N)c(/N=N/c2ccccc2)cc1O]

To a stirred solution of (E)-3-(phenyldiazenyl)-5-(4,4,5,5-tetramethyl-1,3,2-dioxaborolan-2-yl)pyridine-2,6-diamine (2.2 g) in tetrahydrofuran (40 mL) was added hydrogen peroxide (0.424 g) and resulting solution was stirred at room temperature for 2 h. After completion of the reaction as monitored by TLC, reaction mass was diluted with ethyl acetate (40 mL). The organic layers were washed with water (100 mL×2), dried over anhydrous sodium sulfate and concentrated under reduced pressure to obtain the crude. The crude was purified by preparative HPLC using trifluoroacetic acid as a modifier to afford 0.44 g of (E)-2,6-diamino-5-(phenyldiazenyl)pyridin-3-ol as a red solid.

LCMS purity: 97% (m/z: 230.03, [M+1]⁺, 1.43 min (6 min run), 214 nm).

¹H NMR (400 MHz, DMSO-d₆): (δ 7.68 (d, 2H), 7.37 (t, 2H), 7.10 (t, 1H), 7.06 (s, 1H), (s, 3H).

### Step-4: Synthesis of (E)-2,6-diamino-5-(phenyldiazenyl)pyridin-3-yl methyl carbonate

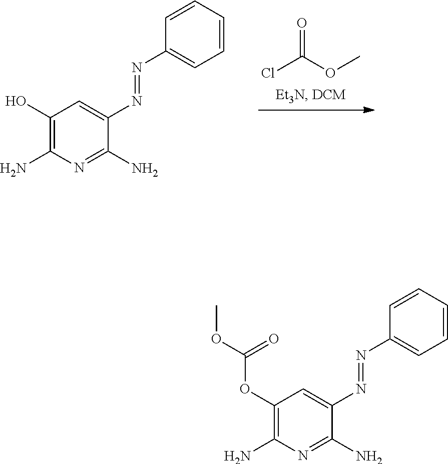

To a stirred solution of (E)-2,6-diamino-5-(phenyldiazenyl)pyridin-3-ol (0.08 g) in dichloromethane (3 mL) were added triethylamine (0.045 g) and methyl chloroformate (0.039 g) at 0° C. temperature and the resulting reaction mixture was stirred at room temperature for 10 min. After completion of the reaction as monitored by TLC, reaction mass was quenched with water (3 mL), extracted with dichloromethane (3×30 mL), washed with brine (2×100 mL), dried over anhydrous sodium sulfate, and concentrated under reduced pressure to obtain the crude. The crude was purified by preparative HPLC using trifluoroacetic acid as a modifier to obtain 0.018 g of (E)-2,6-diamino-5-(phenyldiazenyl)pyridin-3-yl methyl carbonate as a brown solid.

LCMS purity: 98.09% (m/z: 288.10 [M+1]⁺, 4.84 min (10 min run), 214 nm).

¹H NMR (400 MHz, DMSO-d₆-D₂O): δ 7.81 (d, J=8.00 Hz, 2H), 7.52-7.49 (m, 3H), 7.42 (t, J=7.20 Hz, 1H), 3.65 (s, 3H).

### Example 69. Gene Expression Analysis

Study Design: With objective of evaluating effect of text compounds on the pain related markers we have utilized human urothelial “RT4 (Human Urinary bladder carcinoma cells)” cells, treated with test compounds in combination with cyclophosphamide (CYP) and performed gene expression analysis of pain related gene. For gene expression study, 0.5×10\hat{ }6 cells were treated with cyclophosphamide and test compounds. Cells were incubated for 4 h at 37° C. Experimental Groups: Groups 1: Untreated cells, Group 2: CYP treated (100 μM CYP), Group 3: CYP+Compound IX (100 μM CYP+10 μM Compound IX).

At the end of 4 hr incubation, medium was removed from all wells and cells were washed once with sterile PBS. RNA isolation was performed as per the manufacturer's instruction utilizing Qiagen RNA mini kit (Cat #74104). Followed by cDNA synthesis using 3 μg of total RNA each sample utilizing Superscript III First strand cDNA synthesis system (Thermo fisher, Cat No 18080051). qRT-PCR was performed using the SYBR green master mix (PCR Biosystems, Cat No:—PB20.14) and primers specific for P2RX3 gene. Upon the completion of PCR, the data was analysed using the CFX manager software. The Fold change of the expression was calculated using the following formula: 2\hat{ }−ddCT=2\hat{ }−((CT target −CT internal control)

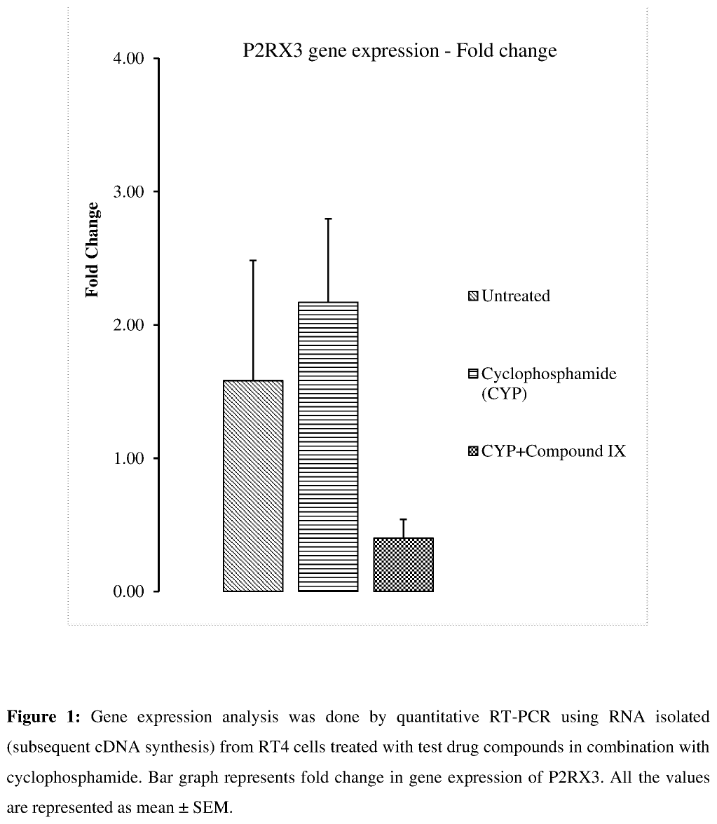

Results: As showed in FIG. 1, CYP treatment for 4 hr to RT4 cells increased the gene expression of P2RX3 when compared to control cells (untreated). Interestingly, CYP treated cells exposed to test Compound IX at 10 μM concentration exhibited reduction in P2RX3 gene expression when compared to CYP control.

### Example 70. Pain Assessment-CFA Model

Study Design: With objective of evaluating effect of test compounds on inflammatory pain we have used Complete Freund's Adjuvant (CFA) as inducing agent to develop inflammation and measured pain response based on the Paw Withdrawal Threshold (PWT). CFA emulsion (75 μg/150 μl/rat) was injected in intraplantar surface of rat hind paw to induce mechanical allodynia. Post-CFA injection, baseline ipsilateral PWT (iPWT) was measured using Von Frey apparatus. Rats were acclimatized to the testing chambers ˜30 min. before initiating the experiments, and prior to Von Frey testing. To determine the lowest mechanical threshold, 2 g filaments are applied to the hind paw and absence of withdrawal response up to 3 secs would prompt to use of next filament with increasing force. Conversely, a hind paw withdrawal response within 3 seconds would prompt use of the next filament of decreasing force. The animals with iPWT ≤4 g were considered to be hyperalgesic and used in the study. Group 1: Vehicle control, Group 2: CFA control (75 μg/150 μl, Per Oral (PO)), Group 3: CFA+compound XI (20 mg/kg, PO), Group 4: CFA+compound XI (5 mg/kg, IV), Group 5: CFA+Naproxen (20 mg/kg, PO), Group 6: CFA+Gabapentin (100 mg/kg, IP). Naproxen and Gabapentin were taken as reference standards. The PWT was recorded at 0.5 hour and 4 hour post-dosing. Calculation of % Maximum Possible Effect (MPE) of Mechanical Allodynia was done using following formula:

$$\% M P E = \frac{( Log P W T of test ) - ( Log Avg . P W T of Vehicle )}{( Log ( 15 ) - Log Avg . P W T of Vehicle )} \times 100$$

Where 15=cut off threshold of mechanical allodynia for mice (highest force).

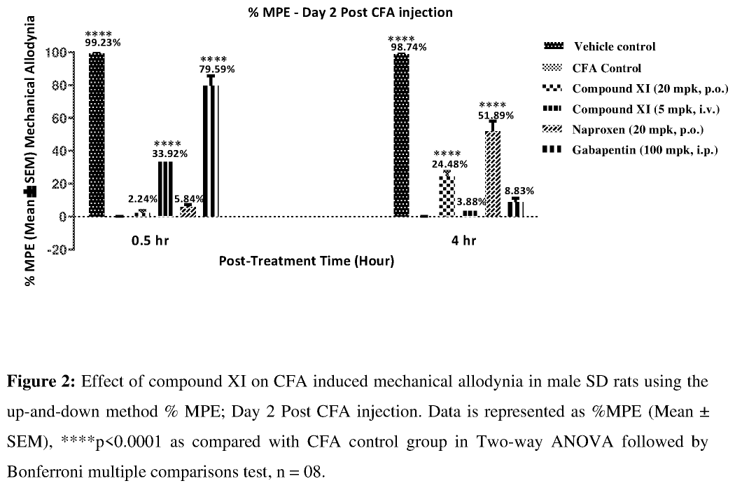

% MPE represented in FIG. 2

### Example 71. Pain Assessment-SNL Model

Study Design: With objective of evaluating effect of test compounds on the neuropathic pain we have used Spinal Nerve Ligation (SNL) technique and measured pain response based on the Paw Withdrawal Threshold (PWT).

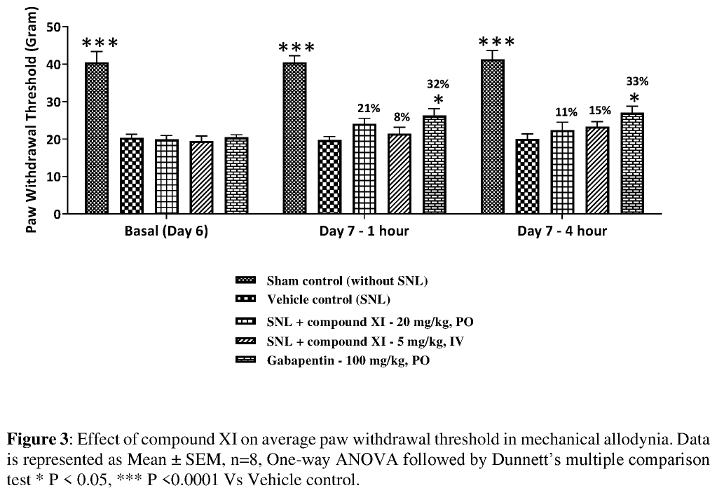

Longitudinal incision was made on lower lumber and sacral levels, followed by removal of muscles and connective tissues. The L6 transverse process covering the L4 and L5 spinal nerves was removed and the L5 and L6 spinal nerves were tightly ligated in the SNL groups whereas only longitudinal incision was made in the Sham control group. Anti-biotic treatment was done post-surgery. For measurement of PWT, the tip of transducer was placed on middle of rat's hind paw and the pressure was applied linearly until clear withdrawal observed. The same was repeated 3 times per animal at an interval of 5 mins per reading and average pressure value was considered as the final PWT recording. On 6^{th} day post-SNL surgery, rats were placed inside the dynamic plantar aesthesiometer apparatus for 30 mins for acclimatization and the baseline response was taken and animals responding to −20 g pain force were selected and randomized in different groups like Group 1: Sham control (without SNL), Group 2: Vehicle control (SNL), Group 3: SNL+compound XI (20 mg/kg, PO), Group 4: SNL+compound XI (5 mg/kg, IV), Group 5: SNL+Gabapentin (100 mg/kg, PO). After single-dose of compound XI, on 7^{th} day post-surgery, rats were acclimatized for 15 mins and mechanical allodynia-Paw withdrawal threshold (PWT) recorded at 1 hour and 4 hour post-dosing using dynamic plantar aesthesiometer. The maximum force was 50 g and ramp 20 secs. Upon the completion of average PWT recording, the % change in average PWT compared to vehicle control was calculated (FIG. 3).

### Example 72—Efficacy of Compounds Against Acute Cyclophosphamide (CYP) Induced Cystitis Model

Experimental System—Study was Performed on Female SD Rats (6-8 Weeks of Age).

Study protocol—Acute interstitial cystitis was induced in experimental animals by a single intraperitoneal administration of cyclophosphamide at a dose of 150 mg/kg body weight. Study groups, dose and route of administration (RoA) is mentioned in the table. The normal control group was administered saline. The test compounds were administered 30 min prior to Cyclophosphamide administration. The Reference compound Ibuprofen was administered 1 h prior to Cyclophosphamide administration. The Control group was administered the vehicle alone. Nociceptive response was evaluated, before CYP or saline injection (basal, 0 h) and at 4 h after CYP administration. Von Frey filaments of increasing forces were applied to the lower abdomen, close to the urinary bladder. Animals were sacrificed, the bladder was removed through a lower midline abdominal incision.

| Treatment Groups | Cyclophosphamide (CYP) | Dose and RoA |
|---|---|---|
| Normal control | Saline, 10 mL/kg, IP | Vehicle |
| CYP control | Cyclophosphamide, | NA |
| CYP + Ibuprofen | 150 mg/kg, IP | 300 mg/kg, PO |
| CYP + Compound No. 449 | (Single dose administration) | 25 mg/kg, PO |
| CYP + Compound No. 54 | | 27 mg/kg, PO |

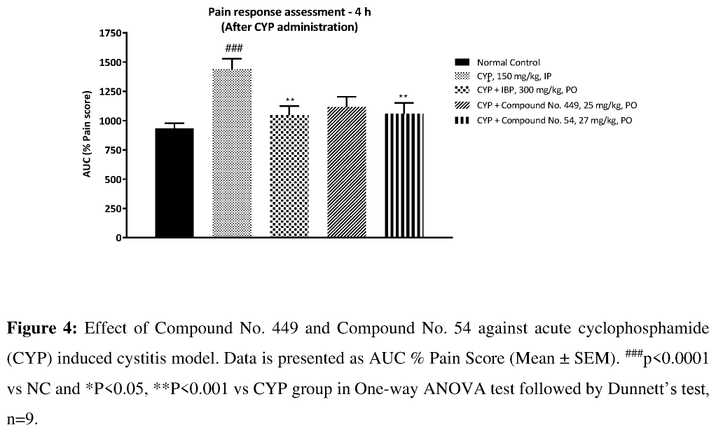

FIG. 4 shows AUC of nociceptive pain scores at 4 hours post CYP dosing.

### Example 73—Efficacy of Compound XI Against Chronic Cyclophosphamide (CYP) Induced Cystitis Model

Experimental System—Study was Performed on Female SD Rats (6-7 Weeks of Age).

Study protocol—Chronic Interstitial cystitis was induced by intraperitoneal administration of cyclophosphamide at a dose of 50 mg/kg body weight on day 0, 3 and Day 6. The normal control group was administered saline. Animals were subjected to evaluation of nociceptive response on Day 7 by using Von Frey Filaments. Prior to testing, the lower abdominal area of each animal was shaved for mechanical stimulation. Von Frey filaments with grading force from 1 to 60 g were applied serially and pain behavior was recorded according to a pain behavior rating scale. Animals were randomized into different treatment groups based on body weight as the primary criteria and pain score as secondary criteria. Animals that exhibited an optimal pain response were selected for randomization and the average pain score across the groups was found to be within ±10%. The test items, dose, dose frequency and route of administration is mentioned in the table below. The Test or Reference compounds were administered for a period of 3 days from Day 8 to Day 10. The test compounds were administered once or twice daily via oral route as indicated in the study design table. Ibuprofen was administered orally once daily for a period of 3 days. The Control group was administered the Vehicle alone. After last dosing on day 10, nociceptive behavior was recorded 1 hr post dose.

| | Cyclophosphamide | Dose, RoA & |
|---|---|---|
| Treatment Groups | (CYP) | Frequency |
| Normal control | Saline, 10 mL/kg, IP | vehicle |
| CYP control | Cyclophosphamide, | NA |
| CYP + Ibuprofen | 50 mg/kg, IP on | 300 mg/kg, PO, QD |
| CYP + Compound XI | day 0, 3, 6 | 20 mg/kg, PO, QD |
| CYP + Compound XI | | 20 mg/kg, PO, BID |

IntraPeritoneal,
QD—once a day,
BID—Twice a day

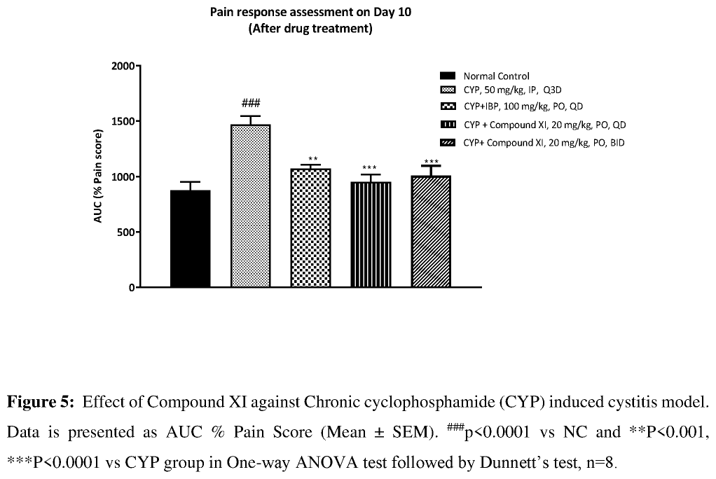

FIG. 5 shows AUC pain score on day 10 (after three days of treatment).

### Example 74—Efficacy of Compounds Against Acute Cyclophosphamide (CYP) Induced Cystitis Model

Experimental System—Study was Performed on Female SD Rats (6-7 Weeks of Age).

Study protocol—Acute interstitial cystitis was induced in experimental animals by a single intraperitoneal administration of cyclophosphamide at a dose of 150 mg/kg body weight. Study groups, dose and route of administration (RoA) is mentioned in the table. The normal control group was administered saline. The test compound were administered 30 min prior to Cyclophosphamide administration. The Reference compound Ibuprofen was administered 1 h prior to Cyclophosphamide administration. The Control group was administered the vehicle alone. Nociceptive response was evaluated, before CYP or saline injection (basal, 0 h) and at 4 h after CYP administration. Von Frey filaments of increasing forces were applied to the lower abdomen, close to the urinary bladder. Animals were sacrificed, the bladder was removed through a lower midline abdominal incision.

| Treatment Groups | Cyclophosphamide (CYP) | Dose and RoA |
|---|---|---|
| Normal control | Saline, 10 mL/kg, IP | Vehicle |
| CYP control | Cyclophosphamide, | NA |
| CYP + Ibuprofen | 150 mg/kg, IP | 300 mg/kg, PO |
| CYP + Compound No. 43 | (Single dose administration) | 25 mg/kg, PO |

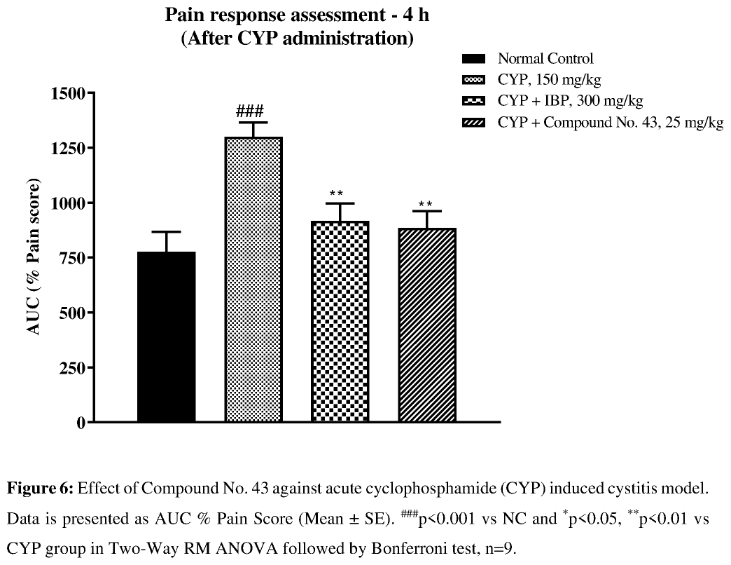

FIG. 6 shows AUC pain score after 4 hr of CYP injection. Example—Efficacy of compounds against Acute cyclophosphamide (CYP) induced cystitis model

Experimental System—Study was Performed on Female SD Rats (6-7 Weeks of Age).

Study protocol—Acute interstitial cystitis was induced in experimental animals by a single intraperitoneal administration of cyclophosphamide at a dose of 150 mg/kg body weight. Study groups, dose and route of administration (RoA) is mentioned in the table. The normal control group was administered saline. The test compounds were administered 30 min prior to Cyclophosphamide administration. The Reference compound Ibuprofen was administered 1 h prior to Cyclophosphamide administration. The Control group was administered the vehicle alone. Nociceptive response was evaluated, before CYP or saline injection (basal, 0 h) and at 4 h after CYP administration. Von Frey filaments of increasing forces were applied to the lower abdomen, close to the urinary bladder. Animals were sacrificed, the bladder was removed through a lower midline abdominal incision.

| Treatment Groups | Cyclophosphamide (CYP) | Dose and RoA |
|---|---|---|
| Normal control | Saline, 10 mL/kg, IP | NA |
| CYP control | Cyclophosphamide, | NA |
| CYP + Ibuprofen | 150 mg/kg, IP | 300 mg/kg, PO |
| CYP + Compound XIII | (Single dose administration) | 20 mg/kg, PO |

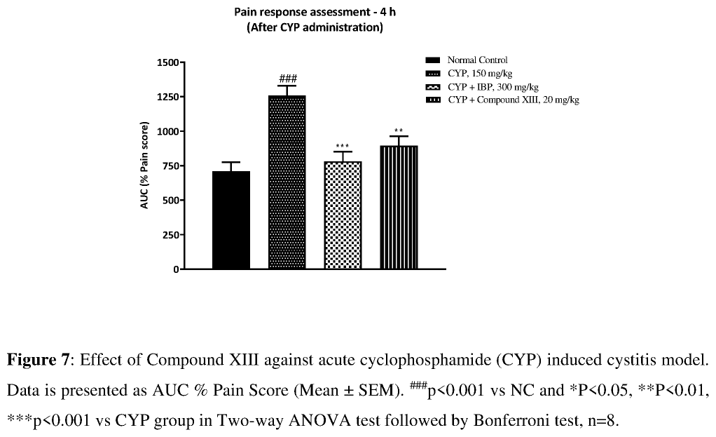

FIG. 7 shows AUC pain score after 4 hr of CYP injection.

### Example 75: Pharmacokinetic Data in Rat

The objective of the study was to evaluate pharmacokinetic exposure of Compound XI along with Compound No. 12/Compound No. 43/Compound No. 446/Compound No. 449/Compound XIII/Compound No. 54 after per oral dosing at 31.2 mg/kg of Compound No. 12, 32.8 mg/kg of Compound No. 43, 34.8 mg/kg of Compound No. 446, 25 mg/kg of Compound No. 449, 21.2 mg/kg of Compound XIII and 27 mg/kg of Compound No. 54 in female Sprague Dawley rat. This study was performed in overnight fasted female Sprague Dawley rat. Dose formulation was prepared freshly on the day of dosing. 0.1% tween-80+99.9% of 0.5% CMC was used as vehicle for the preparation of dose formulation.

Blood samples were collected through retro orbital plexus puncture at pre-dose, 0.25, 0.5, 1, 2, 4, 8, 12 and 24 h post-dose. At each time point, blood was withdrawn and transferred into a pre-labeled of micro centrifuge tubes containing anti-coagulant. Tube was mixed gently by inverting the tube to facilitate mixing of anticoagulant with the blood. All blood/plasma samples were analysed using fit-for purpose LC-MS/MS method.

The pharmacokinetic parameters were calculated using standard non-compartmental analysis (Phoenix® software, version 8.3, Pharsight Corporation, Mountain View, California 94040/USA) using linear trapezoidal method with linear interpolation.

TABLE
Arithmetic Mean pharmacokinetic parameters of Compound No. 12/Compound
No. 43/Compound No. 446/Compound No. 449/Compound XIII/Compound
No. 54 and Compound XI following a single oral route of administration
of Compound No. 12/Compound No. 43/Compound No. 446/Compound No.
449/Compound XIII/Compound No. 54 to Sprague Dawley rat

| | | PK parameters | | | | |
|---|---|---|---|---|---|---|
| | | Cmax | Tₘₐₓ | AUCₗₐₛₜ | AUC_{inf}_{_{—}}_{obs} | AUC_{% Extrap}_{_{—}}_{obs} |
| | Analytes | (ng/mL) | (h) | (h · ng/mL) | (h · ng/mL) | (%) |
| Compound | Compound | 601.32 | 3.33 | 3268.17 | 3324.61 | 1.92 |
| No. 12 | XI | | | | | |
| | Compound | 501.38 | 2.33 | 2698.54 | 2777.74 | 3.39 |
| | No. 12 | | | | | |
| Compound | Compound | 77.62 | 0.58 | 275.38 | 296.66 | 7.15 |
| No. 43 | XI | | | | | |
| | Compound | 1631.82 | 1.00 | 3585.43 | 3671.66 | 2.48 |
| | No. 43 | | | | | |
| Compound | Compound | 93.11 | 0.67 | 173.54 | 179.49 | 3.28 |
| No. 446 | XI | | | | | |
| | Compound | 145.19 | 0.25 | 150.54 | 170.18 | 11.73 |
| | No. 446 | | | | | |
| Compound | Compound | 98.10 | 0.25 | 90.68 | 100.29 | 9.64 |
| No. 449 | XI | | | | | |
| | Compound | 9.26 | 0.25 | 5.98 | 8.32 | 15.39 |
| | No. 449 | | | | | |
| Compound | Compound | NC | NC | NC | NC | NC |
| XIII | XI | | | | | |
| | Compound | 1193.48 | 0.33 | 1848.23 | 1854.30 | 0.33 |
| | XIII | | | | | |
| Compound | Compound | 647.47 | 0.25 | 338.07 | 345.58 | 2.16 |
| No. 54 | XI | | | | | |
| | Compound | NC | NC | NC | NC | NC |
| | No. 54 | | | | | |

NC—Not Calculated

### Example 76—Effect of Compound XI on Bladder Pressure

Experimental System—Study was Performed on Female SD Rats (7-9 Weeks of Age).

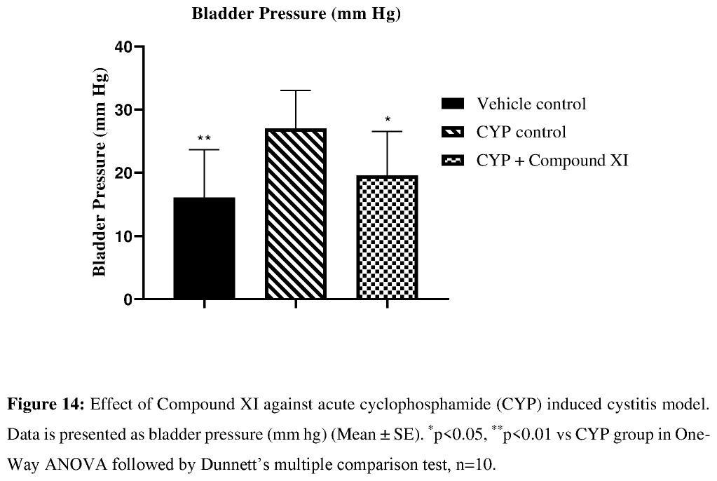

Study protocol—Acute interstitial cystitis was induced in experimental animals by a single intraperitoneal administration of cyclophosphamide at a dose of 150 mg/kg body weight. Study groups, dose, and route of administration (RoA) is mentioned in the table. The normal control group was administered saline. The test compounds were administered 30 min prior to Cyclophosphamide administration. The Control group was administered the vehicle alone. Rats were anesthetized, and body temperature constantly monitored and kept at 36.5° C.±0.2° C. by means of a temperature probe and a heating lamp. The bladder was cannulated and connected to the pressure transducer on one side and an infusion pump on the other (Harvard Apparatus). The pressure transducer was connected via an amplifier to the data acquisition system and a computer. Lab chart Version 8.8.2 software which was used for recording. Infusion pump was started, enabling the infusion of saline at, 100 μl/min. As such the bladder was filled at a constant rate, mean bladder pressure was recorded and shown in FIG. 14.

1. A compound of formula I, pharmaceutically acceptable salts or solvates thereof: 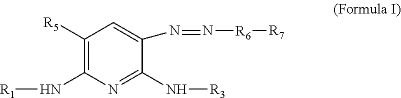
wherein; R₁ and R₃ are the same or different and independently selected from hydrogen, 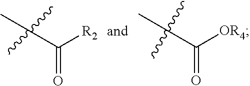
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl; where n is from 1-20;
R₅ is selected from hydrogen, —OH, —OR₈, and 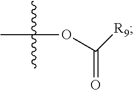
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ—, or NR₁₀R₁₁, where n is from 1-20, R₁₀ and Ru are the same or different and independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₆ is a phenylene group; wherein the phenylene group is zero to four times substituted by R₇, wherein R₇ is selected from hydrogen, —OH, —OR₁₂, 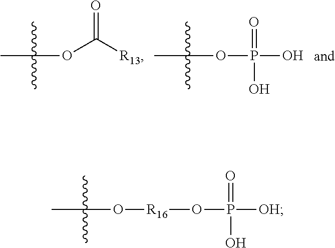
wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group; with the proviso that all substitutions at R₁, R₃, R₅, R₇ are not hydrogen when R₆ is phenylene; when anyone substitution at R₁ or R₃ is other than hydrogen and R₆ is phenylene, then R₅ and R₇ is not hydrogen; when substitution at R₁, R₃, R₇ is hydrogen then R₅ is not —OH; when substitution at R₁, R₃, R₅ is hydrogen and R₆ is phenylene then single R₇ substitution on phenylene is not —OH or —O-(alkyl₁₋₃) at either ortho or para position; when substitution at R₁, R₃ is hydrogen, R₅ is —OH and R₆ is phenylene then R₇ substitution on phenylene is not —OH para position.

2. A compound of formula I-A, pharmaceutically acceptable salts or solvates thereof: 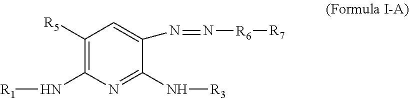
wherein; R₁ and R₃ are the same or different and independently selected from hydrogen, 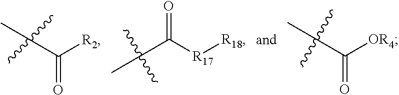
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH₂—CH₂—O)ₙ—, cycloalkyl, heterocycloalkyl, aryl, or heteroaryl, where n is from 1-20; R₁₇ is selected from optionally substituted linear or branched alkylene group, wherein alkylene is optionally substituted with amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₅ is selected from hydrogen, —OH, —OR₈, and 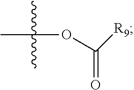
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH₂—CH₂—O)ₙ—, or NR₁₀R₁₁, where n is from 1-20; R₁₀ and R₁₁ are the same or different and independently selected from hydrogen or optionally substituted alkyl, cycloalkyl, aryl, heterocycloalkyl, and heteroaryl group; R₈ is selected from the group consisting of optionally substituted alkyl, alkenyl, alkynyl, cycloalkyl, heterocycloalkyl, aryl, or heteroaryl;
R₆ is a phenylene group, wherein the phenylene group is zero to four times substituted by R₇, wherein R₇ is selected from hydrogen, —OH, —OR₁₂, 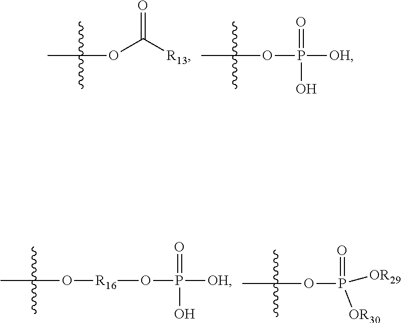
and—R₁₉; wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH₂—CH₂—O)ₙ—, or NR₁₄R₁₅, where n is from 1-20; R₁₄ and R₁₅ are each independently selected from hydrogen, optionally substituted alkyl, heterocycloalkyl, aryl group; R₁₆ selected from optionally substituted alkylene_{(C1-6)} group; R₁₉ is selected from —O—R₂₃—O—C(O)—R₂₄, —O—C(O)—R₂₃—R₂₇, and —O—R₂₃— R₂₈, wherein R₂₃ is optionally substituted linear or branched alkylene; R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group;
R₂₉ and R₃₀ are each independently selected from hydrogen, and —R₂₃—O—C(O)—O—R₃₁; wherein R₃₁ is selected from optionally substituted alkyl, cycloalkyl, heterocycloalkyl, aryl, and heteroaryl group; with the proviso that all substitutions at R₁, R₃, R₅, R₇ are not hydrogen when R₆ is phenylene; when anyone substitution at R₁ or R₃ is other than hydrogen and R₆ is phenylene, then R₅ and R₇ is not hydrogen; when substitution at R₁, R₃, R₇ is hydrogen then R₅ is not —OH; when substitution at R₁, R₃, R₅ is hydrogen and R₆ is phenylene then single R₇ substitution on phenylene is not —OH or —O-(alkyls-3) at either ortho or para position; when substitution at R₁, R₃ is hydrogen, R₅ is —OH and R₆ is phenylene then R₇ substitution on phenylene is not —OH para position.

3. The compound according to claim 1, wherein R4 is alkyl group, may have one or more substituents selected from amino, alkylamino, dialkylamino or optionally substituted heterocycloalkyl, or wherein R4 is aryl group, may have one or more substituents selected from optionally substituted amino acid side chain.

4. The compound according to claim 1, wherein R9 is alkyl group, may have one or more substituents selected from alkylester, amino, and optionally substituted heterocycloalkyl, or R₉ is a heterocycloalkyl group, may have one or more substituents selected from alkyl, amino, alkylamino, dialkylamino, aryl, or optionally substituted heterocyclic ring wherein the optionally substituted heterocyclic ring is substituted by alkyl; or R₉ is a aryloxy group, may have one or more substituents selected from optionally substituted with amino acid residue or amino acid side chain.

5. The compound according to claim 1, wherein R10 and R11 is alkyl group, may have one or more substituents selected from amino, alkylamino, dialkylamino and optionally substituted heterocycloalkyl.

6. The compound according to claim 1, wherein R₁₃ is alkyl group which is optionally substituted with one or more substituents selected from hydroxy, aryl, amino, alkylamino, dialkylamino, alkylalcohol, heterocycloalkyl or substituted heterocycloalkyl; or R₁₃ is alkoxy group which is optionally substituted with one or more substituents selected from amino, alkylamino, dialkylamino, heterocycloalkyl or substituted heterocycloalkyl; or R₁₃ is heterocycloalkyl which is optionally substituted with one or more substituents selected from hydroxy, amino, carboxy, alkyl, alkylamino, dialkylamino, heterocyclic ring, substituted heterocyclic ring wherein the substituted heterocyclic ring is substituted with alkyl; or R13 is heterocycloalkyloxy which is optionally substituted with one or more substituents selected from alkyl group.

7. The compound according to claim 1, wherein R₁₄ and R₁₅ is alkyl group which is optionally substituted with one or more substituents selected from carboxy, amino, alkylamino, dialkylamino, heterocycloalkyl, or substituted heterocycloalkyl group; or R₁₄ and R₁₅ is heterocycloalkyl which is optionally substituted with one or more substituents selected from alkyl, heterocyclic ring or substituted heterocyclic ring, wherein the substituted heterocyclic ring is substituted with alkyl; or R₁₄ and R₁₅ is aryl group which is optionally substituted with one or more substituents selected from heterocyclic or substituted heterocycloalkyl group, wherein the substituted heterocyclic ring is substituted with alkyl.

8. The compound of formula II, pharmaceutically acceptable salts or solvates thereof according to claim 1: 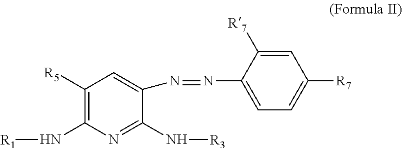
wherein;
R₁ and R₃ are independently selected from hydrogen, 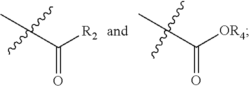
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, cycloalkyl, heterocycloalkyl, aryl, —(CH2-CH2-O)n-, or heteroaryl; where n is from 1-20;
R5 is selected from hydrogen, OH, —OR₈, and 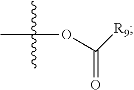
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁, where n is from 1-20; wherein R₁₀ and R₁₁ are the same or different and independently selected from hydrogen or optionally substituted alkyl; R₈ is selected from the group consisting of optionally substituted alkyl, alkenyl, alkynyl, cycloalkyl, heterocycloalkyl, aryl, or heteroaryl; R₇ and R′₇ are the same or different and independently selected from hydrogen, OH, —OR₁₂, 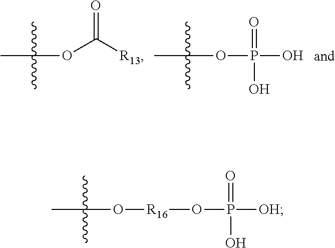
wherein R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, heterocycloalkyloxy, aminoaryl, or amino acid linked via ester or amide linkage at the point of attachment, —(CH2-CH2-O)n-, or NR₁₄R₁₅, where n is from 1-20; wherein R₁₄ and R₁₅ each independently selected from hydrogen, optionally substituted alkyl, heterocycloalkyl, aryl group; R₁₆ is selected from optionally substituted alkylene(cl-6) group;
with the proviso that all substitutions at R₁, R₃, R₅, R₇ and R′₇ is not hydrogen at the same time; when any one substitution at R₁ or R₃ is not hydrogen then R₅, R₇ and R′₇ is not hydrogen; when substitution at R₁, R₃, R₇ and R′₇ is hydrogen then R₅ is not —OH; when substitution at R₁, R₃, R₅ and R′₇ is hydrogen then R₇ is not —OH or —O-(alkyl₁₋₃); when substitution at R₁, R₃, R₅ and R₇ is hydrogen then R′₇ is not —OH or —O-(alkyl₁₋₃); and when substitution at R₁, R₃ and R₇ is hydrogen then both R₅ and R′₇ is not —OH.

9. The compound of formula III, pharmaceutically acceptable salts or solvates thereof according to claim 1: 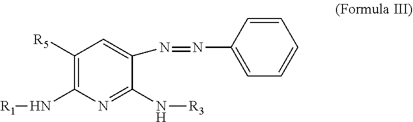
R₁ selected from hydrogen 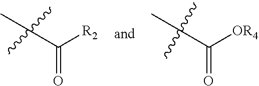
and R₃ is selected from 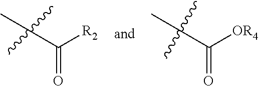
wherein R₂ is selected from the group consisting of optionally substituted alkyl alkenyl, alkynyl, cycloalkyl, heterocycloalkyl, or heteroaryl; R₄ is selected from the group consisting of optionally substituted alkyl, alkenyl, alkynyl, —(CH2-CH2-O)n-, cycloalkyl, heterocycloalkyl, or heteroaryl; where n is from 1-20;
R₅ selected from —OR₈, and 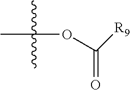
wherein R₉ is selected from the group consisting of optionally substituted alkyl, alkenyl, alkynyl, cycloalkyl, heterocycloalkyl, aryl, heteroaryl, alkyl-ester, alkoxy, heterocycloalkoxy, heteroaryloxy, cycloalkoxy, aryloxy, amino acid linked via ester or amide linkage at the point of attachment, —(CH2-CH2-O)n-, or N₁₀R₁₁, wherein R₁₀ repand R₁₁ are the same or different and are independently selected from hydrogen, optionally substituted alkyl; where n is from 1-20; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl alkenyl, alkynyl, cycloalkyl, heterocycloalkyl, aryl, or heteroaryl.

10. The compound according to claim 9, selected from group consisting of: 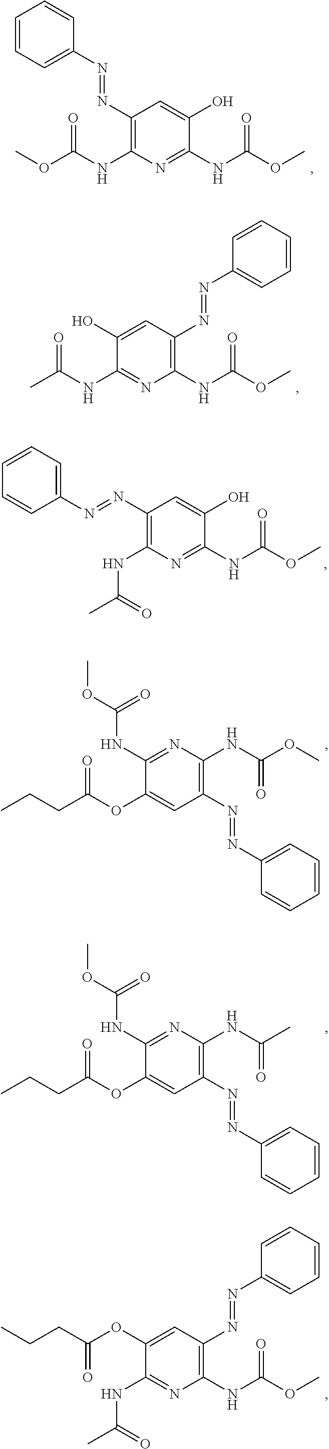 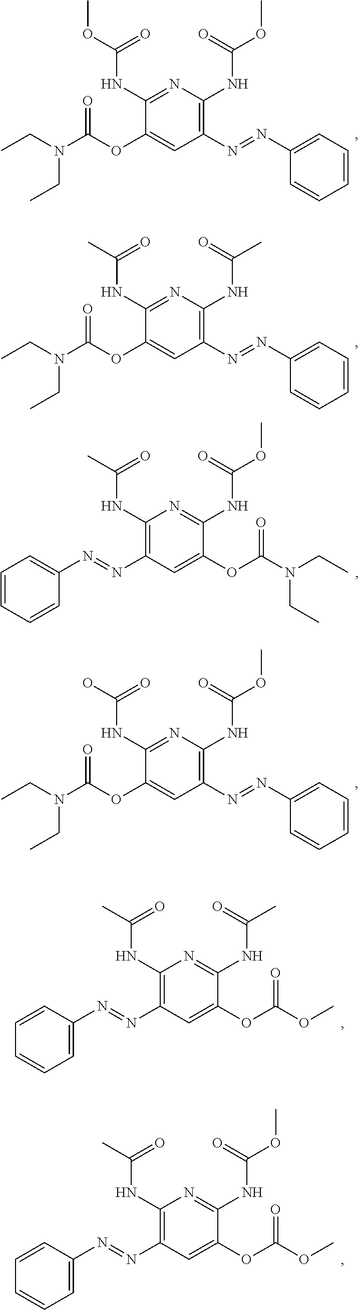 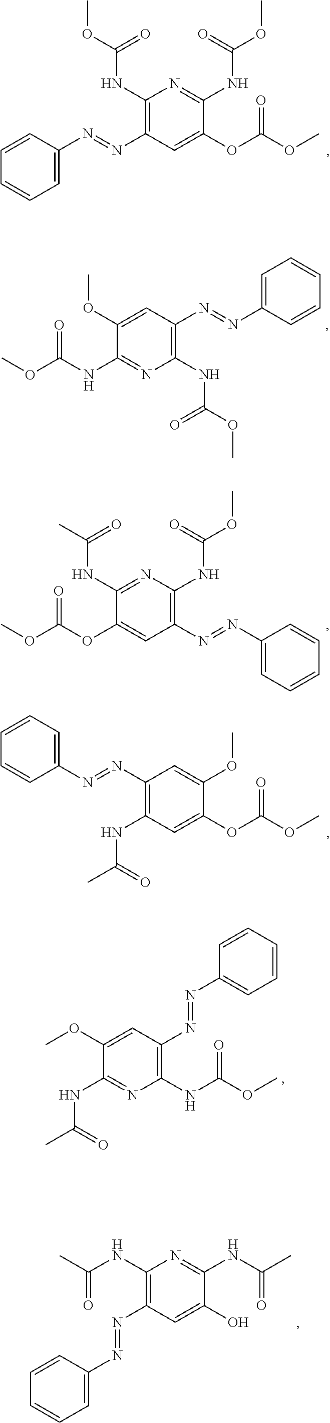 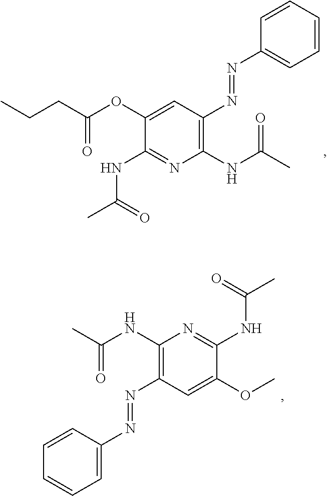 [SMILES: CCCC(=O)Oc1cc(/N=N/c2ccccc2)c(NC(C)=O)nc1NC(C)=O ; COc1cc(/N=N/c2ccccc2)c(NC(C)=O)nc1NC(C)=O]
pharmaceutically acceptable salts or solvates thereof.

11. The compound of formula III-A, pharmaceutically acceptable salts or solvates thereof according to claim 1: 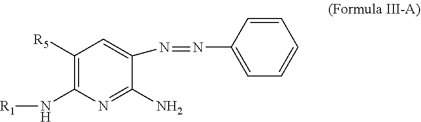
wherein;
R₁ selected from 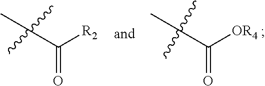
wherein R₂ is selected from the group consisting of optionally substituted alkyl, alkenyl, alkynyl, cycloalkyl, heterocycloalkyl, or heteroaryl and; R₄ is selected from the group consisting of optionally substituted alkyl, alkenyl, alkynyl, —(CH2-CH2-O)n-, cycloalkyl, heterocycloalkyl, or heteroaryl; where n is from 1-20;
R₅ selected from —OR₈, and 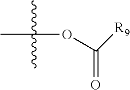
wherein R₉ is selected from the group consisting of optionally substituted alkyl, alkenyl, alkynyl, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment, —(CH2-CH2-O)n-, or N₁₀R₁₁, where n is from 1-20; wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl, alkenyl, alkynyl, cycloalkyl, heterocycloalkyl, aryl, or heteroaryl.

12. The compound according to claim 11, selected from group consisting of: 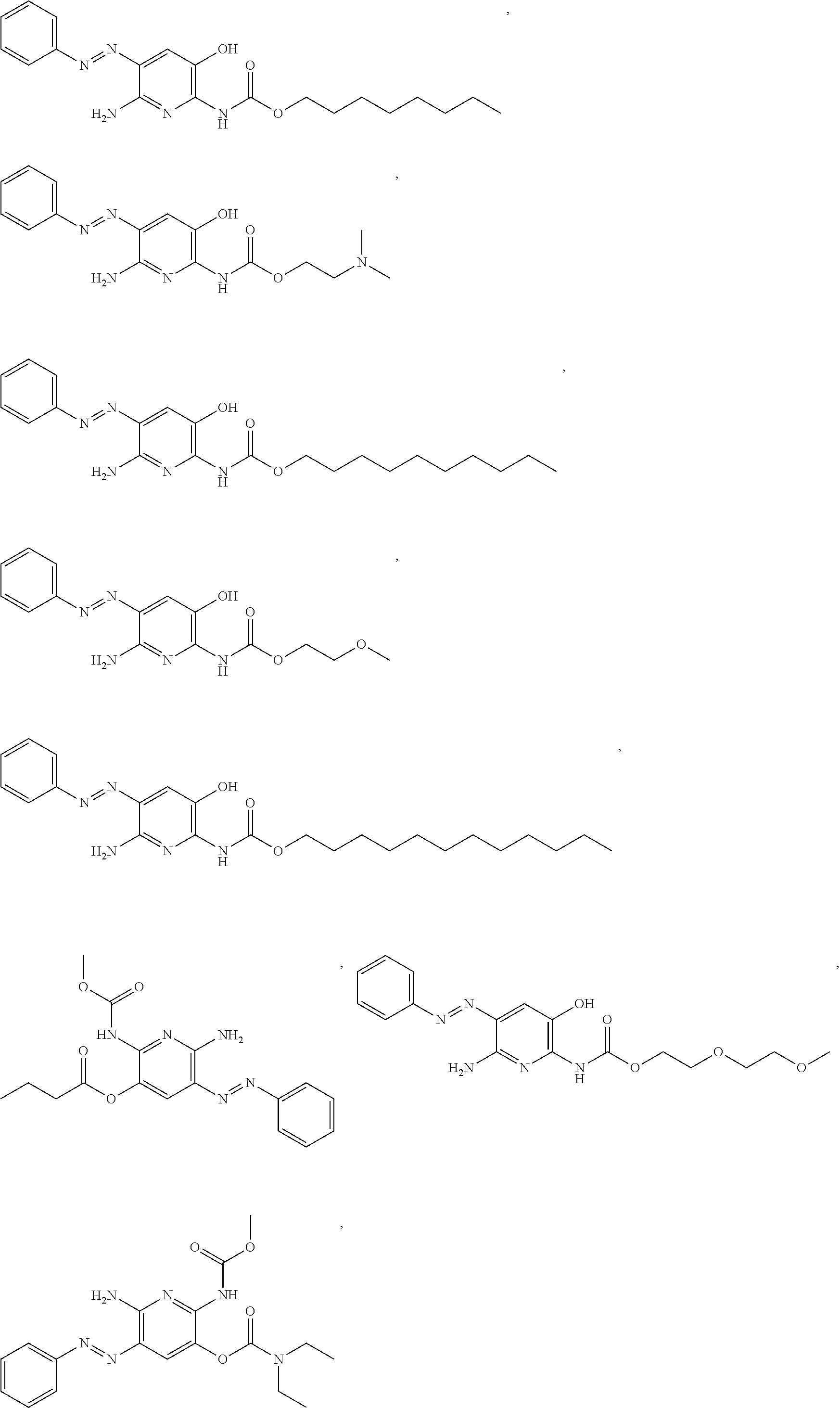 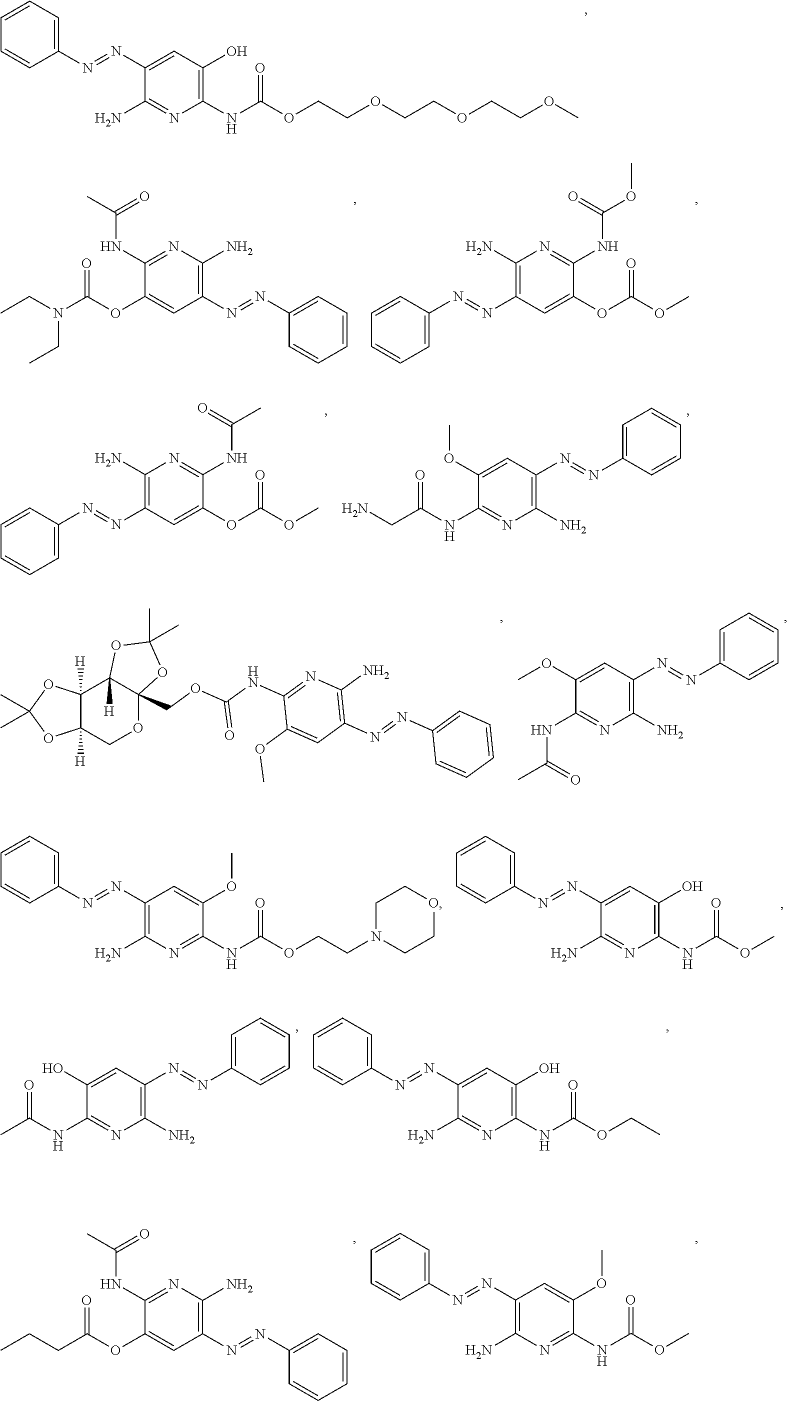 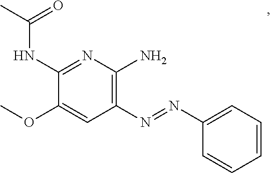 [SMILES: COc1cc(/N=N/c2ccccc2)c(N)nc1NC(C)=O]
pharmaceutically acceptable salts or solvates thereof.

13. A compound of formula III-B, pharmaceutically acceptable salts or solvates thereof according to claim 1: 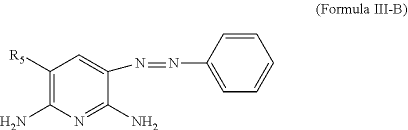
wherein; R₅ is selected from —OR₈, and 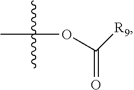
wherein R₉ is selected from the group consisting of optionally substituted alkyl, alkenyl, alkynyl, —(CH2-CH2-O)n-, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment or N₁₀R₁₁; where n is from 1-20; wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of optionally substituted alkyl, alkenyl, alkynyl, cycloalkyl, heterocycloalkyl, aryl, or heteroaryl.

14. The compound according to claim 13, selected from group consisting of: 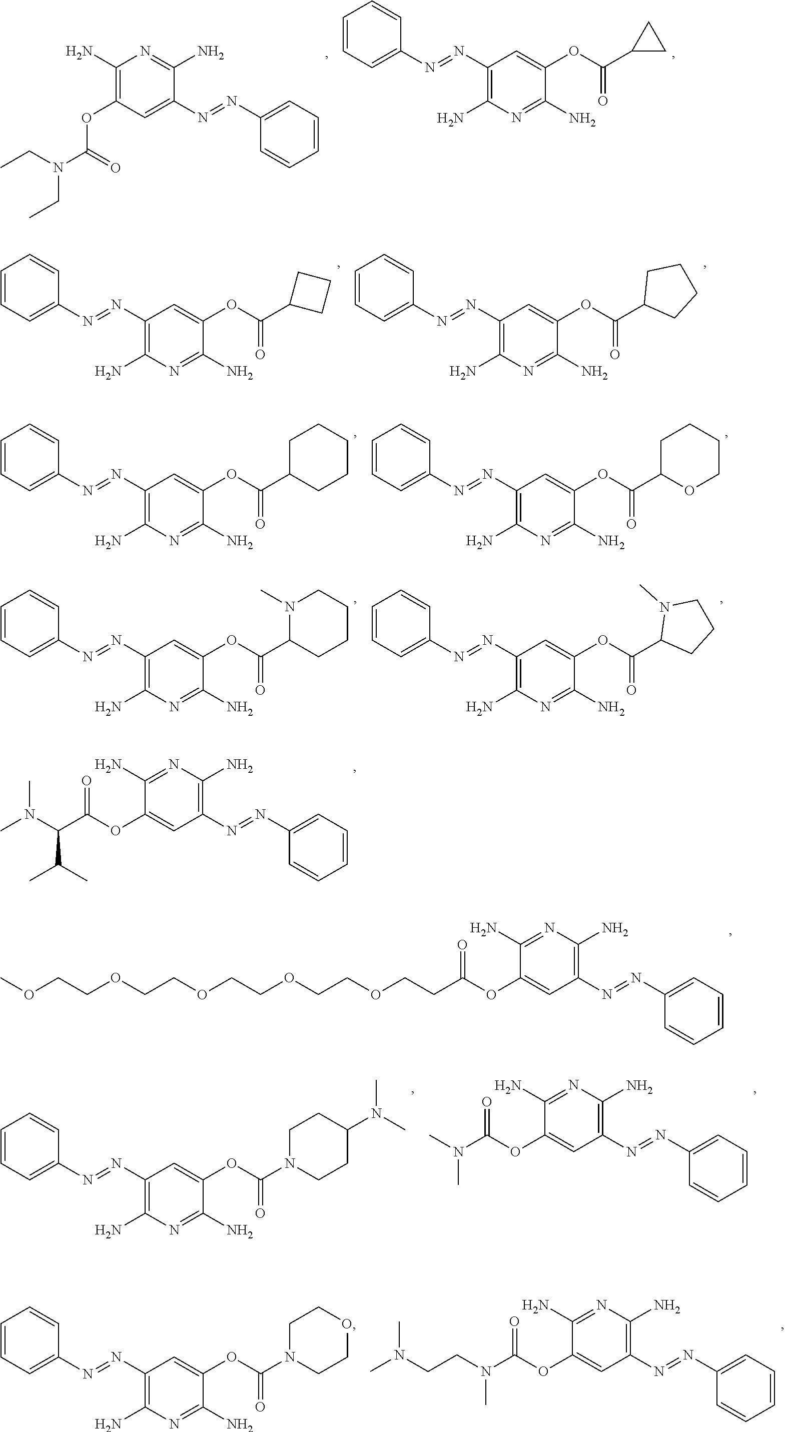 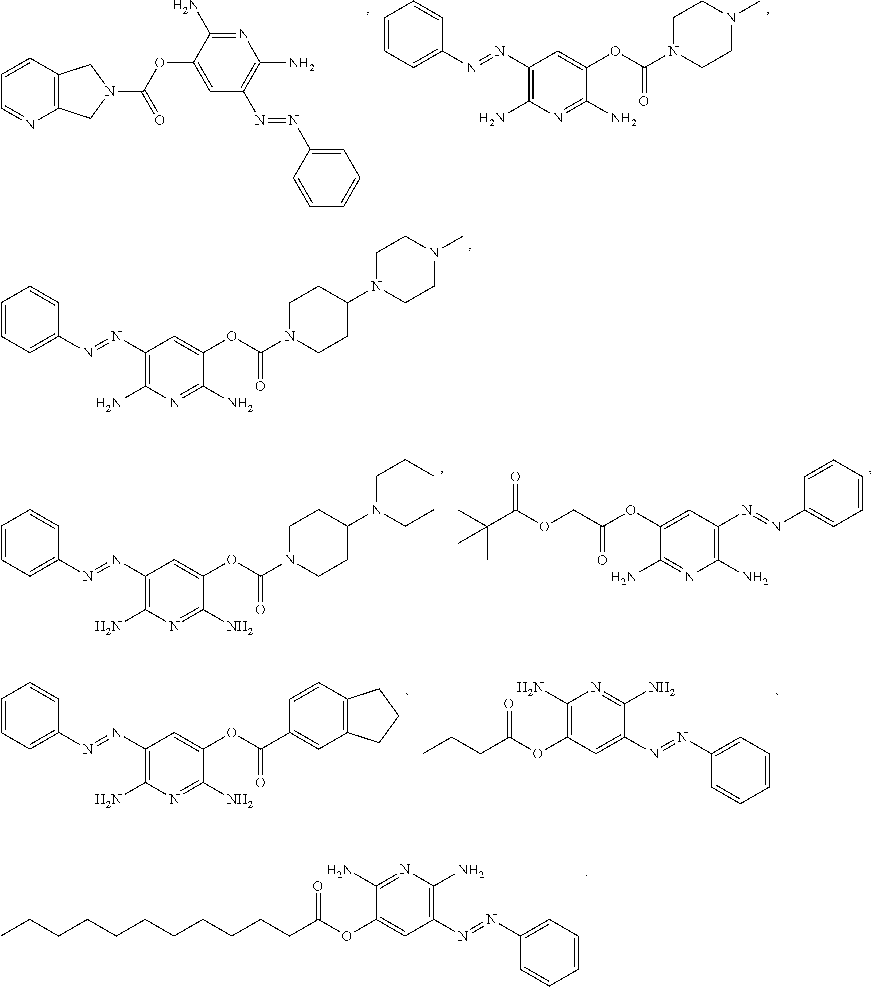
pharmaceutically acceptable salts or solvates thereof.

15. A compound of formula III-C, pharmaceutically acceptable salts or solvates thereof according to claim 1: 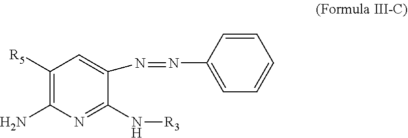
wherein;
R₃ is selected from 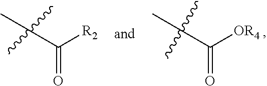
wherein R₂ is selected from the group consisting of optionally substituted alkyl, alkenyl, alkynyl, cycloalkyl, heterocycloalkyl, or heteroaryl and, R₄ is selected from the group consisting of optionally substituted alkyl, alkenyl, alkynyl, —(CH2-CH2-O)n-, cycloalkyl, heterocycloalkyl, or heteroaryl; where n is from 1-20;
R5 selected from —OR₈, and 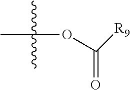
wherein R₉ is selected from the group consisting of optionally substituted alkyl, alkenyl, alkynyl, cycloalkyl, heterocycloalkyl, aryl, heteroaryl, alkoxy, heterocycloalkoxy, heteroaryloxy, cycloalkoxy, aryloxy, alkyl-ester, amino acid linked via ester or amide linkage at the point of attachment, —(CH2-CH2-O)n-, or N₁₀R₁₁; where n is from 1-20; wherein R₁₀ and R₁₁ are independently selected from hydrogen, optionally substituted alkyl; wherein alkyl group represented by R₁₀ and R₁₁ may optionally substituted with the group selected from amino, alkylamino, dialkylamino and optionally substituted heterocycloalkyl; R₈ is selected from the group consisting of hydrogen; optionally substituted alkyl, alkenyl, alkynyl, cycloalkyl, heterocycloalkyl, aryl, or heteroaryl.

16. The compound according to claim 15, selected from group consisting of: 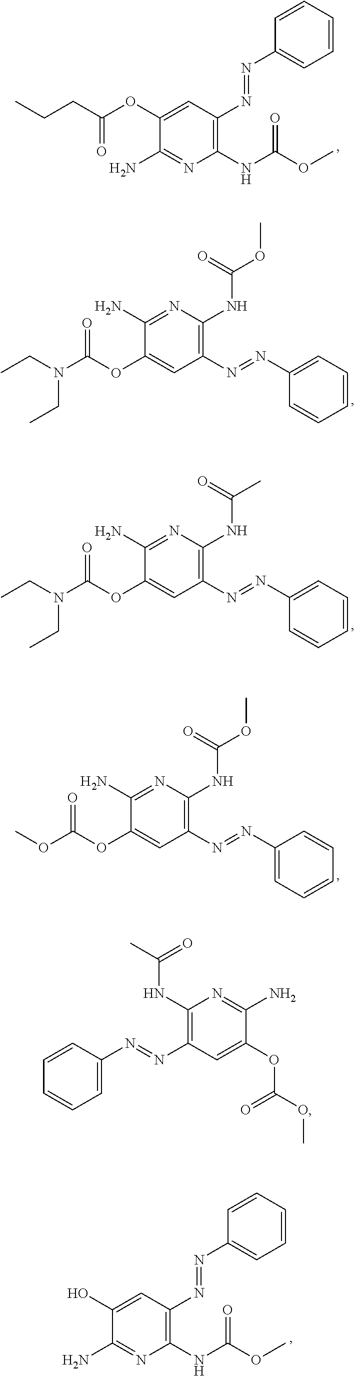 [SMILES: CCCC(=O)Oc1cc(/N=N/c2ccccc2)c(NC(=O)OC)nc1N ; CCN(CC)C(=O)Oc1cc(/N=N/c2ccccc2)c(NC(=O)OC)nc1N ; CCN(CC)C(=O)Oc1cc(/N=N/c2ccccc2)c(NC(C)=O)nc1N ; COC(=O)Nc1nc(N)c(OC(=O)OC)cc1/N=N/c1ccccc1 ; COC(=O)Oc1cc(/N=N/c2ccccc2)c(NC(C)=O)nc1N ; COC(=O)Nc1nc(N)c(O)cc1/N=N/c1ccccc1] 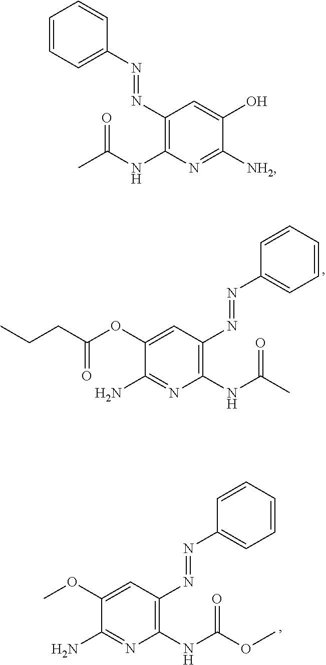 [SMILES: CC(=O)Nc1nc(N)c(O)cc1/N=N/c1ccccc1 ; CCCC(=O)Oc1cc(/N=N/c2ccccc2)c(NC(C)=O)nc1N ; COC(=O)Nc1nc(N)c(OC)cc1/N=N/c1ccccc1] 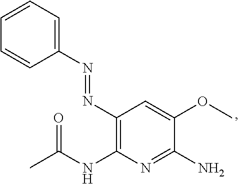 [SMILES: COc1cc(/N=N/c2ccccc2)c(NC(C)=O)nc1N]
pharmaceutically acceptable salts or solvates thereof.

17. A compound of formula IV, pharmaceutically acceptable salts or solvates thereof according to claim 1: 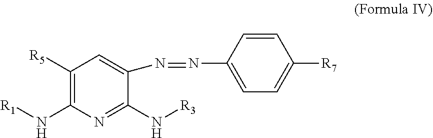
wherein;
R₁ and R₃ are independently selected from hydrogen, 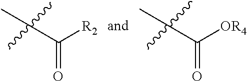
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from hydrogen, —OR₈, halogen and 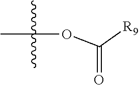
wherein, R₉ is selected from the group consisting of optionally substituted alkyl, alkenyl, alkynyl, cycloalkyl, heterocycloalkyl, aryl, heteroaryl, amino, alkoxy, heterocycloalkoxy, heteroaryloxy, cycloalkoxy, aryloxy, alkyl-ester, alkoxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R₇ is selected from hydrogen, OH, —OR₁₂, 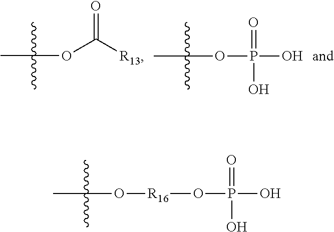
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group;
with the proviso that all substitutions at R₁, R₃, R₅ and R₇ is not hydrogen at the same time; when any one substitution at R₁ or R₃ is not hydrogen then R₅ and R₇ is not hydrogen; when substitution at R₁, R₃, R₇ is hydrogen then R₅ is not —OH; when substitution at R₁, R₃, R₅ is hydrogen then R₇ is not —OH or —O-(alkyl₁₋₃); when substitution at R₁, R₃ is hydrogen then both R₅ and R₇ is not —OH.

18. A compound of formula IV-A, pharmaceutically acceptable salts or solvates thereof according to claim 1: 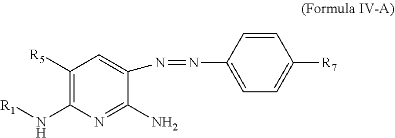
wherein;
R₁ is selected from 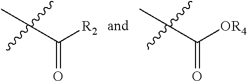
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from —OR₈, and 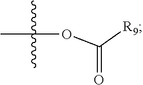
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R₇ is selected from —OH, —OR₁₂, 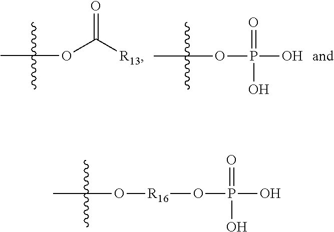
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅, where n is from 1-20; where R₁₄ and R₁₅ are each independently selected from hydrogen, optionally substituted alkyl, heterocycloalkyl, aryl group; R₁₆ is optionally substituted alkylene(C₁₋₆) group.

19. The compound according to claim 18, selected from group consisting of: 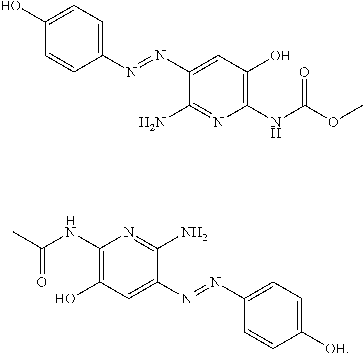
pharmaceutically acceptable salts or solvates thereof.

20. A compound of formula IV-B, pharmaceutically acceptable salts or solvates thereof according to claim 1: 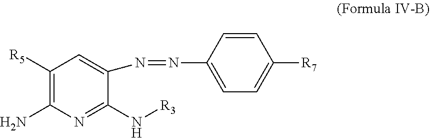
wherein;
R₃ is selected from 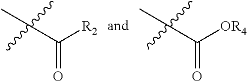
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from —OH, —OR₈, and 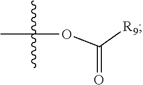
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₇ is selected from —OH, —OR₁₂, 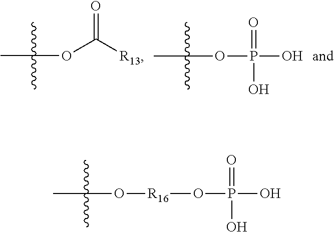
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is optionally substituted alkylene(C₁₋₆) group.

21. A compound of formula IV-C, pharmaceutically acceptable salts or solvates thereof according to claim 1: 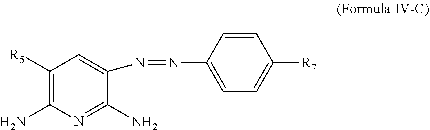
wherein;
R₅ is selected from —OH, —OR₈, and 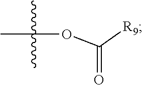
wherein R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)ₙ—, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R₇ is selected from —OH, —OR₁₂, 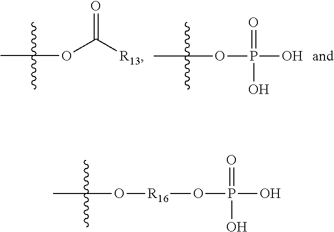
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₆; where n is from 1-20; where R₁₄ and R₁₅ are each independently selected from hydrogen, optionally substituted alkyl, heterocycloalkyl, aryl group; R₁₆ is optionally substituted alkylene(C₁₋₆) group;
with proviso that both R₅ and R₇ is not —OH.

22. The compound according to claim 21, selected from group consisting of: 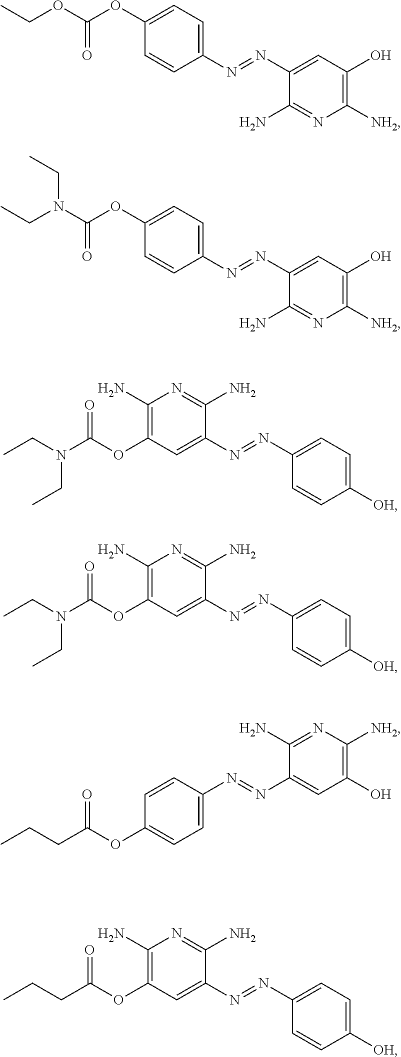 [SMILES: CCOC(=O)Oc1ccc(/N=N/c2cc(O)c(N)nc2N)cc1 ; CCN(CC)C(=O)Oc1ccc(/N=N/c2cc(O)c(N)nc2N)cc1 ; CCN(CC)C(=O)Oc1cc(/N=N/c2ccc(O)cc2)c(N)nc1N ; CCN(CC)C(=O)Oc1cc(/N=N/c2ccc(O)cc2)c(N)nc1N ; CCCC(=O)Oc1ccc(/N=N/c2cc(O)c(N)nc2N)cc1 ; CCCC(=O)Oc1cc(/N=N/c2ccc(O)cc2)c(N)nc1N]
pharmaceutically acceptable salts or solvates thereof.

23. A compound of formula V, pharmaceutically acceptable salts or solvates thereof according to claim 1: 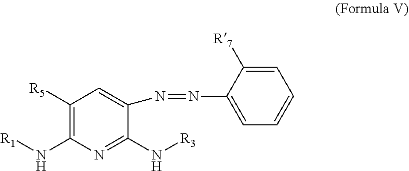
wherein;
R₁ and R₃ are the same or different and are independently selected from hydrogen, 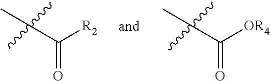
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from hydrogen, OH, —OR₈, halogen and 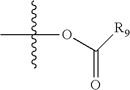
wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl;
R′₇ is selected from hydrogen, OH, —OR₁₂, 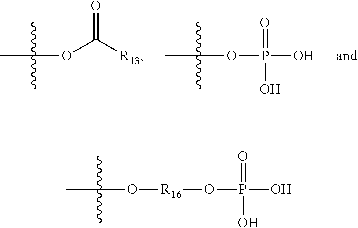
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ are each independently selected from hydrogen, optionally substituted alkyl, heterocycloalkyl, aryl group; R₁₆ is selected from optionally substituted alkylene(C₁₋₆) group;
with the proviso that all substitutions at R₁, R₃, R₅ and R′₇ is not hydrogen at the same time; when any one substitution at R₁ or R₃ is not hydrogen then R₅ and R′₇ is not hydrogen; when substitution at R₁, R₃, R′₇ is hydrogen then R₅ is not —OH; when substitution at R₁, R₃, R₅ is hydrogen then R′₇ is not —OH or —O-(alkyl₁₋₃).

24. A compound of formula V-A, pharmaceutically acceptable salts or solvates thereof according to claim 1: 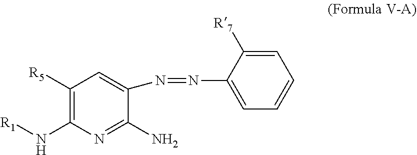
wherein;
R₁ is selected from 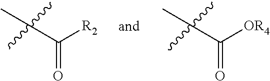
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from —OR₈ and 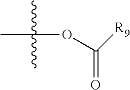
wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl;
R′₇ is selected from —OH, —OR₁₂, 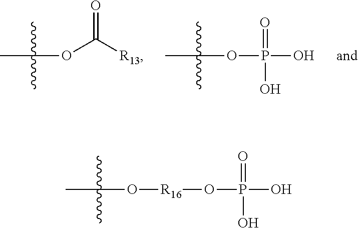
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group; R₁₆ is selected from optionally substituted alkylene(C₁₋₆) group.

25. A compound of formula V-B, pharmaceutically acceptable salts or solvates thereof according to claim 1: 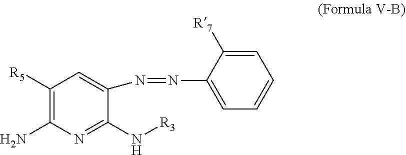
wherein;
R₃ is selected from 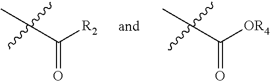
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₅ is selected from —OR₈ and 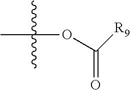
wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁; where n is from 1-20; wherein R₁₀ and R₁₁ are the same or different and are independently selected from hydrogen or optionally substituted alkyl; R₈ is selected from the group consisting of hydrogen, optionally substituted alkyl, alkenyl, alkynyl, cycloalkyl, heterocycloalkyl, aryl, or heteroaryl; R′₇ is selected from —OH, —OR₁₂, 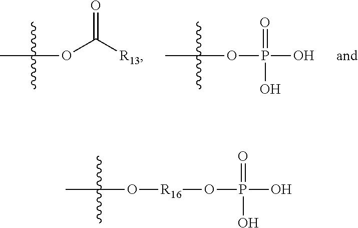
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment; —(CH2-CH2-O)n-, or NR₁₄R₁₅; where n is from 1-20; where R₁₄ and R₁₅ are each independently selected from hydrogen, optionally substituted alkyl, heterocycloalkyl, aryl group; R₁₆ is selected from optionally substituted alkylene(C₁₋₆) group.

26. A compound of formula V-C, pharmaceutically acceptable salts or solvates thereof according to claim 1: 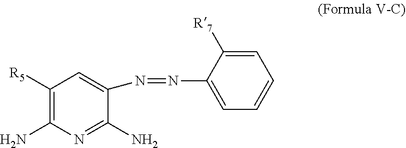
wherein;
R₅ is selected from —OH, —OR₈ and 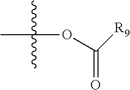
wherein, R₉ is selected from the group consisting of optionally substituted alkyl, alkenyl, alkynyl, cycloalkyl, heterocycloalkyl, aryl, heteroaryl, amino, alkyl-ester, alkoxy, heterocycloalkoxy, heteroaryloxy, cycloalkoxy, aryloxy, amino acid linked via ester or amide linkage at the point of attachment, —(CH2-CH2-O)n-, or NR₁₀R₁₁, wherein R₁₀ and R₁₁ are the same or different and are independently selected from hydrogen or optionally substituted alkyl; R₈ is selected from the group consisting of optionally substituted alkyl, alkenyl, alkynyl, cycloalkyl, heterocycloalkyl, aryl, or heteroaryl; where n is from 1-20;
R′₇ is selected from —OH, —OR₁₂, 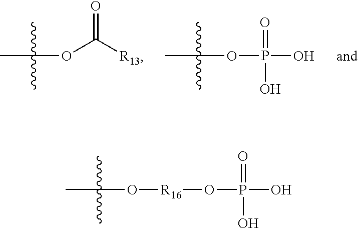
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)ₙ—, or NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C₁₋₆) group.

27. A compound of formula VI, pharmaceutically acceptable salts or solvates thereof according to claim 1: 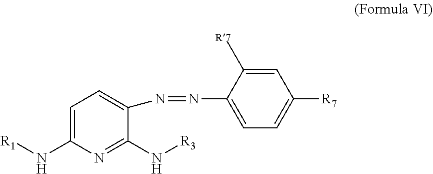
wherein;
R₁ and R₃ are the same or different and are independently selected from hydrogen, 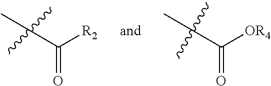
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ and R′₇ is selected from hydrogen, OH, —OR₁₂, 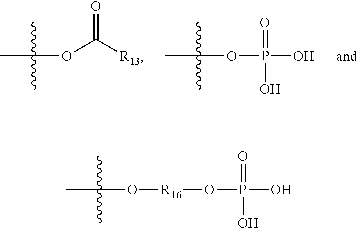
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or R₁₃ selected from by NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C1-6) group;
with the proviso that all substitutions at R₁, R₃, R₇ and R′₇ is not hydrogen at the same time; when any one substitution at R₁ or R₃ is not hydrogen then R₇ and R′₇ is not hydrogen; when substitution at R₁, R₃, R₇ is hydrogen then R′₇ is not —OH or —O-(alkyl₁₋₃); when substitution at R₁, R₃, R′₇ is hydrogen then R₇ is not —OH or —O-(alkyl₁₋₃).

28. A compound of formula VI-A, pharmaceutically acceptable salts or solvates thereof according to claim 1: 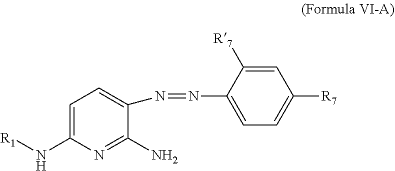
wherein;
R₁ is selected from 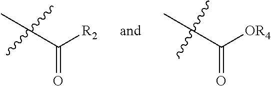
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)ₙ—, cycloalkyl, heterocycloalkyl, aryl, or heteroaryl; where n is from 1-20;
R₇ and R′₇ is selected independently at each point of substitution from —OH, —OR₁₂, 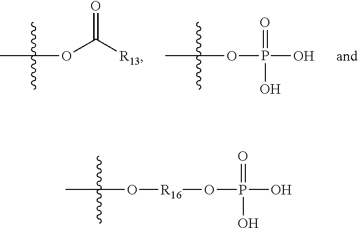
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group.

29. A compound of formula VI-B, pharmaceutically acceptable salts or solvates thereof according to claim 1: 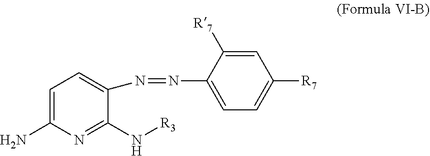
wherein;
R₃ is selected from 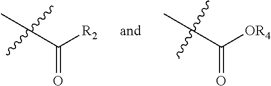
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ and R′₇ is selected independently at each point of substitution from —OH, —OR₁₂, 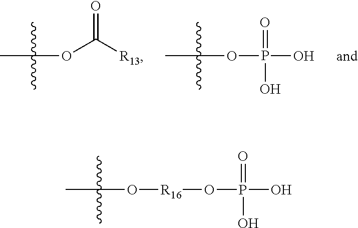
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group; R₁₆ is selected from optionally substituted alkylene(C₁₋₆) group.

30. A compound of formula VI-C, pharmaceutically acceptable salts or solvates thereof
according to claim 1: 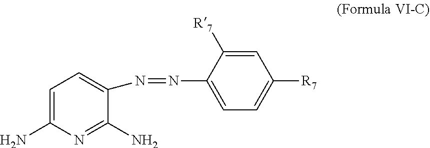
wherein;
R₇ and R′₇ are selected independently at each point of substitution from —OH, —OR₁₂, 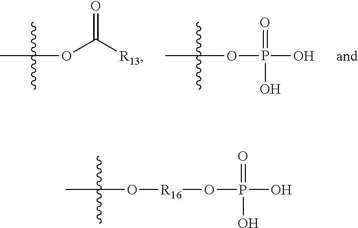
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene_{(C1-6)} group.

31. A compound of formula VII, pharmaceutically acceptable salts or solvates thereof according to claim 1: 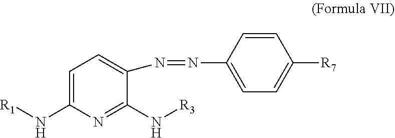
wherein;
R₁ and R₃ are independently selected from hydrogen, 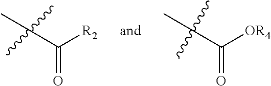
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ is selected from hydrogen, OH, —OR₁₂, 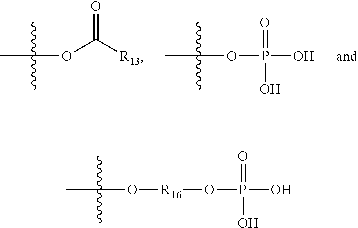
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group;
with the proviso that all substitutions at R₁, R₃ and R₇ is not hydrogen at the same time; when any one substitution at R₁ or R₃ is not hydrogen then R₇ is not hydrogen; when substitution at R₁, R₃ is hydrogen then R₇ is not —OH, —O-(alkyl₁₋₃).

32. A compound of formula VII-A, pharmaceutically acceptable salts or solvates thereof according to claim 1: 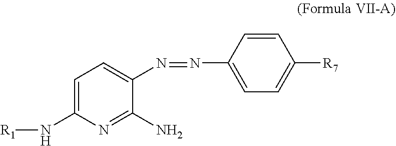
wherein;
R₁ is selected from 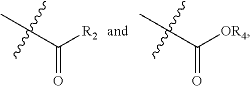
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ is selected from —OH, —OR₁₂, 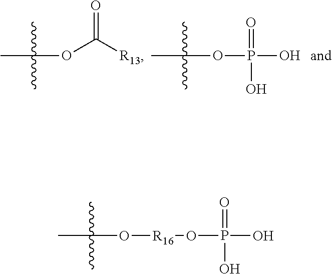
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group. R₁₆ is selected from optionally substituted alkylene(C₁₋₆) group.

33. The compound according to claim 32, selected from group consisting of: 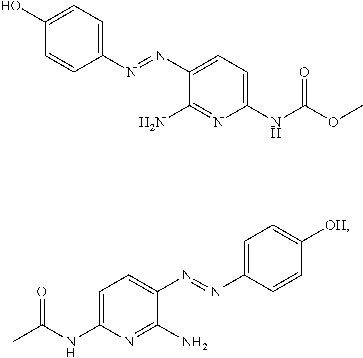
pharmaceutically acceptable salts or solvates thereof.

34. A compound of formula VII-B, pharmaceutically acceptable salts or solvates thereof according to claim 1: 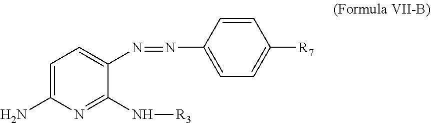
wherein;
R₃ is selected from 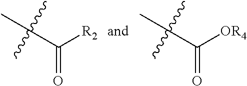
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R₇ is selected from —OH, —OR₁₂, 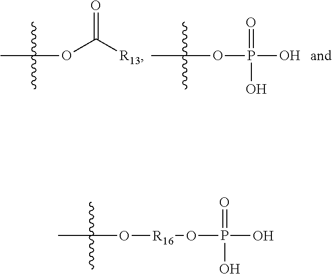
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group;

35. A compound of formula VII-C, pharmaceutically acceptable salts or solvates thereof according to claim 1: 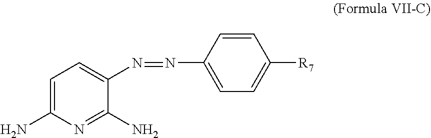
Wherein R₇ is selected from —OR₁₂, 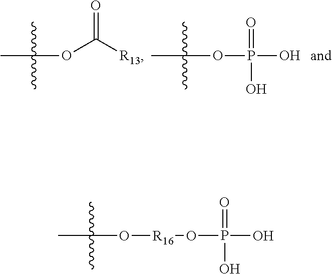
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group, R₁₆ is selected from optionally substituted alkylene(C₁₋₆) group.

36. The compound according to claim 35, selected from group consisting of: 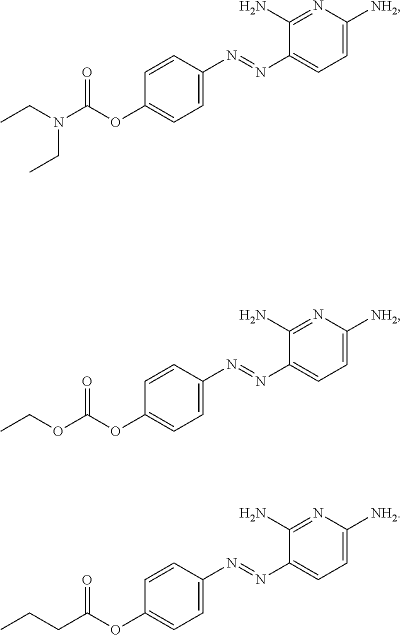 [SMILES: CCN(CC)C(=O)Oc1ccc(/N=N/c2ccc(N)nc2N)cc1 ; CCOC(=O)Oc1ccc(/N=N/c2ccc(N)nc2N)cc1 ; CCCC(=O)Oc1ccc(/N=N/c2ccc(N)nc2N)cc1]
pharmaceutically acceptable salts or solvates thereof.

37. A compound of formula VIII, pharmaceutically acceptable salts or solvates thereof according to claim 1: 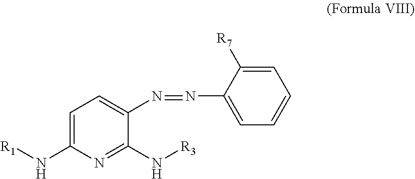
wherein;
R₁ and R₃ are independently selected from hydrogen, 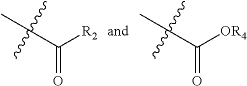
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R′₇ is selected from hydrogen, OH, —OR₁₂, 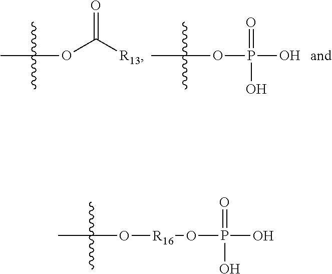
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene_{(C1-6)} group;
with the proviso that all substitutions at R₁, R₃ and R′₇ is not hydrogen at the same time; when any one substitution at R₁ or R₃ is not hydrogen then R′₇ is not hydrogen; when substitution at R₁, R₃ is hydrogen then R′₇ is not —OH or —O-(alkyl₁₋₃).

38. The compound according to claim 37, selected from group consisting of: 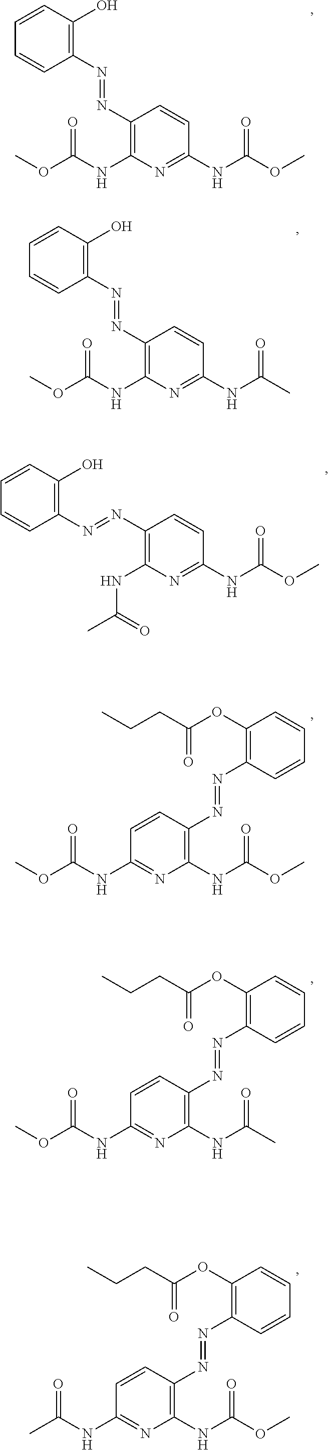 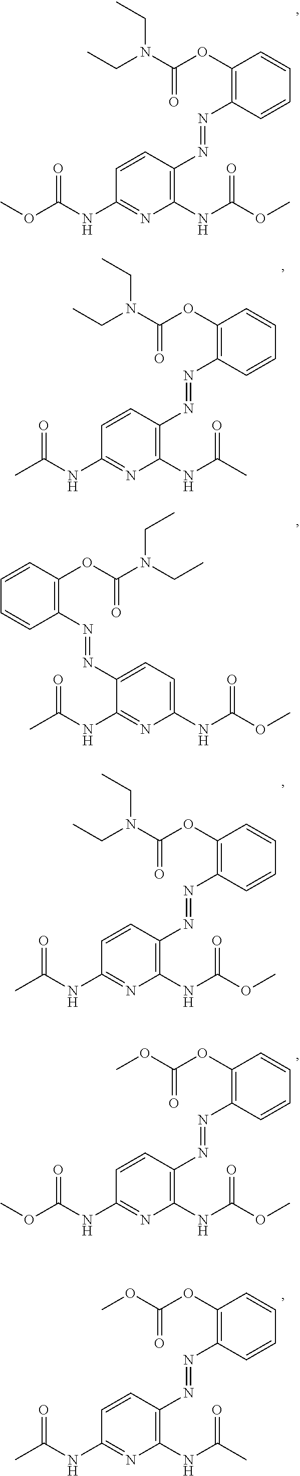 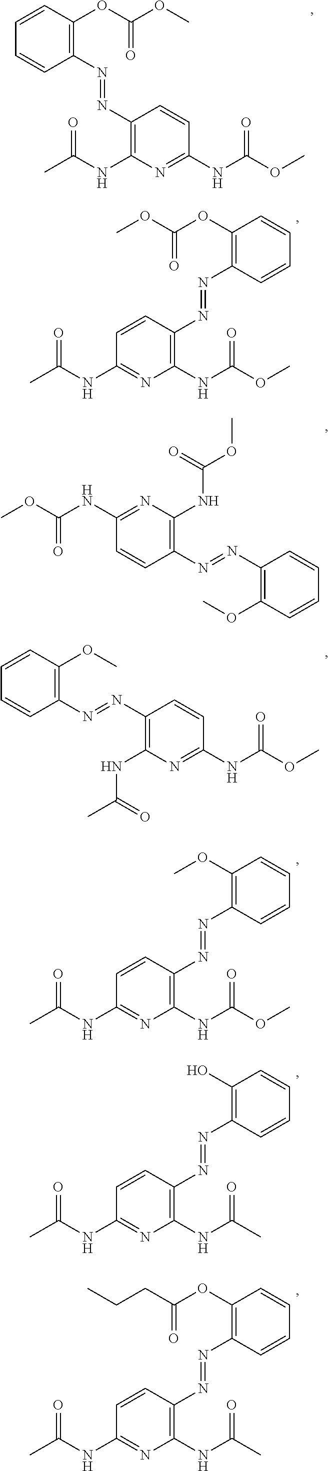 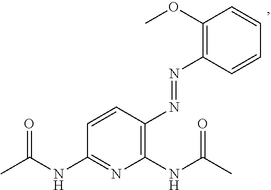 [SMILES: COc1ccccc1/N=N/c1ccc(NC(C)=O)nc1NC(C)=O]
pharmaceutically acceptable salts or solvates thereof.

39. A compound of formula VIII-A, pharmaceutically acceptable salts or solvates thereof according to claim 1:
wherein; 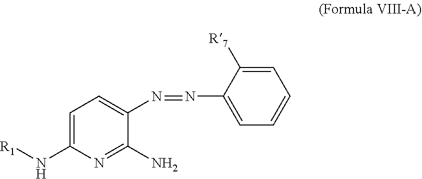
R₁ is selected from 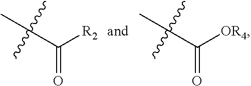
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20;
R′₇ is selected from —OH, —OR₁₂, 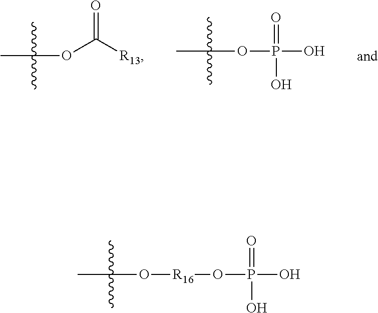
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group. R₁₆ is selected from optionally substituted alkylene(C₁₋₆) group.

40. The compound according to claim 39, selected from group consisting of: 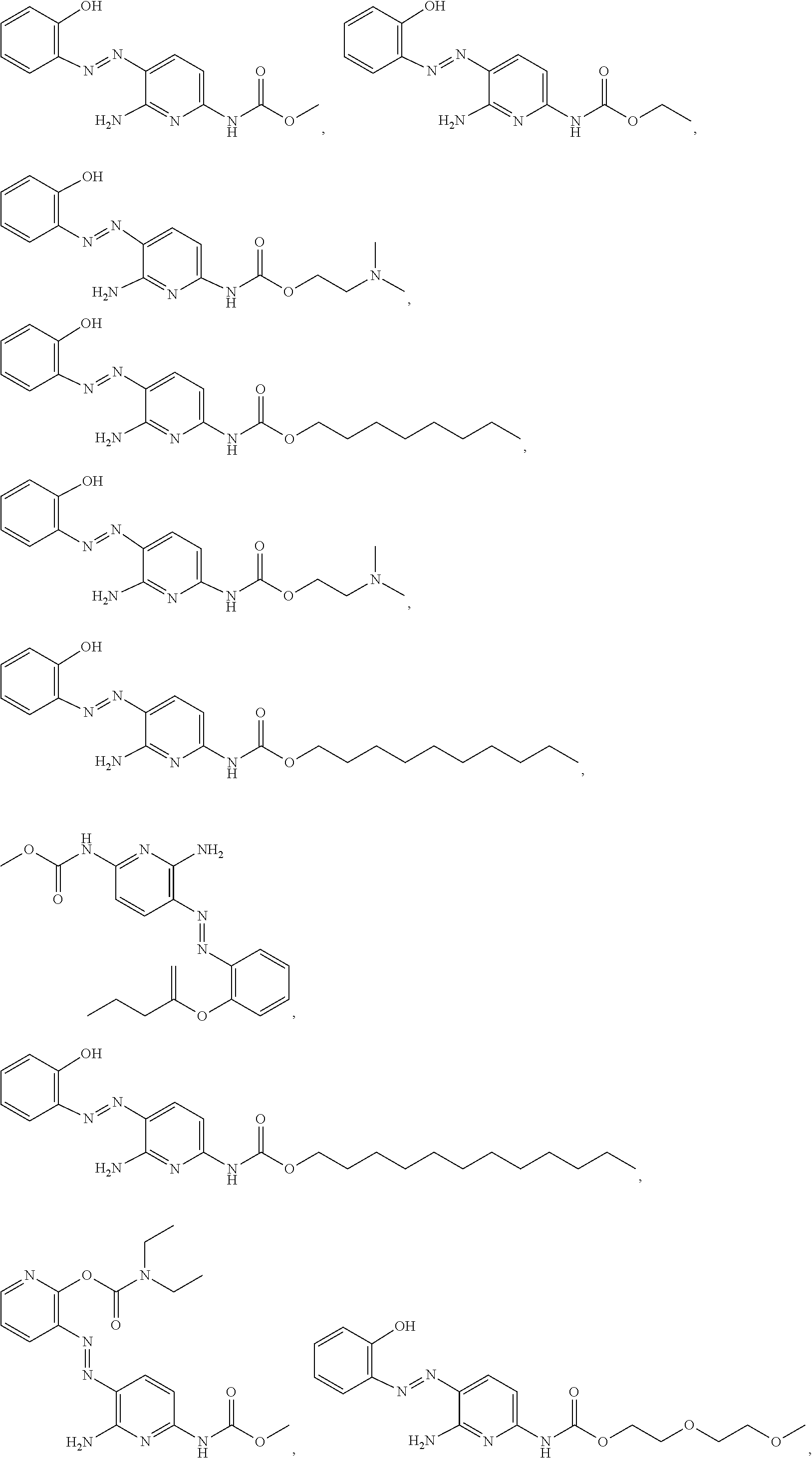 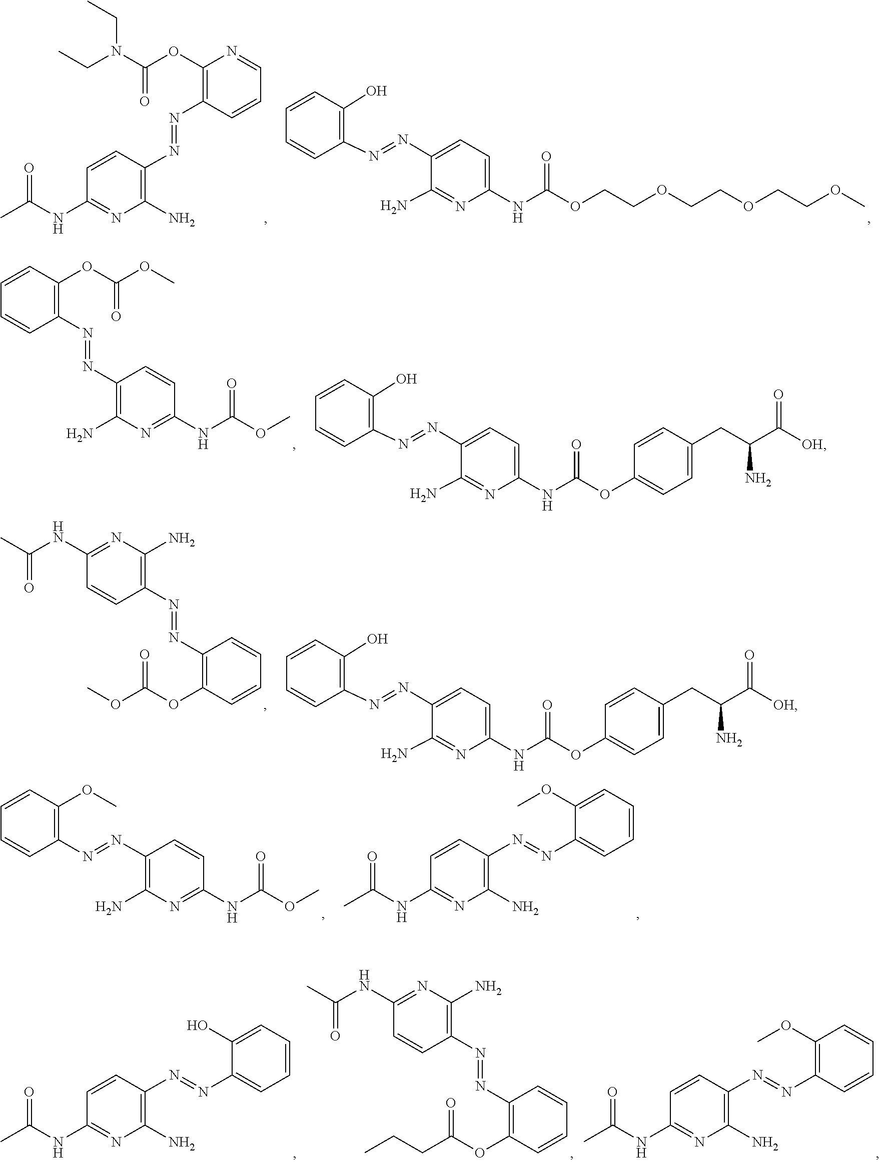
pharmaceutically acceptable salts or solvates thereof.

41. A compound of formula VIII-B, pharmaceutically acceptable salts or solvates thereof according to claim 1: 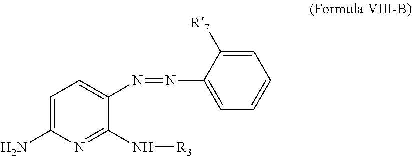
wherein;
R₃ is selected from 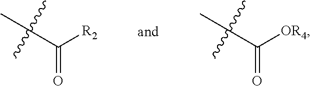
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl, where n is from 1-20;
R′₇ is selected from —OH, —OR₁₂, 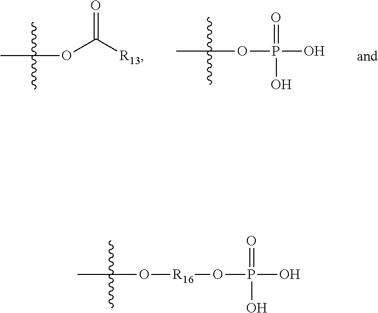
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ are each independently selected from hydrogen, optionally substituted alkyl, heterocycloalkyl, aryl group. R₁₆ is selected from optionally substituted alkylene(C₁₋₆) group.

42. The compound according to claim 41, selected from group consisting of: 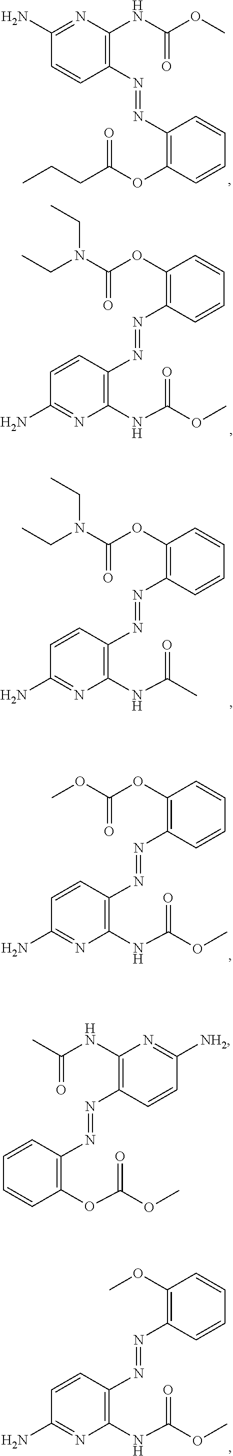 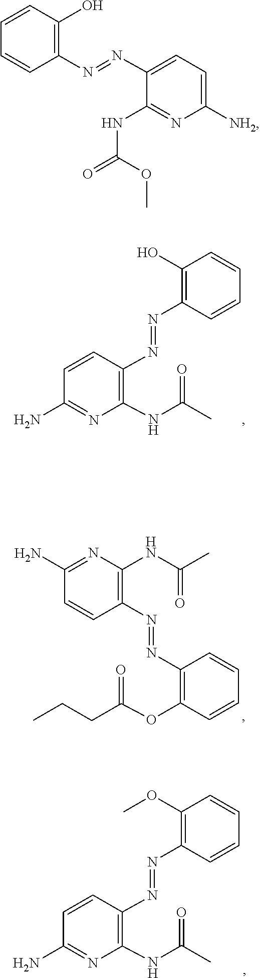
pharmaceutically acceptable salts or solvates thereof.

43. A compound of formula VIII-C, pharmaceutically acceptable salts or solvates thereof according to claim 1: 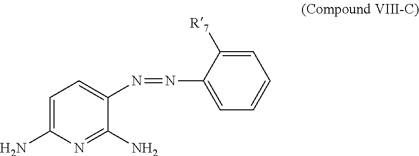
wherein R′₇ is selected from —OR₁₂, 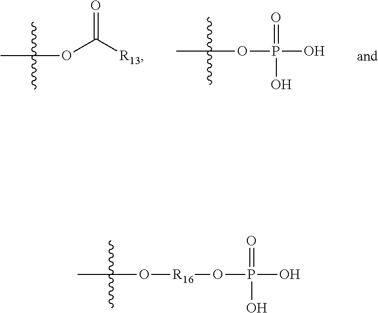
wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; where R₁₄ and R₁₅ each independently selected from hydrogen, optionally substituted alkyl, heterocycloalkyl, aryl group. R₁₆ is selected from optionally substituted alkylene(C₁₋₆) group; with proviso that when substitution at R₁, R₃ is hydrogen then R′₇ is not —OH or —O-(alkyl₁₋₃).

44. The compound according to claim 43, selected from group consisting of: 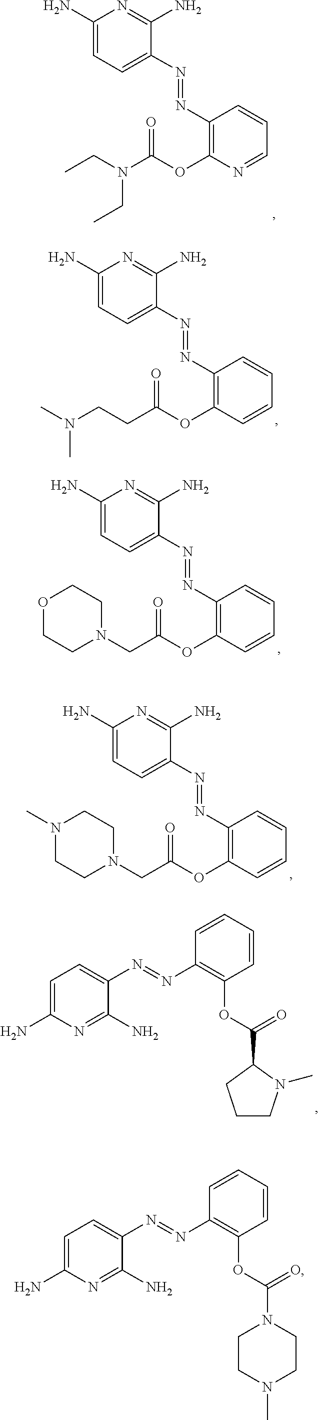 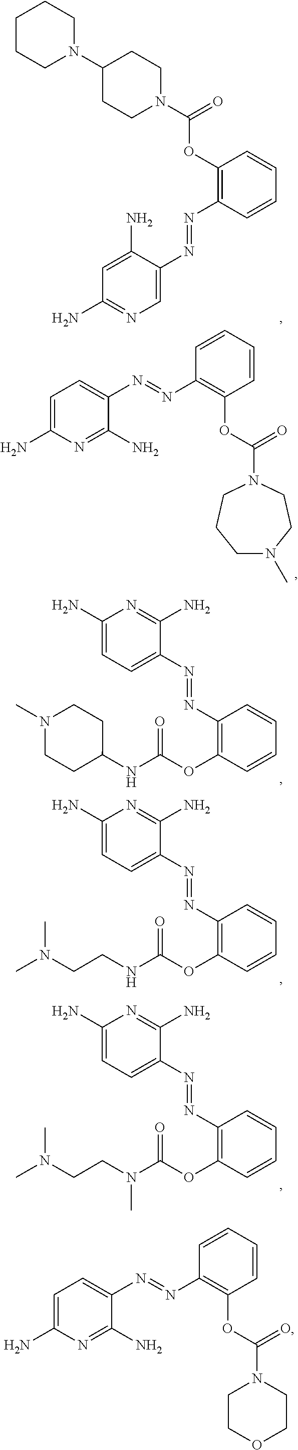 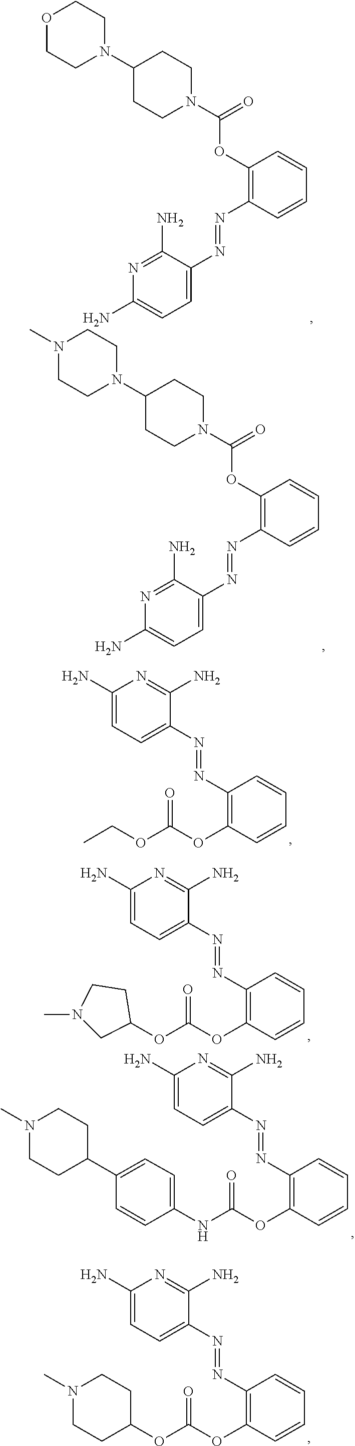 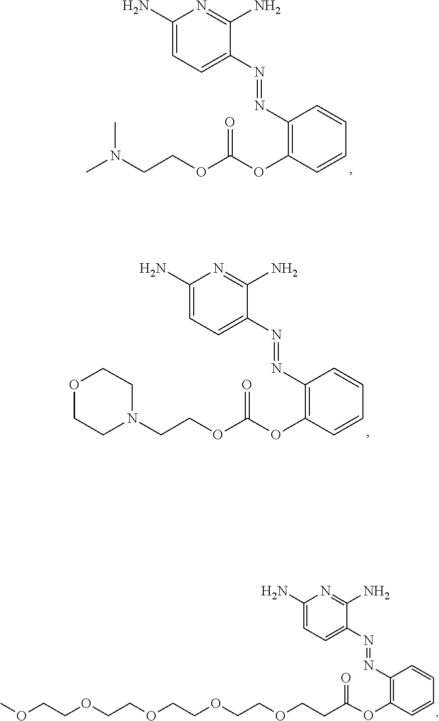 [SMILES: CN(C)CCOC(=O)Oc1ccccc1/N=N/c1ccc(N)nc1N ; Nc1ccc(/N=N/c2ccccc2OC(=O)OCCN2CCOCC2)c(N)n1 ; COCCOCCOCCOCCOCCC(=O)Oc1ccccc1/N=N/c1ccc(N)nc1N]
pharmaceutically acceptable salts or solvates thereof.

45. A compound of formula IX, pharmaceutically acceptable salts or solvates thereof according to claim 2: 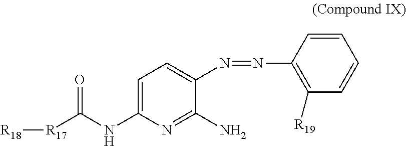
wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₁₉ is selected from —O—R₂₃—O—C(O)—R₂₄, —O—C(O)—R₂₇, and —O—R₂₃—R₂₈, wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group; R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group.

46. A compound of formula IX-A, pharmaceutically acceptable salts or solvates thereof according to claim 2: 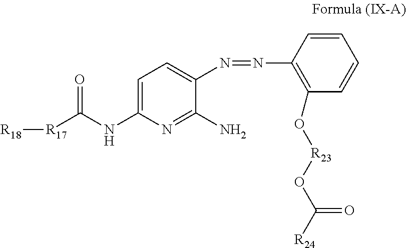
wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
Wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group.

47. The compound according to claim 46, selected from group consisting of: 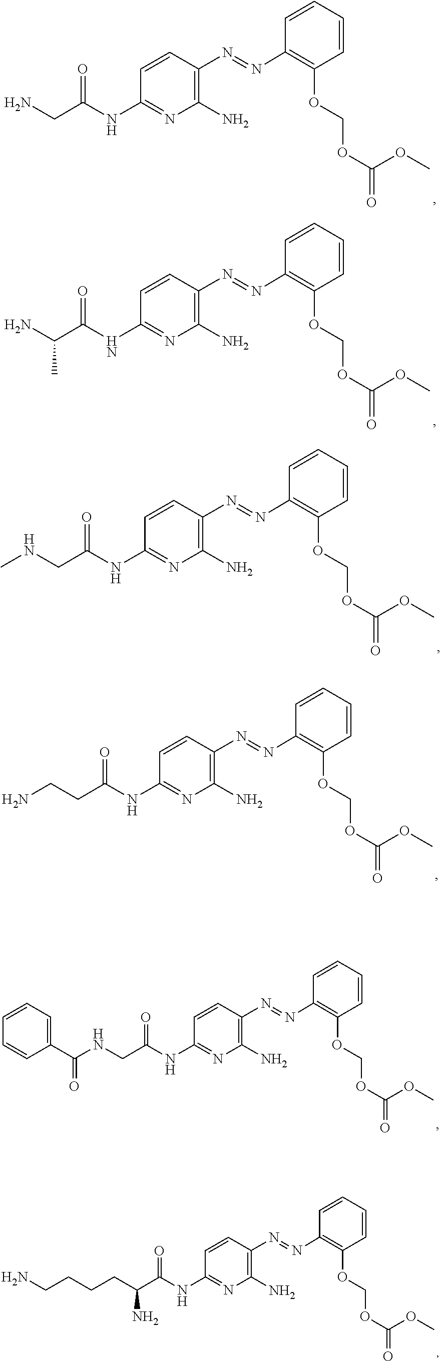 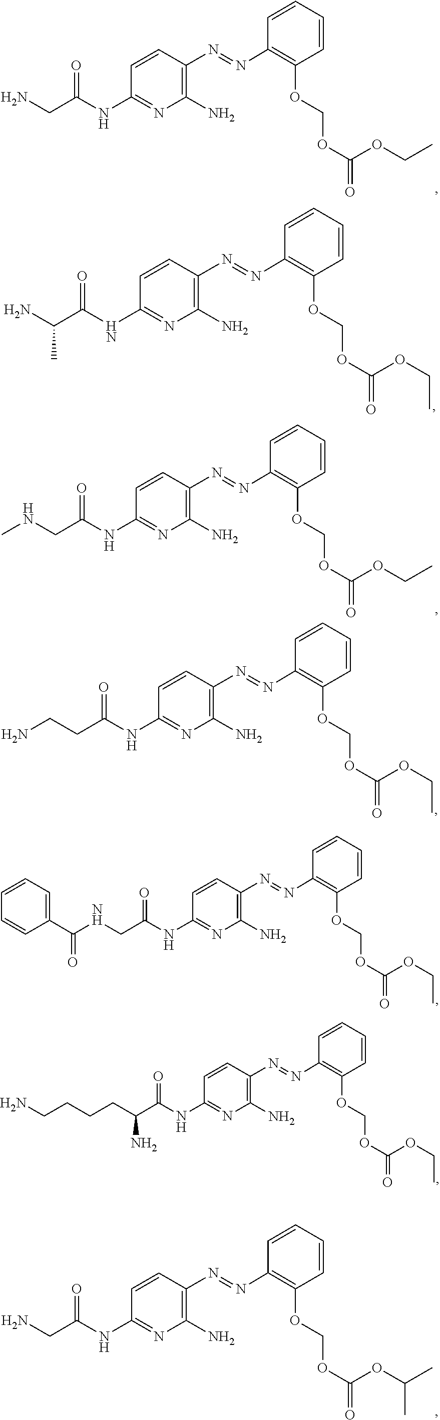 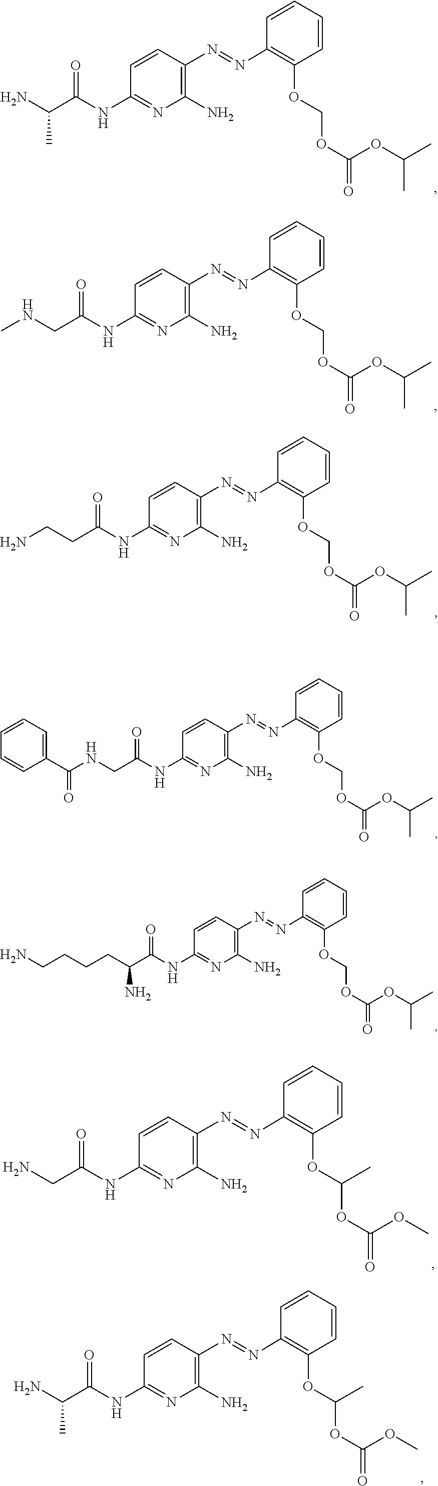 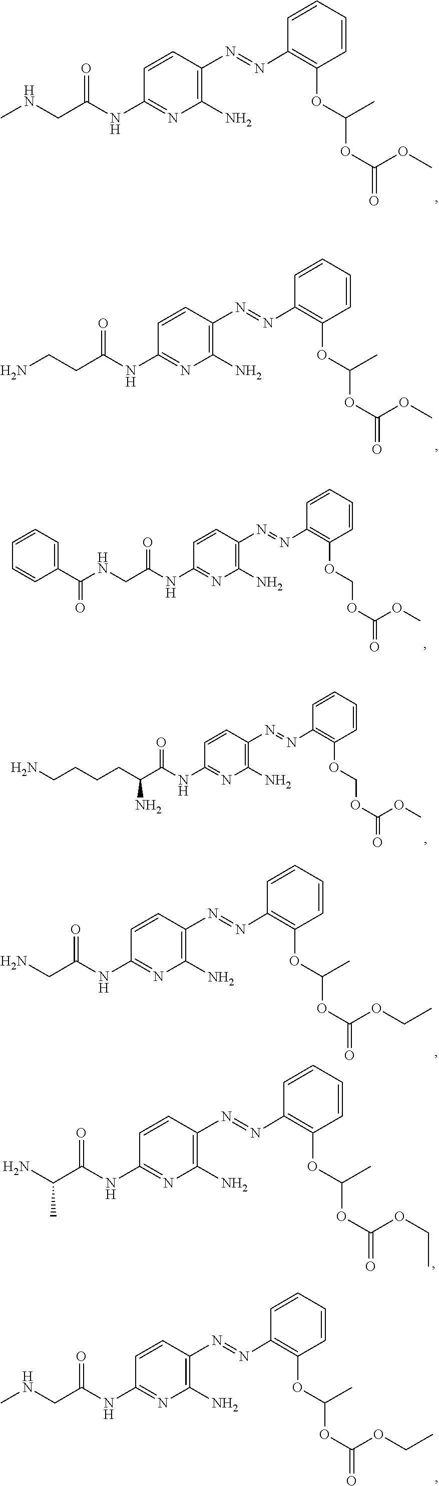 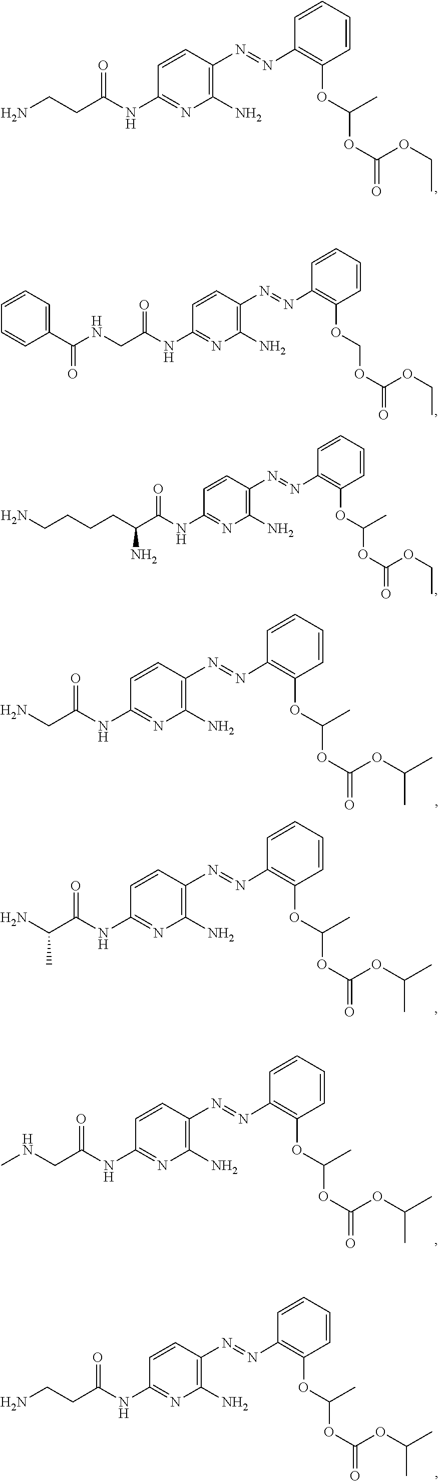 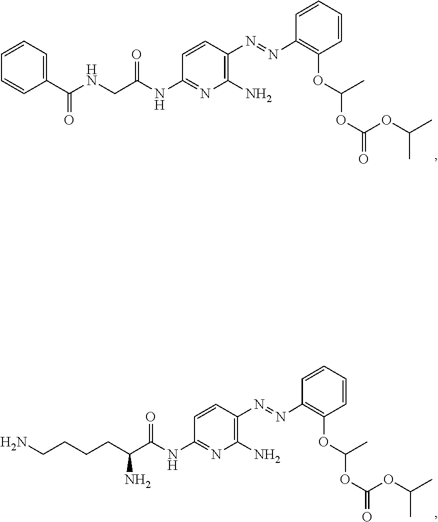 [SMILES: CC(C)OC(=O)OC(C)Oc1ccccc1/N=N/c1ccc(NC(=O)CNC(=O)c2ccccc2)nc1N ; CC(C)OC(=O)OC(C)Oc1ccccc1/N=N/c1ccc(NC(=O)[C@@H](N)CCCCN)nc1N]
pharmaceutically acceptable salts or solvates thereof.

48. A compound of formula IX-B, pharmaceutically acceptable salts or solvates thereof according to claim 2: 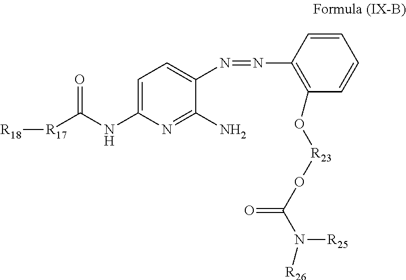
wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group; wherein R₂₃ is optionally substituted linear or branched alkylene; wherein R₂₅ and R₂₆ are each independently selected from hydrogen, optionally substituted alkyl and aryl group, wherein alkyl and aryl is optionally substituted with OH, SH, F, Cl, Br, I, and optionally substituted, hydroxyalkyl, amino group; or R₂₅ and R₂₆ is taken together form an optionally substituted heterocycloalkyl ring, wherein the heterocycloalkyl ring is optionally substituted by optionally substituted alkyl, hydroxyalkyl group, OH, SH, F, Cl, Br, I, and optionally substituted amino group.

49. The compound according to claim 48, selected from group consisting of: 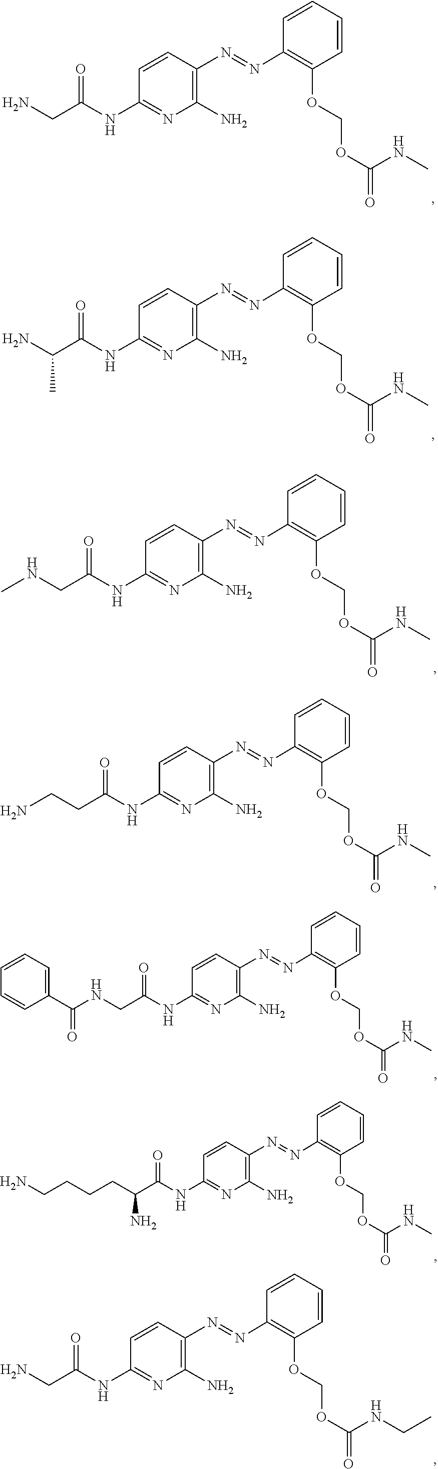 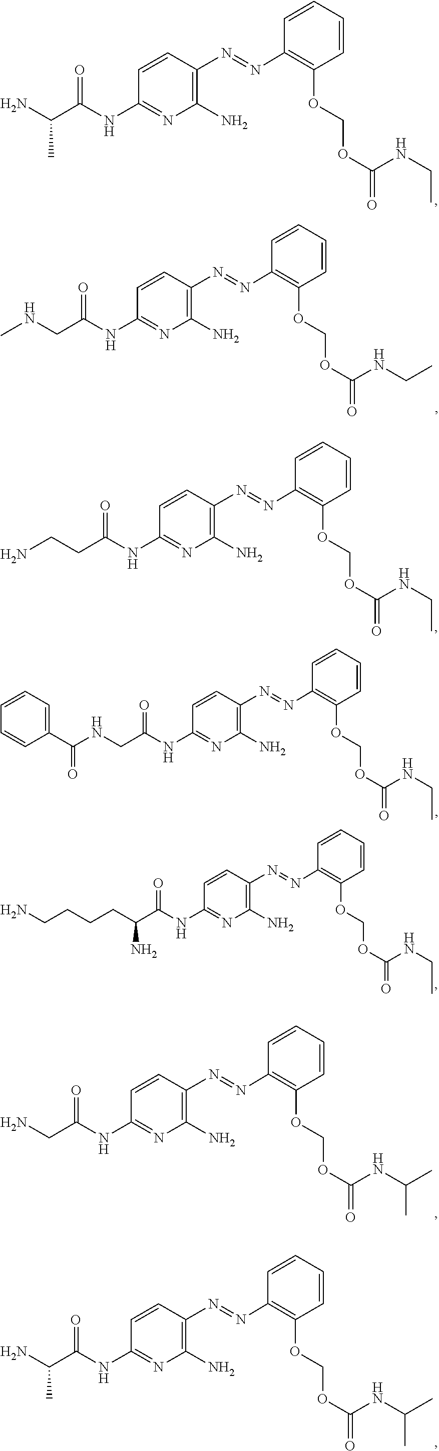 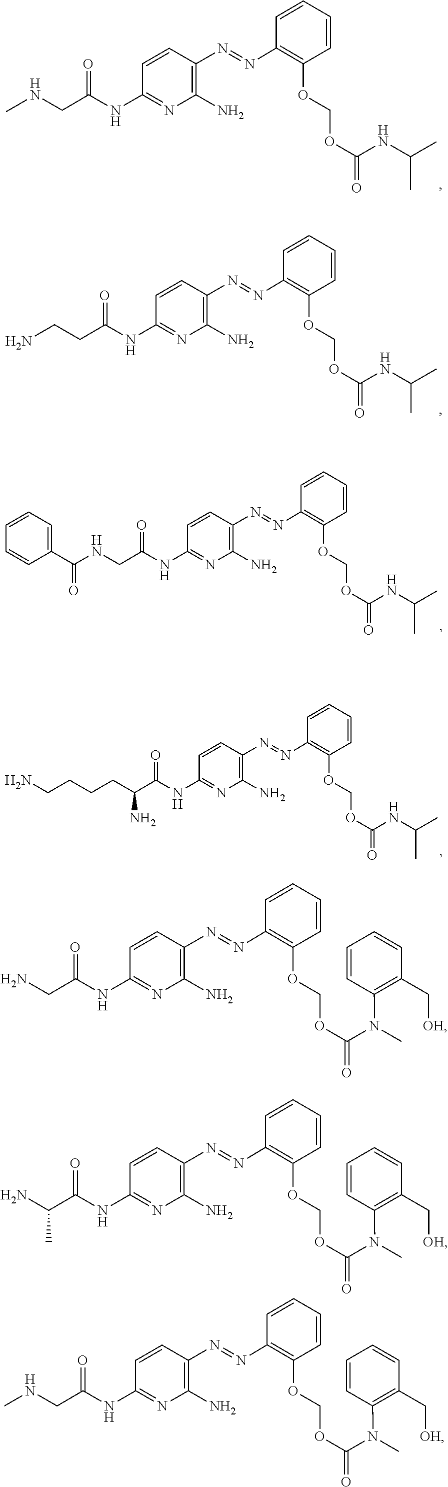 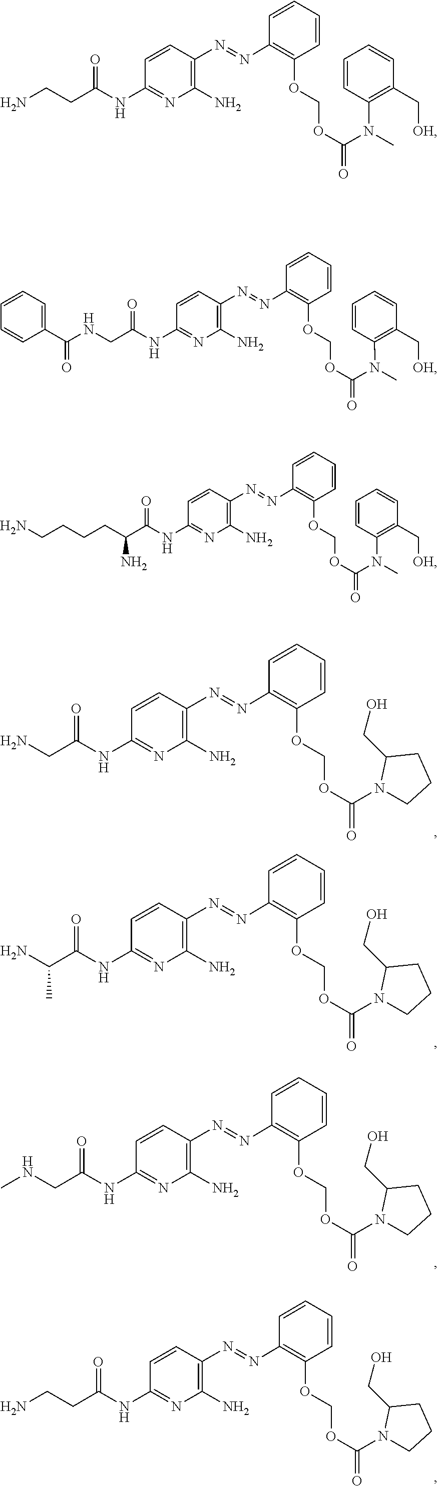 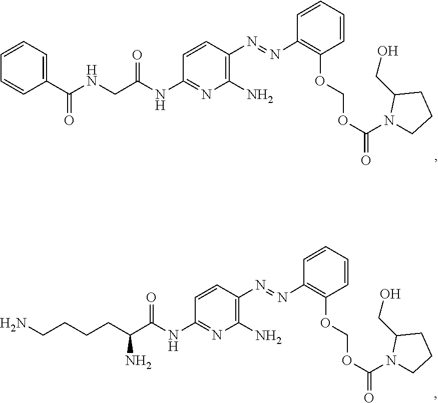 [SMILES: Nc1nc(NC(=O)CNC(=O)c2ccccc2)ccc1/N=N/c1ccccc1OCOC(=O)N1CCCC1CO ; NCCCC[C@H](N)C(=O)Nc1ccc(/N=N/c2ccccc2OCOC(=O)N2CCCC2CO)c(N)n1]
pharmaceutically acceptable salts or solvates thereof.

50. A compound of formula IX-C, pharmaceutically acceptable salts or solvates thereof according to claim 2: 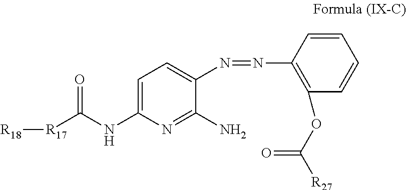
wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group;
R₂₇ is selected from optionally substituted alkyl, cycloalkyl, heterocycloalkyl, heteroaryl, aryl and —N(R₂₅R₂₆) group; wherein R₂₅ and R₂₆ are each independently selected from hydrogen, optionally substituted alkyl and aryl group, wherein alkyl and aryl is optionally substituted with OH, SH, F, Cl, Br, I, and optionally substituted, hydroxyalkyl, amino group; or R₂₅ and R₂₆ is taken together form an optionally substituted heterocycloalkyl ring, wherein the heterocycloalkyl ring is optionally substituted by optionally substituted alkyl, hydroxyalkyl group, OH, SH, F, Cl, Br, I, and optionally substituted amino group.

51. The compound according to claim 50, selected from group consisting of: 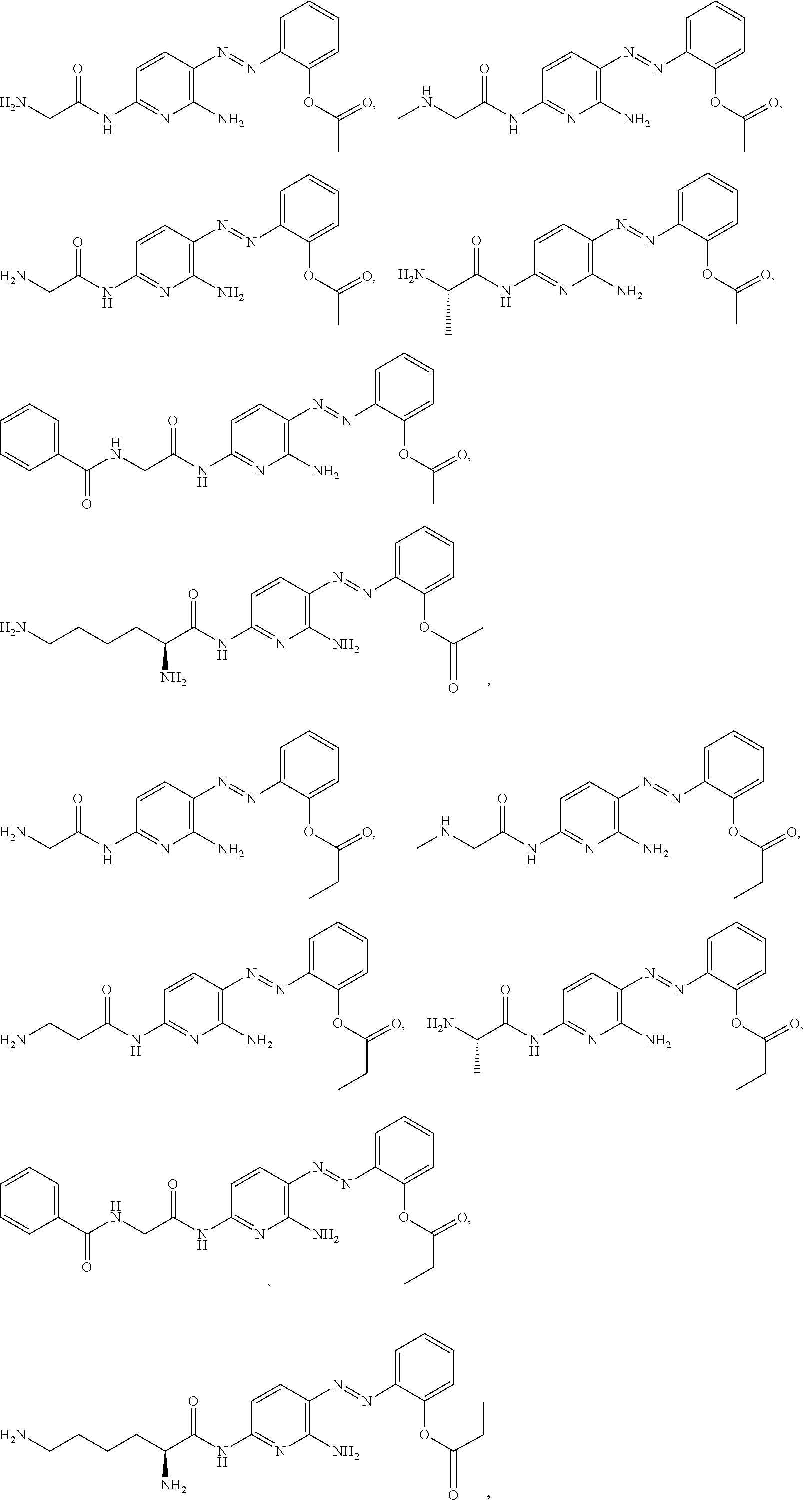 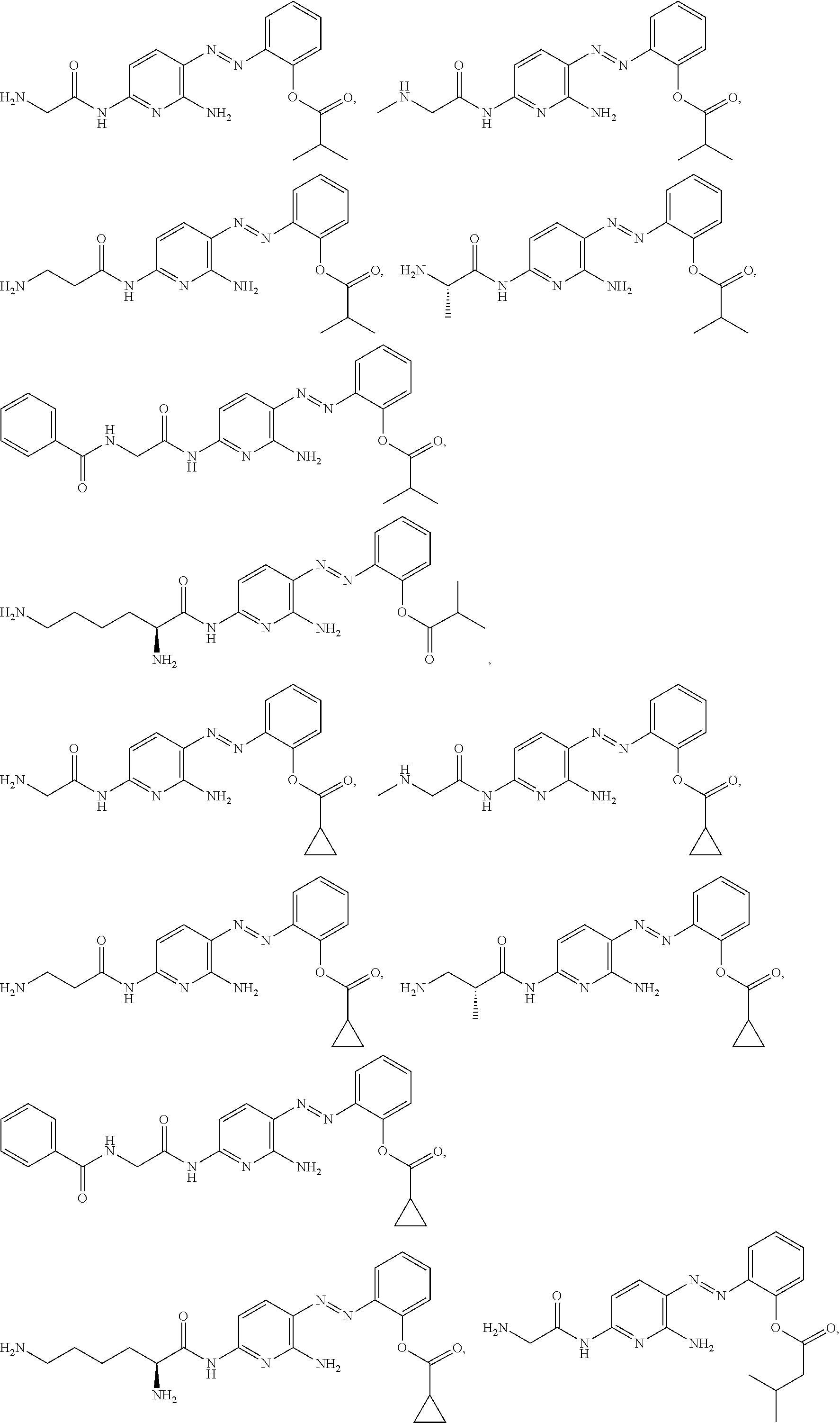 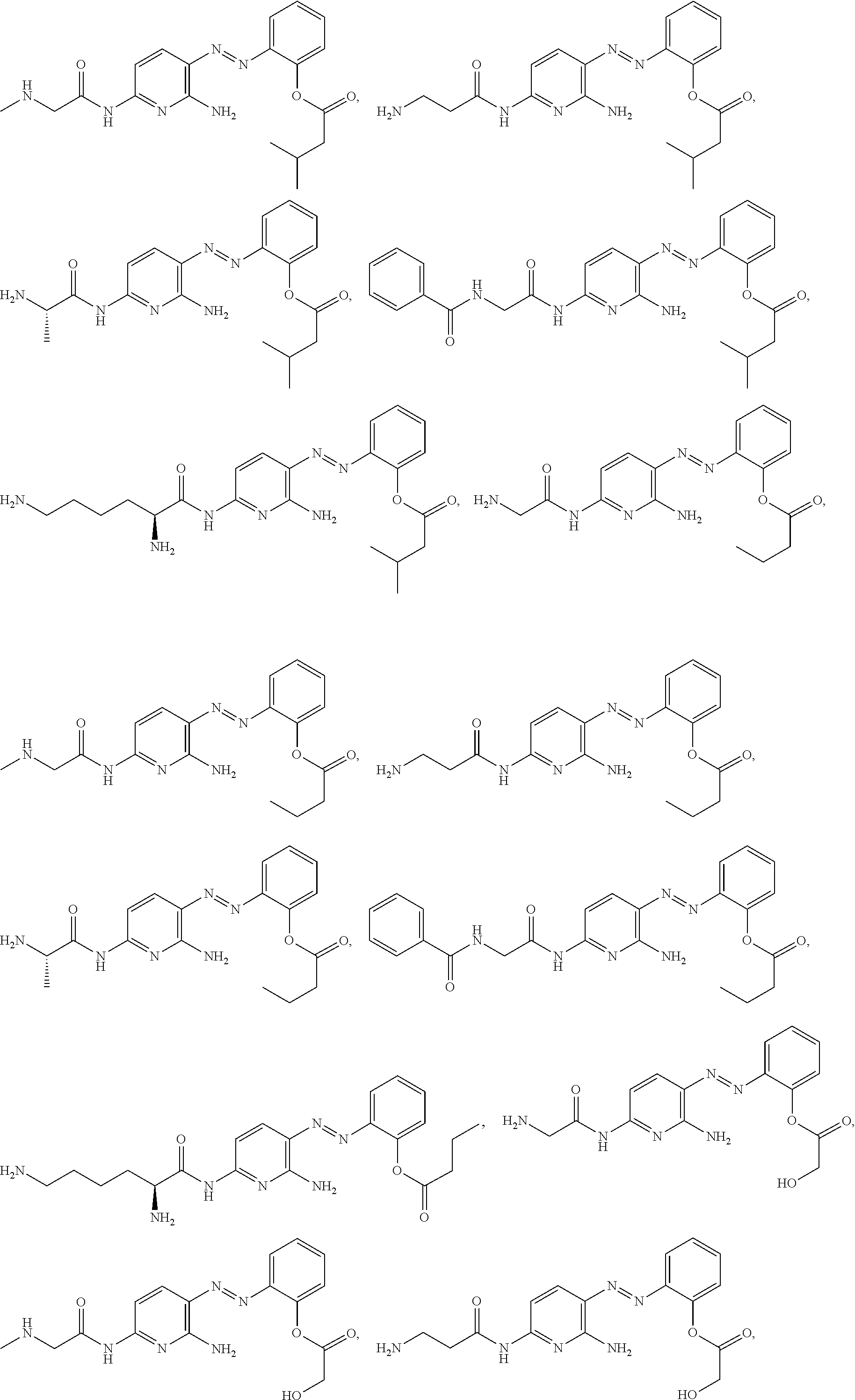 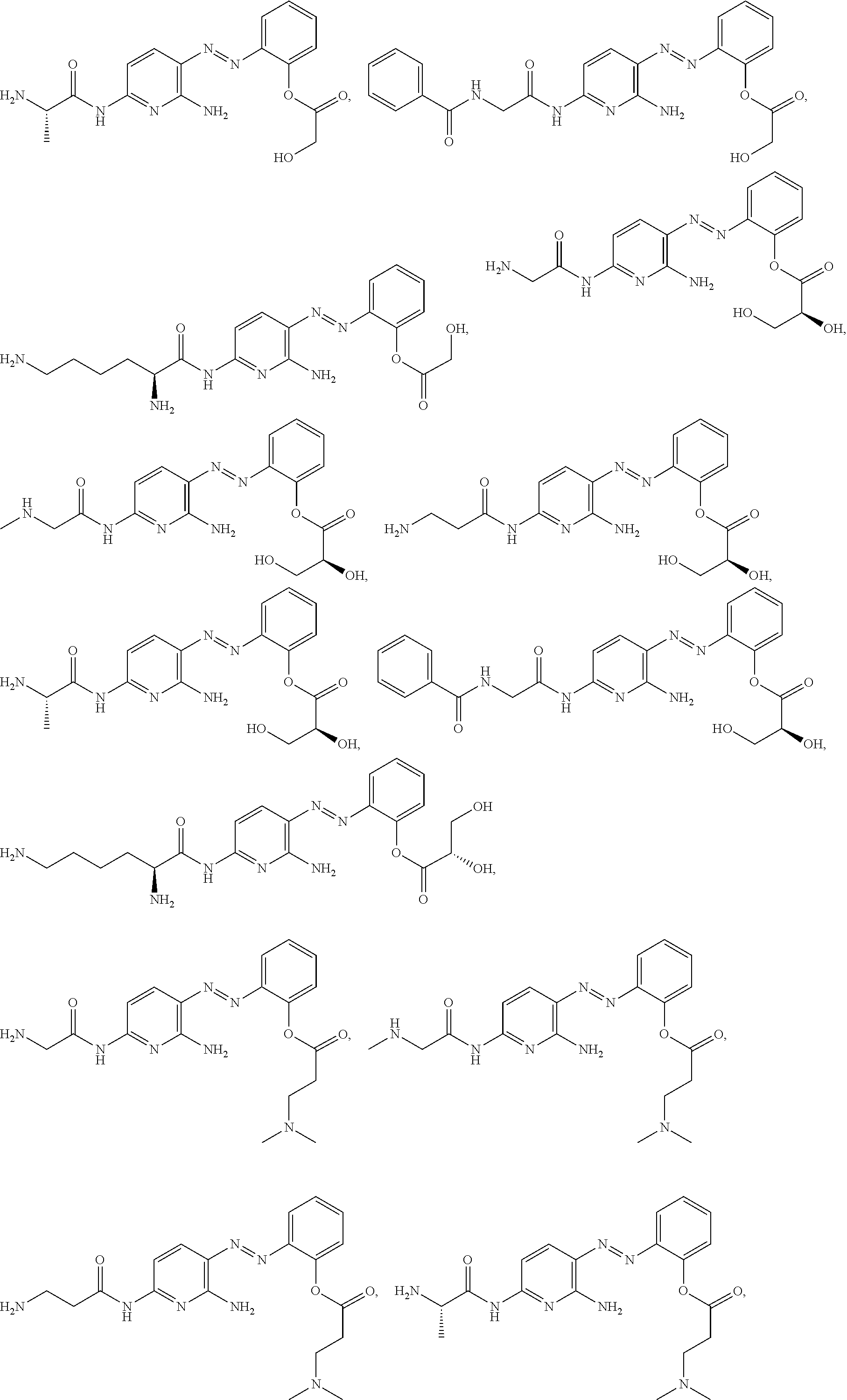 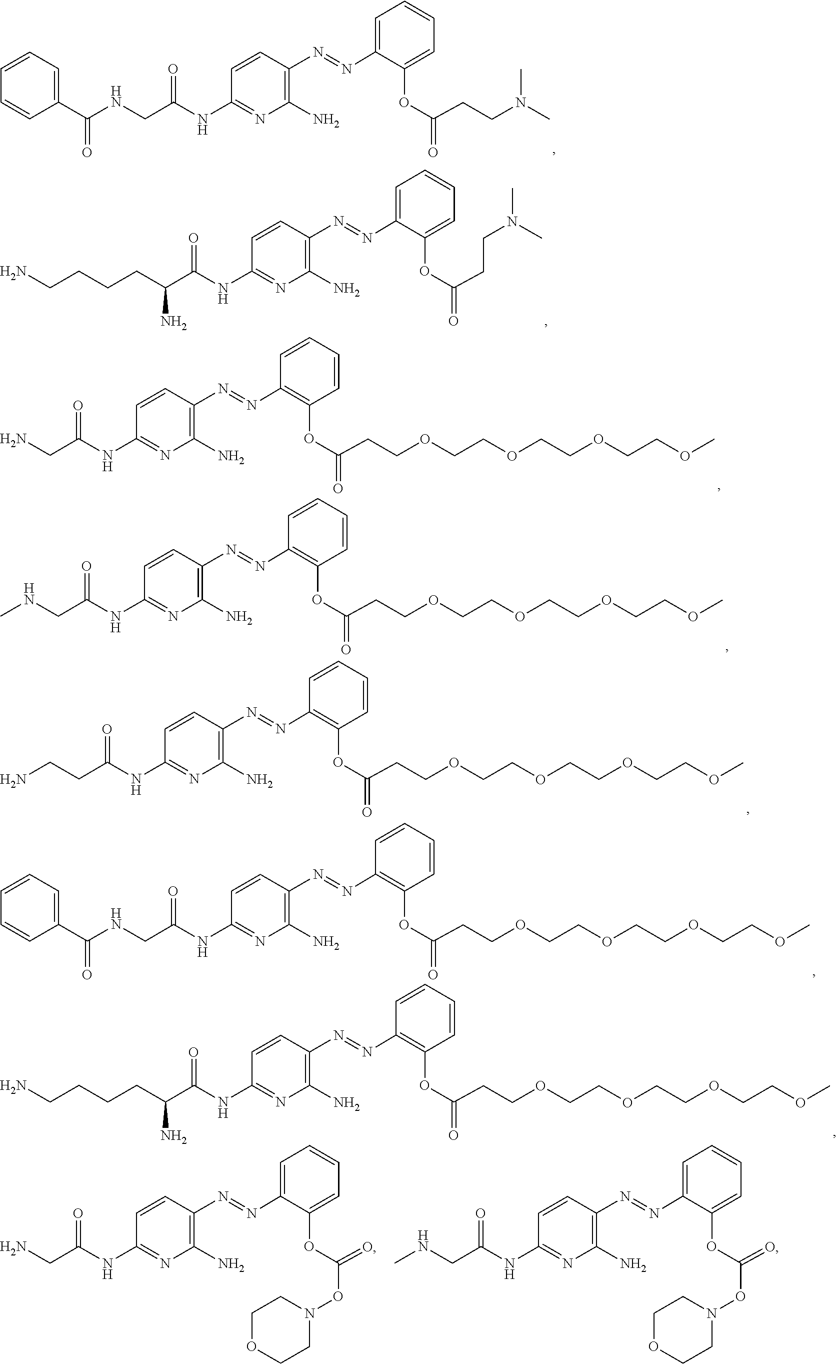 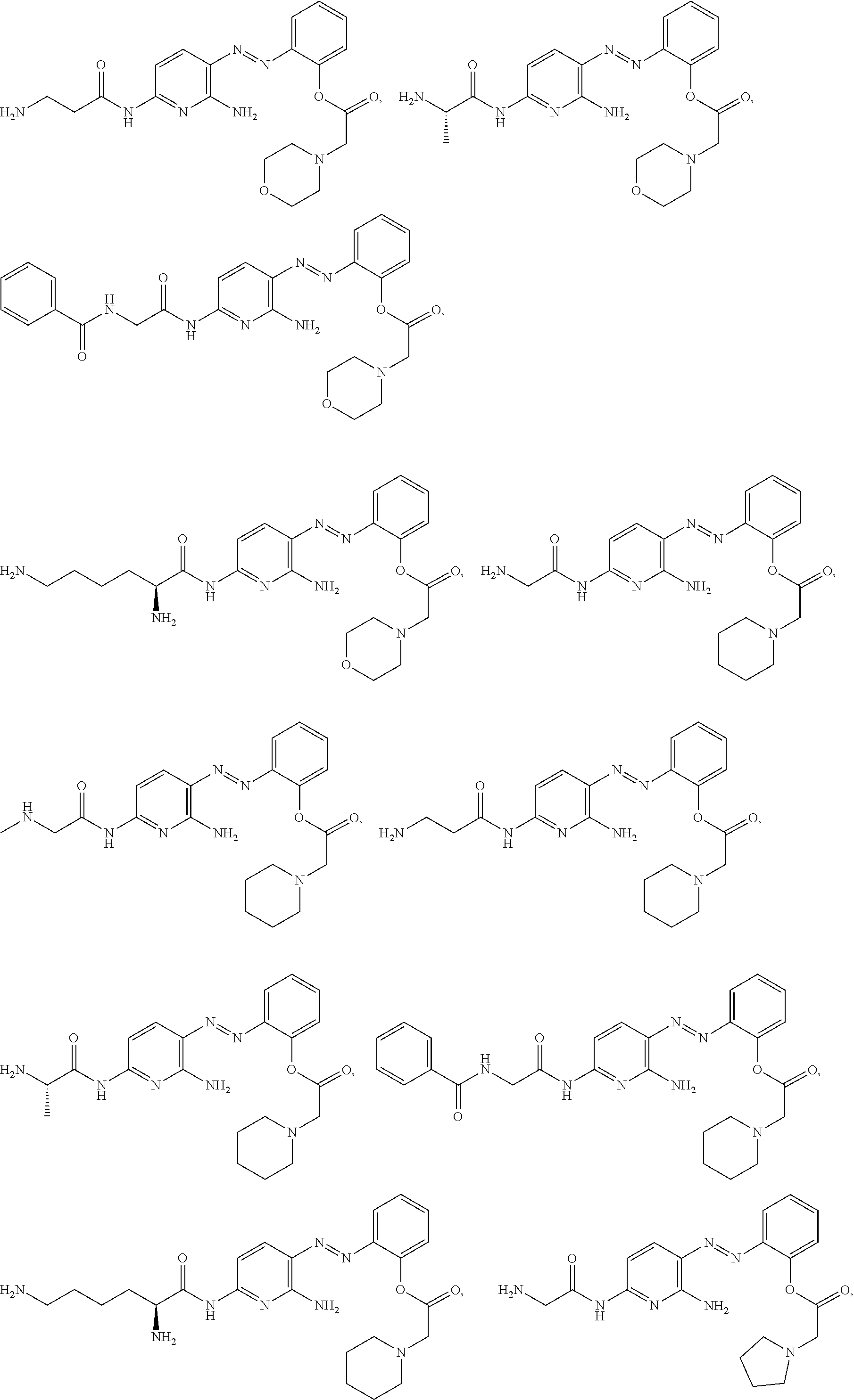 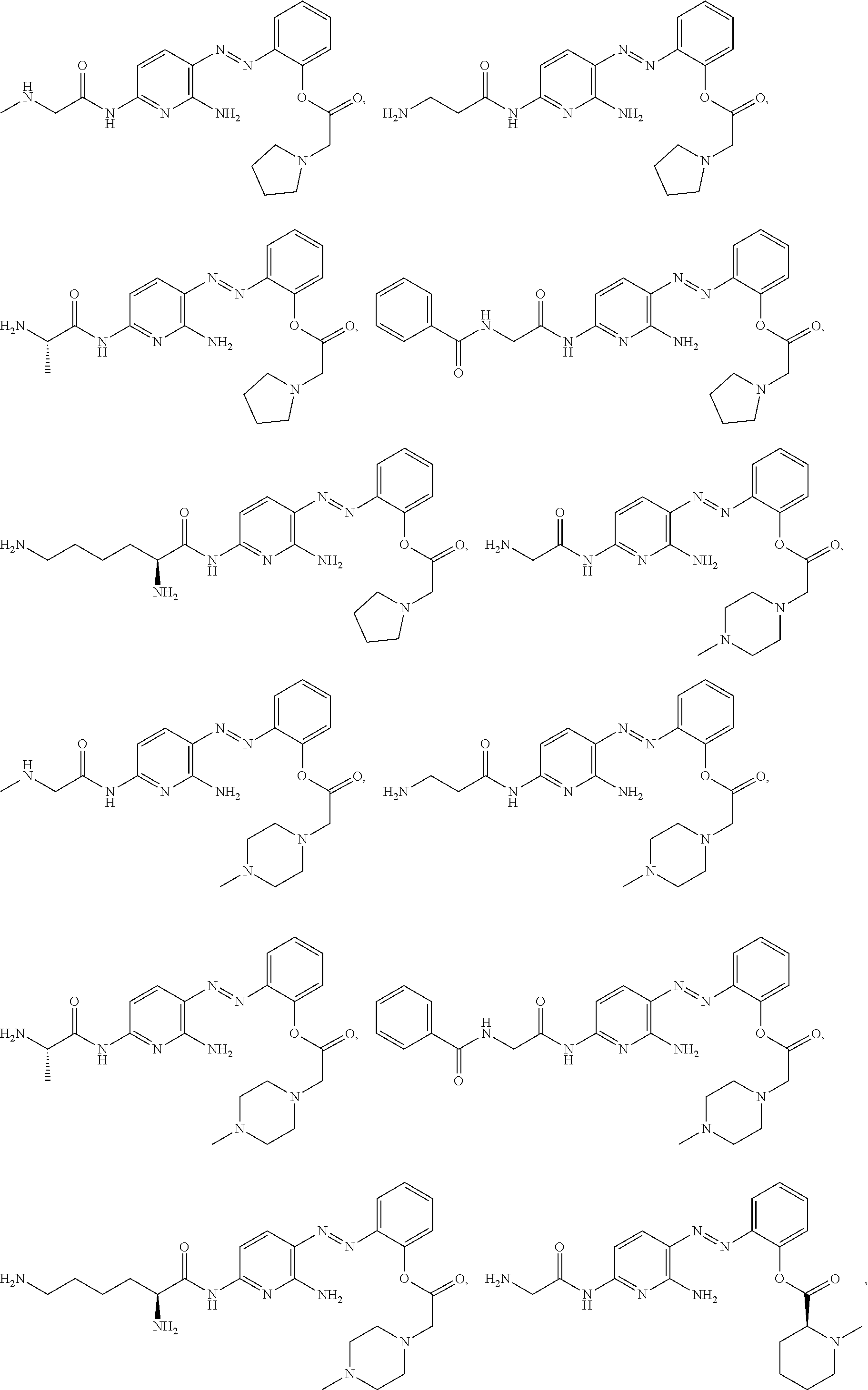 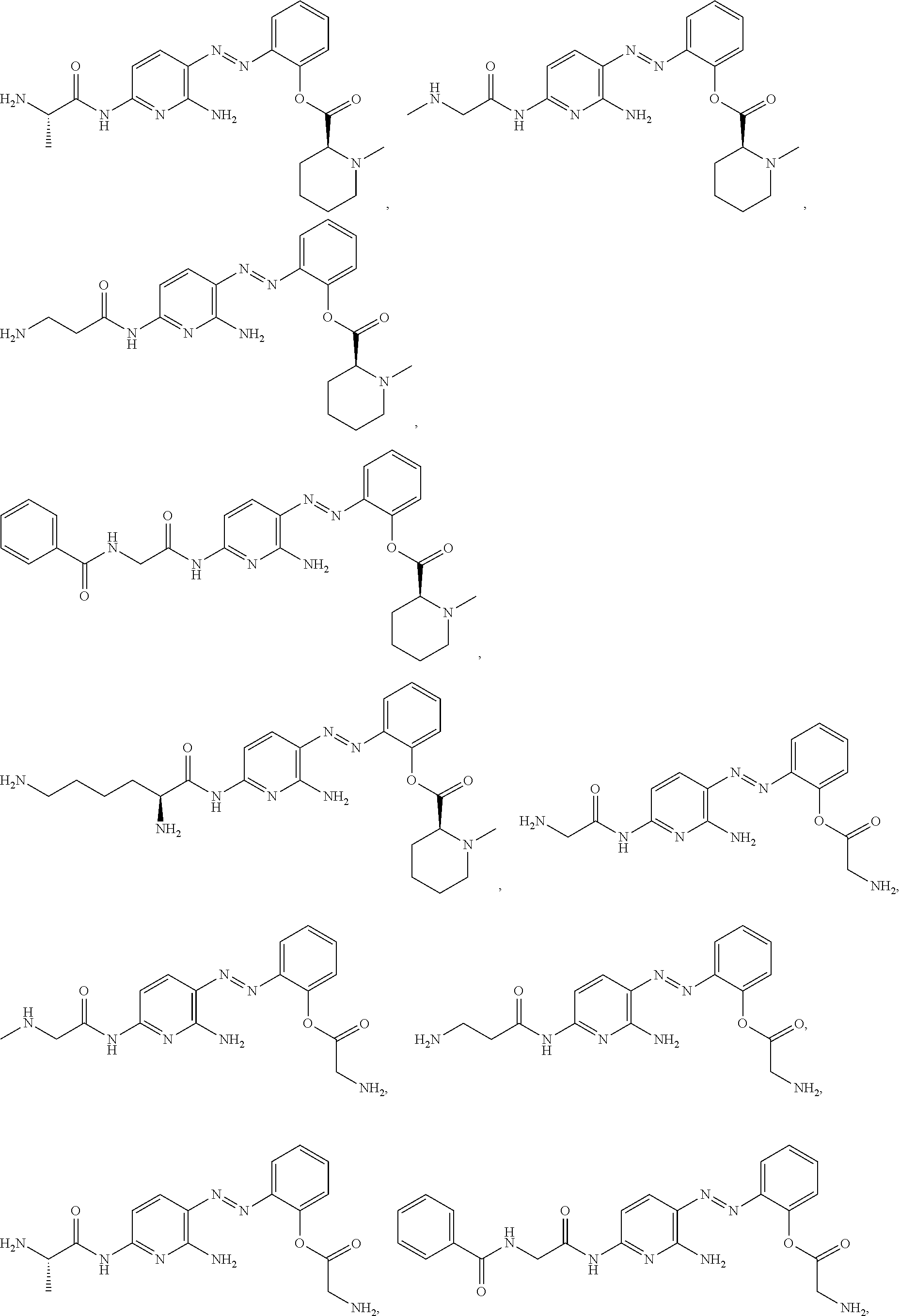 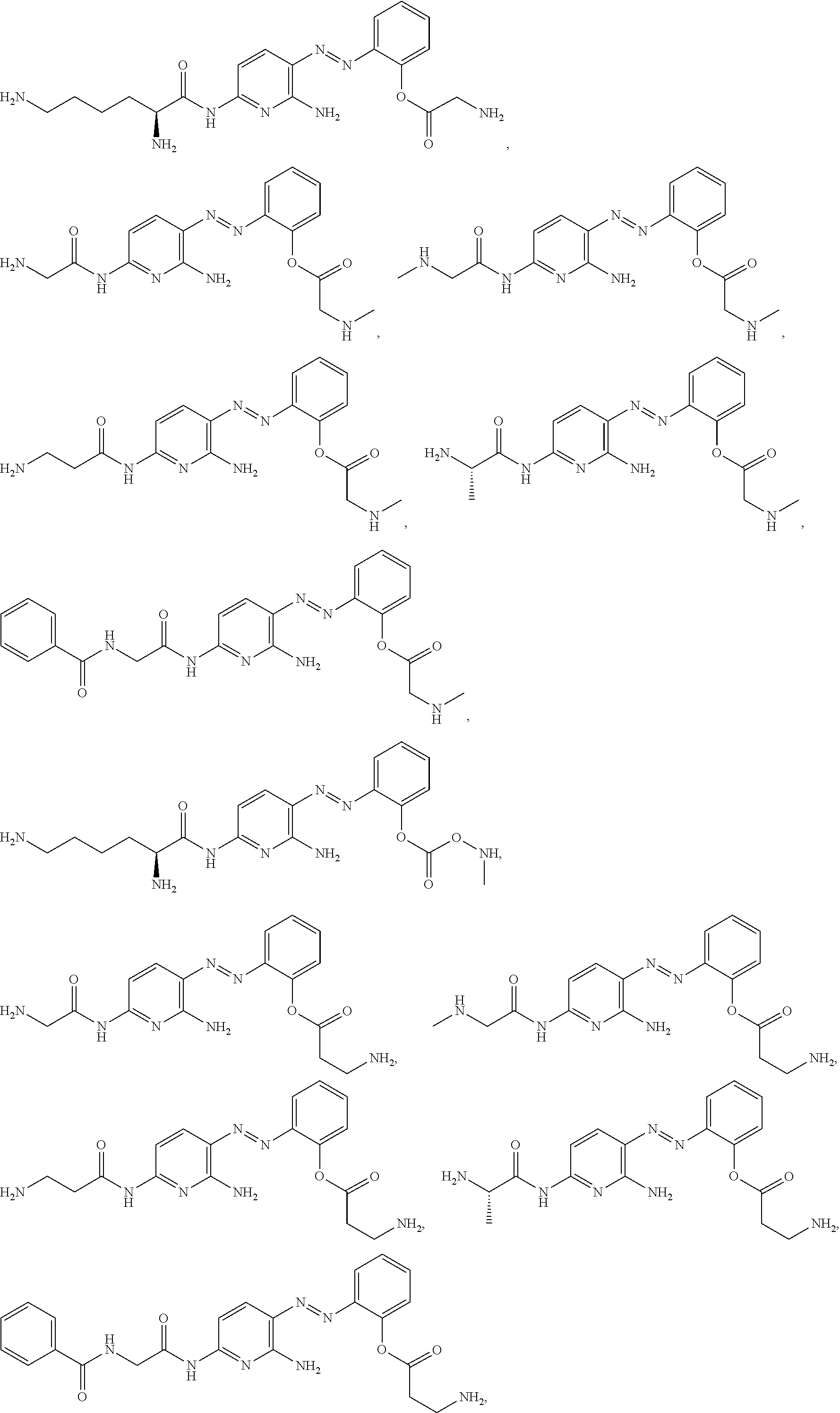 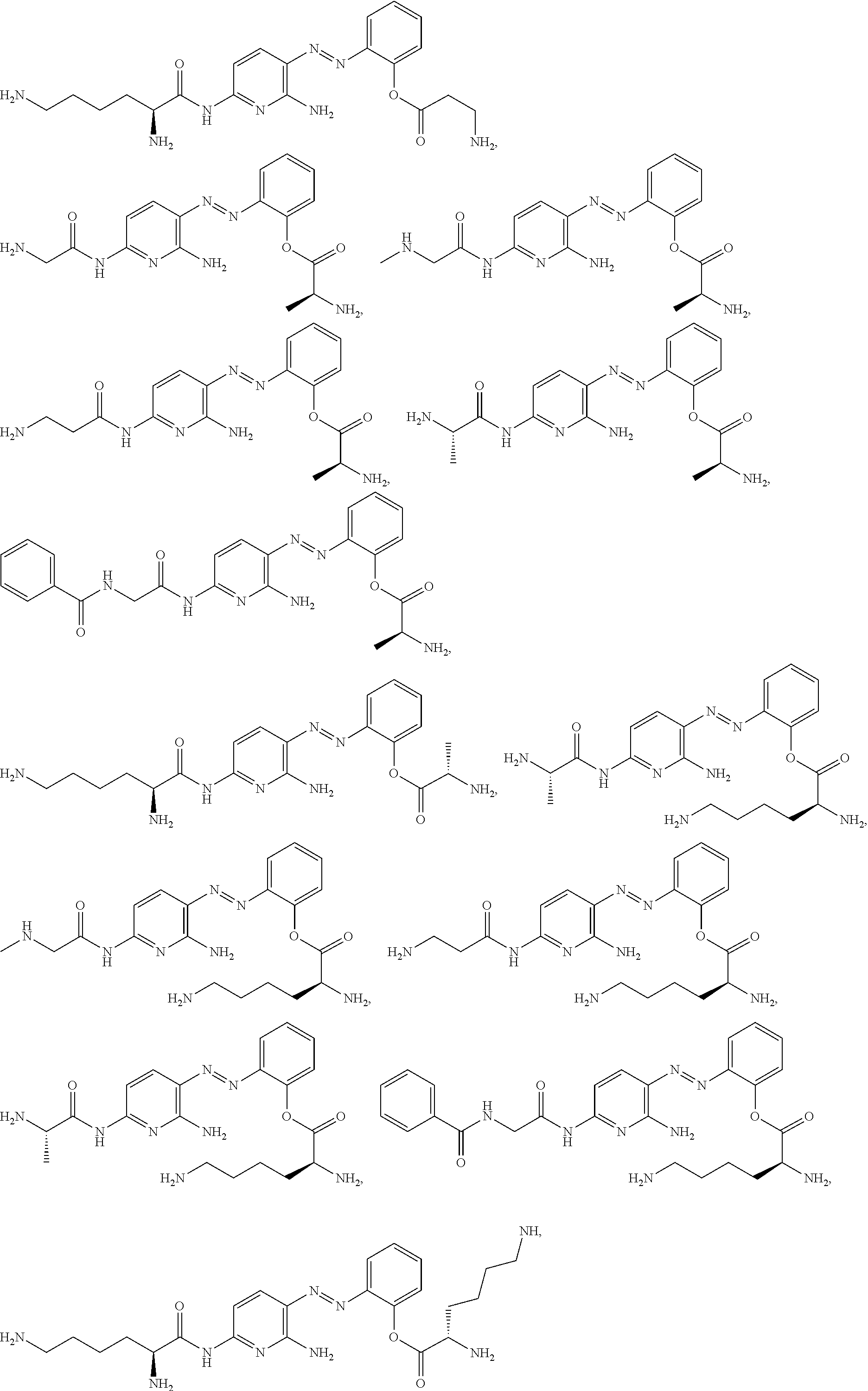 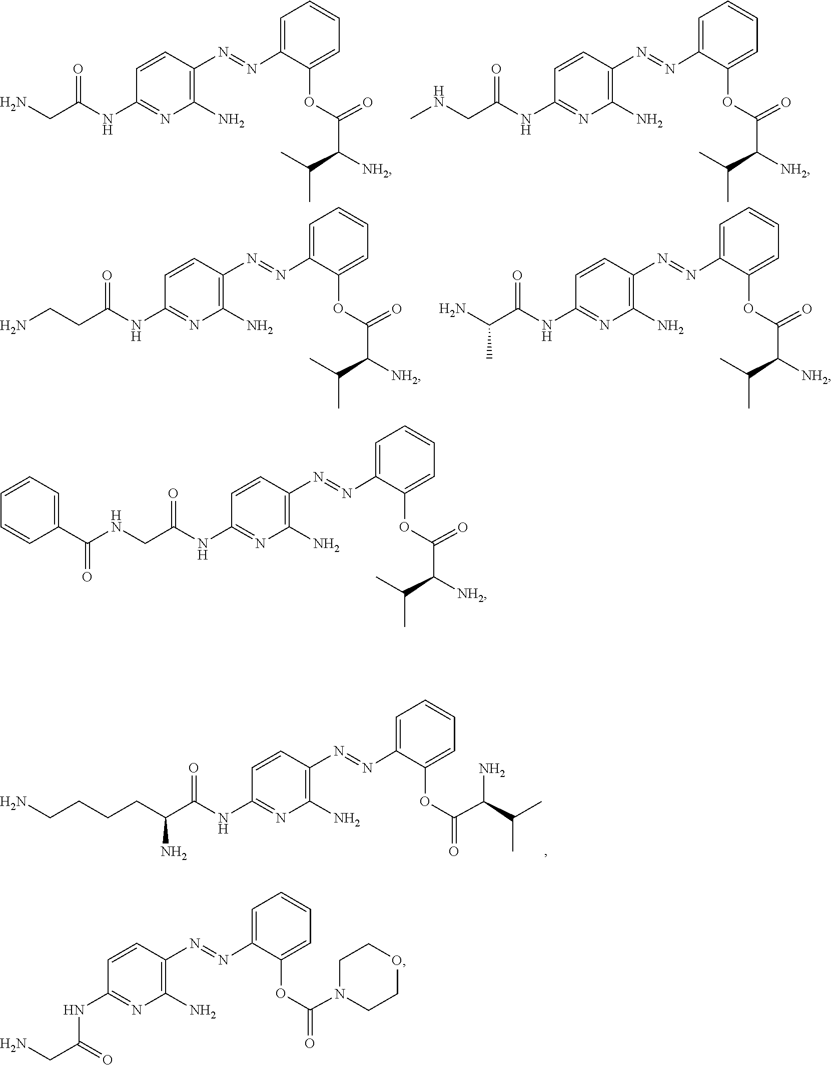
pharmaceutically acceptable salts or solvates thereof.

52. A compound of formula IX-D, pharmaceutically acceptable salts or solvates thereof according to claim 2: 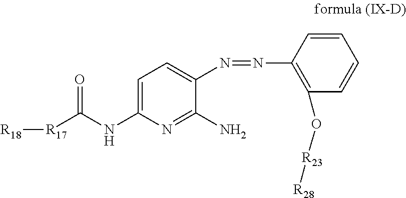
wherein R₁₇ is selected from optionally substituted linear or branched alkylene group, wherein alkylene is optionally substituted by amino, alkylamino and dialkylamino group; R₁₈ is selected from NH₂, —NR₂₀R₂₁, wherein R₂₀ and R₂₁ are each independently selected from hydrogen, optionally substituted alkyl, —C(O)—R₂₂, wherein R₂₂ is selected from optionally substituted alkyl, cycloalkyl, heterocycloalkyl, heteroaryl, and aryl group; R₂₃ is optionally substituted linear or branched alkylene; R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group.

53. The compound according to claim 52, selected from group consisting of: 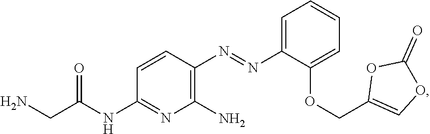 [SMILES: NCC(=O)Nc1ccc(/N=N/c2ccccc2OCc2coc(=O)o2)c(N)n1]
pharmaceutically acceptable salts or solvates thereof.

54. A compound of formula X, pharmaceutically acceptable salts or solvates thereof according to claim 2: 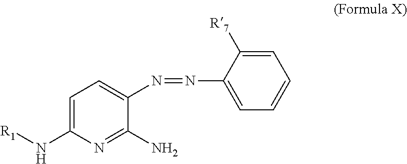
wherein;
R₁ is selected from 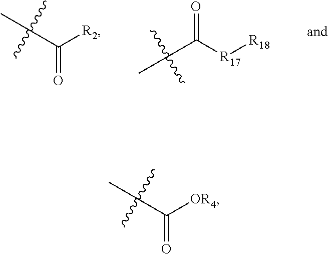
wherein R₂ is selected from the group consisting of optionally substituted alkyl, alkenyl, alkynyl, cycloalkyl, heterocycloalkyl, aryl, or heteroaryl; R₄ is selected from the group consisting of optionally substituted alkyl, alkenyl, alkynyl, —(CH2-CH2-O)n-, cycloalkyl, heterocycloalkyl, aryl, or heteroaryl; where n is from 1-20; Wherein R₁₇ is selected from optionally substituted linear or branched alkylene group, wherein alkylene is optionally substituted by amino, alkylamino and dialkylamino group; R₁₈ is selected from NH₂, —NR₂₀R₂₁ wherein R₂₀ and R₂₁ is each independently selected from hydrogen, optionally substituted alkyl and —C(O)—R₂₂ wherein R₂₂ is selected from optionally substituted alkyl, cycloalkyl, heterocycloalkyl, heteroaryl, and aryl group;
R′₇ is selected from —OH, —OR₁₂, 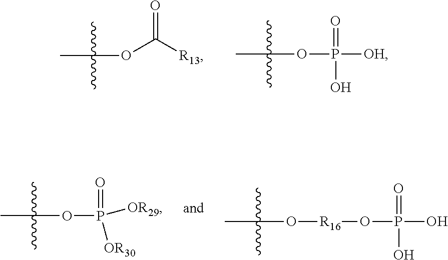
and R₁₉, wherein, R₁₂ is selected from the group consisting of optionally substituted alkyl, alkenyl, alkynyl, cycloalkyl, heterocycloalkyl, aryl, or heteroaryl; R₁₃ is selected from group consisting of optionally substituted alkyl, alkenyl, alkynyl, cycloalkyl, heterocycloalkyl, aryl, heteroaryl, alkyl-ester, amino, alkoxy, heterocycloalkoxy, heteroaryloxy, cycloalkoxy, aryloxy, aminoaryl, or amino acid linked via ester or amide linkage at the point of attachment, —(CH2-CH2-O)n-, or NR₁₄R₁₅; where n is from 1-20; wherein R₁₄ and R₁₅ each independently selected from hydrogen, optionally substituted alkyl, heterocycloalkyl, aryl group; R₁₆ is selected from optionally substituted alkylene(C₁₋₆) group; R₁₉ is selected from —O—R₂₃—O—C(O)—R₂₄, —O—C(O)—R₂₃—R₂₇, and —O—R₂₃—R₂₈, wherein R₂₃ is optionally substituted linear or branched alkylene;
R₂₄ is selected from optionally substituted alkoxy, cycloalkoxy, heterocycloalkoxy, heteroaryloxy, aryloxy group and —N(R₂₅R₂₆), wherein R₂₅, R₂₆ are each independently selected from hydrogen, optionally substituted alkyl and aryl, wherein alkyl and aryl are optionally substituted with OH, SH, F, Cl, Br, I, and optionally substituted hydroxyalkyl, amino group, or R₂₅ and R₂₆ is taken together to form an optionally substituted heterocycloalkyl ring, wherein the heterocycloalkyl ring is optionally substituted with alkyl, hydroxyalkyl, —OH, —SH, F, Cl, Br, I, and optionally substituted amino group;
R₂₇ is selected from optionally substituted alkyl, cycloalkyl, heterocycloalkyl, heteroaryl, aryl and —N(R₂₅R₂₆) group;
R₂₈ is selected from optionally substituted alkyl, cycloalkyl, heterocycloalkyl, heteroaryl, and aryl group; wherein heterocycloalkyl, heteroaryl in R₂₈ is optionally substituted with alkyl, hydroxyalkyl group, OH, SH, F, Cl, Br, I, and optionally substituted, amino and oxo group;
R₂₉ and R₃₀ are each independently selected from hydrogen, and —R₂₃—O—C(O)—O—R₃₁; wherein R₃₁ is selected from optionally substituted alkyl, cycloalkyl, heterocycloalkyl, aryl, and heteroaryl group.

55. The compound according to claim 54, selected from group consisting of: 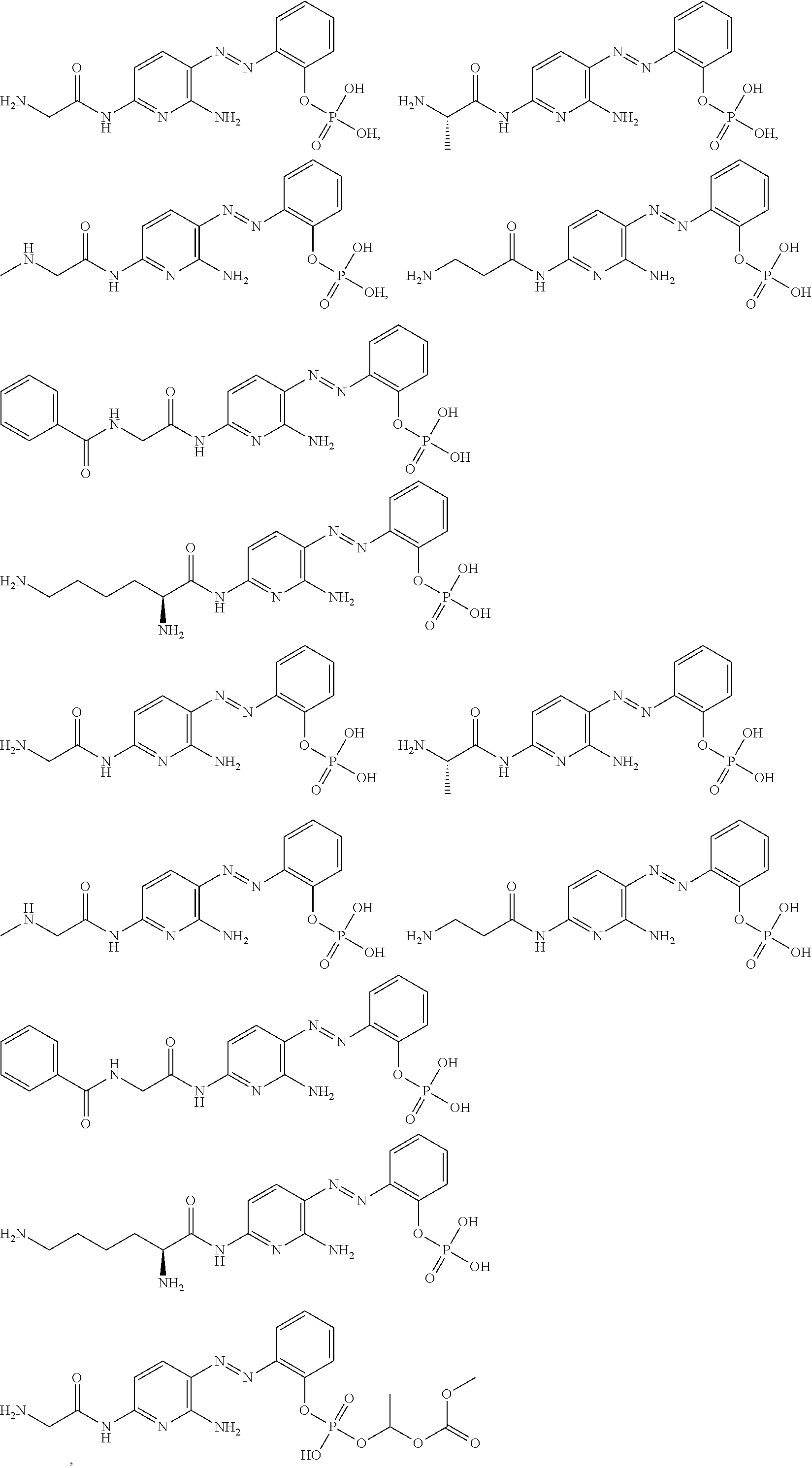 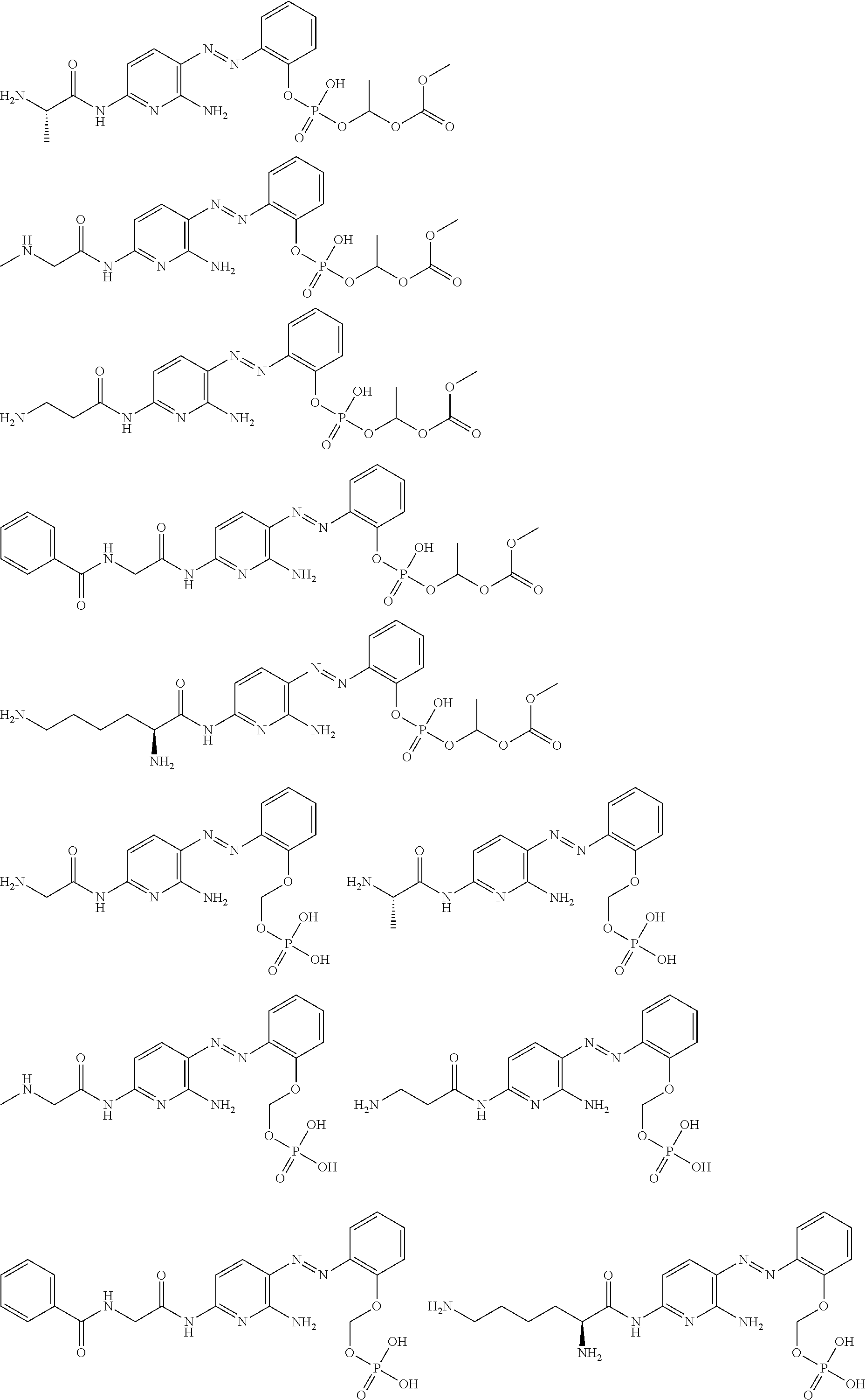 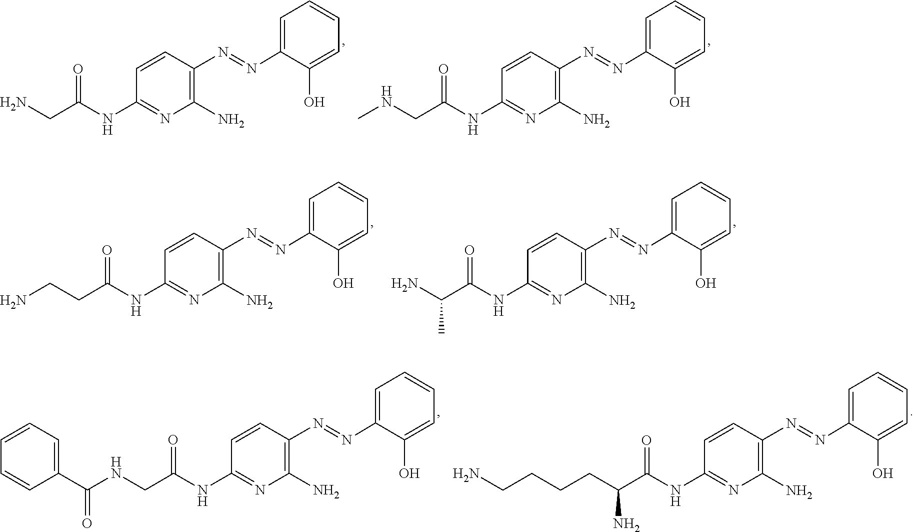 [SMILES: NCC(=O)Nc1ccc(/N=N/c2ccccc2O)c(N)n1 ; CNCC(=O)Nc1ccc(/N=N/c2ccccc2O)c(N)n1 ; NCCC(=O)Nc1ccc(/N=N/c2ccccc2O)c(N)n1 ; C[C@H](N)C(=O)Nc1ccc(/N=N/c2ccccc2O)c(N)n1 ; Nc1nc(NC(=O)CNC(=O)c2ccccc2)ccc1/N=N/c1ccccc1O ; NCCCC[C@H](N)C(=O)Nc1ccc(/N=N/c2ccccc2O)c(N)n1]
pharmaceutically acceptable salts or solvates thereof.

56. A pharmaceutical composition comprising a compound selected from group consisting of formula (I-A), formula (I), formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), formula (X) pharmaceutically acceptable salts or solvates thereof and a pharmaceutically acceptable excipient.

57. A method of treating condition selected from interstitial cystitis/bladder pain syndrome (IC/BPS), over active bladder (OAB), chronic cough, endometriosis, neuropathic pain, migraine, pruritus, postherpetic neuralgia, urinary tract infection related pain, osteoarthritis, arthritis and urinary incontinence, comprises administering a compound selected from compounds of general formula (I), formula (I-A), formula (II), formula (III), formula (III-A), formula (III-B), formula (III-C), formula (IV), formula (IV-A), formula (IV-B), formula (IV-C), formula (V), formula (V-A), formula (V-B), formula (V-C), formula (VI), formula (VI-A), formula (VI-B), formula (VI-C), formula (VII), formula (VII-A), formula (VII-B), formula (VII-C), formula (VIII), formula (VIII-A), formula (VIII-B), formula (VIII-C), formula (IX), formula (IX-A), formula (IX-B), formula (IX-C), formula (IX-D), formula (X) pharmaceutically acceptable salts or solvates thereof.
